(19) United States
(12) Patent Application Publication
(10) Pub. No.: US 2025/0073244 A1
(43) Pub. Date: Mar. 6, 2025

(54) CRYSTALLINE FORMS OF PENTAAZA MACROCYCLIC RING COMPLEX

(71) Applicant: Galera Labs, LLC, Malvern, PA (US)

(72) Inventors: Jeffrey A. SCHOLTEN, Chesterfield, MO (US); Stephan D. PARENT, West Lafayette, IN (US); Yike SI, Shanghai (CN); Tianjing ZHAO, Shanghai (CN); Xiaoyang WANG, Shanghai (CN); Travis L. HOUSTON, Lafayette, IN (US)

(21) Appl. No.: 18/598,269

(22) Filed: Mar. 7, 2024

Related U.S. Application Data
(60) Provisional application No. 63/451,111, filed on Mar. 9, 2023.

(51) Int. Cl. A61K 31/555; C07F 13/00

(52) U.S. Cl. CPC A61K 31/555; C07F 13/005; C07B 2200/13

(57) ABSTRACT

The present invention provides crystalline forms of a pentaaza macrocyclic ring complex according to the following formula:

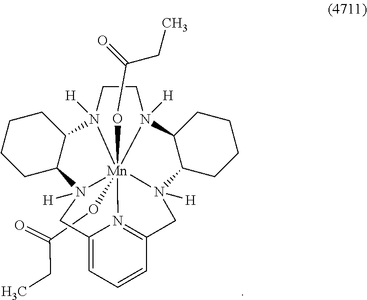

Also provided are pharmaceutical compositions that include the provided crystalline forms and methods of using the provided crystalline forms and pharmaceutical compositions for the treatment of disease states.

### CROSS-REFERENCE TO RELATED APPLICATIONS

The present application claims benefit of priority to U.S. Provisional Patent Application No. 63/451,111, filed Mar. 9, 2023, the entire content of which is hereby incorporated by reference herein in its entirety, as if recited in full herein.

### FIELD OF THE DISCLOSURE

The present disclosure relates to crystalline forms of a pentaaza macrocyclic ring complex, which are useful in the treatment of various cancers and inflammatory disorders such as oral mucositis, among other conditions.

### BACKGROUND OF THE DISCLOSURE

Transition metal-containing pentaaza macrocyclic ring complexes having the macrocyclic ring system corresponding to the formula below have been shown to be effective in a number of animal and cell models of human disease, as well as in treatment of conditions afflicting human patients.

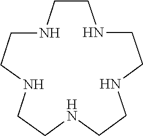 [SMILES: C1CNCCNCCNCCNCCN1]

For example, GC4711 is one of such compounds:

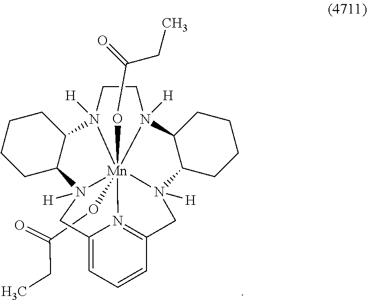

The mirror image of GC4711 is yet another one of these compounds, the chemical structure of which (GC4748) is shown below:

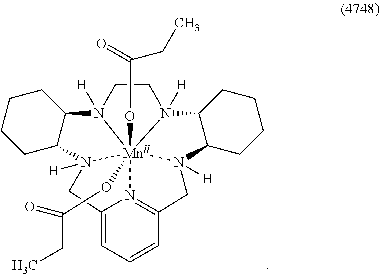

Pharmaceutical compositions are often formulated with a crystalline solid of the active pharmaceutical ingredient (API). The specific crystalline form of the API can have significant effects on properties such as stability and solubility/bioavailability. Instability and solubility characteristics can limit the ability to formulate a composition with an adequate shelf life or to effectively deliver a desired amount of a drug over a given time frame.

There exists an unmet need for crystalline forms of GC4711 (and GC4748), which exhibit improved properties for formulation of pharmaceutical compositions. The present disclosure is directed to meeting this and other needs.

### SUMMARY OF THE DISCLOSURE

The present disclosure provides polymorph screens that identified crystalline forms of GC4711 with good physical properties for therapeutics. It provides (1) preliminary characterization of amorphous and crystalline materials as-received, (2) further assessment of the polymorphic complexity of GC4711 through additional screening, (3) limited characterization of observed forms, and (4) an evaluation of the thermodynamic relationships between the forms through competitive slurry experiments at various water activities or exposure to different relative humidity.

Furthermore, while specific examples are provided herein for crystalline forms and characterizations of GC4711, it is expected that its mirror image form, GC4748, which differs only in its chirality from GC4711, but does not otherwise differ in terms of chemical structure, would exhibit the same or substantially similar polymorphs. For example, GC4748 would be expected to have crystalline forms with the same or substantially similar characteristics of the GC4711 crystalline forms (such as the same or similar characteristic XRPD peaks), and would be expected to be obtainable by the same or substantially similar process described herein for GC4711. Accordingly, where crystalline forms are described and characterized herein for GC4711, it is understood that the same and/or substantially similar crystalline forms exist for GC4748.

The chemical structure of GC4711 as provided in FIG. 1 has the propionato moieties located at the axial ligand positions of the chelating pentaazamacrocycle; through single crystal structure elucidation of several forms, it has been determined that solvents, such as water, may occupy the axial ligand position, forcing the propionate(s) to reside elsewhere within the crystal structure. Regardless of the bonding nature between the propionate moiety and the chelating molecule, the compound designation of GC4711 was retained for all forms identified within this study.

GC4711 readily forms hydrates and mixed solvate/hydrates. A previous study identified five crystalline phases, designated Patterns A through E. In the present disclosure, amorphous GC4711 and ten unique crystalline materials were observed:

| Description | Identifier | Comments |
|---|---|---|
| anhydrate | E | Tₘₑₗₜ ≈ 234° C. (w/ decomp), low hygroscopicity up to 45% RH and crystallizes to |
| | | Form A above, single crystal structure known |
| sesquihydrate | A | Most commonly observed, low hygroscopicity b/w 5 and 85% RH, |
| | | single crystal structure known |
| dihydrate | D | reproduced only by seeding, low hygroscopicity b/w 5 and 85% RH |
| hydrate | K | precipitated from highly concentrated aqueous solutions |
| — | B | Not observed within this study |
| purported | C | sesquihydrate hemiethanolate, single crystal structure known |
| mixed | F | from ACN, only observed as mixture w/ Form A |
| solvate/hydrates | G | from DCM/heptane |
| | H | from MEK/heptane |
| | I | from EtOH/heptane, only observed as mixture w/ Forms A and C |
| | J | from t-butanol/heptane and water, water is necessary to crystallize form |
| — | amorphous | T_{g} ≈ 42° C., spontaneously crystallizes to Form E above 65° C., significantly |
| | | hygroscopic, kinetically stable below 43% RH and crystallizes to Form A above |

Form E Anhydrate, the only anhydrous form identified, exhibits a concomitant melt/decomposition onset near 234° C. and low hygroscopicity from 5 to 45% RH. However, Form E is significantly hygroscopic above 45% RH, where it hydrates to Form A Sesquihydrate. Form E is formed through the crystallization of amorphous GC4711 or desolvation/dehydration of all solvated/hydrated forms at either elevated temperature or 0% RH.

Form A Sesquihydrate was the most commonly observed form. Form A exhibits low hygroscopicity from 5 to 85% RH and significant hygroscopicity above 85% RH. Although less stable than Pattern D at humidity higher than 23% RH, Form A is kinetically stable in the solid state at these conditions within the time-frame evaluated.

Form D Dihydrate could only be reproduced by seeding saturated 97:03 v/v EtOAc/water solutions. Form D exhibits low hygroscopicity from 5 to 85% RH and significant hygroscopicity above 85% RH. Form D is more stable than Form A above 23% RH.

Form C Sesquihydrate Hemiethanolate and Materials F through J are purported mixed solvate/hydrates. Several were isolated as mixtures with other forms. Most appear metastable at ambient conditions or under brief exposure to dry nitrogen. Regardless, all were shown to desolvate/dehydrate to Form E at elevated temperature.

Dehydration of both hydrated forms to Form E Anhydrate occurs at 0% RH. Form A Sesquihydrate was shown to be the prevailing hydrate, relative to Pattern D Dihydrate, at 11% RH, while Pattern D Dihydrate was the prevailing hydrate at 75% RH and above. Relatively slow kinetics of conversion in the solid state prevented reaching true equilibrium at 43% RH. However, the a_{w} experiments confirm that Pattern D Dihydrate is the prevailing hydrate at and above 0.23 a_{w} (equivalent to 23% RH). Pattern K, a higher hydrate precipitated from highly concentrated aqueous solutions, was not included in the relative hydrate stability assessment. Partial dehydration to a mixture of Form A Sesquihydrate and Pattern D Dihydrate occurred under brief exposure to dry nitrogen and suggests that Pattern K is less stable than either form at that condition. Tentatively, Pattern K is assumed to be prevalent only near a_{w}≈1 but remains unconfirmed. At room temperature, the stable RH regions for these forms can be summarized as follows:

| Water Activity (RT) | 0 < a_{w}^{E↔A} < 0.11 < a_{w}^{A↔D} < 0.23 a_{w}^{→K} = 1 | | | |
|---|---|---|---|---|
| Prevailing Form | E | A | D | K |

Accordingly, the present invention provides crystalline forms of GC4711 as characterized by any of the figures herein.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the chemical structure of GC4711.

FIG. 2 shows the XRPD patterns of GC4711 as received.

FIG. 3 shows the XRPD patterns of anhydrous and hydrate forms of GC4711.

FIG. 4 shows the XRPD patterns of purported solvate or mixed solvate/hydrate forms of GC4711.

FIG. 5 shows the atomic displacement ellipsoid diagram of Form E Anhydrate. Non-hydrogen atoms are represented by 50% probability anisotropic thermal ellipsoids. Asymmetric unit show on top and symmetry expanded molecule shown on bottom.

FIG. 6 shows the packing diagram viewed along the crystallographic a-axis of Form E Anhydrate.

FIG. 7 shows the packing diagram viewed along the crystallographic b-axis of Form E Anhydrate.

FIG. 8 shows the packing diagram viewed along the crystallographic c-axis of Form E Anhydrate.

FIG. 9 shows the hydrogen bonding of Form E Anhydrate.

FIG. 10 shows the calculated XRPD pattern for Form E Anhydrate.

FIG. 11 shows the calculated and experimental XRPD patterns for Form E Anhydrate.

FIG. 12 shows the thermograms of Form E Anhydrate.

FIG. 13 shows the DVS isotherm of Form E Anhydrate.

FIG. 14 shows the atomic displacement ellipsoid diagram of Form A Sesquihydrate. Non-hydrogen atoms are represented by 50% probability anisotropic thermal ellipsoids.

FIG. 15 shows the calculated XRPD pattern for Form A Sesquihydrate.

FIG. 16 shows the calculated and experimental XRPD patterns for Form A Sesquihydrate.

FIG. 17 shows the thermograms of Form A Sesquihydrate.

FIG. 18 shows the dynamic vapor sorption isotherm of Form A Sesquihydrate.

FIG. 19 shows the XRPD pattern of Pattern D Dihydrate as received, lot PS05524-12-G-DRY.

FIG. 20 shows the tentative indexing results of Pattern D Dihydrate.

FIG. 21 shows the thermograms of Pattern D Dihydrate as received, lot PS05524-12-G-DRY.

FIG. 22 shows the hot stage microscopy of Pattern D Dihydrate as received, lot PS05524-12-G-DRY.

FIG. 23 shows the DVS isotherm of Pattern D Dihydrate as received, lot PS05524-12-G-DRY.

FIG. 24 shows the tentative indexing results of Pattern K.

FIG. 25 shows the atomic displacement ellipsoid diagram of Form C Sesquihydrate Hemiethanolate. Non-hydrogen atoms are represented by 50% probability anisotropic thermal ellipsoids.

FIG. 26 shows the calculated XRPD pattern of Form C Sesquihydrate Hemiethanolate.

FIG. 27 shows the calculated and experimental XRPD patterns for Form C Sesquihydrate Hemiethanolate.

FIG. 28 shows the thermograms of Form C Sesquihydrate Hemiethanolate.

FIG. 29 shows the XRPD pattern Pattern F (as a mixture with Form A).

FIG. 30 shows the DSC thermogram of Pattern F (as a mixture with Form A). Conversion of Pattern F to Form A occurs quickly at ambient conditions and may have occurred prior to data acquisition. Therefore, thermal data may not be representative of the form.

FIG. 31 shows the tentative indexing results of Pattern G.

FIG. 32 shows the thermograms of Pattern G.

FIG. 33 shows the tentative indexing results of Pattern H.

FIG. 34 shows the thermograms of Pattern H.

FIG. 35 shows the XRPD pattern of Pattern I (as a mixture with Forms A and C). Shown from 2.5° to 18° 2θ for clarity. Peaks associated with Pattern I are denoted by *.

FIG. 36 shows the tentative indexing results of Pattern J.

FIG. 37 shows the thermograms of Pattern J.

FIG. 38 shows the XRPD pattern of amorphous GC4711 as received, lot JR-C17092208-G19001.

FIG. 39 shows the thermograms of amorphous GC4711 as received, lot JR-C17092208-G19001.

FIG. 40 shows the hot stage microscopy of amorphous GC4711 as received, lot JR-C17092208-G19001.

FIG. 41 shows the DVS isotherm of amorphous GC4711 as received, lot JR-C17092208-G19001.

FIG. 42 shows Form E attempts through desolvation of Form A Sesquihydrate.

FIG. 43 shows Form E generated at ˜200-mg scale compared to calculated powder pattern.

FIG. 44 shows Form E generated at ˜1-gram scale compared to reference pattern.

FIG. 45 shows regional view highlighting the additional peaks not attributed to Form E.

FIG. 46 shows the thermograms of Form E plus peaks, sample 8429-40-01.

FIG. 47 shows the thermograms of Form E plus peaks, sample 8429-73-01.

FIG. 48 shows the raw data of Example 2.

FIG. 49 shows the XRPD pattern of GC4711, batch: PS04106-15-G-WET.

FIG. 50 shows the PLM photograph of GC4711, batch: PS04106-15-G-WET.

FIG. 51 shows the TGA thermogram of GC4711, batch: PS04106-15-G-WET.

FIG. 52 shows the DSC thermogram of GC4711, batch: PS04106-15-G-WET.

FIG. 53 shows the HPLC overlay of GC4711, batch: PS04106-15-G-WET.

FIG. 54 shows the XRPD overlay of Pattern A from equilibration at 25° C.

FIG. 55 shows the XRPD overlay of Pattern A from anti-solvent experiments.

FIG. 56 shows the XRPD overlay of samples from fast cooling experiments.

FIG. 57 shows the XRPD overlay of Pattern B from slow cooling experiments.

FIG. 58 shows the XRPD overlay of samples from slow evaporation experiments.

FIG. 59 shows the XRPD overlay of Pattern A from slow evaporation experiments (protect with N₂).

FIG. 60 shows the XRPD overlay of Pattern B from slow evaporation experiments (protect with N₂).

FIG. 61 shows the XRPD overlay of samples from slow evaporation experiment_MeOH (protect with N₂).

FIG. 62 shows the XRPD overlay of Pattern C from slow evaporation experiments (protect with N₂).

FIG. 63 shows the DSC thermogram of Pattern A from anti-solvent experiments (FR00623-01-190912-02-AS-Acetone-Heptane).

FIG. 64 shows the DSC thermogram of Pattern B from slow cooling-EtOAc (FR00623-01-190923-01).

FIG. 65 shows the DSC thermogram of Pattern B from slow cooling-MEK (FR00623-01-190923-02).

FIG. 66 shows the DSC thermogram of Pattern B from slow evaporation-EtOAc (FR00623-01-190924-04).

FIG. 67 shows the DSC thermogram of Pattern B from slow evaporation-IPAC (FR00623-01-190924-05).

FIG. 68 shows the DSC thermogram of Pattern C from slow evaporation-IPA (FR00623-01-190924-03).

FIG. 69 shows the DSC cycle 1 of GC4711, batch: PS04106-15-G-WET.

FIG. 70 shows the DSC cycle 1 of GC4711, batch: PS04106-15-G-WET_enlarged.

FIG. 71 shows the DSC cycle 2 of GC4711, batch: PS04106-15-G-WET.

FIG. 72 shows the DSC cycle 2 of GC4711, batch: PS04106-15-G-WET_enlarged.

FIG. 73 shows the XRPD overlay of Pattern A before and after TGA experiments.

FIG. 74 shows the XRPD overlay of Pattern B before and after TGA experiments.

FIG. 75 shows the XRPD overlay of Pattern C before and after TGA experiments.

FIG. 76 shows the DSC thermogram of Pattern A after TGA and stored at ambient for 7 days.

FIG. 77 shows the DSC thermogram of Pattern B after TGA and stored at ambient for 7 days.

FIG. 78 shows the DSC thermogram of Pattern C after TGA and stored at ambient for 7 days.

FIG. 79 shows the XRPD overlay of water activity.

FIG. 80 shows the DSC thermogram of Pattern D from water activity.

FIG. 81 shows the TGA thermogram of Pattern D from water activity.

FIG. 82 shows the DSC thermogram of Pattern A_scale up by anti-solvent_EtOAc-Heptane.

FIG. 83 shows the TGA thermogram of Pattern A_scale up by anti-solvent_EtOAc-Heptane.

FIG. 84 shows the XRPD overlay of Scale up Pattern A.

FIG. 85 shows the XRPD overlay of Scale up Pattern D.

FIG. 86 shows the DVS isotherm plot of Pattern A at 25° C., batch FR00623-01-191014-05.

FIG. 87 shows the DVS isotherm plot of Pattern D at 25° C., batch FR00623-01-191014-05.

FIG. 88 shows the XRPD overlay of Pattern A before and after DVS.

FIG. 89 shows the XRPD overlay of Pattern D before and after DVS.

FIG. 90 the raw data of Example 5.

### DEFINITIONS

The term “solid form” is often used to refer to a class or type of solid-state material. One kind of solid form is a “polymorph” which refers to two or more compounds having the same chemical formula but differing in solid-state structure. Salts may be polymorphic. When polymorphs are elements, they are termed allotropes. Carbon possesses the well-known allotropes of graphite, diamond, and buckminsterfullerene. Polymorphs of molecular compounds, such as active pharmaceutical ingredients (“APIs”), are often prepared and studied in order to identify compounds meeting scientific or commercial needs including, but not limited to, improved solubility, dissolution rate, hygroscopicity, and stability.

Other solid forms include solvates and hydrates of compounds including salts. A solvate is a compound wherein a solvent molecule is present in the crystal structure together with another compound, such as an API. When the solvent is water, the solvent is termed a hydrate. Solvates and hydrates may be stoichiometric or non-stoichiometric. A monohydrate is the term used when there is one water molecule, stoichiometrically, with respect to, for example, an API, in the unit cell.

In order to identify the presence of a particular solid form, one of ordinary skill typically uses a suitable analytical technique to collect data on the form for analysis. For example, chemical identity of solid forms can often be determined with solution-state techniques such as ¹³C-NMR or 1H-NMR spectroscopy and such techniques may also be valuable in determining the stoichiometry and presence of “guests” such as water or solvent in a hydrate or solvate, respectively. These spectroscopic techniques may also be used to distinguish, for example, solid forms without water or solvent in the unit cell (often referred to as “anhydrates”), from hydrates or solvates.

Solution-state analytical techniques do not provide information about the solid state as a substance and thus, for example, solid-state techniques may be used to distinguish among solid forms such as anhydrates. Examples of solid-state techniques which may be used to analyze and characterize solid forms, including anhydrates and hydrates, include single crystal X-ray diffraction, X-ray powder diffraction (“XRPD”), solid-state ¹³C-NMR, Infrared (“IR”) spectroscopy, including Fourier Transform Infrared (FT-IR) spectroscopy, Raman spectroscopy, and thermal techniques such as Differential Scanning calorimetry (DSC), melting point, and hot stage microscopy.

Polymorphs are a subset of crystalline forms that share the same chemical structure but differ in how the molecules are packed in a solid. When attempting to distinguish crystalline forms based on analytical data, one looks for data which characterize the form. For example, when there are two crystalline forms of a compound (e.g., Form I and Form II), one can use X-ray powder diffraction peaks to characterize the forms when one finds a peak in a Form I pattern at angles where no such peak is present in the Form II pattern. In such a case, that single peak for Form I distinguishes it from Form II and may further act to characterize Form I. When more forms are present, then the same analysis is also done for the other crystalline form. Thus, to characterize Form I against the other crystalline form, one would look for peaks in Form I at angles where such peaks are not present in the X-ray powder diffraction patterns of the other crystalline form. The collection of peaks, or indeed a single peak, which distinguishes Form I from the other known crystalline form is a collection of peaks which may be used to characterize Form I. If, for example, two peaks characterize a crystalline form then those two peaks can be used to identify the presence of that crystalline form and hence characterize the crystalline form. Those of ordinary skill in the art will recognize that there are often multiple ways, including multiple ways using the same analytical technique, to characterize crystalline forms. For example, one may find that three X-ray powder diffraction peaks characterize a crystalline form. Additional peaks could also be used, but are not necessary, to characterize the crystalline form up to and including an entire diffraction pattern. Although all the peaks within an entire diffractogram may be used to characterize a crystalline form, one may instead, and typically does as disclosed herein, use a subset of that data to characterize such a crystalline form depending on the circumstances.

When analyzing data to distinguish an anhydrate from a hydrate, for example, one can rely on the fact that the two solid forms have different chemical structures—one having water in the unit cell and the other not. Thus, this feature alone may be used to distinguish the forms of the compound and it may not be necessary to identify peaks in the anhydrate, for example, which are not present in the hydrate or vice versa.

X-ray powder diffraction patterns are some of the most commonly used solid-state analytical techniques used to characterize solid forms. An X-ray powder diffraction pattern is an x-y graph with the diffraction angle, 2θ (°), on the x-axis and intensity on the y-axis. The peaks within this plot may be used to characterize a crystalline solid form. The data is often represented by the position of the peaks on the x-axis rather than the intensity of peaks on the y-axis because peak intensity can be particularly sensitive to sample orientation (see Pharmaceutical Analysis, Lee & Web, pp. 255-257 (2003)). Thus, intensity is not typically used by those skilled in the art to characterize solid forms.

As with any data measurement, there is variability in X-ray powder diffraction data. In addition to the variability in peak intensity, there is also variability in the position of peaks on the x-axis. This variability can, however, typically be accounted for when reporting the positions of peaks for purposes of characterization. Such variability in the position of peaks along the x-axis derives from several sources. One comes from sample preparation. Samples of the same crystalline material, prepared under different conditions may yield slightly different diffractograms. Factors such as particle size, moisture content, solvent content, and orientation may all affect how a sample diffracts X-rays. Another source of variability comes from instrument parameters. Different X-ray instruments operate using different parameters and these may lead to slightly different diffraction patterns from the same crystalline solid form. Likewise, different software packages process X-ray data differently and this also leads to variability. These and other sources of variability are known to those of ordinary skill in the pharmaceutical arts.

Due to such sources of variability, it is common to recite X-ray diffraction peaks using the word “about” prior to the peak value in degrees (2θ) (sometimes expressed herein as “2θ-reflections (°)”), which presents the data to within 0.1 or 0.2° (2θ) of the stated peak value depending on the circumstances. The X-ray powder diffraction data corresponding to the solid forms of the present invention were collected on instruments which were routinely calibrated and operated by skilled scientists. In the present invention, XRPD values are preferably obtained using Cu Kα X-ray radiation according to the method described in Example 1. Accordingly, the variability associated with these data would be expected to be closer to ±0.1 ° 2θ than to ±0.2 ° 2θ and indeed likely less than 0.1 with the instruments used herein. However, to take into account that instruments used elsewhere by those of ordinary skill in the art may not be so maintained, for example, all X-ray powder diffraction peaks cited herein have been reported with a variability on the order of ±0.2 ° 2θ and are intended to be reported with such a variability whenever disclosed herein and are reported in the specification to one significant figure after the decimal even though analytical output may suggest higher precision on its face.

Single-crystal X-ray diffraction provides three-dimensional structural information about the positions of atoms and bonds in a crystal. It is not always possible or feasible, however, to obtain such a structure from a crystal, due to, for example, insufficient crystal size or difficulty in preparing crystals of sufficient quality for single-crystal X-ray diffraction.

X-ray powder diffraction data may also be used, in some circumstances, to determine the crystallographic unit cell of the crystalline structure. The method by which this is done is called “indexing.” Indexing is the process of determining the size and shape of the crystallographic unit cell consistent with the peak positions in a suitable X-ray powder diffraction pattern. Indexing provides solutions for the three unit cell lengths (a, b, c), three unit cell angles (α, β, γ), and three Miller index labels (h, k, l) for each peak. The lengths are typically reported in Angstrom units and the angles in degree units. The Miller index labels are unitless integers. Successful indexing indicates that the sample is composed of one crystalline phase and is therefore not a mixture of crystalline phases.

IR spectroscopy, particularly FT-IR, is another technique that may be used to characterize solid forms together with or separately from X-ray powder diffraction. In an IR spectrum, absorbed light is plotted on the x-axis of a graph in the units of “wavenumber” (cm⁻¹), with intensity on the y-axis. Variation in the position of IR peaks also exists and may be due to sample conditions as well as data collection and processing. The typical variability in IR spectra reported herein is on the order of plus or minus 2.0 cm⁻¹. Thus, the use of the word “about” when referencing IR peaks is meant to include this variability and all IR peaks disclosed herein are intended to be reported with such variability.

Thermal methods are another typical technique to characterize solid forms. Different crystalline forms of the same compound often melt at different temperatures. Thus, the melting point of a crystalline form, as measured by methods such as capillary melting point, DSC, and hot stage microscopy, alone or in combination with techniques such as X-ray powder diffraction, IR spectroscopy, including FT-IR, or both, may be used to characterize crystalline forms or other solid forms.

As with any analytical technique, melting point determinations are also subject to variability. Common sources of variability, in addition to instrumental variability, are due to colligative properties such as the presence of other solid forms or other impurities within a sample whose melting point is being measured.

As used herein, “hygroscopicity” is defined as below:

| Term | Definition [13] |
|---|---|
| Low hygroscopicity | Material exhibits < 0.5 wt % water uptake over a specified RH range. |
| Limited hygroscopicity | Material exhibits < 2.0 wt % water uptake over a specified RH range. |
| Significant hygroscopicity | Material exhibits ≥ 2.0 wt % water uptake over a specified RH range. |
| Deliquescence | Spontaneous liquefaction associated with water sorption at a specified RH |
| | condition. |
| Stoichiometric hydrate | Crystalline material with a defined water content over an extended RH range. |
| | Typical stoichiometric hydrates are hemihydrates, monohydrates, |
| | sesquihydrates, dihydrates, etc. |
| Variable hydrate | Crystalline material with variable water content over an extended RH range, |
| | yet with no phase change. |

As used herein, “solubility” is defined as below:

| Term | Definition | |
|---|---|---|
| Low solubility | <1 | mg/mL |
| Limited solubility | 1-20 | mg/mL |
| Intermediate solubility | 20-100 | mg/mL |
| Good solubility | 100-200 | mg/mL |
| High solubility | >200 | mg/mL |

The following examples are provided to further illustrate the compounds, compositions and methods of the present invention. These examples are illustrative only and are not intended to limit the scope of the invention in any way.

### EXAMPLES

### Example 1

### Experimental Settings

### Fast Evaporation

Solutions were prepared in various solvents and, typically, filtered through a 0.2-μm nylon or PTFE filter. Each solution was allowed to evaporate from an open vial at ambient conditions, unless otherwise stated. Solutions were allowed to evaporate to dryness unless designated as partial evaporations (solid present with a small amount of solvent remaining), in which case solids were isolated as described herein.

### Relative Humidity Stressing

Select materials were transferred to a vial, which was then uncapped and placed inside a jar containing a saturated aqueous salt solution. Following jars were utilized: 11% RH lithium chloride, 43% RH potassium carbonate, 75% RH sodium chloride, and 85% potassium chloride [1]. Relative humidity stressing experiments were conducted at stated temperatures.

### Slow Cool

Concentrated solutions were prepared in various solvents at an elevated temperature and, typically, filtered warm through a 0.2-μm nylon or PTFE filter into a warm vial. Each solution was capped and left on the hot plate, and the hot plate was turned off to allow the sample to slowly cool to ambient temperature. If no solids were present after cooling to ambient temperature, the sample was further cooled at subambient temperatures. Any solids present after cooling were isolated as described herein.

### Slow Evaporation

Solutions were prepared in various solvents and, typically, filtered through a 0.2-μm nylon or PTFE filter. Each solution was allowed to evaporate from a covered vial (such as loosely capped or covered with perforated aluminum foil) at ambient conditions. Solutions were allowed to evaporate to dryness unless designated as partial evaporations (solids present with a small amount of solvent remaining), in which case solids were isolated as described herein.

### Slurry Experiments

Suspensions were prepared by adding enough solids to a given solvent at the stated conditions so that undissolved solids were present. The mixture was then agitated (typically by stirring or oscillation) in a sealed vial at a given temperature for an extended period of time. The solids were isolated as described herein.

### Solubility Determination

Aliquots of various solvents were added to measured amounts of a given material with agitation (typically sonication) at stated temperatures until complete dissolution was achieved, as judged by visual observation. If dissolution occurred after the addition of the first aliquot, values are reported as “>”. If dissolution did not occur, values are reported as “<”.

### Solvent/Antisolvent Addition

Solutions were prepared in various solvents and, typically, filtered through a 0.2-μm nylon or PTFE filter. Aliquots of various antisolvents were dispensed with stirring until precipitation occurred. Mixtures were allowed to stir for a specified amount of time. If necessary, samples were placed at subambient temperatures to facilitate precipitation. Solids were isolated as described herein.

### Vapor Diffusion

Concentrated solutions were prepared in various solvents and, typically, filtered through a 0.2-μm nylon or PTFE filter. The filtered solution was dispensed into a small vial, which was then placed inside a larger vial containing antisolvent. The small vial was left uncapped and the larger vial was capped to allow vapor diffusion to occur. Any solids present were isolated as described herein.

### XRPD Indexing

Indexing and structure refinement are computational studies. Within the figure referenced for a given indexed XRPD pattern, agreement between the allowed peak positions, marked with red bars, and the observed peaks indicates a consistent unit cell determination. Successful indexing of a pattern indicates that the sample is composed primarily of a single crystalline phase unless otherwise stated. Space groups consistent with the assigned extinction symbol, unit cell parameters, and derived quantities are tabulated below the figure. To confirm the tentative indexing solution, the molecular packing motifs within the crystallographic unit cells must be determined. No attempts at molecular packing were performed. The XRPD patterns were indexed using proprietary AMRI West Lafayette software [9].

### Differential Scanning Calorimetry (DSC)

DSC was performed using a Mettler-Toledo DSC3+ differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, the weight was accurately recorded, and the sample was inserted into the DSC cell. A weighed aluminum pan configured as the sample pan was placed on the reference side of the cell. The pan lid was pierced prior to sample analysis. The samples were analyzed from −25° C. to 250° C. at 10° C./min.

A cycling DSC experiment was conducted for as-received amorphous, in which the sample was analyzed from −25° C. to 200° C., then cooled to −25° C. and reheated to 250° C. at 10° C./min.

### Dynamic Vapor Sorption (DVS)

Dynamic vapor sorption data was collected on a Surface Measurement System DVS Intrinsic instrument. The sample was equilibrated at ˜43% RH prior to analysis. Sorption and desorption data were collected over a range the following ranges in 10% RH increments under a nitrogen purge: 45-95% RH, 95-5% RH, and 5-45% RH. The equilibrium criteria used for the analyses were 0.001 dm/dt weight change in 5 minutes with a minimum step time of 30 minutes and maximum equilibration time of 180 minutes with a 3 minute data logging interval. Data were not corrected for the initial moisture content of the sample.

### Hot Stage Microscopy

Hot stage microscopy was performed using a Linkam hot stage (FTIR 600) mounted on a Leica DM LP microscope equipped with a SPOT Insight™ color digital camera. Temperature calibrations were performed using USP melting point standards. Samples were placed on a cover glass, and a second cover glass was placed on top of the sample. As the stage was heated, each sample was visually observed using a 20× 0.40 N. A. long distance working objective with crossed polarizers and a first order red compensator. Images were captured using SPOT software (v. 4.5.9).

### Optical Microscopy

Samples were observed under a Motic or Wolfe optical microscope with crossed polarizers or under a Leica stereomicroscope with a first order red compensator with crossed polarizers.

### Proton NMR Spectroscopy

The solution NMR spectra were acquired with an Avance 600 MHz spectrometer. The samples were prepared by dissolving approximately 5-10 mg of sample in DMSO-de containing TMS. The data acquisition parameters are displayed in the first plot of the spectrum in the Data section of this disclosure.

### Thermogravimetry (TGA)

Thermogravimetric analyses were performed using a Mettler-Toledo TGA/DSC3+ analyzer. Temperature and enthalpy adjustments were performed using indium, tin, and zinc, and then verified with indium. The balance was verified with calcium oxalate. The sample was placed in an aluminum pan. The pan was hermetically sealed, the lid pierced, and the pan was then inserted into the TG furnace. A weighed aluminum pan configured as the sample pan was placed on the reference platform. The furnace was heated under nitrogen. Samples were analyzed from 25° C. to 350° C. at 10° C./min.

Thermogravimetric analyses typically experience a period of equilibration at the start of each analysis, indicated by red parentheses on the thermograms. The starting temperature for relevant weight loss calculations is selected at a point beyond this region (typically above 35° C.) for accuracy.

DSC analysis on this instrument is less sensitive than on the DSC3+ differential scanning calorimeter. Therefore, samples with sufficient solids were analyzed by both instruments and only the TGA thermogram from this instrument is reported.

### X-Ray Powder Diffraction (XRPD)

a. Transmission Geometry (Most Samples)

XRPD patterns were collected with a PANalytical X'Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e or 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 5.5. The data acquisition parameters for each pattern are displayed above the image in the Data section of this report. All images have the instrument labeled as X'Pert PRO MPD regardless of the instrument used (except for file 1001359).

b. Reflection Geometry (Samples in Limited Quantity)

XRPD patterns were collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e or 640f) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 5.5. The data acquisition parameters for each pattern are displayed above the image in the Data section of this report including the divergence slit (DS) and the incident-beam SS.

### Example 2

### Results and Discussion

### A. Received Materials

Lots PS05524-12-G-DRY and JR-C17092208-G19001 were received for preliminary characterization and use in screening activities (Table 1). Lot PS05524-12-G-DRY is composed of birefringent fines under polarized light microscopy and identified as Pattern D by X-ray powder diffraction (XRPD). Lot JR-C17092208-G19001 is composed of opaque and non-birefringent fines and is amorphous by XRPD.

TABLE 1
GC4711 As Received.

| Lot No. | Description | Storage | Quantity | LIMS |
|---|---|---|---|---|
| PS05524-12-G-DRY | Pattern D | freezer | 0.6 g | 542739 |
| JR-C17092208-G19001 | amorphous | freezer | 30 g | 542741 |

### B. Screen Experiments

The provided solubility of GC4711 was used to help guide the experimental conditions used within this study. Approximately 75 experiments were conducted exploring a variety of solvent systems, temperatures, crystallization conditions, and starting materials (summarized in Tables 2 through 14). Crystallization techniques employed include slurrying, evaporation, cooling, vapor diffusion, anti-solvent precipitation, vapor stressing, drying, and relative humidity stressing [1]. In some instances, solids were purposefully analyzed wet to further increase the likelihood of identifying hydrated or solvated forms. Water activity slurries were also utilized to evaluate the propensity of GC4711 to form hydrates [2]. Non-solvent methods consisting of heat-induced transformations were also attempted.

TABLE 2
Crystallization Experiments of Amorphous GC4711 Lot JR-C17092208-G19001.

| Solvent^{a} | Method^{b} | Observation^{c} | Result | Sample | LIMS | File | Page |
|---|---|---|---|---|---|---|---|
| acetone | 1. dissolved, precip | 1. plates and blades | E + A | 8235-06-01 | 542808 | 1000848 | 80 |
| | 2. isolated single xtal | 2. — | E | 8235-06-01 | 542808 | 1000787 | 32 |
| acetone/water | dissolved then precip | fines, B | A | 8235-48-14 | 546472 | 1007367 | 81 |
| 96:04 v/v | | | | | | | |
| 0.50 a_{w} | | | | | | | |
| acetone/water | dissolved then precip | fines, B | A | 8235-89-03 | 546473 | 1007368 | 82 |
| 97:03 v/v | | | | | | | |
| 0.44 a_{w} | | | | | | | |
| acetone/water | 1. dissolution | 1. >994 mg/mL | A | 8235-07-11 | 543769 | 1002688 | 83 |
| 50:50 v/v | 2. fast evaporation | 2. wet flakes, B | | | | | |
| ACN | 1. dissolved | 1. yellow | A + F | 8235-06-02 | 542891 | 1000844 | 84 |
| | 2. treated w/charcoal | 2. no changes | | | | | |
| | 3. fast evaporation | 3. blades, singles | | | | | |
| | slow evaporation | blades, singles | A + F | 8235-08-02 | 542892 | 1000845 | 85 |
| | 1. vapor diffusion; Et₂O | 1. clear | A | 8235-08-01 | 543585 | 1002282 | 86 |
| | 2. freezer, 6 d | 2. fines, B | | | | | |
| t-butanol | solvent/antisolvent | | J | 8235-48-06 | 544516 | 1004120 | 87 |
| | 1. 08:92 v/v heptane | 1. no changes | | | | | |
| | 2. drops of water | 2. no changes | | | | | |
| | 3. 02:98 v/v heptane | 3. wispy aciculars | | | | | |
| | 4. ambient, 3 d | 4. white | | | | | |
| | solvent/antisolvent | | J | 8235-48-10 | 544692 | 1004454 | 88 |
| | 1. 02:98 v/v water | 1. clear solution | | | | | |
| | 2. heptane seeded with | 2. seed retained | | | | | |
| | 8235-48-06 added | | | | | | |
| | 3. added to 95:05 v/v | 3. turbid, fine wispy | | | | | |
| | heptane/t-butanol | solids, no oil | | | | | |
| | 4. ambient, 1 d | 4. fine wispy acicular | | | | | |
| | 8235-48-10 filtrate | | J | 8235-66-02 | 545162 | 1005102 | 89 |
| | 1. refrigerated, 2 d | 1. wispy, sheets | | | | | |
| | 2. freezer, 4 d | 2. — | | | | | |
| | solvent/antisolvent | | J | 8235-56-01 | 544697 | 1004457 | 90 |
| | 1. 02:98 v/v heptane | 1. no changes | | | | | |
| | 2. ambient, 3 d | 2. irregular, B | | | | | |
| | 3. refrigerated, 1 d | 3. fines, B | | | | | |
| | 4. freezer, 1 d | 4. — | | | | | |
| | 8235-56-01 filtrate | fines, B | J | 8235-67-01 | 544905 | 1004729 | 91 |
| | freezer, 2 d | | | | | | |
| DCM | slow evaporation | thin lamellae, B | A, E + | 8235-09-01 | 542890 | 1000843 | 92 |
| | | | peaks | | | | |
| | solvent/antisolvent | | G | 8235-06-07 | 543179 | 1001450 | 93 |
| | 1. added to heptane | 1. few aciculars | | | | | |
| | 2. refrigerated, 4 d | 2. fine aciculars, B | | | | | |
| | 3. centrifuge | 3. — | | | | | |
| | 8233-06-07 supernatant | white fine aciculars, B | A + G | 8235-21-01 | 543178 | 1001449 | 94 |
| | with additional heptane | | | | | | |
| | solvent/antisolvent | | G | 8235-48-08 | 544690 | 1004452 | 95 |
| | 1. heptane seeded with | 1. seed retained | | | | | |
| | 8235-06-07 | | | | | | |
| | 2. DCM added, 1 d | 2. blades and aciculars | | | | | |
| | 3. filtered | 3. — | | | | | |
| | 8235-48-08 filtrate | filamentous, fine | A + D | 8235-66-01 | 544695 | 1004455 | 96 |
| | under reduced pressure | aciculars | | | | | |
| diethyl ether | contacted, filtered | partial dissolution, fines | A + E | 8235-06-06 | 542893 | 1000846 | 97 |
| 1,4-dioxane | contacted, centrifuged | fines, B | A + E | 8235-06-05 | 542899 | 1000853 | 98 |
| EtOH | fast evaporation | lamellae, B | C | 8235-07-04 | 543117 | 1001293 | 99 |
| | slow evaporation | rosettes of aciculars, B | C | 8235-11-02 | 543116 | 1001292 | 100 |
| | vapor diffusion, Et₂O | rosettes | A | 8235-31-02 | 543768 | 1002687 | 101 |
| | solvent/antisolvent | | A + C | 8235-07-12 | 544144 | 1012343 | 102 |
| | 1. added Et₂O | 1. oil | | | | | |
| | 2. stored in freezer | 2. yellow oil, fines, B | | | | | |
| | 3. fast evaporation | 3. fines, B | | | | | |
| EtOH/heptane | 1. 112 mg/0.5 mL | 1. readily dissolved | A, C, I | 8235-07-17 | 544143 | 1012342 | 103 |
| 50:50 v/v | 2. 7 mL beptane | 2. very faint turbidity | | | | | |
| | 3, ambient, 2 d | 3 oil in base | | | | | |
| | 4. refrigerated, 4 d | 4. yellow brown oil | | | | | |
| | 5. fast evaporation | 5. blades B | | | | | |
| EtOH/heptane | 8235-07-17 @ step 3 | | A + | 8235-34-01 | 544141 | 1012340 | 104 |
| 03:97 v/v | 1. seed w/8235-11-02 | 1. turbid | peaks | | | | |
| | 2. ambient, 1 d | 2. clear solution | | | | | |
| | 3. refrigerated, 4 d | 3. yellow oil | | | | | |
| | 4. fast evaporation | 4. biades B | | | | | |
| EtOH/heptane | 1. dissolved | 1. clear solution | A, C, I | 8235-07-18 | 544142 | 1012341 | 105 |
| 33:77 v/v | 2. spiked with water | 2. phase separated | | | | | |
| | 3. EtOH added slowly | 3. single phase | | | | | |
| | 4. ambient, 1 d | 4. clear solution | | | | | |
| | 5. refrigerated, 4 d | 5. yellow oil | | | | | |
| | 6. fast evaporation | 6. aciculars, B | | | | | |
| EtOAc | dissolved, precip | fines, B | A + E | 8235-06-04 | 542894 | 1000847 | 106 |
| | added Et₂O, filtered | opaque, NB | E + A | 8235-07-13 | 543586 | 1002283 | 107 |
| | | | peaks | | | | |
| | fast evaporation, N₂ | plates, B | E | 8235-06-08 | 542990 | 1001004 | 108 |
| | slow evaporation, N₂ | dendritics, tablets, B | E + A | 8235-09-02 | 542999 | 1001047 | 109 |
| | | | minor | | | | |
| EtO Ac/water | 1. contacted, sonicated | 1. dissolution, slurry | A | 8235-07-09 | 542997 | 1001042 | 110 |
| 97:03 v/v | 2. Et₂O added, filtered | 2. free flowing powder | | | | | |
| | 1. contacted, sonicated | 1. thick paste | A | 8235-48-02 | 544410 | 1003968 | 111 |
| | 2. analyzed wet | 2. — | | | | | |
| | 1. seed w/Pattern D | 1. — | A + D | 8235-48-05 | 544411 | 1003969 | 112 |
| | 2. contacted, sonicated | 2. thick paste | | | | | |
| | 3. analyzed wet | 3. — | | | | | |
| | cooling of solution | blades, B, single xtal | A | 8235-07-10 | 543403 | 3002686 | 114 |
| | 1. cool | 1. yellow oil, fines B | A | 8235-07-16 | 544145 | 1012344 | 115 |
| | 2. fast evaporation | 2. fines, B | | | | | |
| | cool w/Pattern D seed | acicular, B, wet | D | 8235-07-15 | 543673 | 1002455 | 116 |
| | 8235-07-15 | | A + D | 8235-35-01 | 543771 | 1003206 | 117 |
| | 1. 55° C. shaker, 1 d | 1. singles (indexed A) | | | | | |
| | 2. ambient, 1 d | 2,.blades, B. | | | | | |
| | cool. | fine blades/aciculars | D | 8235-07-14 | 544408 | 1003966 | 118 |
| | seed w/8235-07-15 | | | | | | |
| | slurry, ambient, 11 d | opaque and fines, B | D | 8235-69-02 | 545644 | 1005942 | 119 |
| | 8235-48-07 solution | | | | | | |
| | 8235-48-12 amorphous | | | | | | |
| | 8235-63-01 Pattern D | | | | | | |
| heptane | 1. slurry, 85° C., 2 hrs | 1. brown slurry | A + E | 8235-07-01 | 542988 | 1001002 | 120 |
| | 2. cooled to RT | 2. no changes | | | | | |
| | 3. filtered | 3, tan opaque solids | | | | | |
| MEK | contacted, filtered | fines, B | A + E | 8235-06-09 | 542976 | 1000980 | 121 |
| | slow cool | large layered tablets | A | 8235-07-02 | 543770 | 1002689 | 122 |
| | solvent/antisolvent | | A + E | 8235-48-09 | 544691 | 1004453 | 123 |
| | 1. heptane seeded with | 1. seed retained | | | | | |
| | 8235-07-03 | | | | | | |
| | 2. MEK added | 2. no changes | | | | | |
| | 3. keptane added | 3. no changes | | | | | |
| | 4. refrigerated. 1 d | 4. blades, B | | | | | |
| | 8235-48-09 freezer, 2 d | fines, B | A + E | 8235-66-03 | 544904 | 1004728 | 124 |
| MEK/heptane | slurry, ambient, 13 d | tan fines, B | H | 8235-07-03 | 543675 | 1002477 | 125 |
| 50:50 v/v | slurry, ambient, 13 d | — | A, E, H | 8235-48-11 | 545643 | 1005941 | 126 |
| | seed w/8235-07-03 | | | | | | |
| toluene | contacted, centrifuged | tablets, B | A + E | 8235-06-03 | 542898 | 1000852 | 127 |
| | solvent/antisolvent | | A + E | 8235-11-01 | 542987 | 1001001 | 128 |
| | 1. keptane, sonicated | 1. fines, B | | | | | |
| water | 1. dissolved, precip | 1. fines, B | — | 8235-48-03 | — | — | — |
| | 2. sced 8235-07-10 (A) | 2. seed remained | | | | | |
| | 3, ambient, 3 d | 3. seed remained | | | | | |
| | 4. utilized for | 4. — | | | | | |
| | competitive slurry | | | | | | |
| | 1. dissolved, precip | 1. fines, B | K | 8235-48-04 | 547257 | 1008549 | 129 |
| | 2. seed 8235-07-10 (D) | 2. seed remained, | | | | | |
| | 3. ambient, 3 d | 3. seed remained | | | | | |
| | 4. refrigerated, 30 d | 4, limited fines, B | | | | | |
| | 5. freezer, 8 d | 5. wispy aciculars, B | | | | | |
| | 6. pipetted solution | 6. left solids damp | | | | | |
| | 1. 620 mg in 0.6 mL | 1. clear solution | K | 8296-13-01 | 548192 | 1010139 | 130 |
| | heated briefly. 55° C. | | | | | | |
| | 2. filtered, cooled to | 2. clear solution | | | | | |
| | ambient | | | | | | |
| | 3. seed 8235-48-04 (K) | 3. seed retained | | | | | |
| | 4. freezer, 4 d | 4. fine aciculars | | | | | |
| | 5. ambient, 7d | 5. no increase in size | | | | | |
| | 1. 300 mg in 0.3 mL | 1. — | K | 8296-13-02 | 547671 | 1009294 | 131 |
| | of 8296-13-01 step 2 | | | | | | |
| | 2. seed 8233-48-04 (K) | 2. thick suspension | | | | | |
| | 3. mixed for 10 min. | 3. damp solid | | | | | |

^{a}Water activities calculated using UNIFAC calculator.
^{b}Times and temperatures are approximate unless noted.
^{c}B = birefringent and NB = non birefringent when material viewed using polarized light microscopy.

Generated solids were observed by polarized light microscopy (PLM) and/or analyzed by XRPD. Materials exhibiting unique crystalline XRPD patterns, based on visual inspection of peaks associated with these materials, are assigned sequential Roman alphabetical characters as the default designation, if no other character types already pertain to the compound. Each uniquely-identified material is assigned a new designation. The nomenclature convention from previous studies was retained for continuity. Therefore, the designation is tentatively associated with the term ‘Pattern’ until the phase purity and chemical composition is determined through single crystal structure analysis. Verification of phase purity and chemical composition is necessary before the word ‘Form’ is used. In some sections of this report, identifiers are added parenthetically to Pattern/Form designations to provide additional information regarding the conditions that yielded a particular sample.

If possible, single crystal structures were obtained [3, 4, 5, 6, 7, 8]. In the absence of suitable single crystals for structure elucidation, representative XRPD patterns were indexed [9, 10]. Indexing is the process of determining the size and shape of the crystallographic unit cell given the peak positions in a diffraction pattern. The term gets its name from the assignment of Miller index labels to individual peaks. XRPD indexing serves several purposes. If all of the peaks in a pattern are indexed by a single unit cell, this is strong evidence that the sample contains a single crystalline phase. Given the indexing solution, the unit cell volume may be calculated directly. Indexing is also a robust description of a crystalline form and provides a concise summary of all available peak positions for that phase at a particular thermodynamic state point.

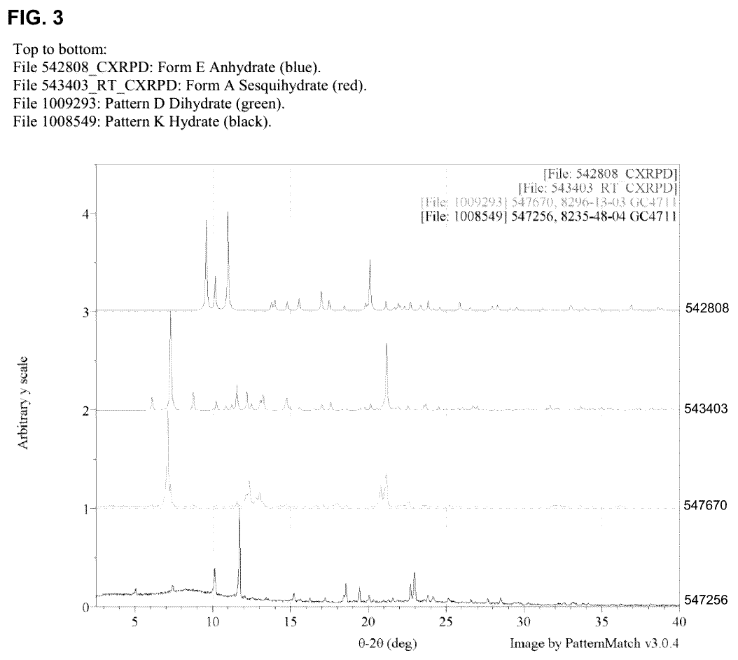

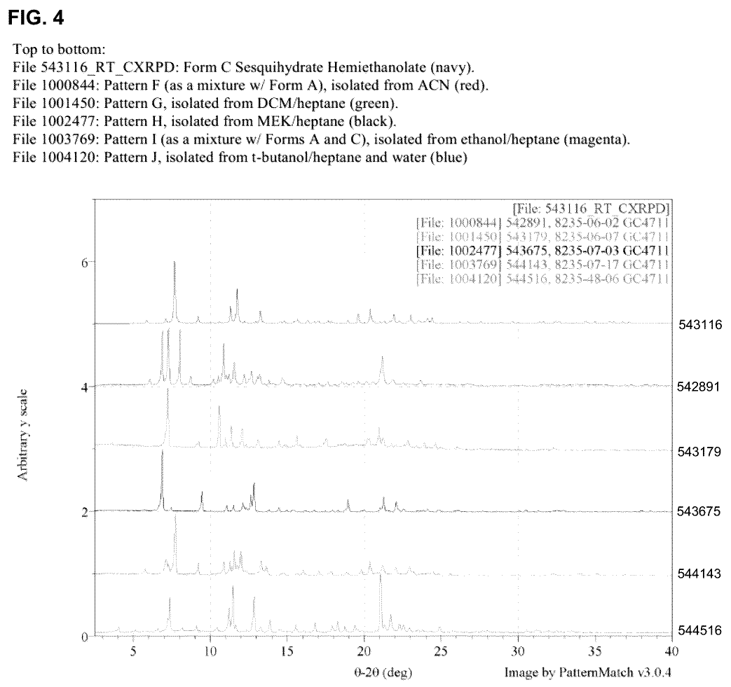

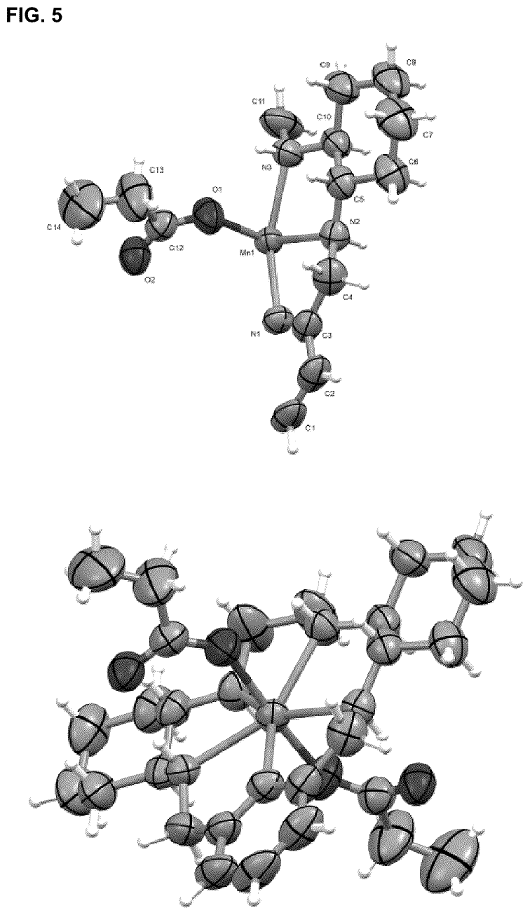

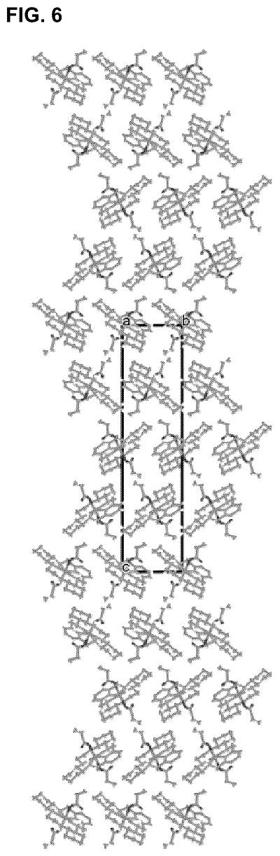

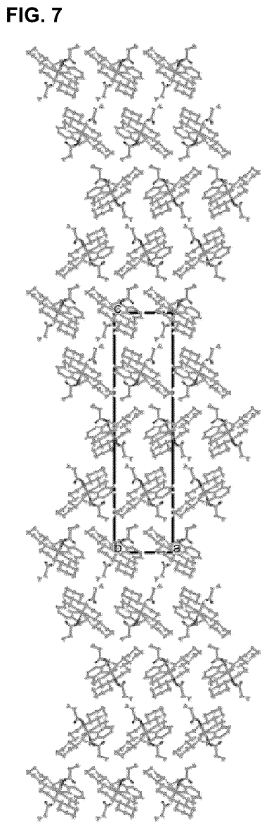

GC4711 readily forms hydrates and mixed solvate/hydrates. Amorphous GC4711 (Section II.C.4) and ten unique crystalline materials were observed. Form E Anhydrate is the only anhydrous form identified within this study (Section II.C.1). Form A Sesquihydrate, Pattern D Dihydrate, and Pattern K were identified as hydrates (Section II.C.2). The XRPD patterns of the anhydrate and hydrates are compared in FIG. 3. Form C Sesquihydrate Hemiethanolate and Materials F through J, shown in FIG. 4 are purported mixed solvates/hydrates (Section II.C.3). Pattern B, a unique XRPD pattern of GC4711 identified in a previous study, was not observed.

### C. Characterization

### 1. Form E Anhydrate

Form E is the only anhydrous form identified within this study (Table 3). Form E Anhydrate exhibits a concomitant melt/decomposition onset near 234° C. and low hygroscopicity from 5 to 45% RH. However, Form E is significantly hygroscopic above 45% RH, where it hydrates to Form A Sesquihydrate. Form E is formed through the crystallization of amorphous GC4711 or desolvation/dehydration of all solvated/hydrated forms at elevated temperature. Form E is also generated when Form A Sesquihydrate or Pattern D Dihydrate is exposed to 0% RH.

TABLE 3
Characterization Data for Form E Anhydrate.

| | | | Sample | | | |
|---|---|---|---|---|---|---|
| Technique | Details | Result | No. | LIMS | File | Page |
| SCXRPD | ambient | Form E Anhydrate | 8235-06-01 | 542808 | 1000787 | 27 |
| XRPD | — | Form E Anhydrate | 8235-14-01 | 543120 | 1001296 | 146 |
| TGA | ambient −350° C. | 0.86% wt. loss up to 185° C. | 8235-14-01 | 543120 | 1001455 | 29 |
| DSC | −30 to 250° C. | minor endotherm onset 55° C. | 8235-14-01 | 543120 | 1001454 | 29 |
| | | endotherm onset of 234° C. | | | | |
| DVS | 5-95-5% RH | 0.25% wt gain from 5 to 45% RH | 8235-14-01 | 543120 | 1002475 | 30 |
| | | 3.97% wt gain from 45-85% RH | | | | |
| | | 23.1% wt gain from 85-95% RH | | | | |
| | | 22.2% wt loss from 95 to 75% RH | | | | |
| | | 1.02% wt loss from 75 to 5% RH | | | | |
| | | hysteresis observed with 4.1 wt % | | | | |
| | | retained | | | | |
| post DVS | — | Form A Sesquihydrate + | 8237-17-01 | 544119 | 1003409 | 168 |
| XRPD | | Pattern D dihydrate | | | | |

A single crystal was isolated from sample 8235-06-1 and the structure was successfully determined. The crystal system is tetragonal and the space group is P43212. The cell parameters and calculated volume are: a=8.95236(18) Å, b=8.95236(18) Å, c=36.9052(15) Å, α=90°, β=90°, γ=90°, V=2957.76(17) Å³. The formula weight is 558.62 g mol⁻¹ with Z=4, resulting in a calculated density of 1.254 g cm⁻³. Further details of the crystal data and crystallographic data collection parameters are summarized in Table 4.

TABLE 4
Crystal Data and Data Collection
Parameters for Form E Anhydrate.

| | |
|---|---|
| Empirical formula | C₂₇H₄₅MnN₅O₄ |
| Formula weight (g mol⁻¹) | 558.62 |
| Temperature (K) | 299.6(3) |
| Wavelength (Å) | 1.54184 |
| Crystal system | tetragonal |
| Space group | P4₃2₁2 |
| Unit cell parameters | |
| a = 8.95236(18) Å | α = 90° |
| b = 8.95236(18) Å | β = 90° |
| c = 36.9052(15) Å | γ = 90° |
| Unit cell volume (Å³) | 2957.76(17) |
| Cell formula units, Z | 4 |
| Calculated density (g cm⁻³) | 1.254 |
| Absorption coefficient (mm⁻¹) | 3.941 |
| F(000) | 1196 |
| Crystal size (mm³) | 0.12 × 0.08 × 0.04 |
| Reflections used for cell measurement | 3282 |
| θ range for cell measurement | 4.7150°-73.0700° |
| Total reflections collected | 7414 |
| Index ranges | −7 ≤ h ≤ 8; −11 ≤ |
| | k ≤ 9; −43 ≤ l ≤ 46 |
| θ range for data collection | θₘᵢₙ = 4.793°, |
| | θₘₐₓ = 77.515° |
| Completeness to θₘₐₓ | 94.8% |
| Completeness to θ_{full} = 67.684° | 98% |
| Absorption correction | multi-scan |
| Transmission coefficient range | 0.809-1.000 |
| Refinement method | full matrix |
| | least-squares on F² |
| Independent reflections | 2905 [Rᵢₙₜ = 0.0242, |
| | R_{σ} = 0.0280] |
| Reflections [I > 2σ(I)] | 2260 |
| Reflections/restraints/parameters | 2905/0/178 |
| Goodness-of-fit on F² | S = 1.02 |
| Final residuals [I > 2σ(I)] | R = 0.0361, R_{w} = 0.0880 |
| Final residuals [all reflections] | R = 0.0518, R_{w} = 0.0984 |
| Largest diff. peak and hole (e Å⁻³) | 0.154, −0.177 |
| Max/mean shift/standard uncertainty | 0.000/0.000 |
| Absolute structure determination | Flack parameter: −0.013(4) |

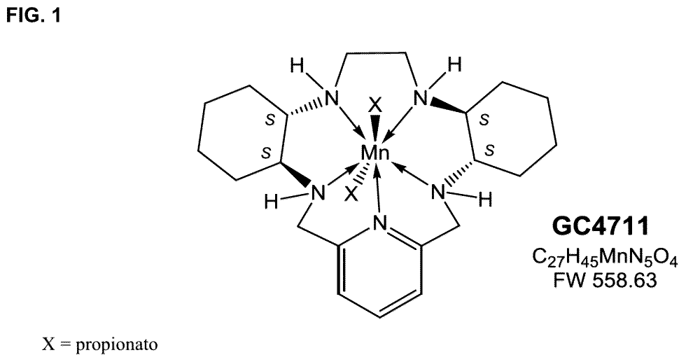

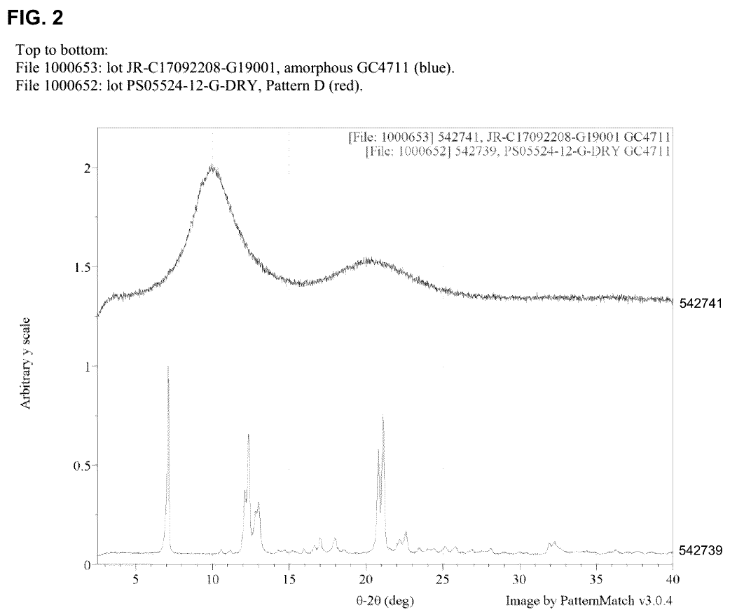

An atomic displacement ellipsoid drawing of Form E Anhydrate is shown in FIG. 5. The asymmetric unit shown contains ½ of the manganese and chelating pentaazamacrocycle and one propionate. GC4711 is symmetric and is divided by a 2-fold axis, which generates the other half of the manganese and chelating pentaazamacrocycle and the other propionate. Four chiral centers are shown in FIG. 1. However, half of the complex is symmetry generated, and therefore this structure contains two chiral centers located at C5 and C10 (refer to FIG. 5) which both bond in the S configuration. This is consistent with the proposed configuration in FIG. 1.

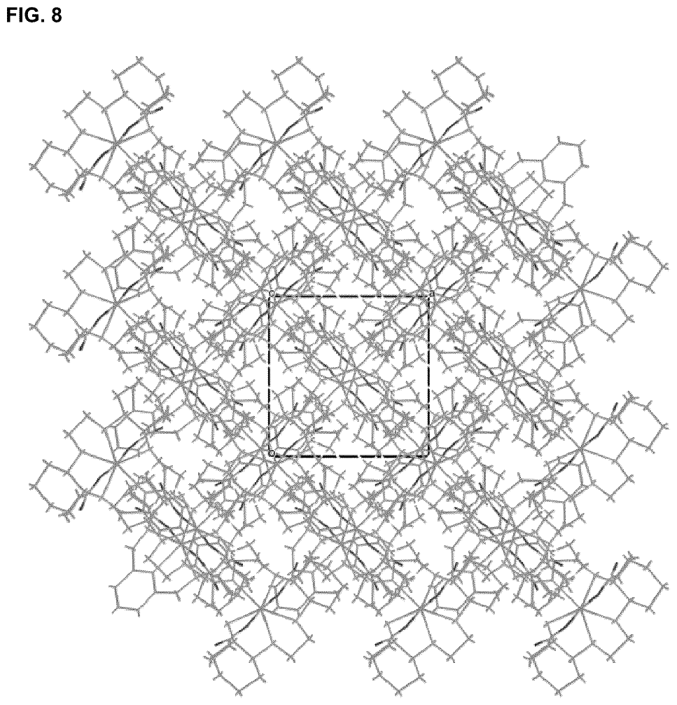

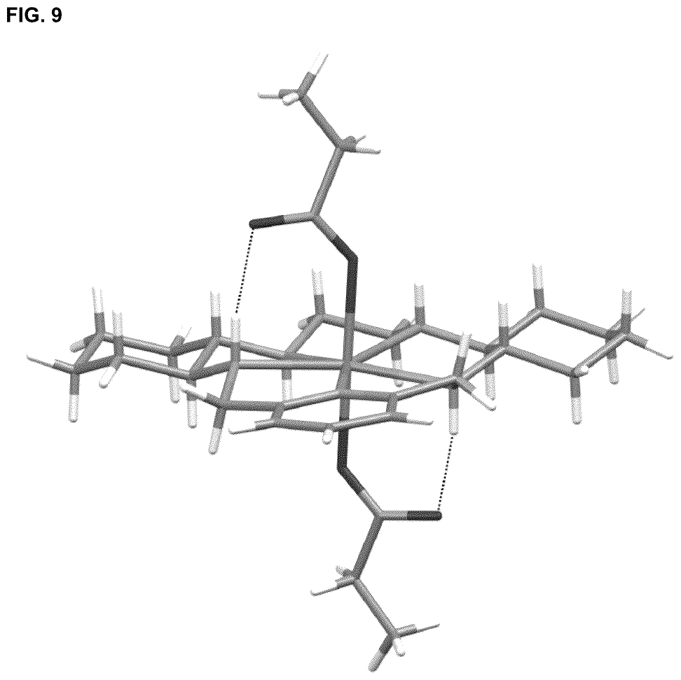

Packing diagrams viewed along the a, b, and c crystallographic axes are shown in FIGS. 6-8 respectively. The manganese is coordinated by the five nitrogen atoms from the chelating molecule and two propionates. One oxygen atom of the propionate is coordinated to manganese and the other is hydrogen bonded to the amine that links the cyclohexane and pyridine moieties, shown in FIG. 9.

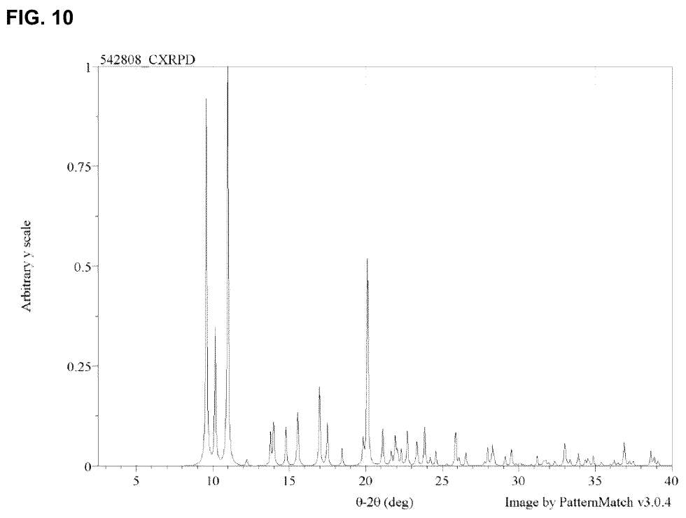

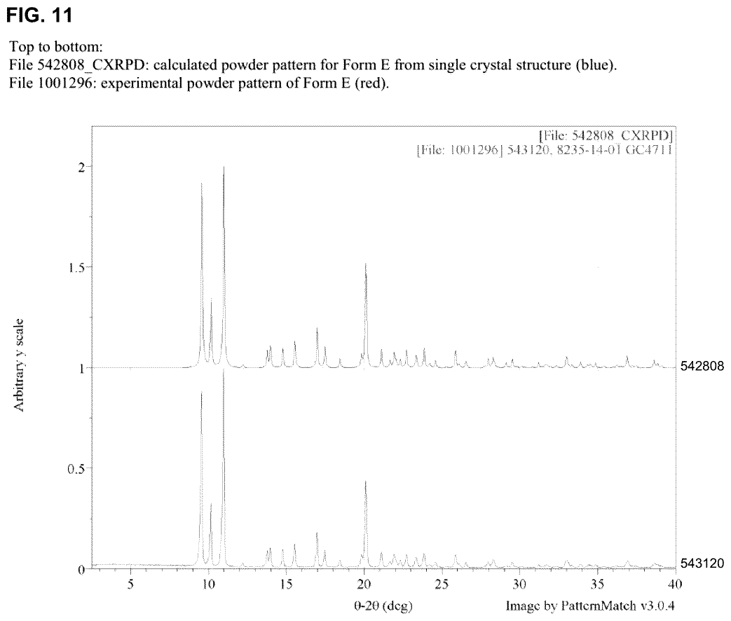

FIG. 10 shows a calculated XRPD pattern of Form E Anhydrate, generated from the single crystal structure. The pattern is compared to an experimental powder pattern in FIG. 11.

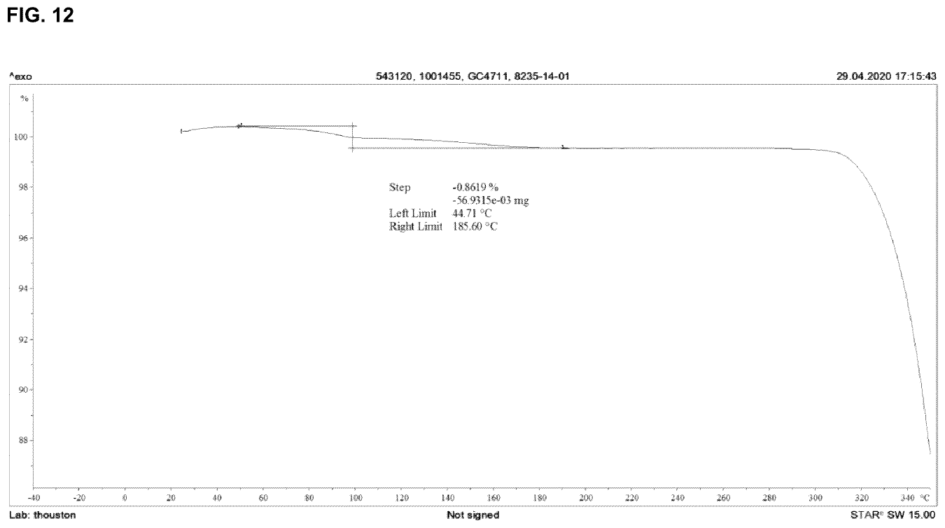

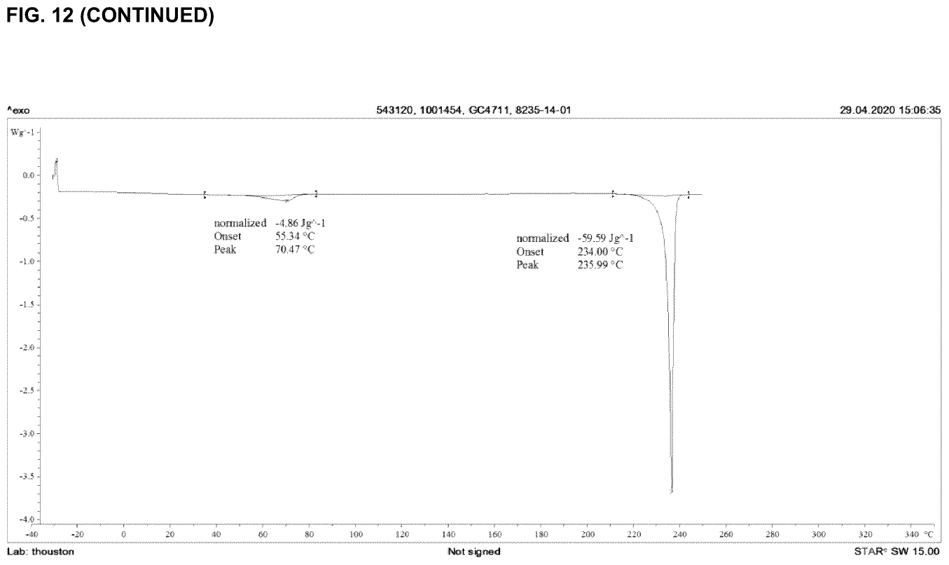

Thermograms for Form E are presented in FIG. 12. The TGA thermogram exhibits 0.87% weight loss up to 187° C. concurrent with a weak dehydration endotherm in the DSC. Based on the single crystal structure, the form does not contain water and these events are likely a consequence of hygroscopicity and moisture uptake from ambient laboratory conditions during sample preparation. A final DSC endotherm with an onset of 234° C. is due to concomitant melt/decomposition (as confirmed by hot stage microscopy, see Section II.C.2.b).

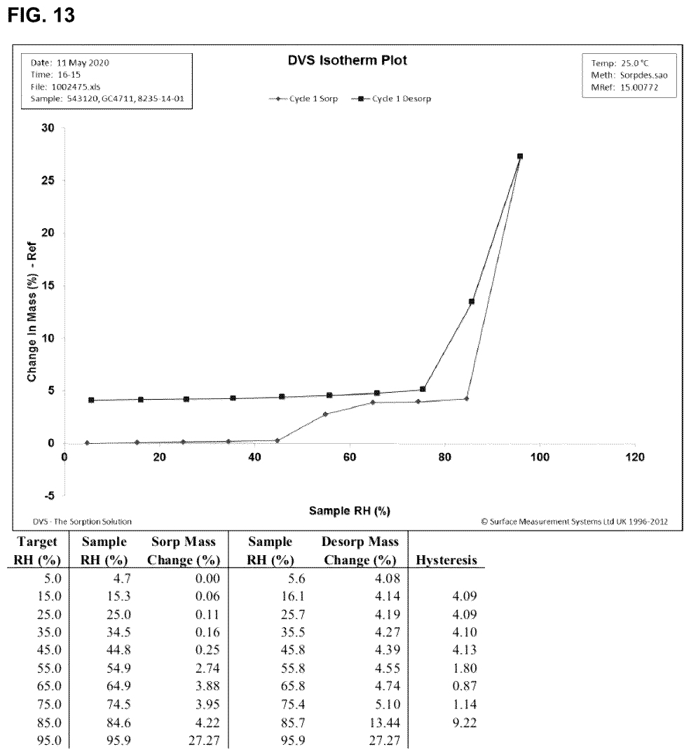

The dynamic vapor sorption (DVS) isotherm shown in FIG. 13 indicates Form E exhibits low hygroscopicity from 5 to 45% RH. However, significant hygroscopicity is evident above 45% RH with approximately 4 wt % gained from 45 to 85% RH and an additional 23 wt % from 85 to 95% RH. Hygroscopicity can be described as low, limited, or significant in part on concepts presented in reference [11]. The instrument timed-out at 85% RH and above, which suggests that additional weight gain would likely occur if left to equilibrate at these conditions further. Hysteresis, the difference between the water vapor uptake between the sorption and desorption isotherms, is consistent with the formation of a hydrate. The material recovered from the DVS experiment was identified as a mixture of Form A Sesquihydrate and Pattern D Dihydrate by XRPD, confirming that a form change did occur. Indeed, Form E Anhydrate was shown to hydrate to Form A Sesquihydrate by XRPD when exposed to 43% RH at room temperature for 6 days (Table 5).

TABLE 5
Physical Stability of Form E Anhydrate.

| Method^{a} | Result | Source | Sample | LIMS | File | Page |
|---|---|---|---|---|---|---|
| 43% RH, RT, 6 d | A | 8235-14-01 | 8235-88-01 | 546752 | 1007817 | 147 |

^{a}Times and temperatures are approximate unless noted.

### 2. Hydrates

a. Form A Sesquihydrate

Form A is a sesquihydrate (Table 6). Form A Sesquihydrate was the most commonly observed form from the crystallization experiments within this study. Form A was shown to be the prevailing hydrate, relative to Pattern D Dihydrate, at 11% RH and, although less stable than Pattern D at higher humidity, is kinetically stable in the solid state within the time-frame evaluated. Form A exhibits low hygroscopicity from 5 to 85% RH and significant hygroscopicity above 85% RH. Form A dehydrates to Form E Anhydrate when exposed to elevated temperature or 0% RH.

TABLE 6
Characterization Data for Form A Sesquihydrate.

| Technique | Details | Result | Sample No. | LIMS | File | Page |
|---|---|---|---|---|---|---|
| SCXRPD | 150K | Form A Sesquihydrate | 8235-07-10 | 543403 | 1008964 | 32 |
| XRPD | — | Form A Sesquihydrate | 8235-07-07 | 543118 | 1001294 | 139 |
| TGA | ambient −350º C. | 4.73% wt. loss up to 176° C. | 8235-07-07 | 543118 | 1001457 | 34 |
| | | loss consistent with 1.5 mol/mol water | | | | |
| DSC | −30 to 250° C. | dehydration endotherm onset 82° C. | 8235-07-07 | 543118 | 1001456 | 34 |
| | | final endotherm onset of 231º C. | | | | |
| DVS | 5-95-5% RH | 0.78% wt gain from 5 to 85% RH | 8235-07-07 | 543118 | 1002474 | 35 |
| | | 12.94% wt gain from 85 to 95% RH | | | | |
| | | 12.28% wt loss from 95 to 75% RH | | | | |
| | | 1.28% wt loss from 75 to 5% RH | | | | |
| | | hysteresis observed | | | | |
| post DVS | — | Form A Sesquihydrate | 8118-58-01 | 543946 | 1003041 | 155 |
| XRPD | | | | | | |

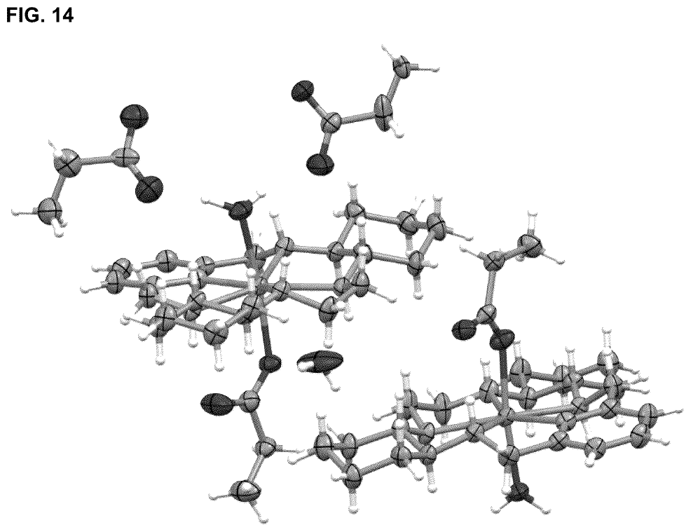

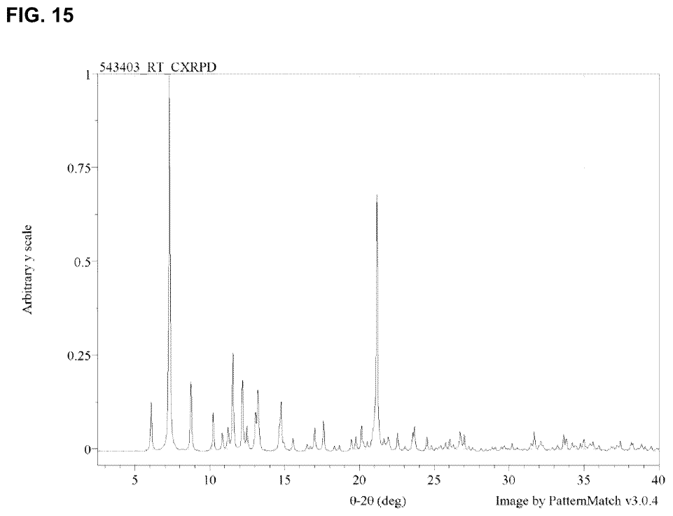

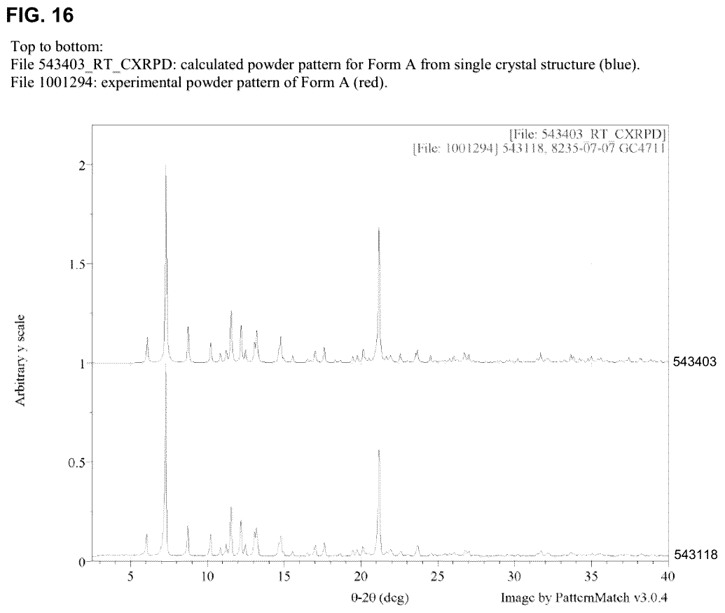

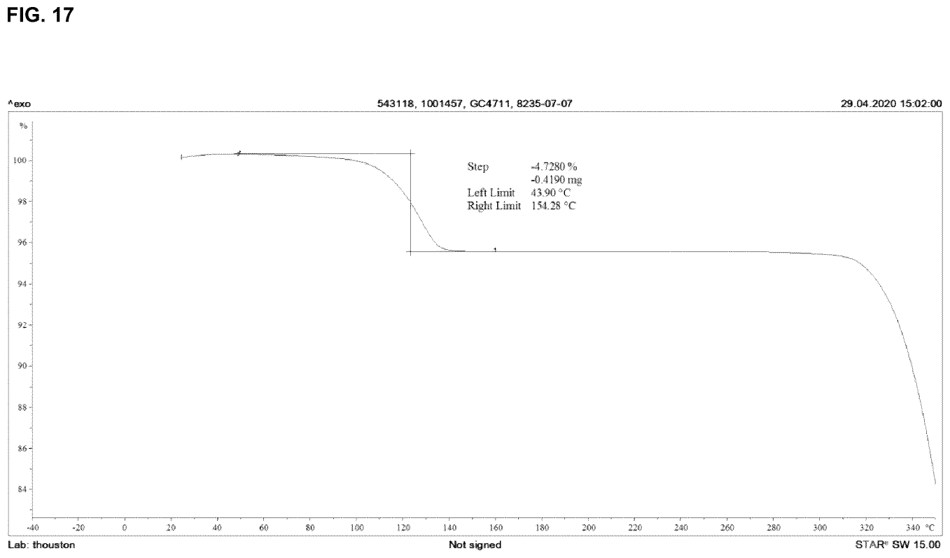

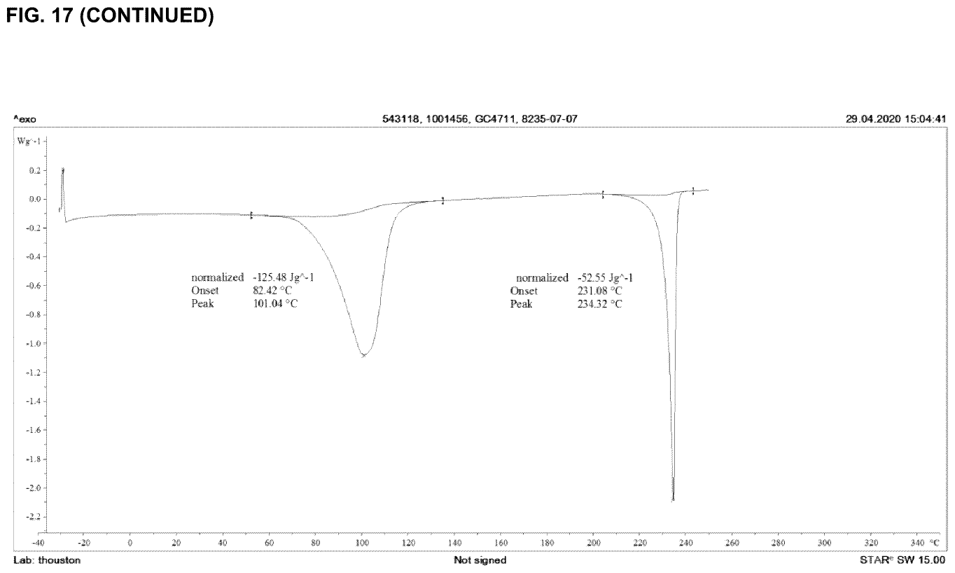

A single crystal was isolated from sample 8235-07-10 and the structure was successfully determined. The crystal system is triclinic and the space group is P1. The cell parameters and calculated volume are: a=8.47305(11) Å, b=12.60925(19) Å, c=14.5880(2) Å, α=97.1497(13)°, β=97.7183(12)°, γ=103.6420(12)°, V=1481.02(4) Å³. The formula weight is 585.64 g mol⁻¹ with Z=2, resulting in a calculated density of 1.313 g cm⁻³. Further details of the crystal data and crystallographic data collection parameters are summarized in Table 7. An atomic displacement ellipsoid drawing of Form A Sesquihydrate is shown in FIG. 14. The asymmetric unit shown contains two manganese and chelating pentaazamacrocycles, four propionates, and three water molecules. Water, instead of a propionate, occupies one of the axial ligand positions. The calculated powder pattern is presented in FIG. 15 and compared to an experimental pattern in FIG. 16.

TABLE 7
Crystal Data and Data Collection Parameters
for GC4711 Form A Sesquihydrate.

| | |
|---|---|
| Empirical formula | C₂₇H₄₈MnN₅O_{5.50} |
| Formula weight (g mol⁻¹) | 585.64 |
| Temperature (K) | 150.00(10) |
| Wavelength (Å) | 1.54184 |
| Crystal system | triclinic |
| Space group | P1 |
| Unit cell parameters | |
| a = 8.47305(11) Å | α = 97.1497(13)° |
| b = 12.60925(19) Å | β = 97.7183(12)° |
| c = 14.5880(2) Å | γ = 103.6420(12)° |
| Unit cell volume (Å³) | 1481.02(4) |
| Cell formula units, Z | 2 |
| Calculated density (g cm⁻³) | 1.313 |
| Absorption coefficient (mm⁻¹) | 3.997 |
| F(000) | 628 |
| Crystal size (mm³) | 0.56 × 0.18 × 0.05 |
| Reflections used for cell measurement | 16178 |
| θ range for cell measurement | 4.3650°-77.8000° |
| Total reflections collected | 26865 |
| Index ranges | −10 ≤ h ≤ 10; −16 ≤ |
| | k ≤ 15; −18 ≤ l ≤ 17 |
| θ range for data collection | θₘᵢₙ = 3.656°, |
| | θₘₐₓ = 78.303° |
| Completeness to θₘₐₓ | 97.7% |
| Completeness to θ_{full} = 67.684° | 99.8% |
| Absorption correction | multi-scan |
| Transmission coefficient range | 0.680-1.000 |
| Refinement method | full matrix |
| | least-squares on F² |
| Independent reflections | 9946 [Rᵢₙₜ = 0.0445, |
| | R_{σ} = 0.0424] |
| Reflections [I > 2σ(I)] | 9193 |
| Reflections/restraints/parameters | 9946/3/760 |
| Goodness-of-fit on F² | S = 1.10 |
| Final residuals [I > 2σ(I)] | R = 0.0549, R_{w} = 0,1517 |
| Final residuals [all reflections] | R = 0.0582, R_{w} = 0,1544 |
| Largest diff. peak and hole (e Å⁻³) | 0.721, −0.792 |
| Max/mean shift/standard uncertainty | 0.000/0.000 |
| Absolute structure determination | Flack parameter: −0.004(4) |

TABLE 8
Physical Stability of Form A Sesquihydrate.

| Details | Result | Source | Sample | LIMS | File | Page |
|---|---|---|---|---|---|---|
| 80 to 110° C. 15 min | Forms A and E | 8235-07-09 | 8235-88-03 | 546468 | 1007358 | 132 |

Thermograms for Form A Sesquihydrate are shown in FIG. 17. The TGA thermogram exhibits a 4.7% weight loss up to 176° C. concurrent with a broad dehydration endotherm in the DSC. This loss is due to the volatilization of ˜1.5 mol/mol of water, consistent with a sesquihydrate. A final DSC endotherm with an onset of 231° C. is the concomitant melt/decomposition of the dehydrated form (Form E Anhydrate). Form A was shown to dehydrate to Form E when exposed to elevated temperature or 0% RH (Tables 8 and 13, respectively).

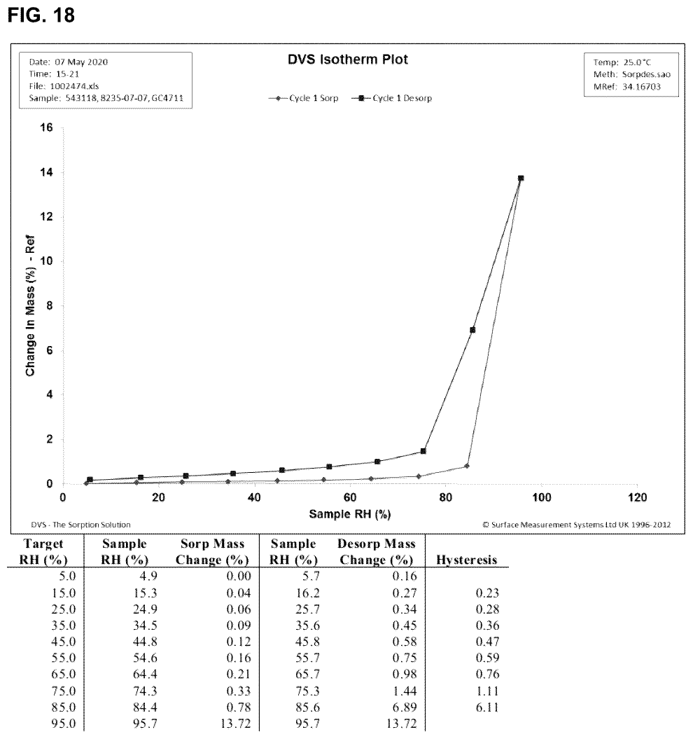

The DVS isotherm (FIG. 18) indicates Form A exhibits low hygroscopicity from 5 to 85% RH. However, significant hygroscopicity is evident above 85% RH with approximately 13 wt % gained. The instrument timed-out at 85% RH and above, which suggests that additional weight gain would likely occur if left to equilibrate at these conditions further. The material recovered from the DVS experiment was identified as Form A by XRPD, suggesting that Form A Sesquihydrate is kinetically stable at these conditions.

b. Pattern D Dihydrate

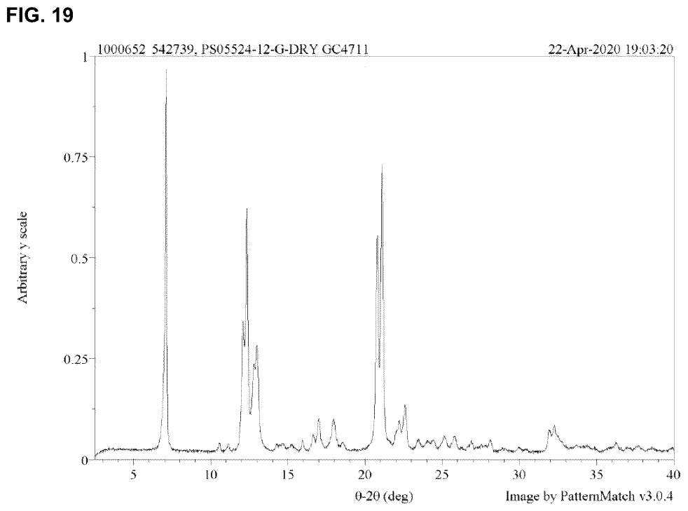

Pattern D Dihydrate was received as lot PS05524-12-G-DRY (FIG. 19 and Table 9). Pattern D could only be reproduced within this study by seeding saturated 97:03 v/v EtOAc/water solutions. Pattern D Dihydrate was shown to be the prevailing hydrate, relative to Form A Sesquihydrate, at 0.23 water activity (equivalent to 23% RH) and higher. Pattern D exhibits low hygroscopicity from 5 to 85% RH and significant hygroscopicity above 85% RH. Pattern D dehydrates to Form E Anhydrate when exposed to elevated temperature or 0% RH.

TABLE 9
Characterization for Lot PS05524-12-G-DRY, Pattern D Dihydrate.

| Technique | Details | | Result | LIMS | File | Page |
|---|---|---|---|---|---|---|
| XRPD | not indexable | | Pattern D | 542739 | 1000652 | 157 |
| microscopy | polarized light | | fines, birefringent | 542739 | — | — |
| TGA | ambient −350° C. | | 6.1% weight loss up to 155° C. | 542739 | 1000654 | 38 |
| | | | loss consistent with 2 mol/mol water | | | |
| DSC | −30 to 250° C. | | dehydration endotherm onset 79° C. | 542739 | 1000656 | 38 |
| | | | final endotherm onset 232° C. | | | |
| hot stage | 23.1° C. | — | birefringent fines | 542739 | 1000658-1 | 158 |
| microscopy | 64.7 | 5° C./min | changes in birefringence | 542739 | 1000658-2 | 159 |
| | 73.8 | | birefringence lost | 542739 | 1000658-3 | 160 |
| | 165.7 | 20° C./min | change in material color | 542739 | 1000658-4 | 161 |
| | 176.0 | 2° C./min | softening/liquefaction | 542739 | 1000658-5 | 162 |
| | 196.9 | 5° C./min | continuation, darkening of material | 542739 | 1000658-6 | 163 |
| | 217.0 | | no change | 542739 | 1000658-7 | 164 |
| | ambient | — | did not crystallize, black solids | 542739 | — | — |

| | | | | | |
|---|---|---|---|---|---|
| KF | coulometric | 7.05% water content | 542739 | 1000662 | 165 |
| | | consistent with ~2.3 mol/mol water | | | |
| DVS | 25-95-5% RH | 0.37% weight gain from 25 to 85% RH | 542739 | 1000660 | 40 |
| | | 11.57% weight gain from 85 to 95% RH | | | |
| | | 11,58% weight gain from 95 to 75% RH | | | |
| | | 0.46% weight loss from 75 to 5% RH | | | |
| post DVS | 8135-100-01 | Pattern D | 543161 | 1001402 | 167 |
| XRPD | | | | | |

TABLE 10
Physical Stability for Lot PS05524-
12-G-DRY, Pattern D Dihydrate.

| Details | Result | Sample | LIMS | File | Page |
|---|---|---|---|---|---|
| 110° C. 35 min | Form E | 8235-57-01 | 544409 | 1003967 | 133 |

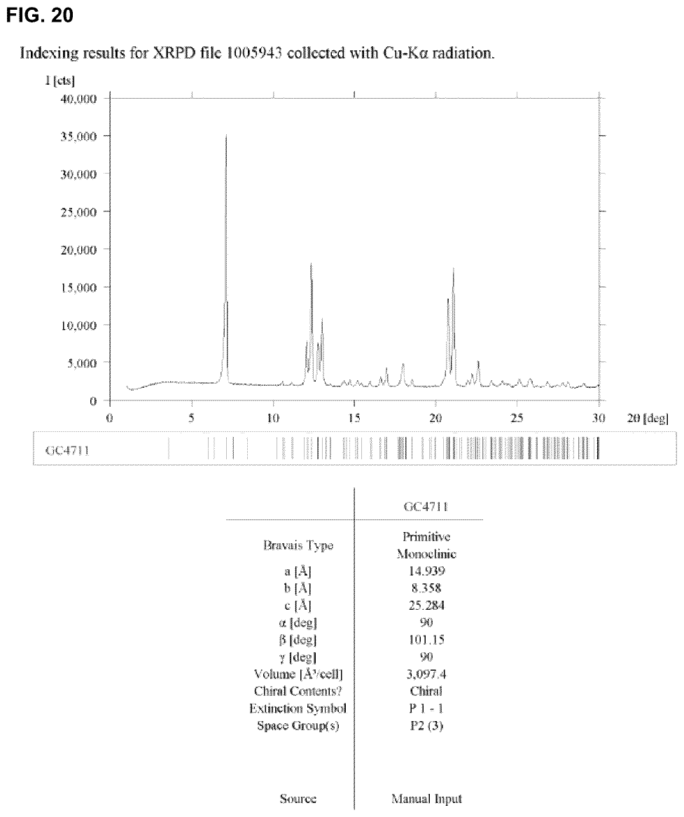

A representative XRPD pattern of Pattern D was successfully indexed by a single primitive monoclinic unit cell and provides a robust description of the crystalline form through tentative crystallographic unit cell parameters and strong evidence that the pattern is representative of a single crystalline phase (FIG. 20). Assuming a multiplicity of 4, the formula unit volume of 774 Å³ calculated from the indexing results provides a free volume of approximately 35 Å³ (relative to the volume of Form E) that can theoretically accommodate up to two mol/mol of water [12].

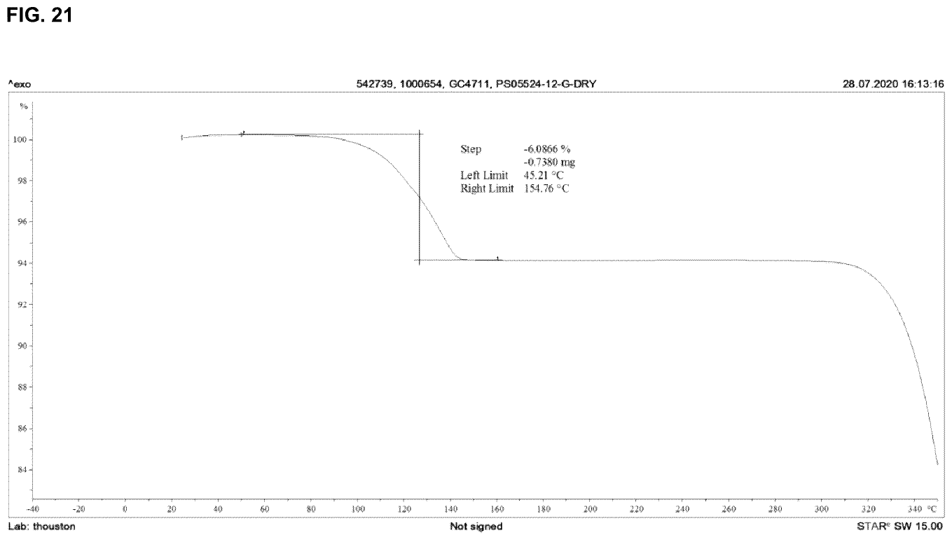

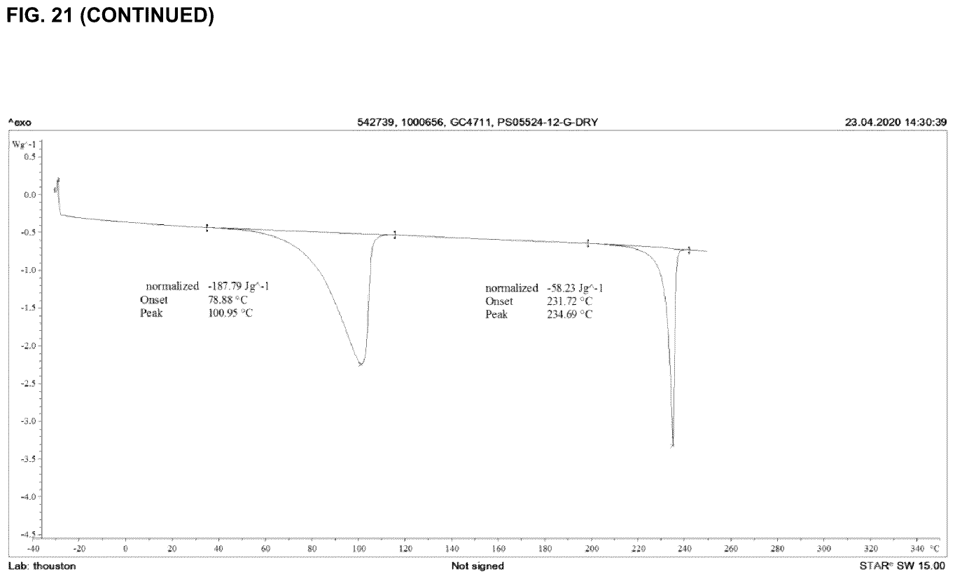

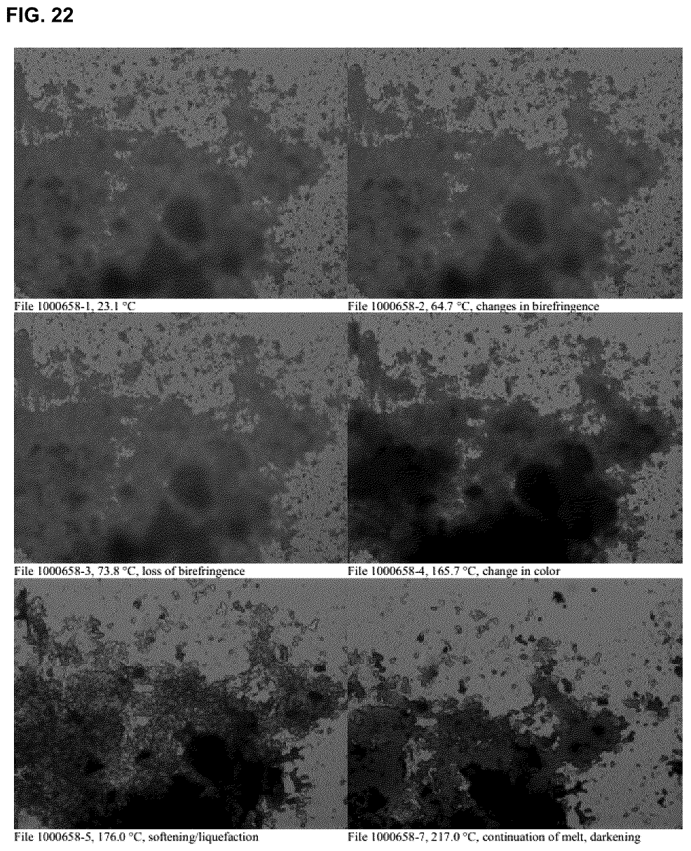

Thermograms for Pattern D are provided in FIG. 21. The TGA thermogram exhibits a 6.1% weight loss up to 155° C. concurrent with a broad dehydration endotherm in the DSC. This loss is consistent with the volatilization of ˜2 mol/mol of water. Karl Fischer titration of 7.05 wt % water (˜2.3 mol/mol water) supports this result. A final DSC endotherm with an onset of 232° C. is the concomitant melt/decomposition of the dehydrated form (Form E Anhydrate). Pattern D was shown to dehydrate to Form E when exposed to elevated temperature or 0% RH (Tables 10 and 13, respectively). Hot stage microscopy, provided in FIG. 22, confirms the thermal events and suggests that decomposition likely occurs at a lower temperature (˜166° C.) than the melt onset when not protected from oxidation.

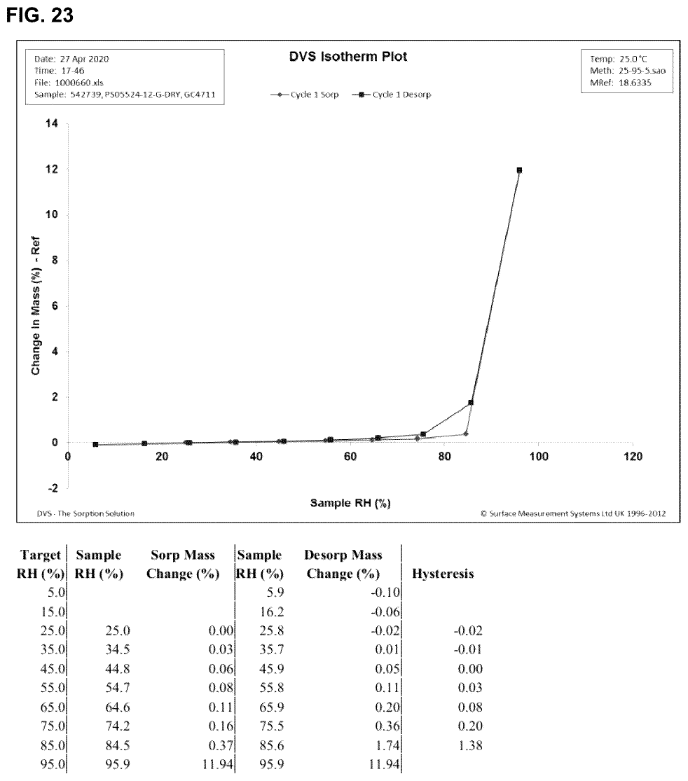

The DVS isotherm (FIG. 23) indicates Pattern D exhibits low hygroscopicity from 5 to 85% RH. However, significant hygroscopicity is evident above 85% RH with more than 11 wt % gained. The instrument timed-out at 85% RH and above, which suggests that additional weight gain would likely occur if left to equilibrate at these conditions further. The material recovered from the DVS experiment was identified as Pattern D by XRPD, suggesting that Pattern D Dihydrate is kinetically stable at these conditions.

c. Pattern K Hydrate

Pattern K appears to be a hydrate that is precipitated from highly concentrated aqueous solutions. Pattern K was isolated near the conclusion of this study and was not included in the relative hydrate stability assessment discussed in the subsequent section below. Characterization data is limited (Table 11). However, indexing results and successive dehydration under nitrogen is suggestive of a much higher hydration state than Pattern D Dihydrate. In addition, Pattern K is less stable than either Form A Sesquihydrate or Pattern D Dihydrate at that condition.

TABLE 11
Characterization Data for Pattern K.

| Technique | Details | Result | Sample | LIMS | File | Page |
|---|---|---|---|---|---|---|
| XRPD | indexed | Pattern K | 8235-48-04 | 547257 | 1008549 | 129 |

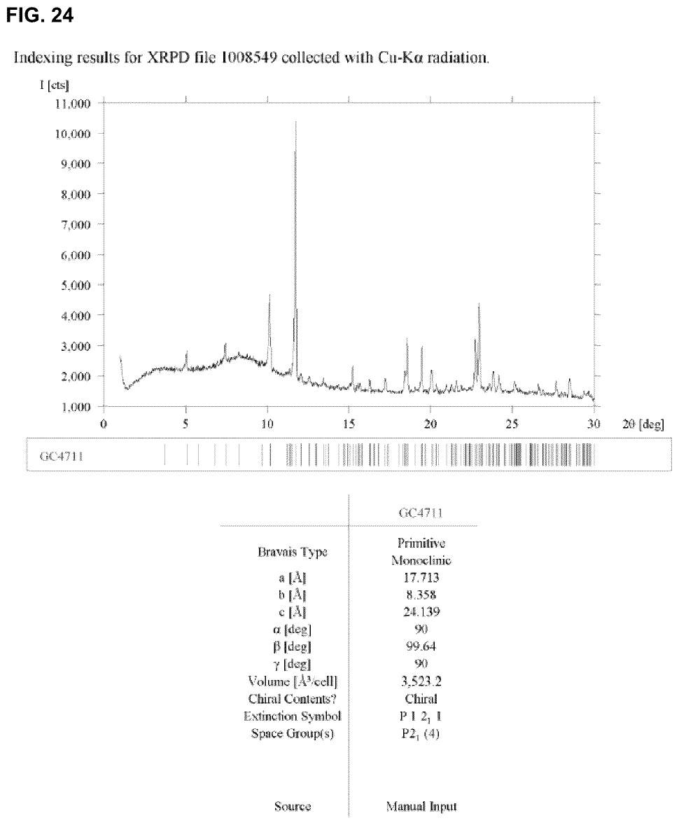

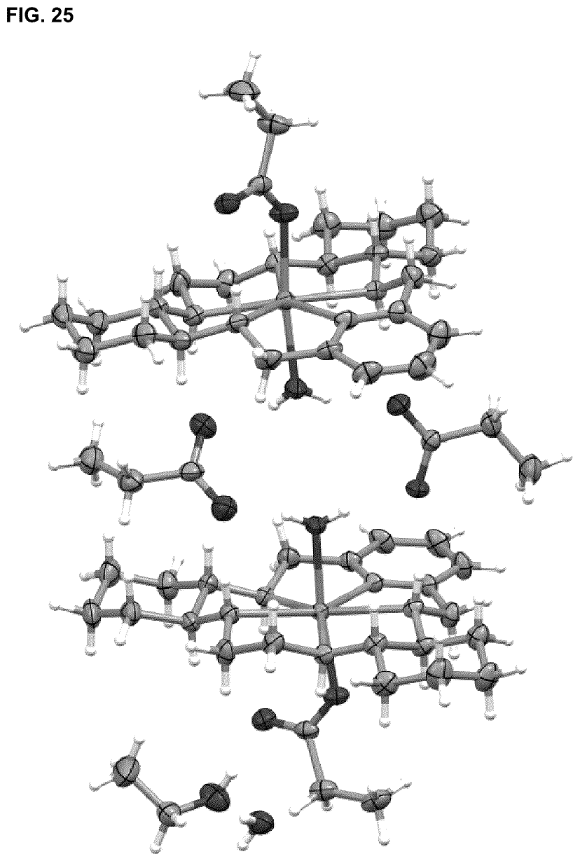

A representative XRPD pattern of Pattern K was successfully indexed by a single primitive monoclinic unit cell (FIG. 24). Assuming a multiplicity of 4, the formula unit volume of 881 Å³ calculated from the indexing results provides a free volume of approximately 141 Å³ (relative to the volume of Form E) that can theoretically accommodate up to 7 mol/mol of water [12].

The physical stability of Pattern K was investigated (Table 12). Partial dehydration to a mixture of Form A Sesquihydrate and Pattern D Dihydrate occurred under brief exposure to dry nitrogen. This suggests that Pattern K is less stable than either Form A Sesquihydrate or Pattern D Dihydrate at that condition.

TABLE 12
Physical Stability of Pattern K.

| Details | Result | Source | Sample | LIMS | File | Page |
|---|---|---|---|---|---|---|
| N₂ dried for ~10 minutes | Form A + Pattern D | 8296-13-02 | 8296-13-03 | 547670 | 1009293 | 175 |

d. Relative Hydrate Stability

The effect of relative humidity (RH) and water activity (a_{w}) on the hydration state of GC4711 was investigated through static exposure to different RH and competitive water activity trituration experiments (slurries) at room temperature (Tables 13 and 14, respectively). The resulting solid phase was characterized by XRPD. Water activity is related to relative humidity in that RH %=a_{w}×100. Therefore, it is possible to directly relate the stability of an anhydrous/hydrate system in slurry experiments to solid state stability. Literature suggests that the slurry technique at controlled water activities provides an accurate method of rapidly predicting the physically stable form in anhydrous/hydrate systems [13, 14, 15, 16]. The method is particularly valuable when relatively slow kinetics of conversion in the solid state prevents reaching true equilibrium in a reasonable timeframe, since solvent-mediated transformation accelerates the conversion process. These experiments were used to establish the stabile relative humidity range for Form E Anhydrate, Form A Sesquihydrate, and Pattern D Dihydrate.

TABLE 13
Physical Stability of Form A Sesquihydrate and Pattern D Dihydrate at
Different Relative Humidities (Room Temperature).

| Source | Method [symbol] | Result^{b} | Source | Sample | LIMS | File | Page |
|---|---|---|---|---|---|---|---|
| Pattern D | 0% RH, P₂O₅, 11 d | E | 542739 | 8235-69-01 | 545641 | 1005939 | 145 |
| Forms A + E | 0% RH, P₂O₅, 3 d | E | 8235-06-06 | 8235-14-01 | 543120 | 1001296 | 146 |
| Form A + Pattern D | 11% RH, 15 d | A↑ + D | 8235-48-05 | 8235-60-05 | 545640 | 1005938 | 141 |
| | 43% RH, 15 d | A + D | 8235-48-05 | 8235-60-03 | 545637 | 1005935 | 142 |
| | 75% RH, 15 d | D↑ + A | 8235-48-05 | 8235-60-02 | 545638 | 1005936 | 143 |
| | 85% RH, 15 d | D↑ + A | 8235-48-05 | 8235-60-01 | 545639 | 1005937 | 144 |

[symbol]

indicates data missing or illegible when filed

TABLE 14
Water Activity Interconversion Slurries between
Form A Sesquihydrate and Pattern D Dihydrate.

| a_{w}^{c} | Solvent System (v/v) | Methoda | Result | Source | Sample | LIMS | File | Page |
|---|---|---|---|---|---|---|---|---|
| 0.23 | 97:03 EtQAc/water | 2-8° C., 15 d | D | 8235-48-05 | 8235-59-02 | 545645 | 1005943 | 152 |
| 0.23 | 97:03 EtOAc/water | ambient, 1 d | D | 8235-48-05 | 8235-59-01 | 544619 | 1004312 | 153 |
| 0.35 | 98:02 acetone/water | ambient, 14 d | D | 8235-48-01 | 8235-89-02 | 547255 | 1008548 | 151 |
| | | | | LIMS 542739 | | | | |
| 0.44 | 97:03 acetone/water | ambient, 14 d | D | 8235-48-01 | 8235-89-04 | 547257 | 1008550 | 150 |
| | | | | LIMS 542739 | | | | |
| 0.50 | 96:04 acetone/water | ambient, 14 d | D | 8235-48-01 | 8235-88-06 | 547254 | 1008547 | 149 |
| | | | | LIMS 542739 | | | | |
| 1.00 | water | ambient, 14 d | D + | 8235-48-05 | 8235-60-04 | 545642 | 1005940 | 154 |
| | | | diffuse | | | | | |
| | | | scatter | | | | | |

aTimes are approximate unless noted.
^{b}Arrows indicate increase in peak intensity of this particular form relative to the other.
^{c}Water activities calculated using UNIFAC calculator.

As discussed in Section II.C.2.c, Pattern K, a higher hydrate precipitated from highly concentrated aqueous solutions, was not included in the relative hydrate stability assessment. Partial dehydration to a mixture of Form A Sesquihydrate and Pattern D Dihydrate occurred under brief exposure to dry nitrogen and suggests that Pattern K is less stable than either form at that condition. Tentatively, Pattern K is assumed to be prevalent only near a_{w}≈1 but remains unconfirmed.

Complete dehydration of both hydrated forms to Form E Anhydrate occurred at 0% RH. Form A was shown to be the prevailing hydrate, relative to Pattern D Dihydrate, at 11% RH, while Pattern D Dihydrate was the prevailing hydrate at 75% RH and above. Relatively slow kinetics of conversion in the solid state prevented reaching true equilibrium at 43% RH. However, the aw experiments confirm that Pattern D Dihydrate is the prevailing hydrate at and above 0.23 aw (equivalent to 23% RH). At room temperature, the stable RH regions for these forms can be summarized as follows:

| Water Activity (RT) | 0 < a_{w}^{E↔A} < 0.11 < a_{w}^{A↔D} < 0.23 | | | a_{w}^{→K} = 1 |
|---|---|---|---|---|
| Prevailing Form | E | A | D | K |

### 3. Other Purported Solvates, Hydrates, or Mixed Solvates/Hydrates

a. Pattern B

A previous study identified a unique XRPD pattern of GC4711 as Pattern B; however, Pattern B was not observed within this study. The nature of Pattern B is unknown.

b. Form C Sesquihydrate Hemiethanolate

Form C is a sesquihydrate hemiethanolate (Table 15). The mixed solvate/hydrate converts to Form E Anhydrate at elevated temperatures or upon brief exposure to dry nitrogen.

TABLE 15
Characterization Data for Form C Sesquihydrate Hemiethanolate.

| Technique | Details | Result | Sample No. | LIMS | File | Page |
|---|---|---|---|---|---|---|
| SCXRPD | 150K | Form C Sesquihydrate Hemiethanolate | 8235-11-02 | 543116 | 1008965 | 43 |
| XRPD | indexed | Form C Sesquihydrate Hemiethanolate | 8235-07-04 | 543117 | 1001293 | 156 |
| TGA | ambient—350 ° C. | 8.27% wt. loss up to 176° C. | 8235-07-04 | 543117 | 1001453 | 45 |
| | | loss consistent with 1.5 mol/mol water | | | | |
| | | and 0.5 mol/mol ethanol | | | | |
| DSC | −30 to 250° C. | desalvation endotherm onset 65° C. | 8235-07-04 | 543117 | 1001452 | 45 |
| | | final endotherm onset of 231° C. | | | | |

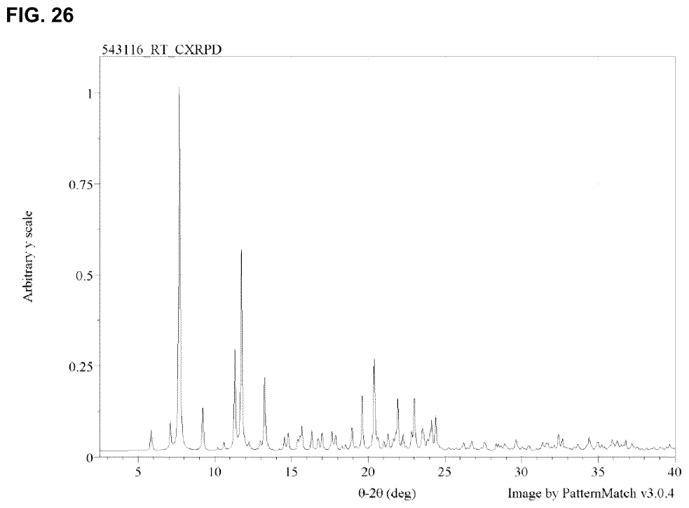

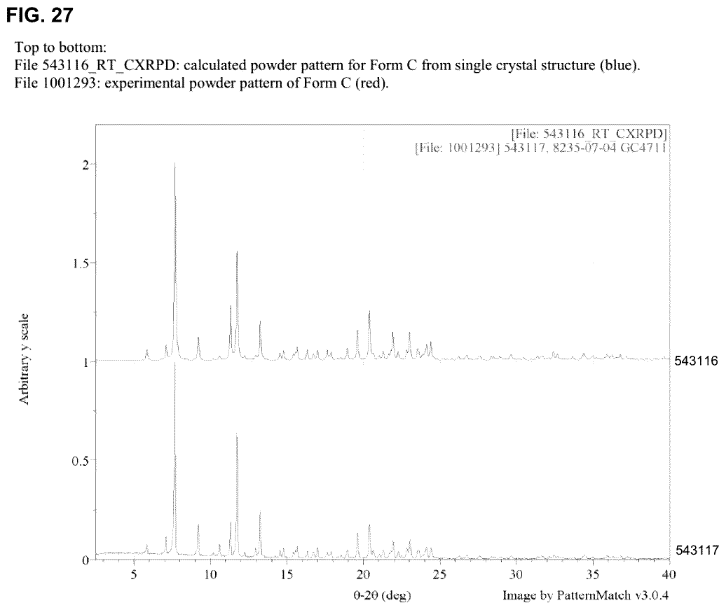

A single crystal was isolated from sample 8235-11-2 and the structure was successfully determined. The crystal system is monoclinic and the space group is P2₁. The cell parameters and calculated volume are: a=8.50076(12) Å, b=30.2477(4) Å, c=12.35774(17) Å, α=90°, β=95.5020(13)°, γ=90°, V=3162.89(7) Å³. The formula weight is 608.67 g mol⁻¹ with Z=4, resulting in a calculated density of 1.278 g cm⁻³. Further details of the crystal data and crystallographic data collection parameters are summarized in Table 16. An atomic displacement ellipsoid drawing of Form C Sesquihydrate Hemiethanolate is shown in FIG. 25. The asymmetric unit shown contains two manganese and pentaazamacrocycle molecules, four propionates, three water molecules, and one ethanol molecule. The calculated powder pattern is shown in FIG. 26 and both the calculated and experimental patterns are shown in FIG. 27.

TABLE 16
Crystal Data and Data Collection Parameters
for Form C Sesquihydrate Hemiethanolate.

| | |
|---|---|
| Empirical formula | C₂₈H₅₁MnN₅O₆ |
| Formula weight (g mol⁻¹) | 608.67 |
| Temperature (K) | 149.99(10) |
| Wavelength (Å) | 1.54184 |
| Crystal system | monoclinic |
| Space group | P2₁ |
| Unit cell parameters | |
| a = 8.50076(12) Å | α = 90° |
| b = 30.2477(4) Å | β = 95.5020(13)° |
| c = 12.35774(17) Å | γ = 90° |
| Unit cell volume (Å³) | 3162.89(7) |
| Cell formula units, Z | 4 |
| Calculated density (g cm⁻³) | 1.278 |
| Absorption coefficient (mm⁻¹) | 3.774 |
| F(000) | 1308 |
| Crystal size (mm³) | 0.35 × 0.1 × 0.03 |
| Reflections used for cell measurement | 17332 |
| θ range for cell measurement | 3.8610°-77.3140° |
| Total reflections collected | 30831 |
| Index ranges | −10 ≤ h ≤ 10; −37 ≤ |
| | k ≤ 37; −14 ≤ l ≤ 15 |
| θ range for data collection | θₘᵢₙ = 3.593°, |
| | θₘₐₓ = 77.675° |
| Completeness to θₘₐₓ | 97.8% |
| Completeness to θ_{full} = 67.684° | 99.6% |
| Absorption correction | multi-scan |
| Transmission coefficient range | 0.516-1.000 |
| Refinement method | full matrix |
| | least-squares on F² |
| Independent reflections | 11758 [Rᵢₙₜ = 0.0435, |
| | R_{σ} = 0.0448] |
| Reflections [I > 2σ(I)] | 10631 |
| Reflections/restraints/parameters | 11758/1/802 |
| Goodness-of-fit on F² | S = 1.07 |
| Final residuals [I > 2σ(I)] | R = 0.0427, R_{w} = 0.1147 |
| Final residuals [all reflections] | R = 0.0477, R_{w} = 0.1182 |
| Largest diff. peak and hole (e Å⁻³) | 0.417, −0.418 |
| Max/mean shift/standard uncertainty | 0.000/0.000 |
| Absolute structure determination | Flack parameter: −0.005(3) |

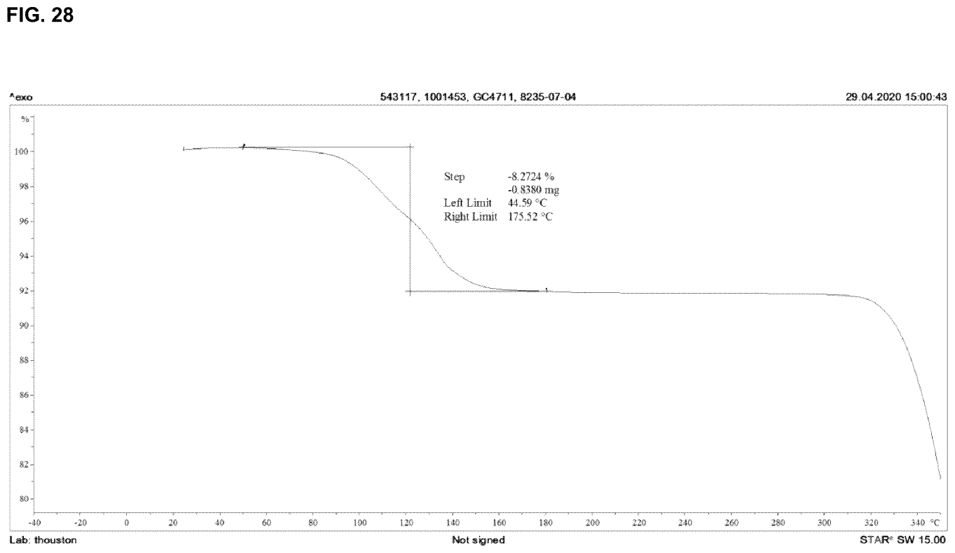

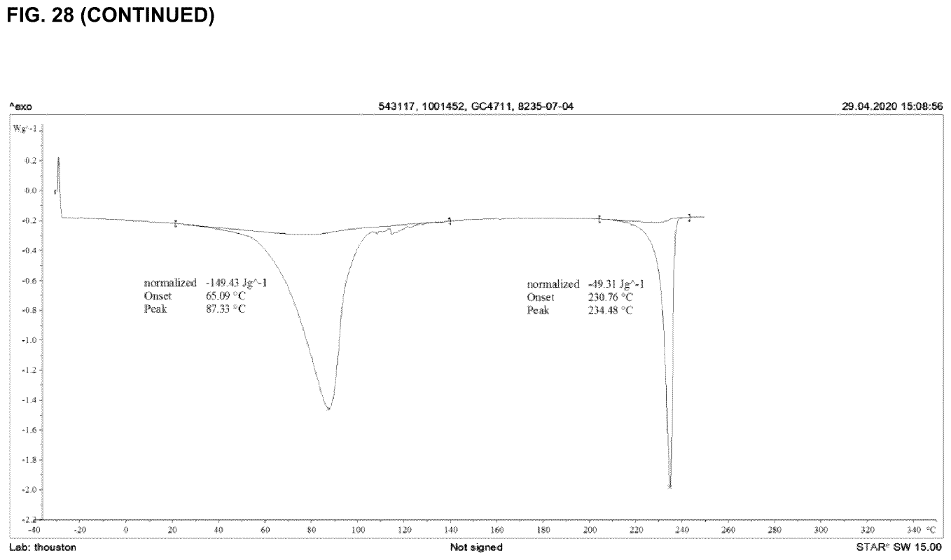

Thermograms for Form C Sesquihydrate Hemiethanolate are shown in FIG. 28. The TGA thermogram exhibits a 8.3% weight loss up to 176° C. concurrent with a broad desolvation endotherm in the DSC. This loss is consistent with the volatilization of ˜1.5 mol/mol of water and ½ mol/mol of ethanol. A final DSC endotherm with an onset of 231° C. is the concomitant melt/decomposition of the desolvated form (Form E Anhydrate). Form C was shown to partially desolvate to Form E when briefly exposed to dry nitrogen (Table 17).

TABLE 17
Physical Stability of Form C Sesquihydrate Hemiethanolate.

| Method^{a} | Result | Source | Sample | LIMS | File | Page |
|---|---|---|---|---|---|---|
| 0% RH, | C + E | 8235-07-04 | 8235-27-01 | 543387 | 1001896 | 148 |
| N₂, 10 min | | | | | | |

^{a}Times and temperatures are approximate unless noted.

c. Pattern F Solvate/Hydrate (as a Mixture with Form a Sesquihydrate)

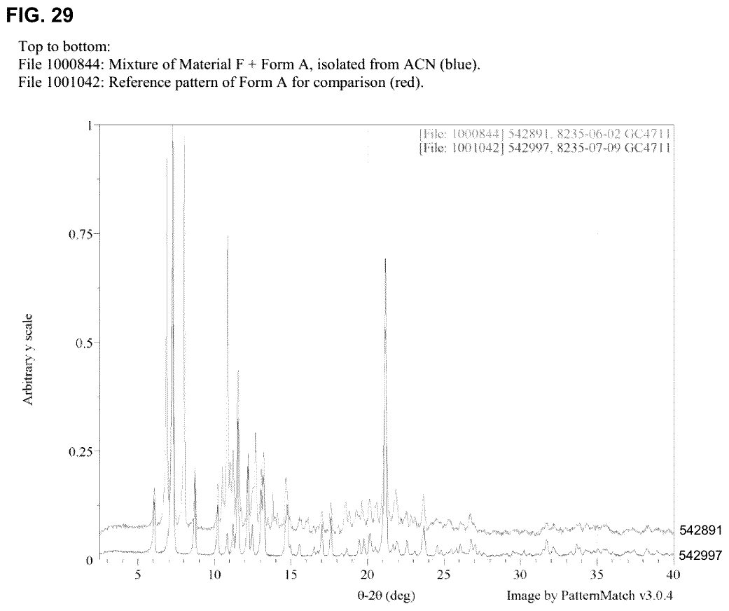

Pattern F was isolated as a mixture with Form A Sesquihydrate from evaporation experiments involving ACN. Based on the method of preparation, Pattern F may be an ACN solvate or mixed ACN/hydrate (Table 18 and FIG. 29). Pattern F is metastable and reanalysis of the mixture after 6 days by XRPD shows that conversion to Form A readily occurred at ambient laboratory conditions within that timeframe.

TABLE 18
Characterization Data for Pattern F (as a Mixture w/Form A).

| Technique | Details | Result | Sample | LIMS | File | Page |
|---|---|---|---|---|---|---|
| XRPD | — | Partem F + Form A Sesquihydrate | 8235-06-02 | 542891 | 1000844 | 84 |
| | re analyzed after 6 d | Form A Sesquihydrate | 8235-06-02 | 542891 | 1001451 | 169 |
| DSC^{a} | −30 to 250° C. | desolvation endotherm onset 65° C. | 8235-06-02 | 542891 | 1001044 | 47 |
| | | final endotherm onset of 231° C. | | | | |

^{a}Conversion of Pattern F to Form A occurs quickly at ambient conditions and may have occurred prior to data acquisition. Therefore, thermal data may not be representative of the form.

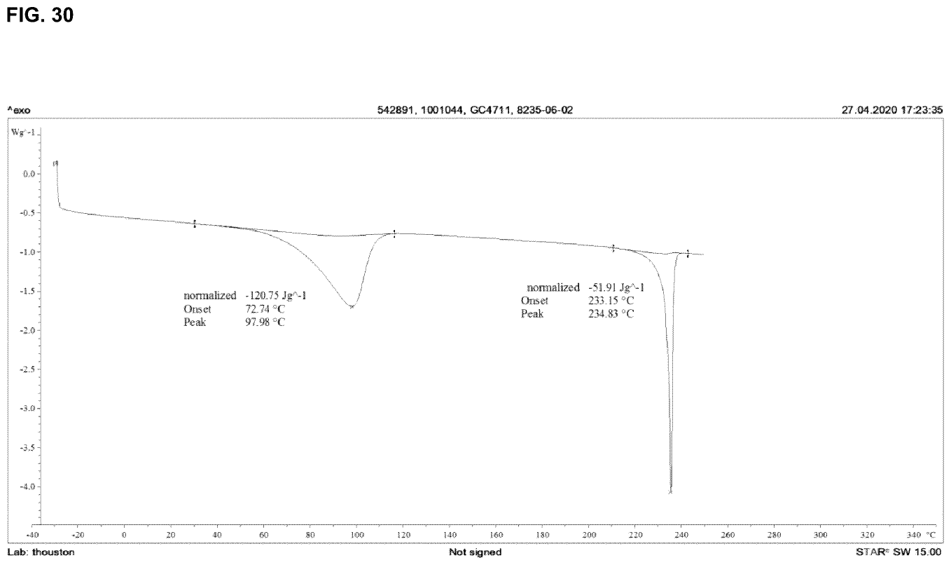

DSC analysis of the mixture was attempted; however, due to metastable nature of the form at ambient conditions, the thermogram may have been acquired after conversion and not representative (FIG. 30). The thermogram exhibits a broad desolvation endotherm followed by the concomitant melt/decomposition of the desolvated form (Form E Anhydrate) near 233° C.

d. Pattern G Solvate/Hydrate

Pattern G was isolated from experiments involving DCM/heptane (Table 19). Based on the method of preparation, Pattern G may be a DCM solvate, hydrate, or mixed DCM/hydrate. The form is physically stable at ambient up to 20 days but desolvates to Form E Anhydrate (disordered) upon exposure to elevated temperature (Table 20).

TABLE 19
Characterization Data for Pattern G.

| Technique | Details | Result | Sample | LIMS | File | Page |
|---|---|---|---|---|---|---|
| XRPD | indexed | Pattern G | 8235-06-07 | 543179 | 1001450 | 170 |
| | 20 d later | Pattern G | 8235-06-07 | 543179 | 1004133 | 171 |
| TGA | ambient—350° C. | 7.4% wt. loss up to 142° C. | 8235-06-07 | 543179 | 1004739 | 49 |
| DSC | −30 to 250° C. | broad endotherm max at 100° C., | 8235-06-07 | 543179 | 1004737 | 49 |
| | | endotherms max 198 and 227° C. | | | | |

TABLE 20
Physical Stability of Pattern G.

| Method^{b} | Result | Source | Sample | LIMS | File | Page |
|---|---|---|---|---|---|---|
| 80-110° C. 15 min | E disordered | 8235-48-08 | 8235-88-02 | 546467 | 1007357 | 134 |

^{b}Times and temperatures are approximate unless noted.

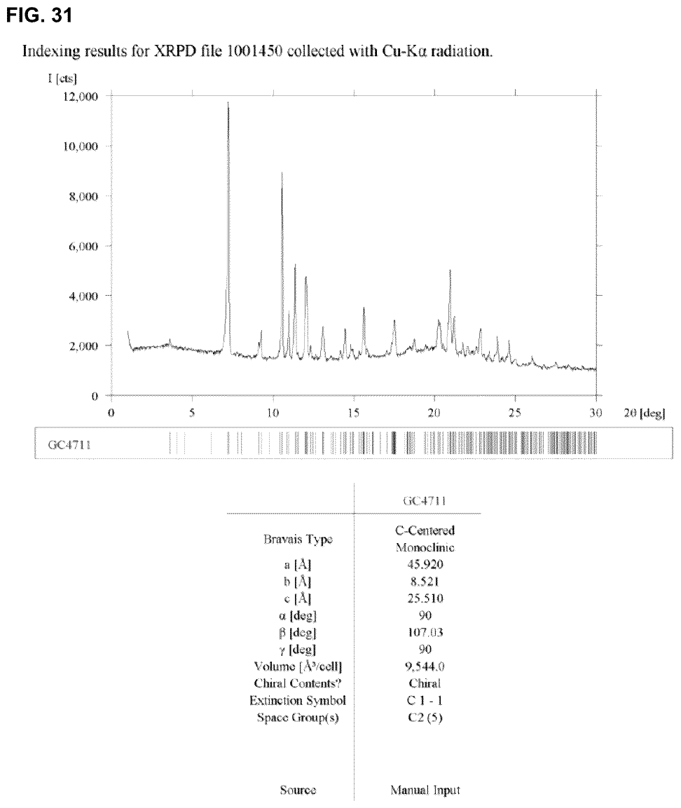

A representative XRPD pattern of Pattern G was successfully indexed by a single C-centered monoclinic unit cell (FIG. 31). Assuming a multiplicity of 12, the formula unit volume of 795 Å³ calculated from the indexing results provides a free volume of approximately 56 Å³ (relative to the volume of Form E) that can theoretically accommodate up to ⅔ mol/mol of DCM (due to symmetry, only ½, ⅓, or ⅔ moles are allowed), up to 2.5 mol/mol water (also in increments of ½ or ⅓ moles), or various combinations thereof.

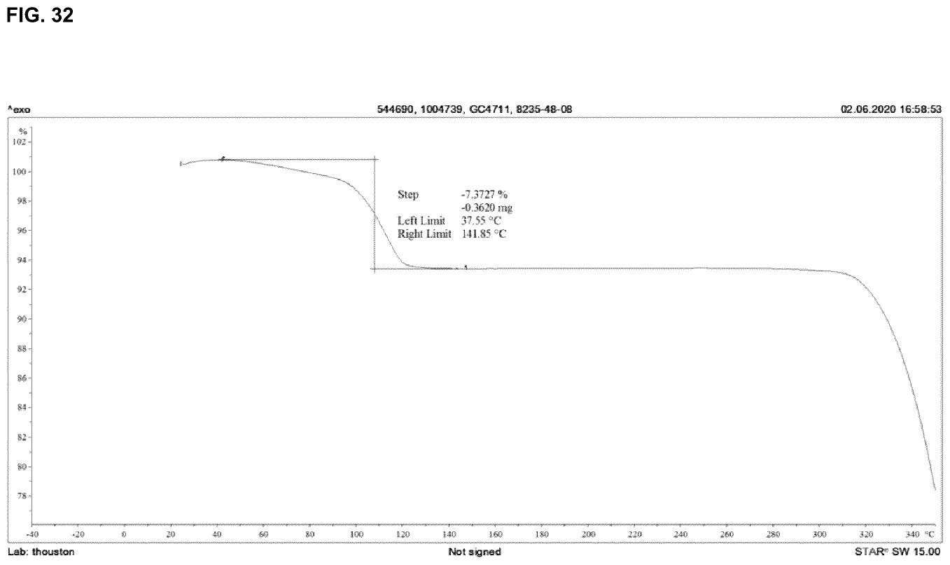

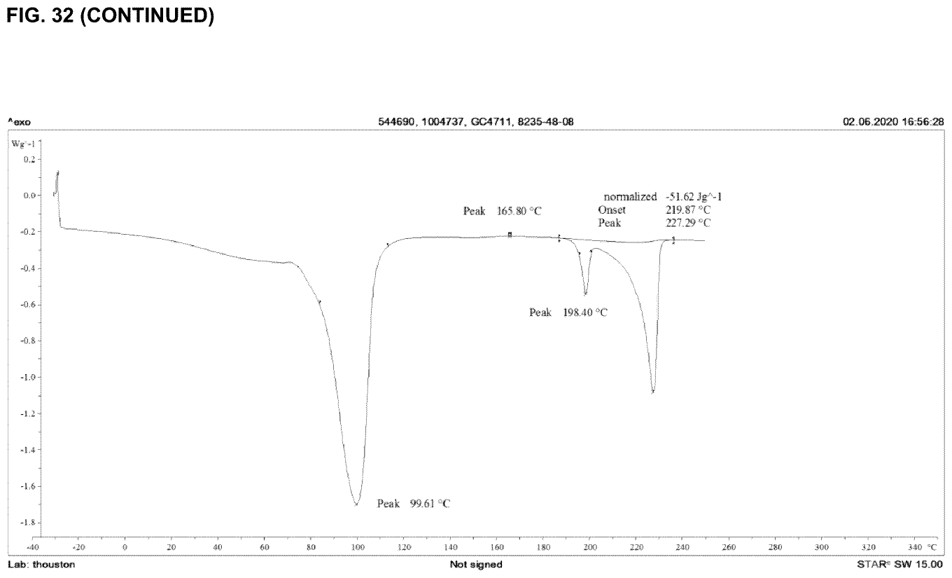

Thermograms for Pattern G are presented in FIG. 32. The TGA thermogram exhibits a 7.4% weight loss up to 142° C. concurrent with a desolvation endotherm in the DSC. The weight loss is equivalent to the loss of 0.5 mol/mol of DCM (˜7.1% wt.) or 2.5 mol/mol water (˜7.5% wt.). Alternatively, some combination thereof is also possible. The concomitant melt/decomposition of the desolvated form (Form E Anhydrate) is observed above 198° C.

e. Pattern H Solvate/Hydrate

Pattern H was isolated from a slurry in MEK/heptane at ambient temperature (Table 21). Based on the method of preparation, Pattern H may be a MEK solvate, hydrate, or mixed MEK/hydrate. Although multiple mixed solvate/hydrate probabilities are possible, the tentative indexing results and thermal characterization data fit more reasonably as a monohydrate. The form is physically stable at ambient conditions up to 13 days but, based on DSC, appears to desolvate to Form E Anhydrate upon exposure to elevated temperature. Attempts to generate additional material failed to provide Pattern H as a single crystalline phase for further characterization.

TABLE 21
Characterization Data for Pattern H.

| Technique | Details | Result | Sample | LIMS | File | Page |
|---|---|---|---|---|---|---|
| XRPD | indexed | Pattern H | 8235-07-03 | 543675 | 1002477 | 125 |
| | 13 d Inter | Pattern H | 8235-07-03 | 543675 | 1004134 | 172 |
| TGA | ambient—350 ° C. | 3.3% wt. loss up to 144° C. | 8235-07-03 | 543675 | 1007356 | 51 |
| DSC | −30 to 250° C. | broad endotherm onset 88° C. | 8235-07-03 | 543675 | 1007355 | 51 |
| | | final endotherm onset 221° C. | | | | |

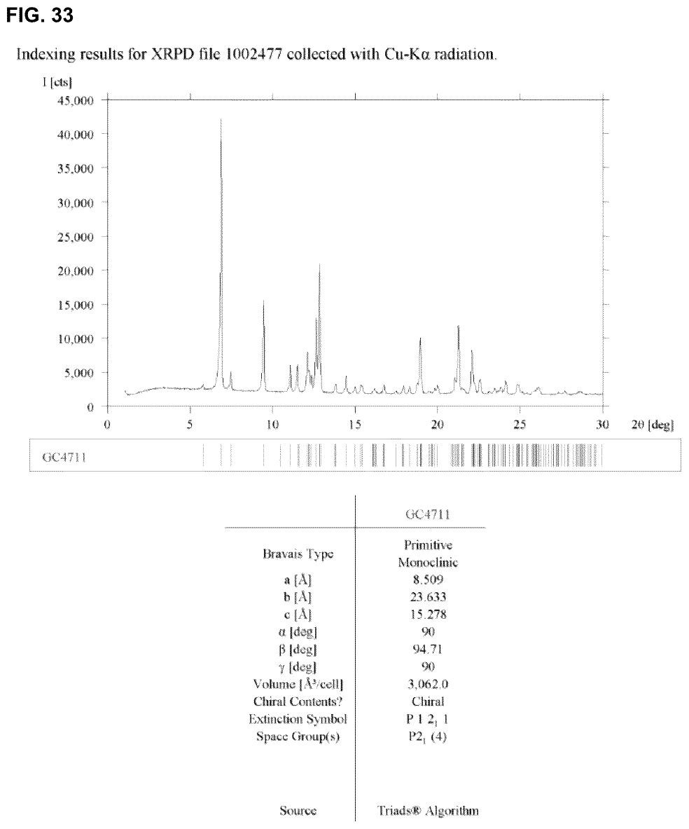

A representative XRPD pattern of Pattern H was successfully indexed by a single primitive monoclinic unit cell (FIG. 33). Assuming a multiplicity of 4, the formula unit volume of 765 Å³ calculated from the indexing results provides a free volume of approximately 26 Å³ (relative to the volume of Form E) that is large enough to only theoretically accommodate up to 1 mol/mol water [12]. A ½ of a MEK molecule is still too large to reside within the projected free volume and, due to symmetry limitations for the assumed asymmetric unit, mole fractions of less than ½ are improbable.

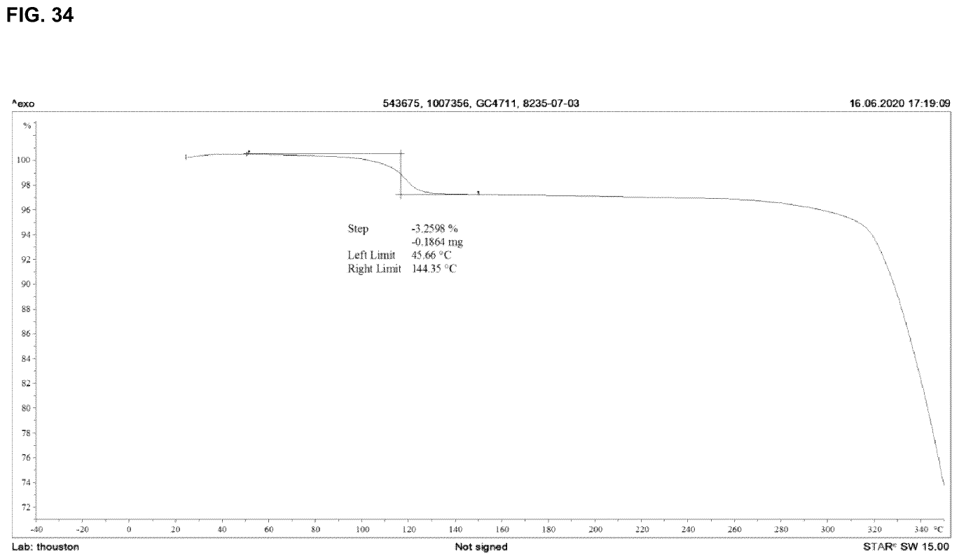

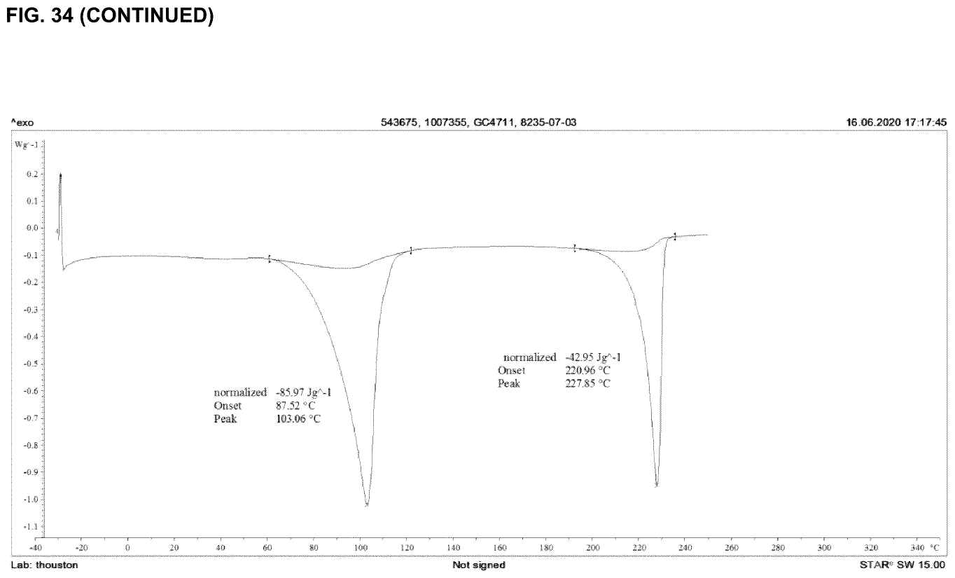

Thermograms for Pattern H are presented in FIG. 34. The TGA thermogram exhibits a 3.3% weight loss up to 144° C. concurrent with a desolvation endotherm in the DSC. The weight loss is equivalent to the loss of 0.25 mol/mol of MEK (˜3.1% wt.) or 1 mol/mol water (˜2.9% wt.). Alternatively, some combination thereof is also possible. The tentative indexing results discussed above, however, suggests that an MEK solvate or mixed solvate/hydrate is unlikely. The concomitant melt/decomposition of the desolvated form (Form E Anhydrate) is observed above 221° C.

f. Pattern I Solvate/Hydrate (as a Mixture with Form a Sesquihydrate and Form C Sesquihydrate Hemiethanolate)

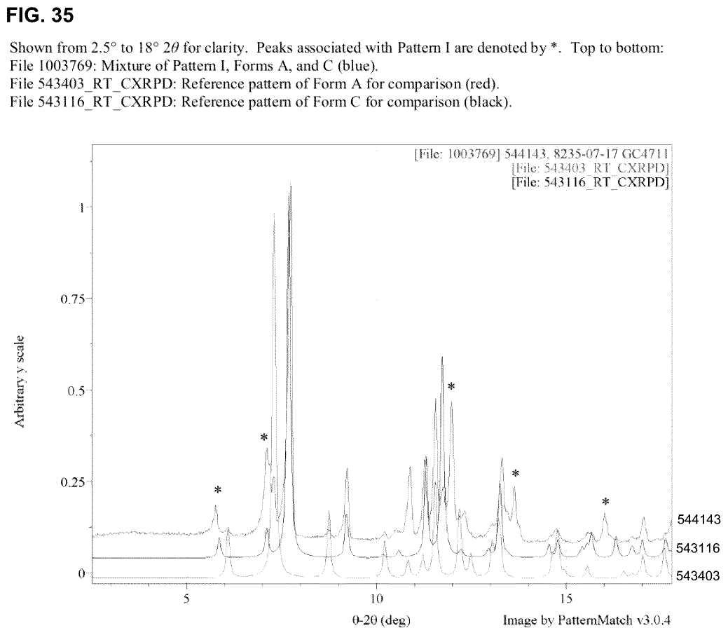

Pattern I is used denote a limited number of additional peaks in a mixture predominately composed of Form A Sesquihydrate and Form C Sesquihydrate Hemiethanolate (Table 22 and FIG. 35). The mixture was observed from evaporative experiments involving ethanol/heptane. The peak intensities for both Pattern I and Form C decreased, relative to Form A, on reanalysis by XRPD after 7 days. This suggests that both Pattern I and Form C are metastable, relative to Form A, at ambient laboratory conditions. Attempts to generate additional material failed to provide Pattern I as a single crystalline phase for further characterization.

TABLE 22
Characterization Data for Pattern I (as a Mixture w/Forms A and C).

| Technique | Details | Result | Sample | LIMS | File | Page |
|---|---|---|---|---|---|---|
| XRPD | — | Forms A, C + Pattern I | 8235-07-18 | 544142 | 1003768 | 106 |
| | 7 d later | Form A increased | 8235-07-18 | 544142 | 1004736 | 173 |
| | | and Form C and | | | | |
| | | Pattern I decrease | | | | |

g. Pattern J Solvate/Hydrate

Pattern J was isolated from experiments involving t-butanol/heptane and water (Table 23). Reproducing the experiment without adding water fails to provide Pattern J, suggesting that water is consequential in crystallizing the form. Based on the method of preparation, Pattern J may be a hydrate or mixed t-butanol/hydrate. The form is physically stable upon brief exposure to dry nitrogen but desolvates to a mixture of Forms A and E at elevated temperature (Table 24).

TABLE 23
Characterization Data for Pattern J.

| Technique | Details | Result | Sample | LIMS | File | Page |
|---|---|---|---|---|---|---|
| XRPD | indexed | Pattern J | 8235-48-06 | 544516 | 1004120 | 87 |
| TGA | ambient—350 ° C. | 8.6% wt. loss up to 122° C. | 8235-48-06 | 544516 | 1004740 | 54 |
| DSC | −30 to 250° C. | broad endotherm max 96° C. | 8235-48-06 | 544516 | 1004738 | 54 |
| | | final endotherm onset 231° C. | | | | |

TABLE 24
Physical Stability of Pattern J.

| Method | Result | Source | Sample | LIMS | File | Page |
|---|---|---|---|---|---|---|
| N₂ dried for ~5 minutes | Pattern J | 8235-48-10 | 8235-66-04 | 544696 | 1004456 | 174 |
| 80-110° C. 15 minutes | Forms E + A | 8235-48-10 | 8235-88-04 | 546469 | 1007359 | 135 |

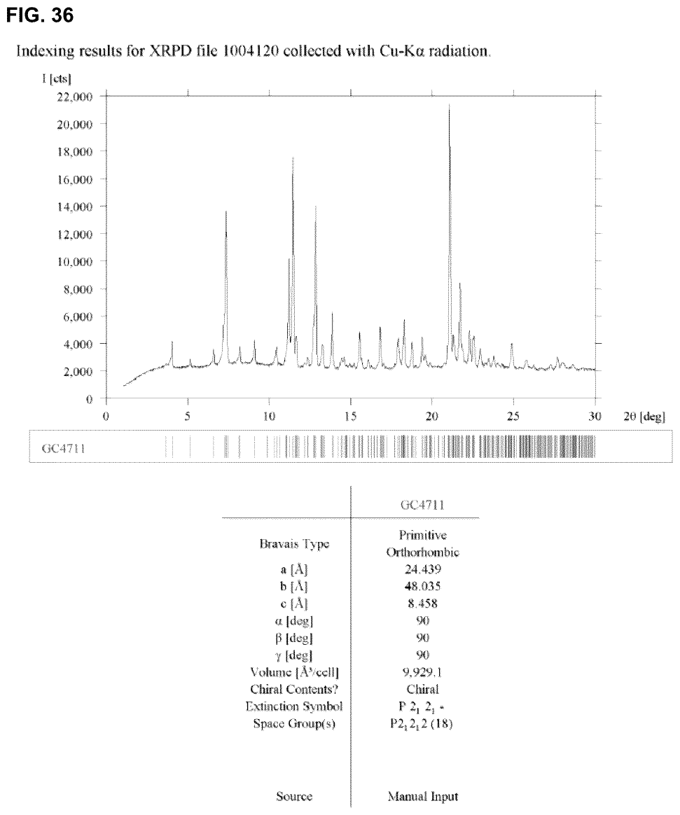

A representative XRPD pattern of Pattern J was successfully indexed by a single primitive orthorhombic unit cell (FIG. 36). Assuming a multiplicity of 12, the formula unit volume of 827 Å3 calculated from the indexing results provides a free volume of approximately 88 A3 (relative to the volume of Form E) that can theoretically accommodate up to ⅔ mol/mol of t-butanol (due to symmetry, only ½, ⅓, or ⅔ moles are allowed), up to 4 mol/mol water (also in increments of ½ or ⅓ moles), or various combinations thereof [12].

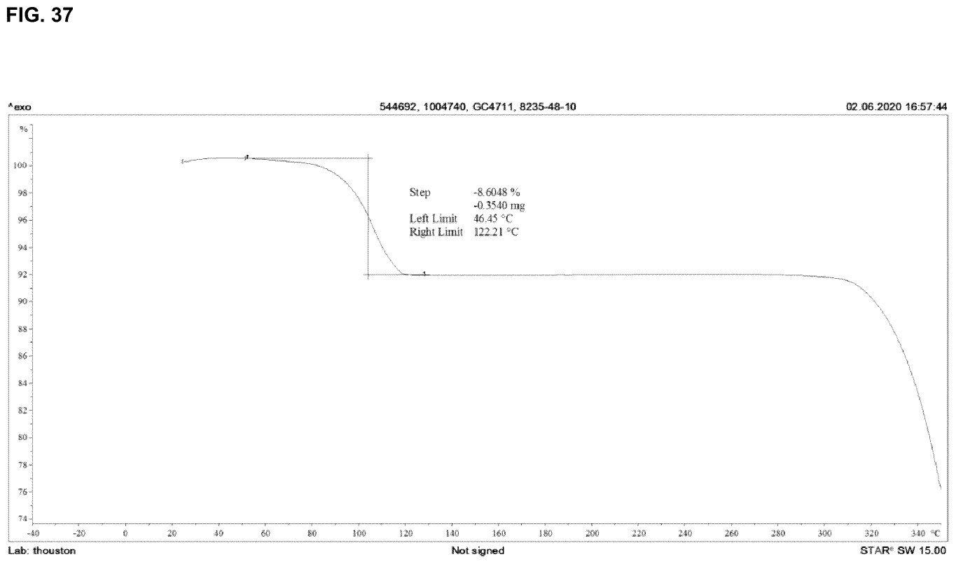

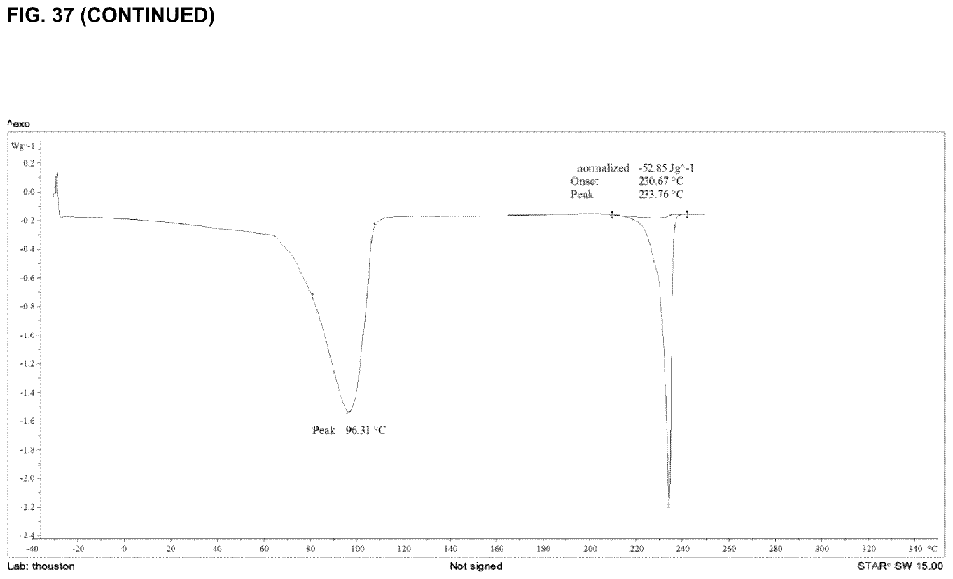

Thermograms for Pattern J are presented in FIG. 37. The TGA thermogram exhibits 8.6% weight loss up to 122° C. concurrent with a desolvation endotherm in the DSC. The weight loss is equivalent to the loss of ⅔ mol/mol of t-butanol (˜8.1% wt.) or 3 mol/mol water (˜8.8% wt.). Alternatively, some combination thereof is also possible. A final DSC endotherm with an onset of 231° C. is the concomitant melt/decomposition of the desolvated form (Form E Anhydrate).

### 4. Amorphous GC4711

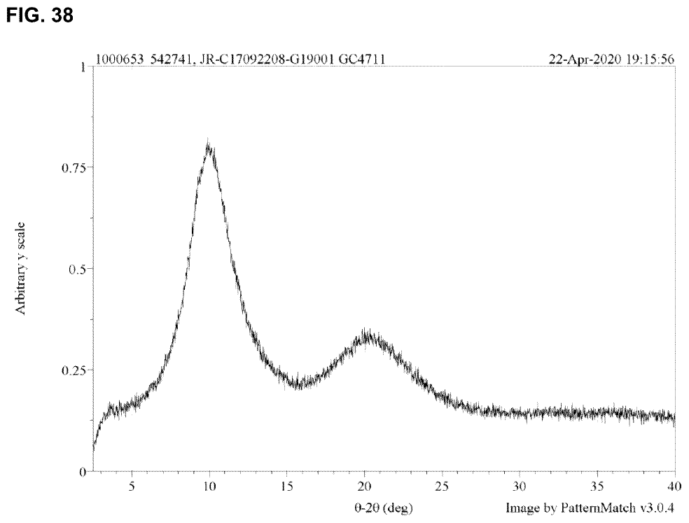

Amorphous GC4711 was received as lot JR-C17092208-G19001 (Table 25 and FIG. 38). The material is pale-yellow and treating with activated charcoal did not visually improve its color. Amorphous GC4711 is kinetically stable at 43% RH and ambient temperature for at least up to 3 days. However, the material exhibits significant hygroscopicity from 5 to 45% RH, above which crystallization to Form A Sesquihydrate occurs. The glass transition is observed near 42° C. and crystallization to Form E Anhydrate spontaneously occurs above 65° C. (Table 26).

TABLE 25
Characterization for Amorphous GC4711 Lot JR-C17092208-G19001.

| Technique | Details | | Result | LIMS | File | Page |
|---|---|---|---|---|---|---|
| XRPD | — | | amorphous | 542741 | 1000653 | 176 |
| microscopy | polarized light | | opaque, no birefringence, pale yellow | 542741 | — | — |
| TGA | ambient—350 ° C. | | 1.3% weight loss up to 114° C. | 542741 | 1000655 | 56 |
| DSC | cyclic | | 1) glass transition midpoint: 42° C., | 542741 | 1000657 | 56 |
| | 1) −30 to 100° C. | | recrystallization near 63° C., along | | | |
| | | | with volatilization | | | |
| | 2) 100 to −30° C. | | 2) no events, recrystallization complete | | | |
| | 3) −30 to 250° C. | | 3) endotherm with onset of 233° C. | | | |
| hot stage | 23.3° C. | 10° C./min | irregular glass, few | 542741 | 1000659-1 | 177 |
| microscopy | | | birefringent particles | | | |
| | 58.5° C. | | softening/liquefaction | 542741 | 1000659-2 | 177 |
| | 65.0° C. | | increase in birefringence | 542741 | 1000659-3 | 179 |
| | 69.4° C. | | recrystallization. | 542741 | 1000659-4 | 187 |
| | 72.7° C. | | | 542741 | 1000659-5 | 187 |
| | 97.7° C. | | | 542741 | 1000659-6 | 187 |
| | 184.4° C. | 20° C./min | darkening of material | 542741 | 1000659-7 | 187 |
| | 195.6° C. | | liquefaction/melt beginning | 542741 | 1000659-8 | 187 |
| | 212.4° C. | 2° C./min | melt continuation | 542741 | 1000659-9 | 187 |
| | 213.3° C. | | darkening, loss of birefringence | 542741 | 1000659-10 | 187 |
| | 217.7° C. | | melt complete | 542741 | 1000659-11 | 187 |
| | ambient | — | did not crystallize, black solids | 542741 | — | — |

| | | | | | |
|---|---|---|---|---|---|
| KF | coulometric | 4.88 wt % water content | 542741 | 1000663 | 188 |
| DVS | 5-95-5% RH | 6.14% water gain from 5to 45% RH | 542741 | 1000661 | 58 |
| | | 4.19% water loss from 45 to 55% RH | | | |
| | | 0.26% weight gain from 55 to 85% RH | | | |
| | | 21.54% gain from 85 to 95% RH | | | |
| | | 16.4% weight loss from 95 to 75% RH | | | |
| | | 2.38% weigh loss from 75 to 5% RH | | | |
| | | hysteresis observed with | | | |
| | | 5.7 wt % retained | | | |
| post DVS | 8237-01-01 | Form A | 543261 | 1001611 | 190 |
| XRPD | | | | | |

TABLE 26
Physical Stability of Amorphous
GC4711 Lot JR-C17092208-G19001.

| Method^{a} | Result | Sample | LIMS | File | Page |
|---|---|---|---|---|---|
| 90° C., 35 min, | E dis- | 8235-07-05 | 542989 | 1001003 | 136 |
| N₂ | ordered | | | | |
| 125° C., 45 | B dis- | 8235-07-06 | 542998 | 1001043 | 137 |
| min, N₂ | ordered | | | | |
| 43% RH, RT, 3 | amorphous | 8235-07-08 | 543119 | 1001295 | 138 |
| days | | | | | |
| 75% RH, RT, | A | 8235-07-07 | 543118 | 1001294 | 139 |
| 3 days | | | | | |
| 85% RH, RT, | A | 8235-48-01 | 544515 | 1004119 | 140 |
| 3 days | | | | | |

^{a}Times and temperatures are approximate unless noted.

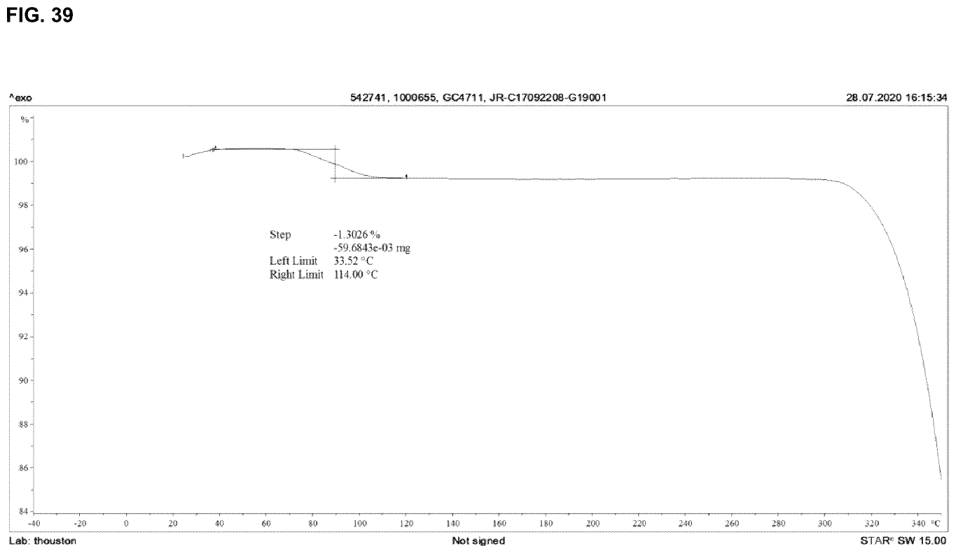

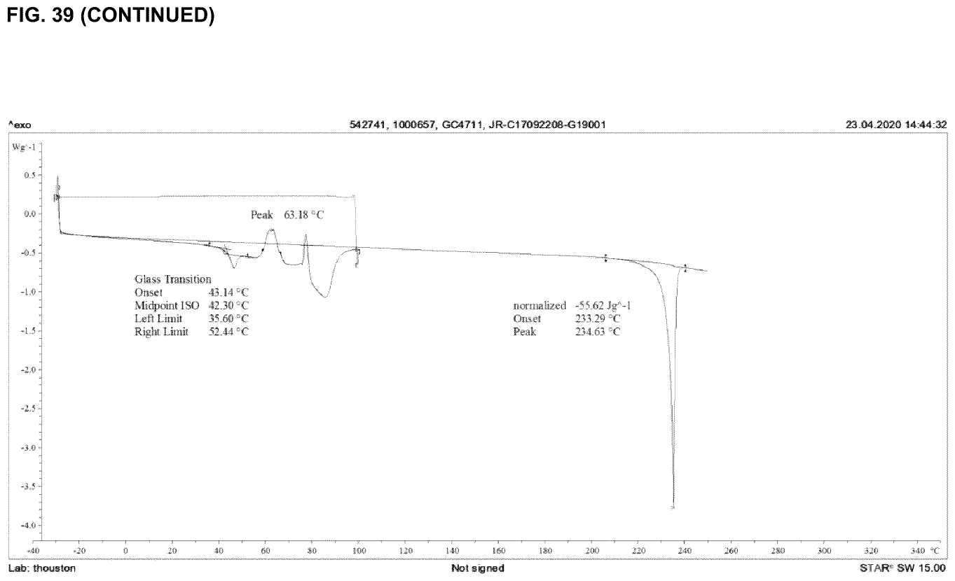

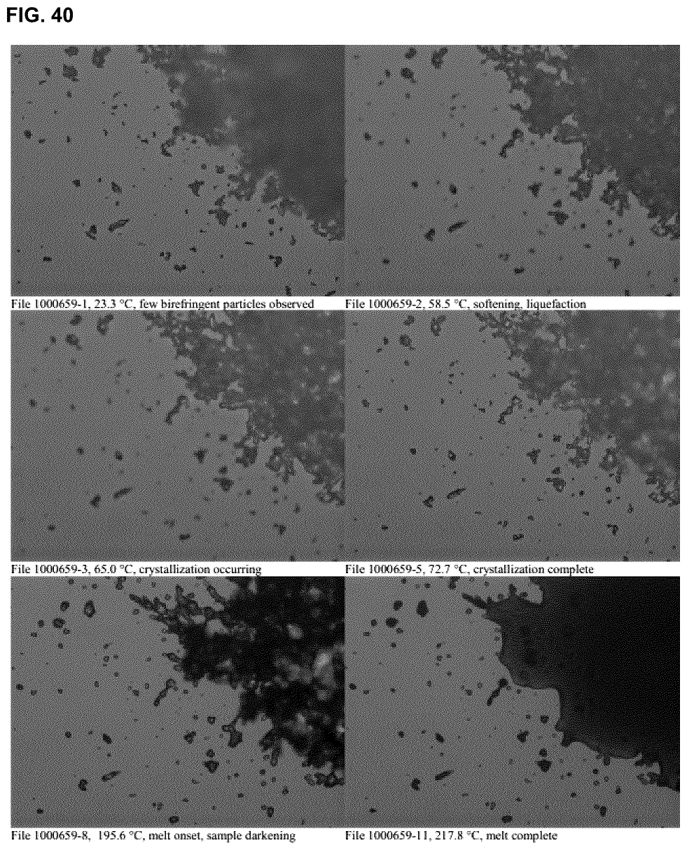

Thermograms are provided in FIG. 39. The TGA thermogram exhibits a 1.3% weight loss up to 114° C. A cycling DSC experiment was conducted in an attempt to volatilize residual moisture in the first cycle and measure a glass transition of the material in its driest state in the second heating cycle; however, the material spontaneously crystallized above 65° C. in the first heating cycle concomitantly with the volatilization of residual moisture. Regardless, a glass transition near 42° C. (midpoint) was measured. A final DSC endotherm with an onset of 231° C. is the concomitant melt/decomposition of the crystalline form (Form E Anhydrate). Hot stage microscopy, provided in FIG. 40, confirms the thermal events and suggests that decomposition likely occurs at a lower temperature (˜184° C.) than the melt onset when not protected from oxidation.

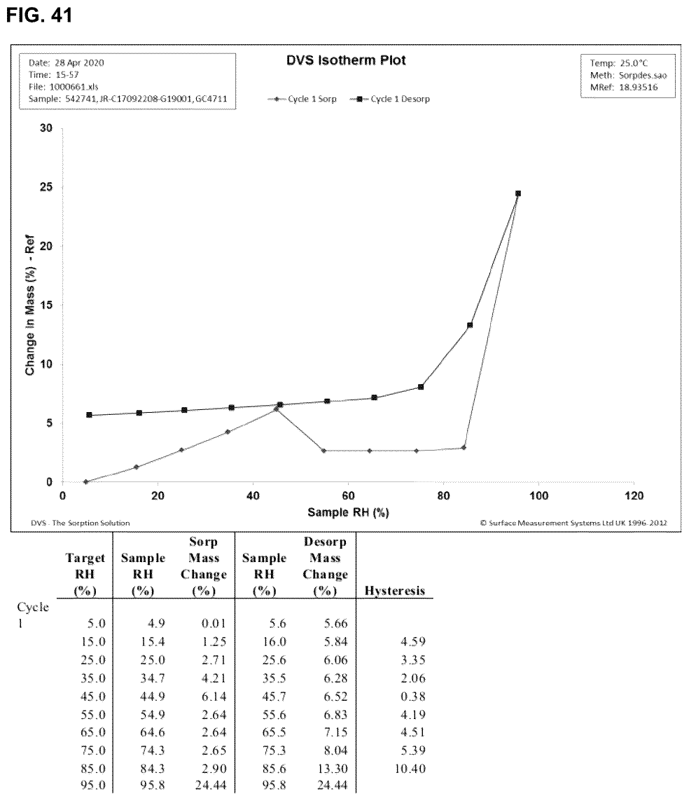

The DVS isotherm (FIG. 41) indicates amorphous GC4711 exhibits significant hygroscopicity from 5 to 45% RH]. The material gains more than 6 wt % before it effloresces 4 wt %. Efflorescence is the process of crystallization and expulsion of water from the crystallized material. The crystalline material retains approximately 2 wt % at 55% RH and exhibits low hygroscopicity up to 85% RH. However, significant hygroscopicity is presented above 85% RH with more than 21 wt % gained. The instrument timed-out at 85% RH and above, which suggests that additional weight gain would likely occur if left to equilibrate at these conditions further. Significant hysteresis was observed on desorption with moisture retention of more than 5 wt % once completed. The material recovered from the DVS experiment was identified as Form A Sesquihydrate by XRPD, confirming that crystallization occurred during the experiment.

Karl Fischer titration measured 4.88 wt % water. The KF analyst noted that the sample appeared hygroscopic during sample preparation. The water content is consistent with the significant hygroscopicity observed between 5 and 45% RH by DVS, above.

### Example 3

### Conclusions

GC4711 readily forms hydrates and mixed solvate/hydrates. Amorphous GC4711 and ten unique crystalline materials were observed.

Form E Anhydrate, the only anhydrous form identified, exhibits a concomitant melt/decomposition onset near 234° C. and low hygroscopicity from 5 to 45% RH. However, Form E is significantly hygroscopic above 45% RH, where it hydrates to Form A Sesquihydrate. Form E is formed through the crystallization of amorphous GC4711 or desolvation/dehydration of all solvated/hydrated forms at either elevated temperature or 0% RH.

Form A Sesquihydrate was the most commonly observed form. Pattern D Dihydrate could only be reproduced by seeding saturated 97:03 v/v EtOAc/water solutions. Both hydrated forms exhibit low hygroscopicity from 5 to 85% RH and significant hygroscopicity above 85% RH. Although less stable than Pattern D at humidity higher than 23% RH, Form A is kinetically stable in the solid state at these conditions within the time-frame evaluated.

Dehydration of both hydrated forms to Form E Anhydrate occurs at 0% RH. Form A Sesquihydrate was shown to be the prevailing hydrate, relative to Pattern D Dihydrate, at 11% RH, while Pattern D Dihydrate was the prevailing hydrate at 75% RH and above. Relatively slow kinetics of conversion in the solid state prevented reaching true equilibrium at 43% RH. However, the aw experiments confirm that Pattern D Dihydrate is the prevailing hydrate at and above 0.23 aw (equivalent to 23% RH). Pattern K, a higher hydrate precipitated from highly concentrated aqueous solutions, was not included in the relative hydrate stability assessment. Partial dehydration to a mixture of Form A Sesquihydrate and Pattern D Dihydrate occurred under brief exposure to dry nitrogen and suggests that Pattern K is less stable than either form at that condition. Tentatively, Pattern K is assumed to be prevalent only near aw=1 but remains unconfirmed.

Form C Sesquihydrate Hemiethanolate and Materials F through J are purported mixed solvate/hydrates. Several were isolated as mixtures with other forms. Most appear metastable at ambient conditions or under brief exposure to dry nitrogen. Regardless, all were shown to desolvate/dehydrate to Form E at elevated temperature.

### Example 4

### Additional GC4711 Crystalline Form Screening and Selection

Additional crystalline form screen of GC4711 was performed. In this study, GC4711 was studied in terms of solvent equilibration and evaporation. Additionally, addition of anti-solvent and crystallization from hot solutions were investigated. Water activity, and water sorption/desorption was carried out to determine the relative stability of those forms encountered.

This crystalline form screening for GC4711 was performed with batch PS04106-15-G-WET (Tables 27 and 28).

TABLE 27
Starting material.

| Compound ID | GC4711 | |
|---|---|---|
| Batch No | PS04106-15-G-WET | Polymorph: Weak |
| | | crystal |

| | |
|---|---|
| Received | 36.8 g |
| sample size | |
| C-Code | C17092208-G |

TABLE 28
Properties of the starting material as received.

| Parameter | Method | Result |
|---|---|---|
| Purity | HPLC | 99.89% |
| X-ray diffraction | 3-40° (2 theta) | Weak crystal |
| DSC melting onset | DSC, 10° C./min | 229.46° C.; 45.38 J/g |
| and enthalpy | | |
| Thermogravimetry | TGA, 10° C./min | 1.44% at 120° C. |
| Morphology | PLM | Weak crystal |
| KF | Coulometric | 0.87% |

Polymorphic behaviors of this compound were investigated by equilibration, evaporation, precipitation by addition of anti-solvent and crystallization from hot saturated solution experiments. Relative stability of identified crystalline forms was investigated by water activity study, water sorption and desorption experiments. During this study, hydrate forms of compound, Pattern A, Pattern B, Pattern C and Pattern D were identified. In addition, anhydrous form, Pattern E, was obtained. Relations among these crystalline forms were summarized below:

| Polymorph | Screening experiment | Comments |
|---|---|---|
| Pattern A | Anti-solvent experiments | Convert to Pattern E after TGA |
| | Slow evaporation experiments (Without N₂) | heating to 150° C. |
| | Slow evaporation(Protected with N₂): Acetone, | Convert to Pattern D after DVS |
| | MTBE, THF | test |
| | Fast cooling experiments | Stable form under <30% |
| | Equilibration: Heptane (for 2 weeks) | R.H. condition |
| Pattern B | Slow cooling: EtOAc and MEK | Low crystallinity |
| | Slow evaporation (Protected with N₂): EtOAc, IPAC, | Convert to Pattern E after TGA |
| | MEK, DCM | heating to 150° C. |
| Pattern C | Slow evaporation (Protected with N₂): EtOH, IPA | Low crystallinity |
| | | Convert to Pattern E after TGA |
| | | heat to 150° C. |
| Pattern D | Water activity experiment: Water activity 0.3~0.6 | Stable form under >30% R.H. |
| | | condition |
| Pattern E | Pattern A, Pattern B and Pattern C after TGA test | Hygroscopic |

Water sorption/desorption behaviors of Pattern A and Pattern D were investigated by DVS at 25° C. Pattern A is hygroscopic with 65% water uptake from 40% to 95% RH. After two sorption/desorption cycles, Pattern A converts to Pattern D. Pattern D is hygroscopic with 16% water uptake from 40% to 95% RH. No form changes after two sorption/desorption cycles.

Hydrate Pattern D is recommend for further development. Hydrate Pattern D is stable under >30% R.H conditions.

### Test Conditions

### (1) Approximate Solubility of the Starting Material at 25° C. and 50° C. (Table 29)

About 10 mg of drug substance was weighed to a 2 mL glass vial and aliquot of 20 μL of each solvent was added to determine solubility at 25° C. About 10 mg of drug substance was weighed to a 2 mL glass vial and aliquot of 20 μL of each solvent will be added to determine solubility at 50° C. Approximate solubility was determined by visual observation.

TABLE 29
Approximate solubility at 25° C. and 50° C.

| Exp. | | Solubility (mg/mL) | |
|---|---|---|---|
| ID | Solvent | 25° C. | 50° C. |
| SL1 | Water | S > 314.5 | S > 337.5 |
| SL2 | Methanol | S > 306.0 | S > 332.0 |
| SL3 | Ethanol | S > 322.5 | S > 281.5 |
| SL4 | 2-Propanol | S > 285.5 | S > 292.5 |
| SL5 | Ethyl acetate | 38.8 < S < 94.1 | 82.6 < S < 185.8 |
| SL6 | Isopropyl acetate | 27.1 < S < 49.8 | 69.4 < S < 156.3 |
| SL7 | Acetone | 76.4 < S < 267.5 | 147.8 < S < 295.5 |
| SL8 | Methyl ethyl ketone | 49.2 < S < 84.3 | 158.3 < S < 316.5 |
| SL9 | t-Butyl methyl ether | 3.7 < S < 5.4 | 5.9 < S < 7.5 |
| SL10 | 1,4-Dioxane | 175.5 < S < 351.0 | S > 259.5 |
| SL11 | Tetrahydrofuran | S > 319.0 | S > 319.0 |
| SL12 | Acetonitrile | 97.6 < S < 341.5 | S > 315.5 |
| SL13 | Toluene | 26.3 < S < 48.3 | 79.2 < S < 178.3 |
| SL14 | Heptane | S < 3.7 | S < 3.7 |
| SL15 | Dichloromethane | S > 297.5 | S > 278.5 |

(2) Equilibration with Solvents at 25° C. for 2 Weeks

About 50 mg of drug substance was equilibrated in suitable amount of solvent at 25° C. for 2 weeks with a stirring plate (Table 30). Obtained suspension was filtered. The solid part (wet cake) was investigated by XRPD. When differences were observed, additional investigations were performed (e.g. DSC, TGA. FT-IR, PLM/SEM, etc.).

TABLE 30
Equilibration with solvents at 25° C. for 2 weeks.

| Exp. | | | DSC/TGA if | |
|---|---|---|---|---|
| ID | Solvent | XRPD | XRPD changes | Comments |
| EQ1 | EtOAc | // | // | API degradation, |
| | | | | purity 75.69% |
| EQ2 | IPAC | // | // | API degradation, |
| | | | | purity 84.49% |
| EQ3 | Acetone | // | // | Not enough for test |
| EQ4 | MEK | // | // | Not enough for test |
| EQ5 | MTBE | // | // | API degradation, |
| | | | | purity 88.12% |
| EQ6 | Heptane | Pattern | − | Purity 99.02% |
| | | A | | |
| EQ7 | Toluene | Clear | // | API was soluble |
| | | | | in the solvent |
| EQ8 | Heptane/EtOAc, | // | // | API degradation, |
| | 80/20 | | | purity 92.64% |
| EQ9 | Heptane/EtOH, | Clear | // | API was soluble |
| | 80/20 | | | in the solvent |
| EQ10 | Heptane/IPA, | Clear | // | API was soluble |
| | 80/20 | | | in the solvent |
| EQ11 | Heptane/Acetone, | // | // | API degradation, |
| | 80/20 | | | purity 94.83% |
| EQ12 | MTBE/EIOAc, | // | // | API degradation, |
| | 80/20 | | | purity 89.32% |
| EQ13 | MTBE/EIOH, | Clear | // | API was soluble |
| | 80/20 | | | in the solvent |
| EQ14 | MTBE/IPA, | Clear | // | API was soluble |
| | 80/20 | | | in the solvent |
| EQ15 | MTBE/Acetone, | // | // | API degradation, |
| | 80/20c | | | purity 87.39% |

Explanation
“−”: No change detected.
“+”: Change detected.
“//”: Not carried out.

About 50 mg of drug substance was equilibrated in 0.2 mL of solvent at 25° C. for 1 week with a stirring plate, protected by N₂ (Table 31). The solid part (wet cake) was investigated by XRPD. If differences are observed, additional investigation will be performed (e.g. DSC, TGA, HPLC, etc.)

TABLE 31
Equilibration with solvents at 25° C. for 5 days (protected by N₂).

| | | | DSC/TGA | |
|---|---|---|---|---|
| Exp. | | | if XRPD | |
| ID | Solvent | XRPD | changes | Comments |
| EQ1 | EtOAc | // | // | API degradation, purity 97.78% |
| EQ2 | IPAC | // | // | API degradation, purity 98.30% |
| EQ3 | Acetone | // | // | API degradation, purity 98.41% |
| EQ4 | MEK | // | // | API degradation, purity 97.52% |
| EQ5 | MTBE | // | // | API degradation, purity 98.62% |
| EQ6 | Heptane | Pattern A | − | API degradation, purity 99.85% |
| EQ7 | Toluene | // | // | API degradation, purity 92.88% |

(3) Equilibration with Solvents at 50° C. for 1 Week

About 50 mg of drug substance was equilibrated in minimal amount of solvent at 50° C. for 1 week with a stirring plate (Table 32). Obtained suspension was filtered. The solid part (wet cake) was investigated by XRPD. When differences were observed, additional investigations were performed (e.g. DSC, TGA. FT-IR, PLM/SEM, etc.).

TABLE 32
Equilibration with solvents at 50° C. for 1 week.

| Exp. | | | DSC/TGA If | |
|---|---|---|---|---|
| ID | Solvent | XRPD | XRPD changes | Comments |
| EQ1 | MTBE | // | // | API degradation, purity 92.26% |
| EQ2 | Heptane | // | // | API degradation, purity 94.65% |
| EQ3 | Heptane/EtOAc, 80/20 | // | // | API degradation, purity 85.84% |
| EQ4 | Heptane/EtOH, 80/20 | Clear | // | API was soluble in the solvent |
| EQ5 | Heptane/IPA, 80/20 | Clear | // | API was soluble in the solvent |
| EQ6 | Heptane/Acetone, 80/20 | // | // | API degradation, purity 95.63% |
| EQ7 | MTBE/EtOAc, 80/20 | // | // | API degradation, purity 87.67% |
| EQ8 | MTBE/EtOH, 80/20 | Clear | // | API was soluble in the solvent |
| EQ9 | MTBE/IPA, 80/20 | Clear | // | API was soluble in the solvent |
| EQ10 | MTBE/Acetone, 80/20 | // | // | API degradation, purity 93.80% |

Explanation
“−”: No change detected.
“+”: Change detected.
“//”: Not carried out.

### (4) Precipitation by Addition of Anti-Solvent (Table 33)

About 50 mg of drug substance was dissolved in a good solvent. Anti-solvent was added into the obtained solutions slowly. Precipitates were collected by filtration. The solid part (wet cake) was investigated by XRPD. When differences were observed, additional investigations were performed (e.g. DSC, TGA. FT-IR, PLM/SEM, etc.).

Scale up: About 200 mg of drug substance was dissolved in EtOAc. Heptane was added to the obtained solutions. The solid part (wet cake) was investigated by XRPD. When differences were observed, additional investigations were performed (e.g. DSC, TGA. FT-IR, PLM/SEM, etc.)

TABLE 33
Precipitation by addition of anti-solvent.

| | | | | DSC/TGA | |
|---|---|---|---|---|---|
| Exp. | | Anti- | | if XRPD | |
| ID | Solvent | solvent | XRPD | changes | Comments |
| AS1 | EtOAc | Heptane | Pattern A | − | White solid, |
| | | | | | purity 99.95% |
| AS2 | EtOH | | Clear | // | API was soluble |
| | | | | | in the solvent |
| AS3 | Acetone | | Pattern A | − | White solid, |
| | | | | | purity 99.93% |
| AS4 | MEK | | Pattern A | − | White solid |
| AS5 | IPA | | Pattern A | − | White solid |
| AS6 | THF | | Pattern A | − | White solid |
| AS7 | IPAC | | Clear | // | API was soluble |
| | | | | | in the solvent |
| AS8 | ACN | MTBE | Clear | // | API was soluble |
| AS9 | EtOH | | | // | in the solvent |
| AS10 | MeOH | | | // | |
| AS11 | IPA | | | // | |
| AS12 | Acetone | | | // | |
| AS13 | THF | | | // | |
| AS14 | EtOAc | | | // | |

Explanation
“−”: No change detected.
“+”: Change detected.
“//”: Not carried out.

### (5) Crystallization at Room Temperature by Slow Evaporation

Combined with approximate solubility experiment, solubility samples were filtered by 0.45 μm nylon filter. Obtained solutions were slow evaporated at ambient condition. Solid residues were examined for their polymorphic form (Table 34).

TABLE 34
Crystallization at room temperature by slow evaporation.

| | | | DSC/TGA if | |
|---|---|---|---|---|
| Exp. ID | Solvent | XRPD | XRPD changes | Comments |
| SE1 | Water | Clear | // | API was |
| | | | | soluble |
| | | | | in the solvent |
| SE2 | Methanol | Not sufficient | // | // |
| | | solids | | |
| SE3 | Ethanol | Not sufficient | // | // |
| | | solids | | |
| SE4 | IPA | Not sufficient | // | // |
| | | solids | | |
| SE5 | EtOAc | Pattern A | − | White solid |
| SE6 | IPAC | Pattern A | − | White solid |
| SE7 | Acetone | Not sufficient | // | // |
| | | solids | | |
| SE8 | MEK | Not sufficient | // | // |
| | | solids | | |
| SE9 | MTBE | Not sufficient | // | // |
| | | solids | | |
| SE10 | 1,4-Dioxane | Not sufficient | // | // |
| | | solids | | |
| SE11 | DCM | Pattern A | − | White solid |
| SE12 | Toluene | Pattern A | − | White solid |
| SE13 | ACN | Not sufficient | // | // |
| | | solids | | |
| SE14 | THF | Not sufficient | // | // |
| | | solids | | |

Explanation
“−”: No change detected.
“+”: Change detected.
“//”: Not carried out.

Scale up: About 200 mg of drug substance will be dissolved in EtOAc. Obtained solutions shall be exposed to ambient condition (protected by N₂) to allow slow evaporation of solvents (Table 35).

TABLE 35
Crystallization at room temperature by slow evaporation (protect with N₂).

| | | | DSC/TGA if XRPD | |
|---|---|---|---|---|
| Exp. ID | Solvent | XRPD | changes | Comments |
| SE1 | Methanol | Pattern A + | // | Purity 99.92% |
| | | Pattern C^{LC} | | |
| SE2 | Ethanol | Pattern C^{LC} | // | Purity 99.65% |
| SE3 | IPA | Pattern C^{LC} | Onset: 69.3° C.(134.3 J/g); | Purity 99.63% |
| | | | 223.6° C. (35.0 J/g) | |
| SE4 | EtOAc | Pattern B | Onset: 67.9° C.(114.1 J/g); | Purity 99.42% |
| | | | 222.9° C. (46.3 J/g) | |
| SE5 | IPAC | Pattern B | Onset: 68.2° C.(98.8 J/g); | Purity 99.90% |
| | | | 228.4° C. (48.6 J/g) | |
| SE6 | Acetone | Pattern A | − | Purity 99.39% |
| SE7 | MEK | Pattern B | // | Purity 99.33% |
| SE8 | MTBE | Pattern A | − | Purity 99.88% |
| SE9 | 1,4- | // | // | API degradation, purity |
| | Dioxane | | | 97.76% |
| SE10 | DCM | Pattern B | // | Purity 99.93% |
| SE11 | Toluene | // | // | API degradation, purity |
| | | | | 96.47% |
| SE12 | ACN | // | // | API degradation, purity |
| | | | | 98.98% |
| SE13 | THF | Pattern A | − | Purity 99.12% |

Explanation
“−”: No change detected.
“+”: Change detected.
“//”: Not carried out.

(6) Crystallization from Hot Saturated Solutions by Slow Cooling (Table 36)

Approximate 100 mg of drug substance was dissolved in the minimal amount of selected solvents at 50° C. Obtained solutions were cooled to 5° C. at 0.1° C./min. Precipitates were collected by filtration. The solid part (wet cake) was investigated by XRPD. When differences were observed, additional investigations were performed (e.g. DSC, TGA. FT-IR, PLM/SEM, etc.).

Scale up: About 200 mg of drug substance will be dissolved in MEK. Obtained solutions will be applied with cooling rate of 0.1° C./min for slow cooling.

TABLE 36
Crystallization from hot saturated solutions by slow cooling.

| Exp. | | | DSC/TGA if XRPD | |
|---|---|---|---|---|
| ID | Solvents | XRPD | changes | Comments |
| SC1 | EtOAc | Pattern B | Onset: 78.7° C.(70.2 J/g); | White solid, purity 100% |
| | | | 234.8° C. (54.9 J/g) | |
| SC2 | Acetone | Clear | // | API was soluble in the solvent |
| SC3 | MEK | Pattern B | Onset: 60.0° C.(110.4 J/g); | White solid, purity 100% |
| | | | 235.0° C. (58.1 J/g) | |
| SC4 | IPAC | Clear | // | API was soluble in the solvent |
| SC5 | IPA | Clear | // | API was soluble in the solvent |
| SC6 | Water | Clear | // | API was soluble in the solvent |

Explanation
“−”: No change detected.
“+”: Change detected.
“//”: Not carried out.

(7) Crystallization from Hot Saturated Solutions by Fast Cooling (Table 37)

Approximate 100 mg of drug substance was dissolved in the minimal amount of selected solvents at 50° C. Obtained solutions were put into an ice bath. Precipitates were collected by filtration. The solid part (wet cake) was investigated by XRPD. When differences were observed, additional investigations were performed (e.g. DSC, TGA. FT-IR, PLM/SEM, etc.). When no precipitation was obtained, the solutions were put in −20° C. freezer for crystallization.

TABLE 37
Crystallization from hot saturated solutions by fast cooling.

| | | | DSC/TGA | |
|---|---|---|---|---|
| | | | if XRPD | |
| Exp. ID | Solvents | XRPD | changes | Comments |
| FC1 | EtOAc | Pattern A | − | White solid |
| FC2 | Acetone | Clear | // | API was soluble |
| | | | | in the solvent |
| FC3 | MEK | Pattern A | − | Yellow solid, 94.19% |
| FC4 | IPAC | Pattern A | − | White solid |
| FC5 | IPA | Clear | // | API was soluble |
| | | | | in the solvent |
| FC6 | Water | Clear | // | API was soluble |
| | | | | in the solvent |

Explanation
“−”: No change detected.
“+”: Change detected.
“//”: Not carried out.

### (8) Behavior Under Heating and Cooling (Table 38)

Polymorphic behavior was investigated by two different heating-cooling cycle of DSC. Cycle 1: 30° C. to melt at 10° C./min; melt to −20° C. at 20° C./min; reheat to melt/decomposition at 10° C./min. Cycle 2:30° C. to melt at 10° C./min; melt to −20° C. at 2° C./min; reheat to melt/decomposition at 10° C./min.

TABLE 38
Behavior under heating and cooling.

| Exp. ID | Heating rate | Thermal events |
|---|---|---|
| HCH1 | Cycle 1: 30° C. to 240° C. at 10° C./min; | Endothermic peak Onset: 51.3° C.(8.07 J/g); |
| | 240° C. to −20° C. at 20° C./min; reheat to | 224.5° C. (35.9 J/g); 224.9° C.(48.03 J/g) |
| | 240° C. at 10° C./min. | Exothermic peak Onset: 186.0° C.(52.43 J/g) |
| HCH2 | Cycle 2: 30° C. to 240° C. at 10° C./min; | Endothermic peak Onset: 51.2° C.(13.11 J/g); |
| | 240° C. to −20° C. at 2° C./min; reheat to | 224.9° C.(40.62 J/g); 224.3° C. (40.03 J/g) |
| | 240° C. at 10° C./min. | Exothermic peak Onset: 178.4° C.(51.85 J/g) |

### (9) Water Activity Study at 25° C. (Table 39)

Water activity experiments were conducted at 25° C. in 10 different water activities with 1 set of organic solvent/water mixtures (acetone/water) to determine critical water activity between anhydrate and hydrate.

TABLE 39
Water activity experiments (protected by N₂, 5 days)

| Exp. ID | Solvents | a.w.* | XRPD | Comments |
|---|---|---|---|---|
| AW1 | Acetone | 0 | // | API degradation, purity 97.34% |
| AW2 | & water | 0.103 | Pattern A | Purity 98.27% |
| AW3 | | 0.206 | Pattern A | Purity 99.18% |
| AW4 | | 0.301 | Pattern D | Purity 99.77% |
| AW5 | | 0.400 | Pattern D | Purity 99.49% |
| AW6 | | 0.501 | Pattern D | Purity 99.33% |
| AW7 | | 0.603 | Pattern D | Purity 99.19% |
| AW8 | | 0.703 | // | Clear solution |
| AW9 | | 0.803 | // | Clear solution |
| AW10 | | 0.901 | // | Clear solution |

Explanation
*a.w. by calculation.

### (10) Water Sorption and Desorption Experiments (Tables 40 and 41)

Water sorption and desorption behavior was investigated by DVS at 25° C. with a cycle of 40-95-0-40% RH. dm/dt is 0.002. Min equilibration time is 60 min. Max equilibration time is 360 min. XRPD was measured after DVS test to determine form change.

TABLE 40
Pattern A water sorption and desorption experiments.

| | 1^{st} | 2^{nd} | 2^{nd} |
|---|---|---|---|
| Relative humidity | sorp. Weight % | desorp. Weight % | sorp. Weight % |
| by DVS | change | change | change |
| 0 | | 0.01 | 0.01 |
| 10 | | 0.87 | −0.07 |
| 20 | | 1.95 | −0.11 |
| 30 | | 3.05 | −0.13 |
| 40 | −4.73 | 4.15 | |
| 50 | −4.63 | 5.32 | |
| 60 | −3.57 | 6.62 | |
| 70 | −3.26 | 8.07 | |
| 80 | −3.01 | 11.34 | |
| 90 | 18.76 | 36.14 | |
| 95 | 60.34 | 60.34 | |

XRPD after DVS test
Pattern D

TABLE 41
Pattern D water sorption and desorption experiment.

| | 1^{st} | 2^{nd} | 2^{nd} |
|---|---|---|---|
| Relative humidity | sorp. Weight % | desorp. Weight % | sorp. Weight % |
| by DVS | change | change | change |
| 0 | | 0.00 | 0.00 |
| 10 | | 0.15 | 0.11 |
| 20 | | 0.22 | 0.19 |
| 30 | | 0.29 | 0.26 |
| 40 | 0.47 | 0.48 | |
| 50 | 0.51 | 0.67 | |
| 60 | 0.78 | 0.88 | |
| 70 | 1.00 | 1.26 | |
| 80 | 1.54 | 2.04 | |
| 90 | 5.42 | 12.37 | |
| 95 | 16.82 | 16.82 | |

XRPD after DVS test
Pattern D

### (11) Scale Up Experiment (Tables 42-45)

About 6 g API were used for Pattern A and Pattern D scale up.

About 3 g drug substance were equilibrated in 12 ml of solvent (Water: Acetone, 1:99, v:v) at 25° C. for 1 week with a stirring plate. Obtained suspension was filtered. The solid part (wet cake) was investigated by XRPD.

About 3 g drug substance were equilibrated in 8 mL of solvent (Water: Acetone, 10:90, v:v) at 25° C. for 1 week with a stirring plate. Obtained suspension was filtered. The solid part (wet cake) was investigated by XRPD.

TABLE 42
Scale up experiments.

| Method | Solvent | XRPD | DSC | TGA | Comments |
|---|---|---|---|---|---|
| Anti-solvent | EtOAc- | Pattern A | 73.42° C. | 2.264% at | Purity 99.94% |
| | Heptane | | (108.07 J/g); | 120° C. | Water content 1.982% |
| | | | 227.30° C. | | |
| | | | (33.555 J/g); | | |

| Slow cooling | MEK | Pattern B | Not carried out | API degradation, purity |
|---|---|---|---|---|
| | | | | 93.4% |
| Slow evaporation | EtOAc | Gel | Not carried out | Not carried out |

TABLE 43
Scale up experiments (protected by N₂, 10 days)

| | | | After TGA |
|---|---|---|---|
| | | | heat to |
| Method | Solvent | XRPD | 120° C. |
| Equilibration | Water:Acetone(1:99, v:v) | Pattern A | Pattern E |
| | Water:Acetone(10:90, v:v) | Pattern D | Pattern E |

TABLE 44
Study of identified Patterns.

| | | | XRPD after heating | XRPD patterns | Thermal events of |
|---|---|---|---|---|---|
| Exp. | | | to 150° C., hold for | after stored at | samples after stored at |
| ID | XRPD | Batch No. | 5 min | RT for 7 days | RT for 7 days (DSC) |
| SI1 | Pattern A | FR00623-01- | Pattern E | Similar to | 75.33° C.(118.4 J/g); |
| | | 190912-01 | | Pattern A | 226.08° C. (51.70 J/g); |
| SI2 | Pattern B | FR00623-01- | Pattern E | Similar to | 64.64° C.(156.0 J/g); |
| | | 190923-02 | | Pattern A | 234.78° C. (69.02 J/g); |
| SI3 | Pattern C | FR00623-01- | Pattern E | Similar to | 75.75° C.(126.9 J/g); |
| | | 190924-03 | | Pattern A | 224.84° C. (41.13 J/g); |

TABLE 45
Pattern A stability study.

| | | XRPD after | Purity after |
|---|---|---|---|
| | | stored at RT | stored at RT |
| XRPD | Batch No. | for 1 month | for 1 month |
| Pattern A | FR00623-01-190912-01 | Pattern A | 99.95% |

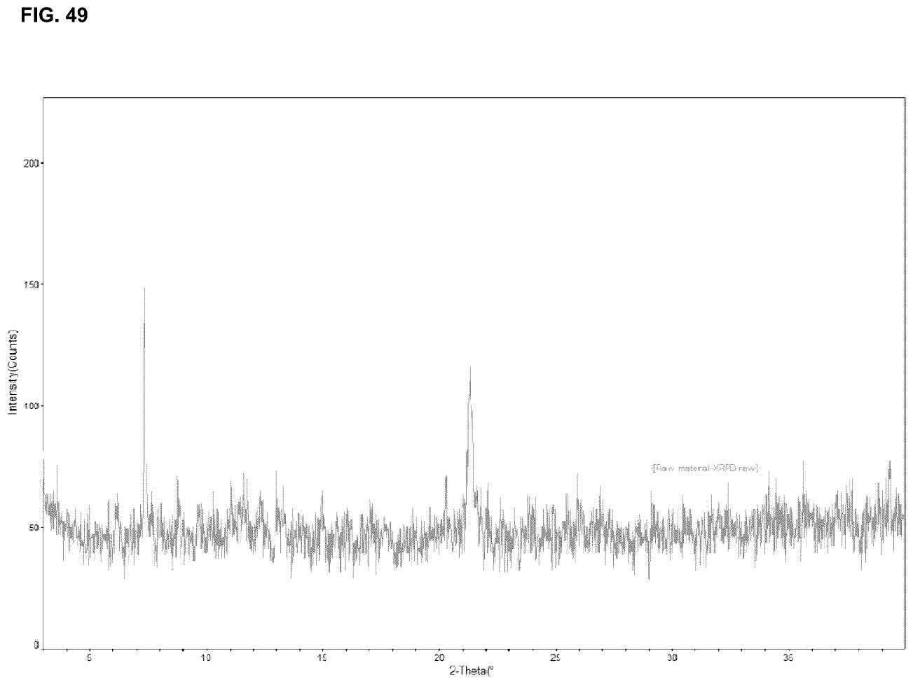

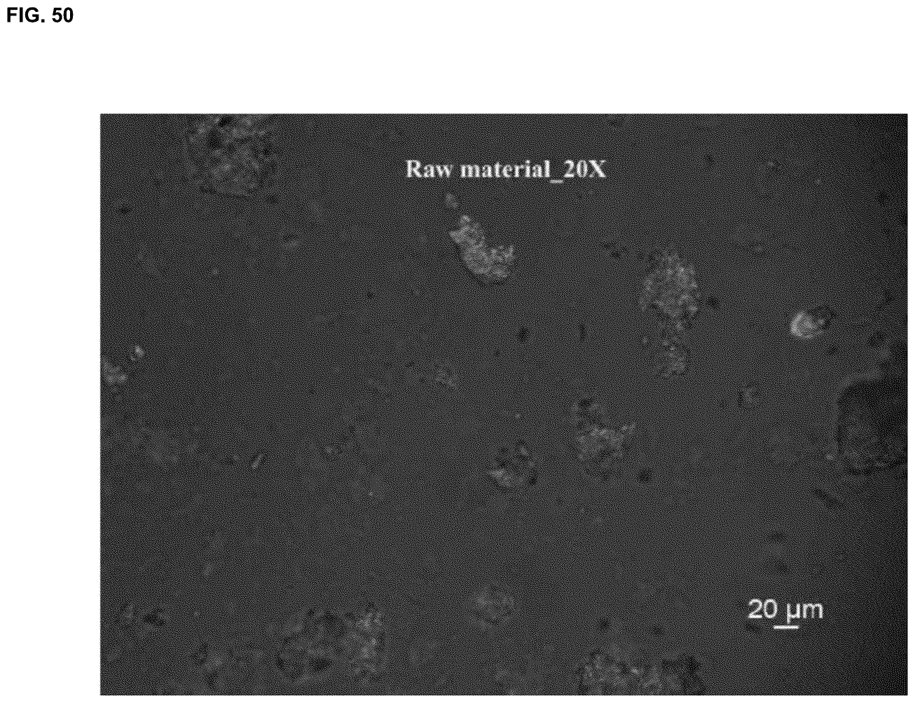

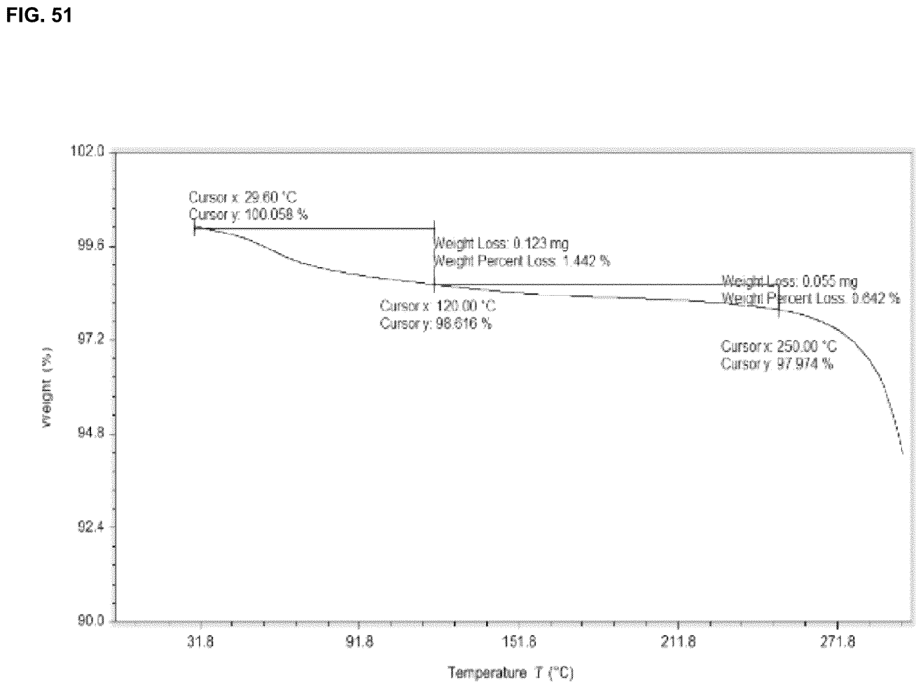

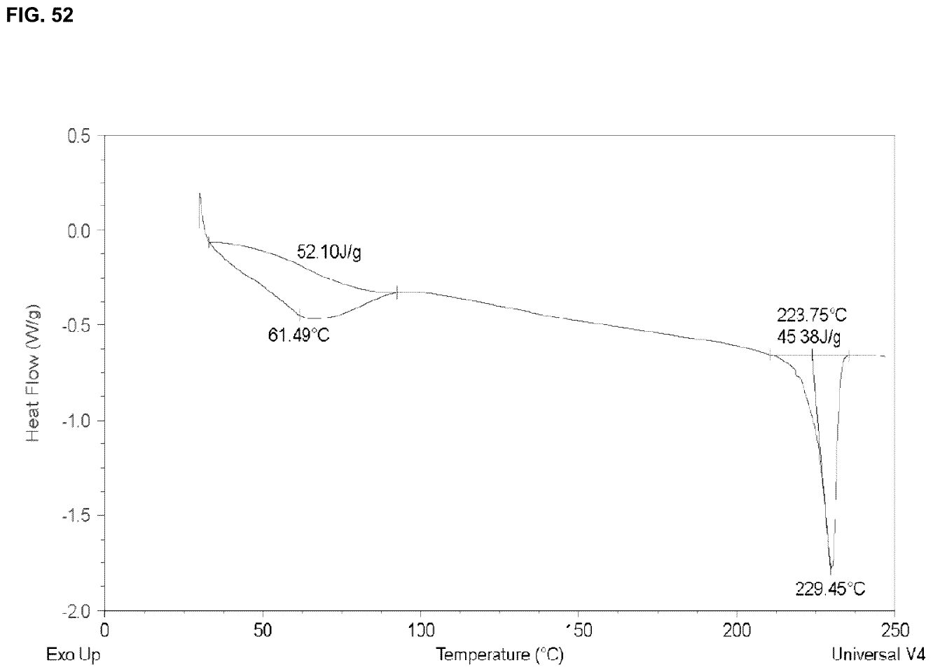

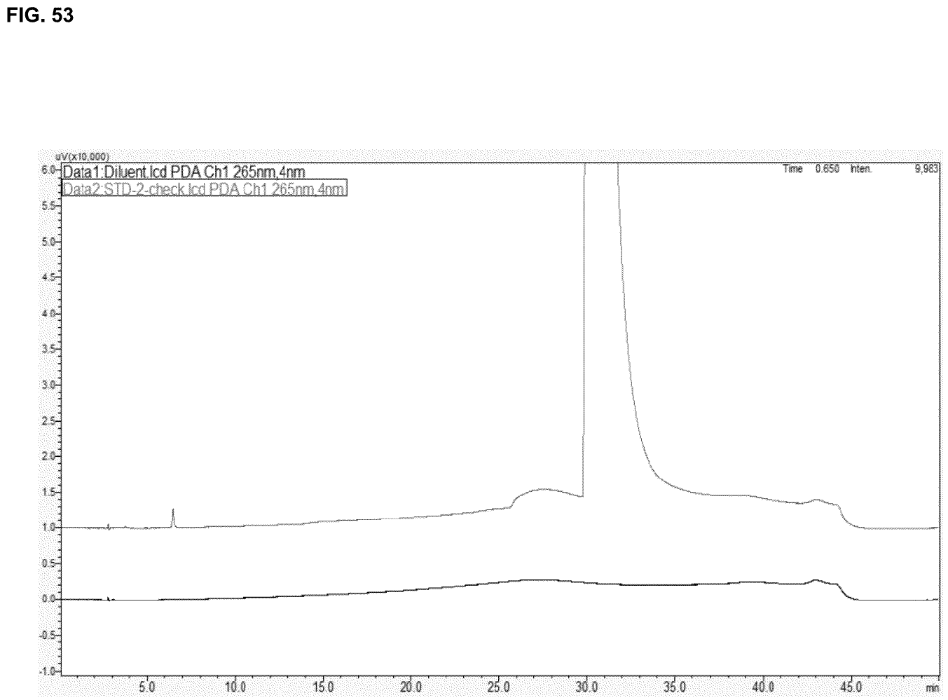

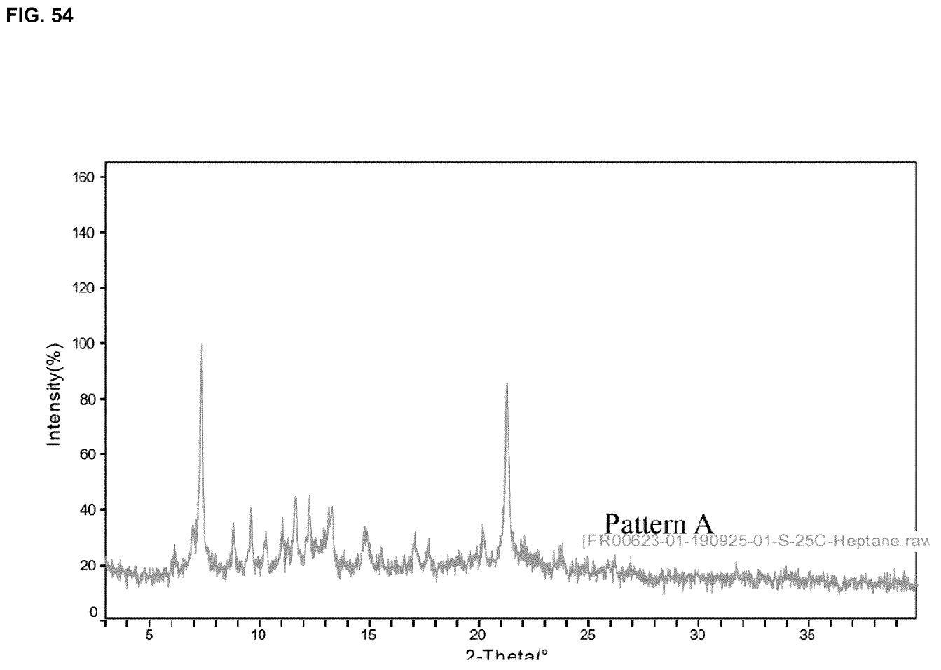

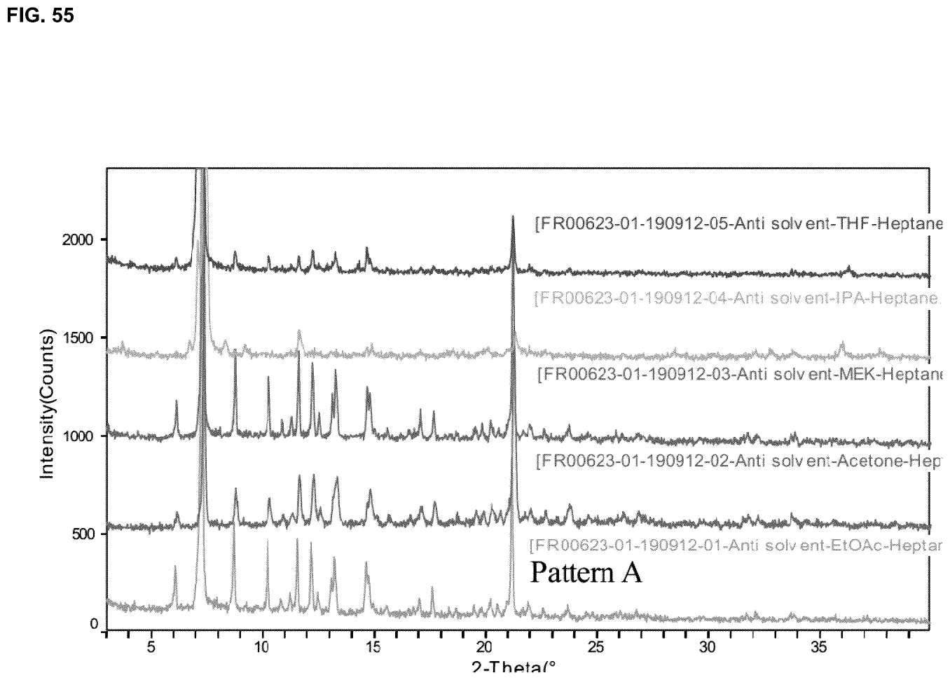

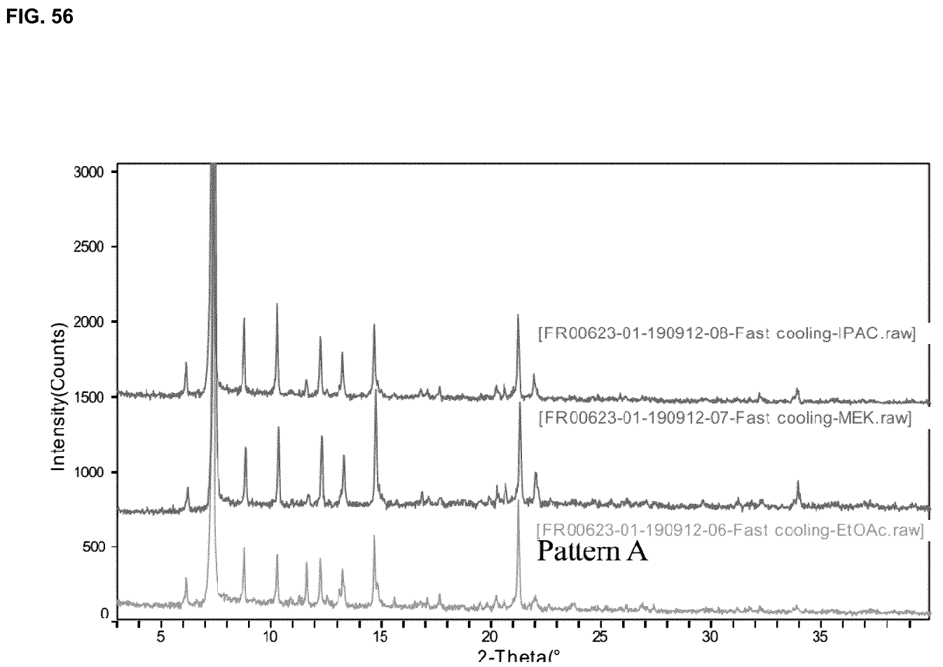

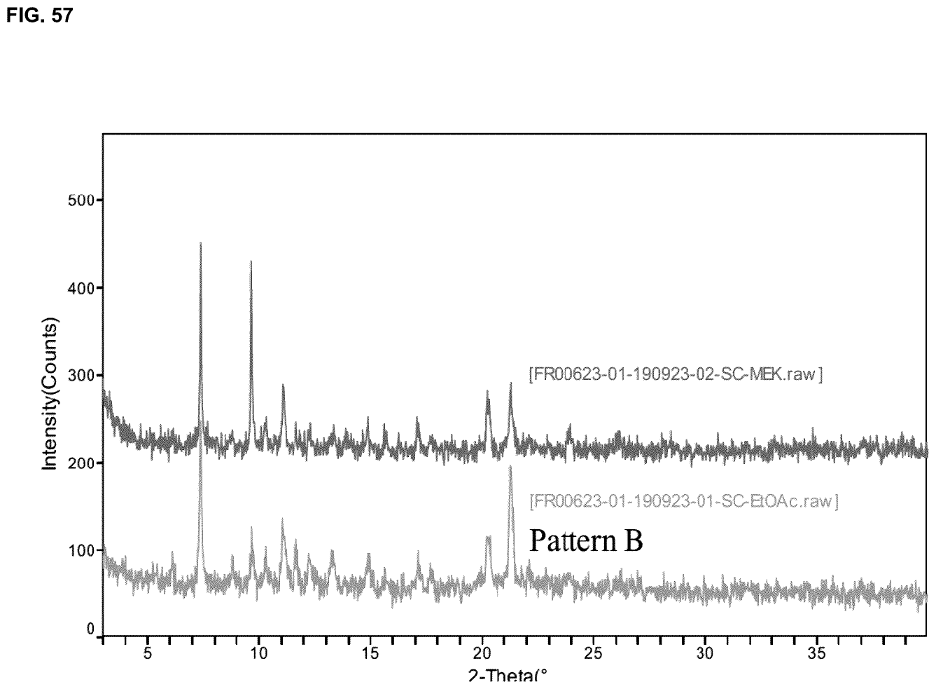

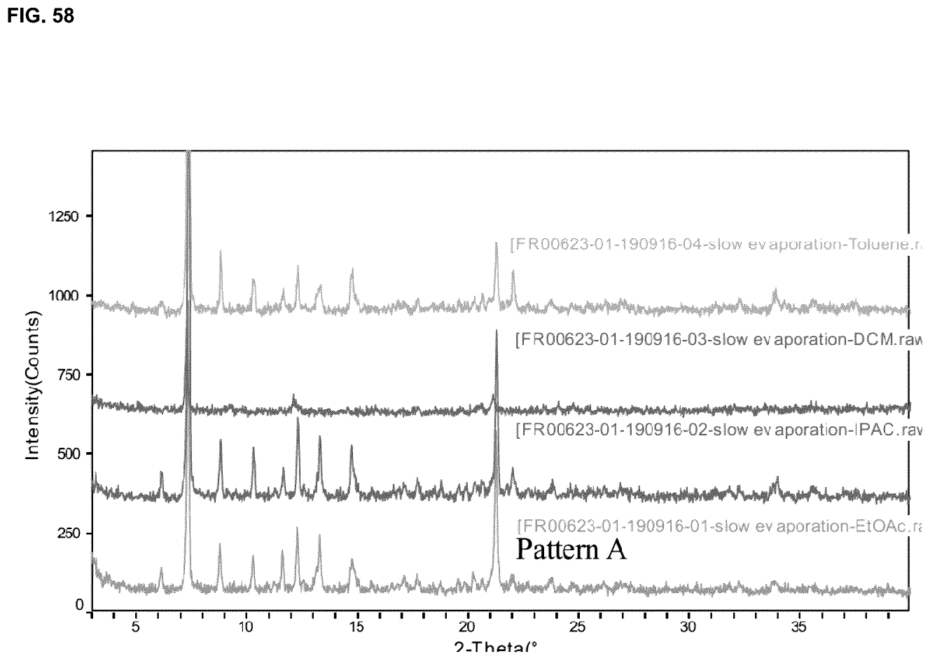

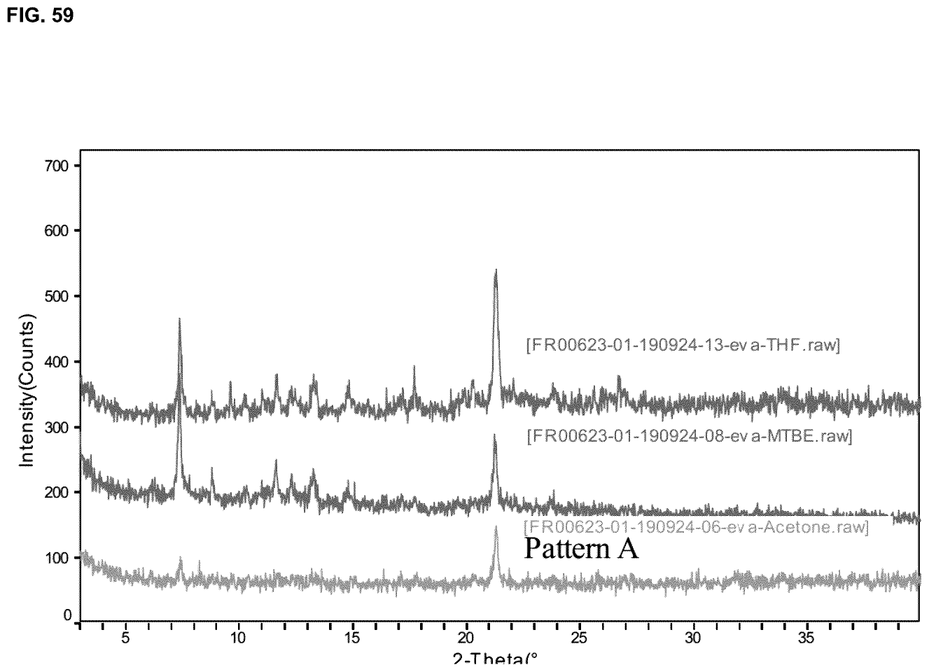

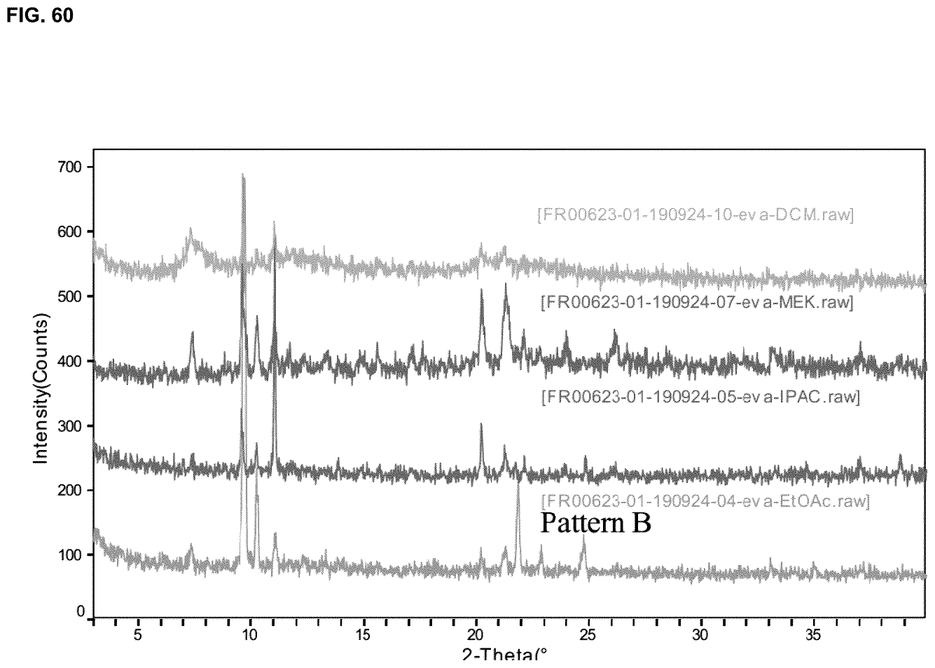

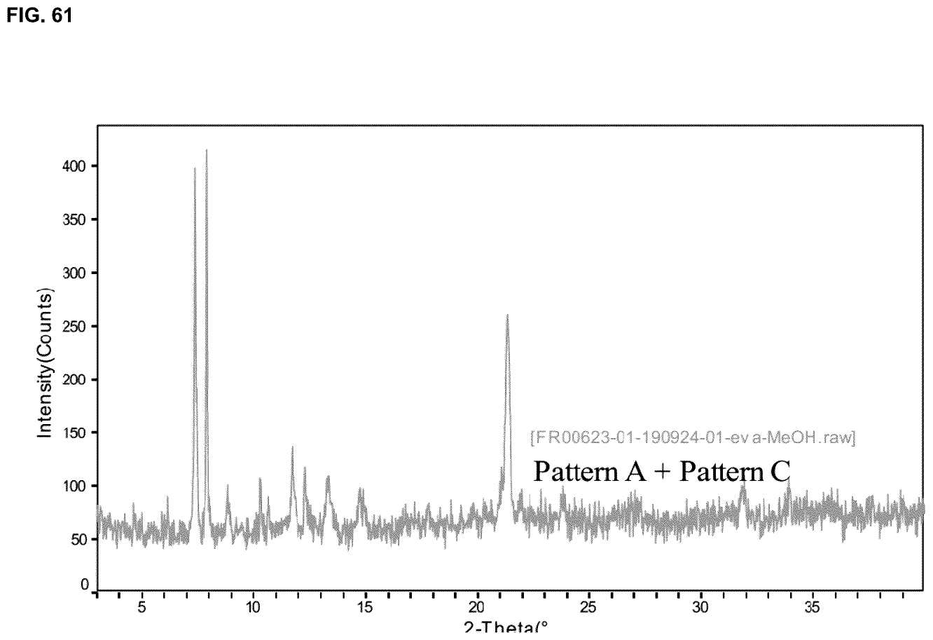

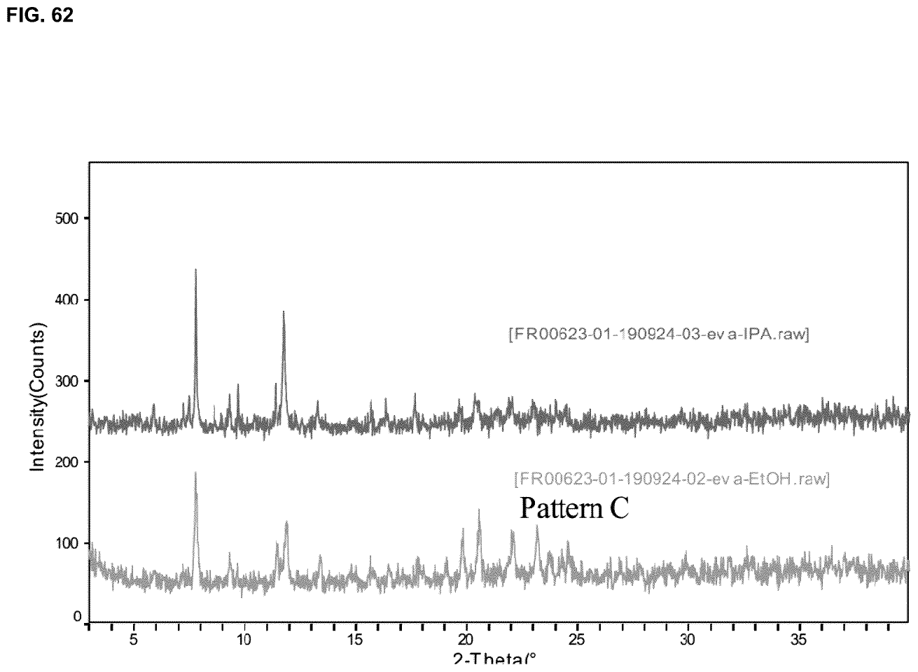

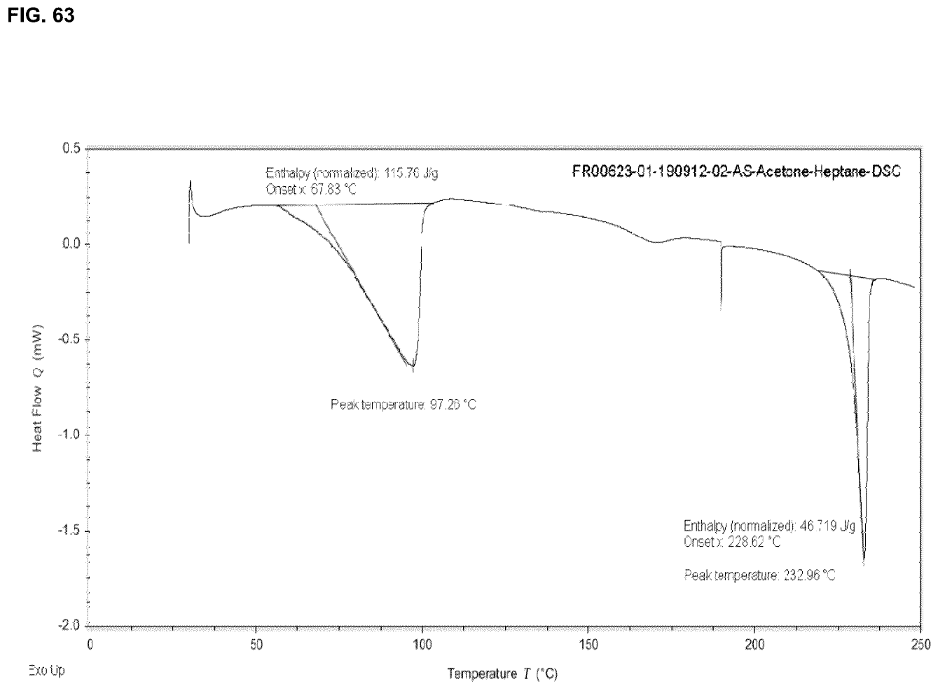

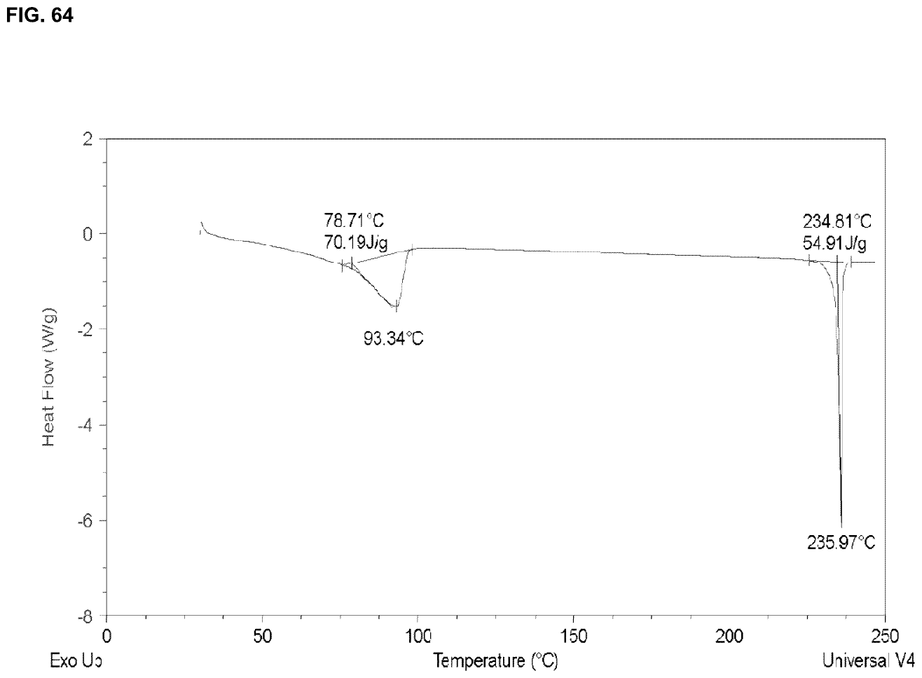

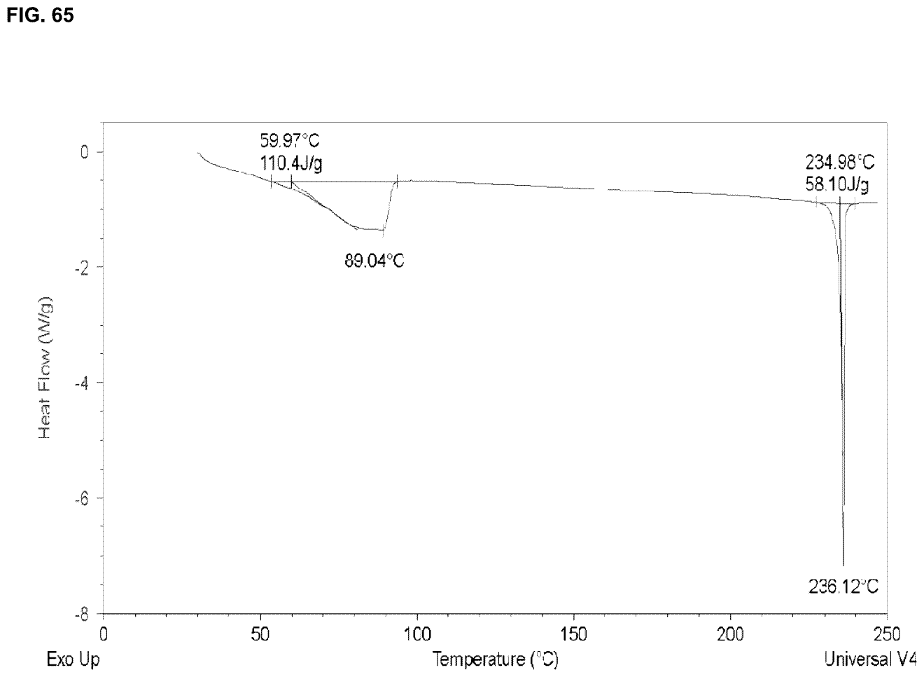

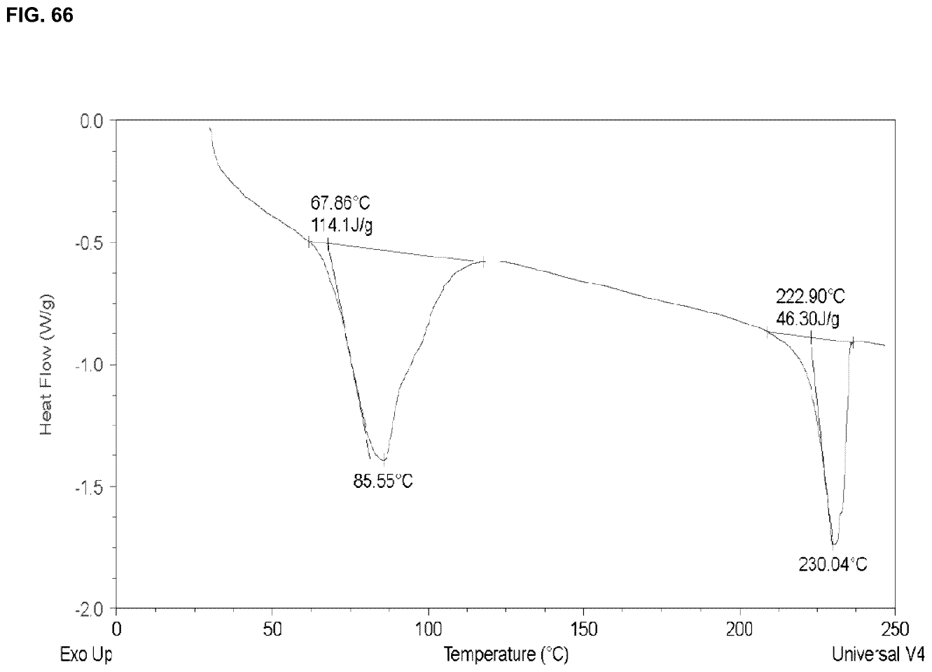

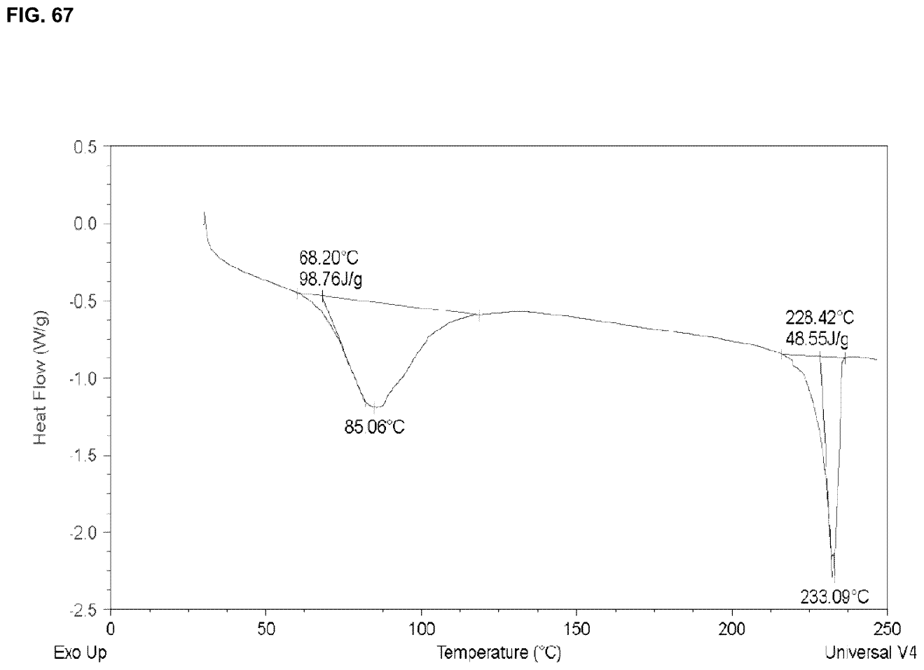

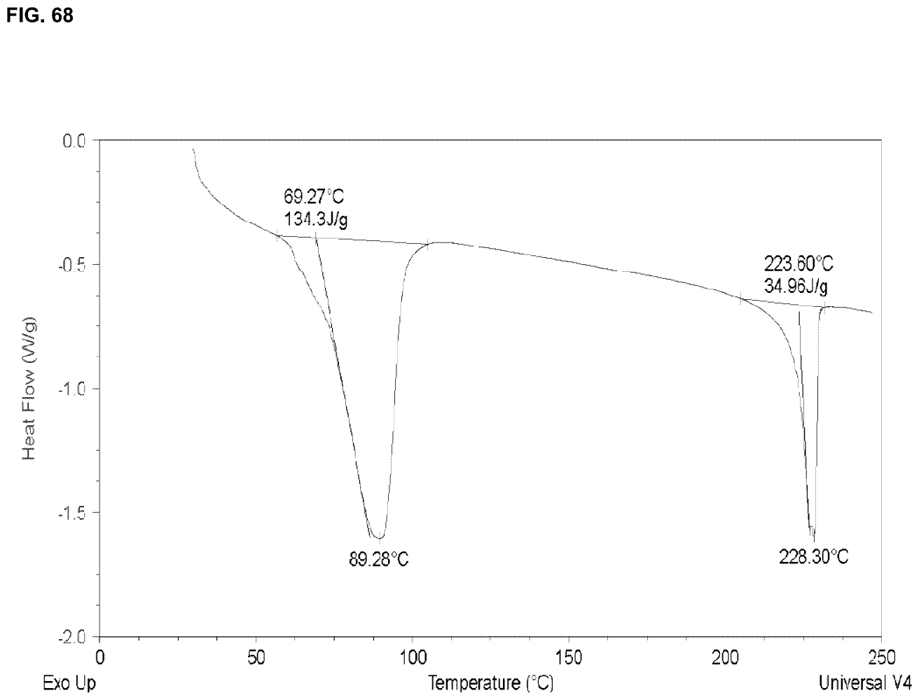

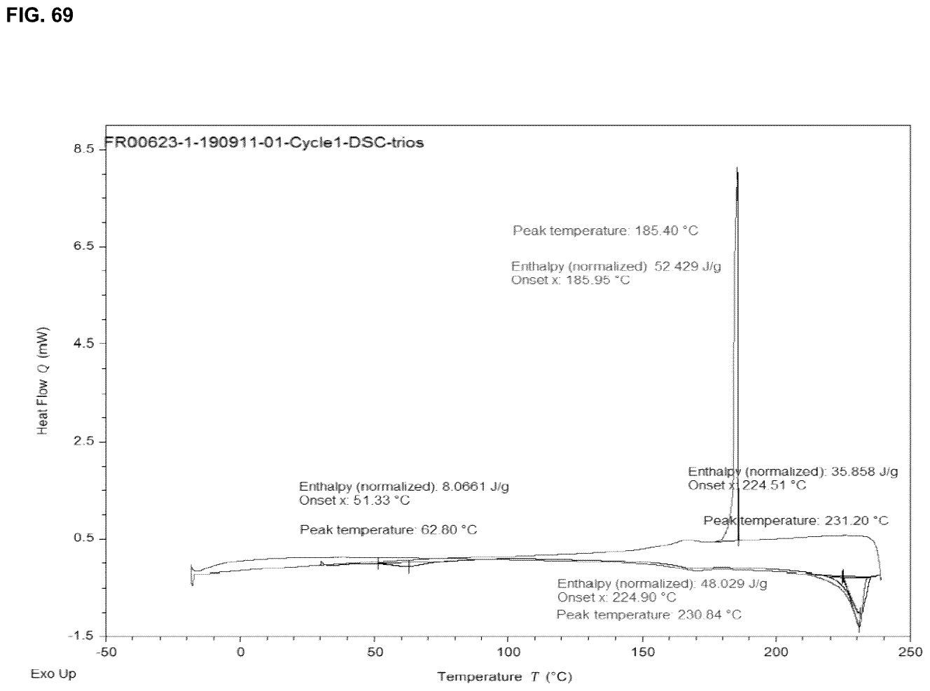

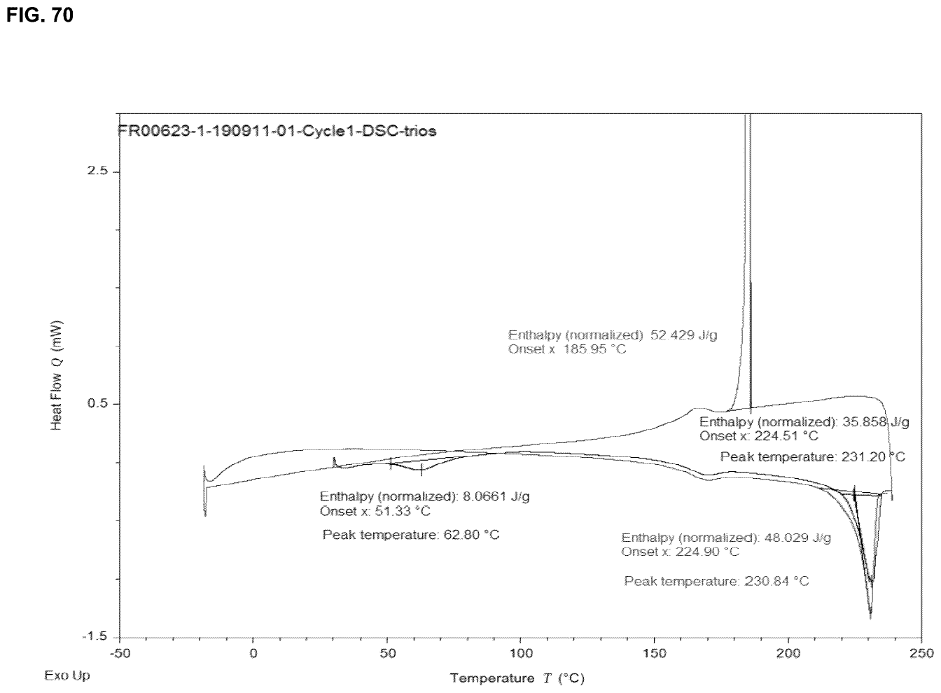

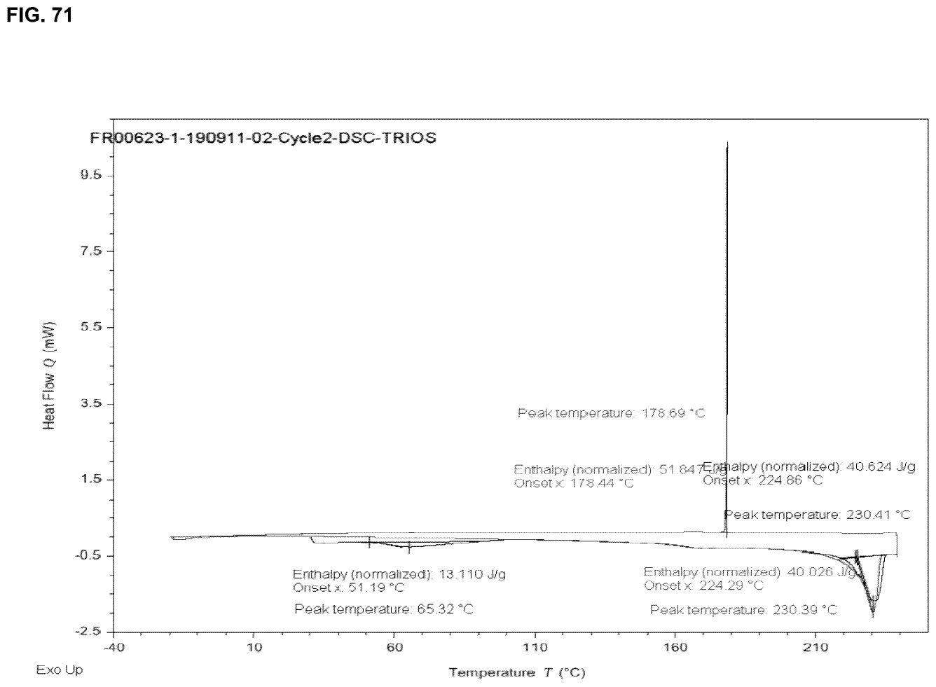

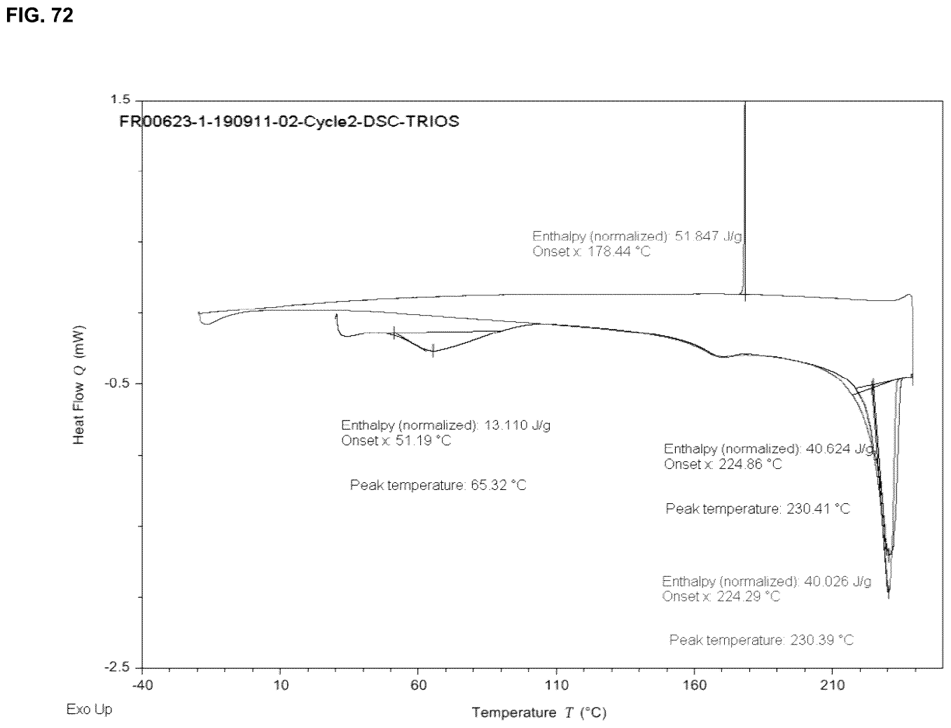

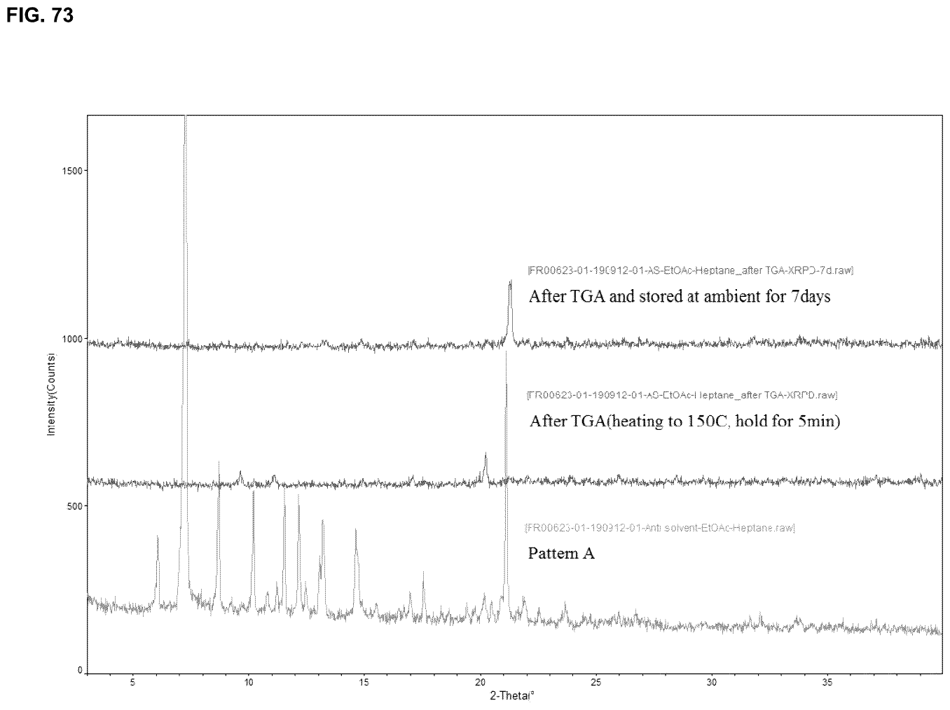

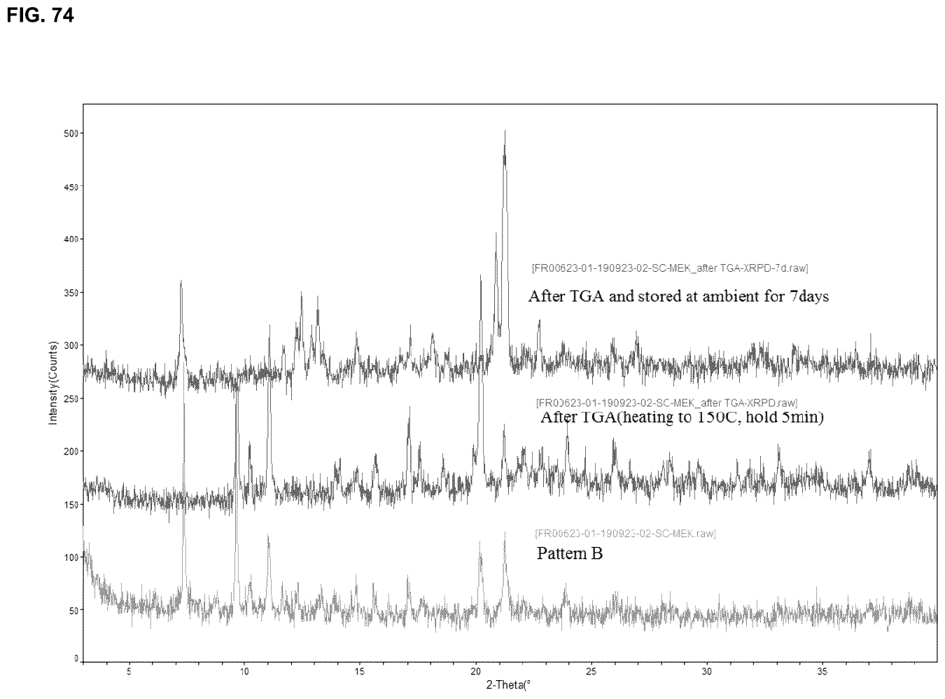

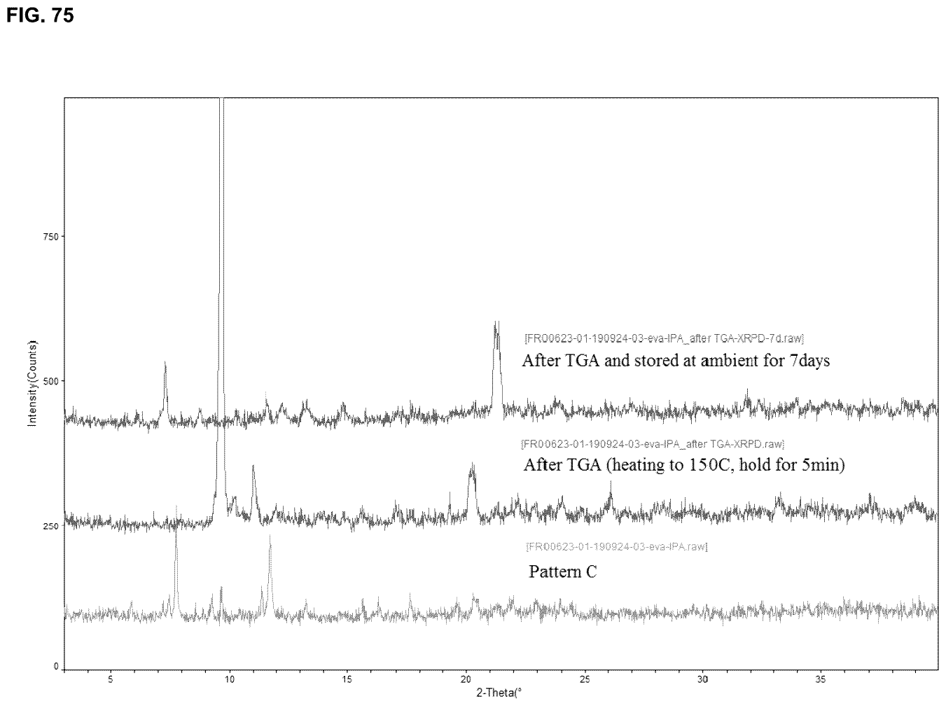

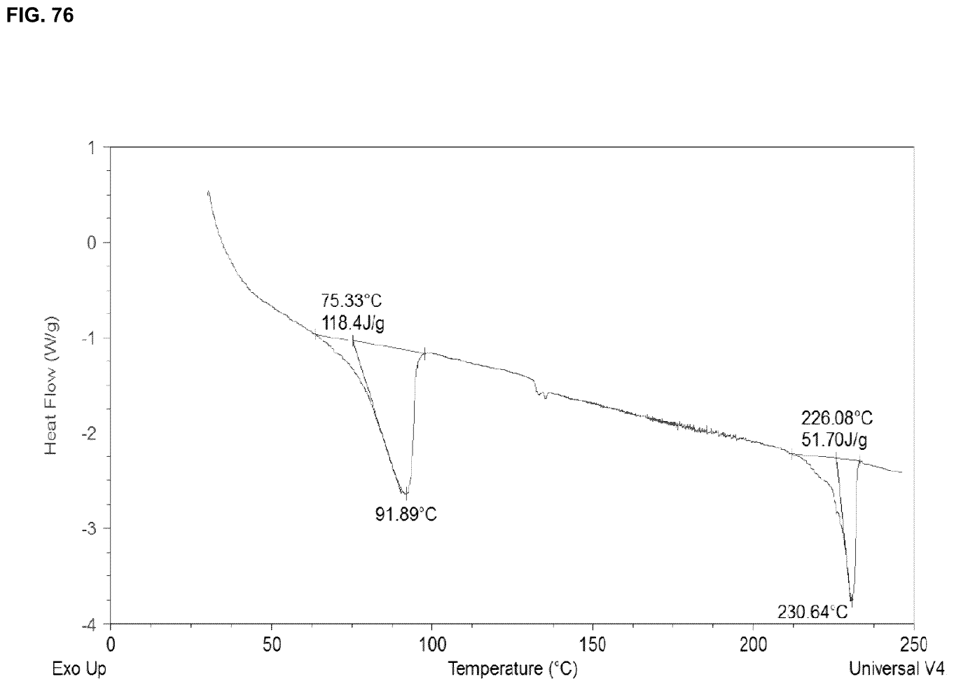

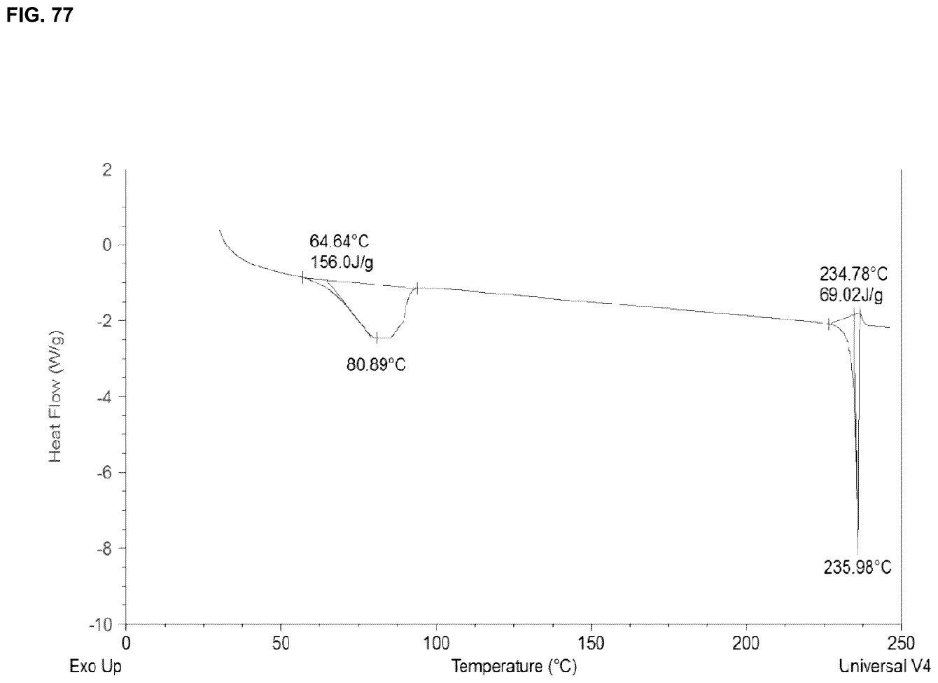

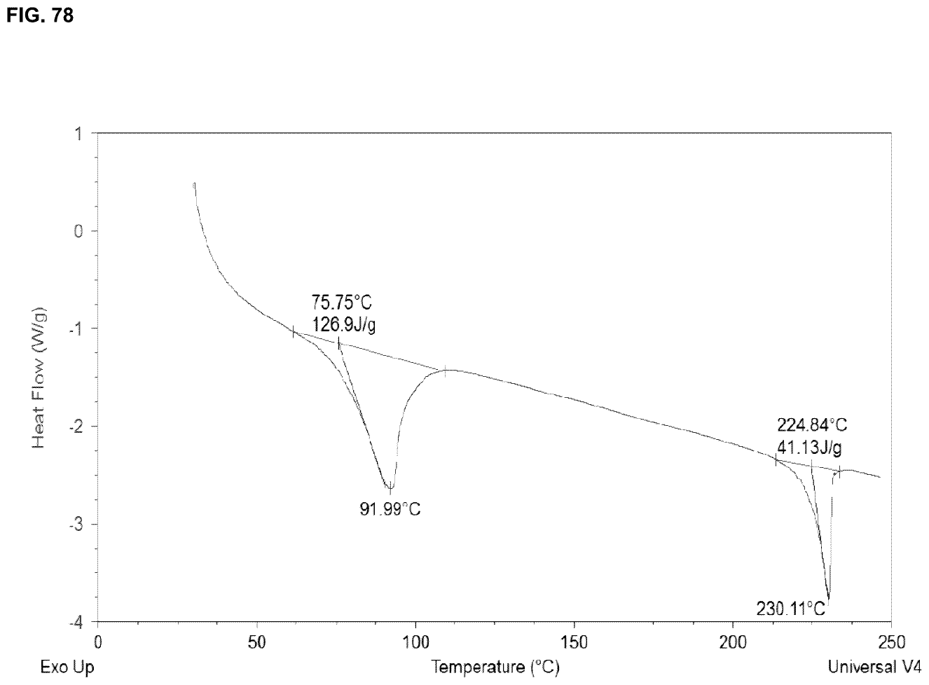

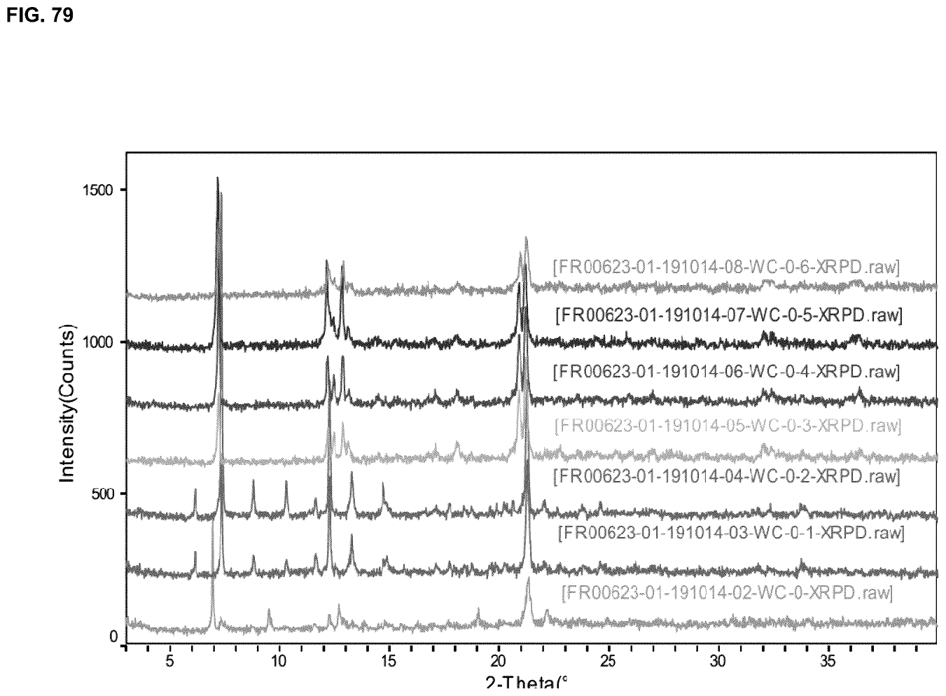

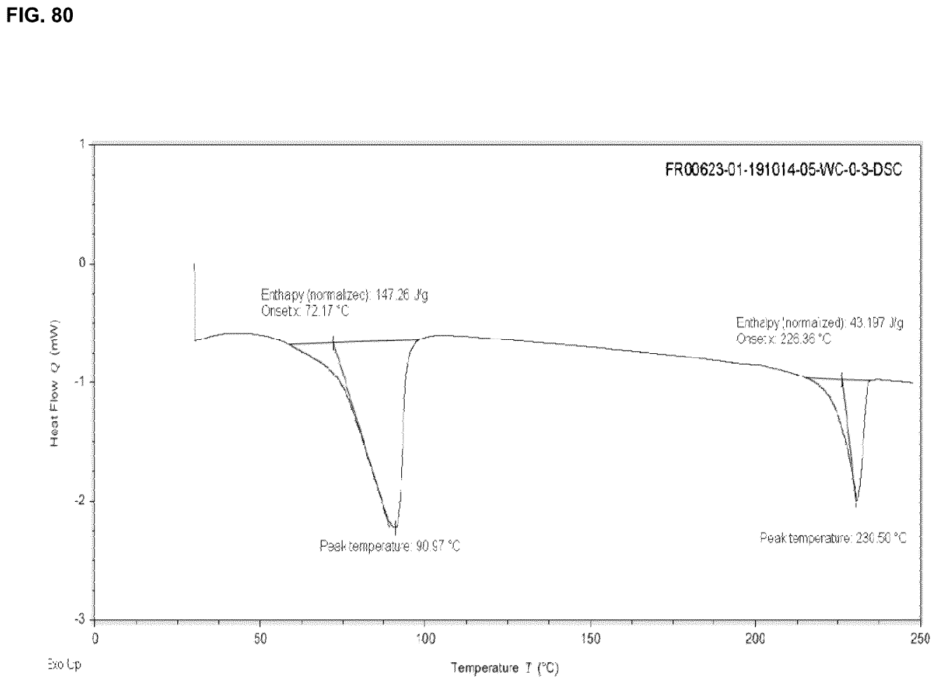

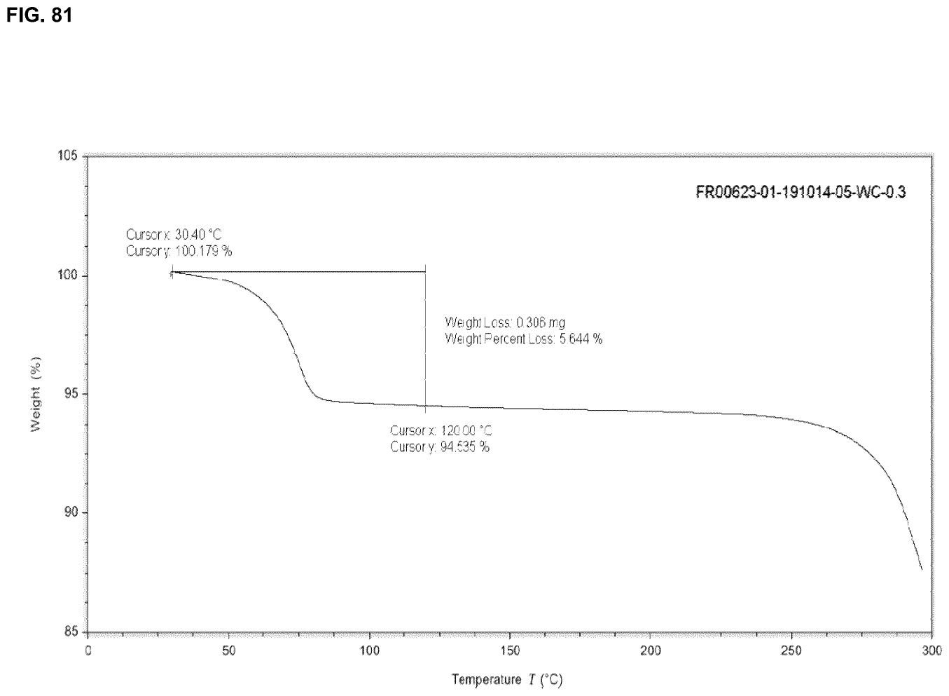

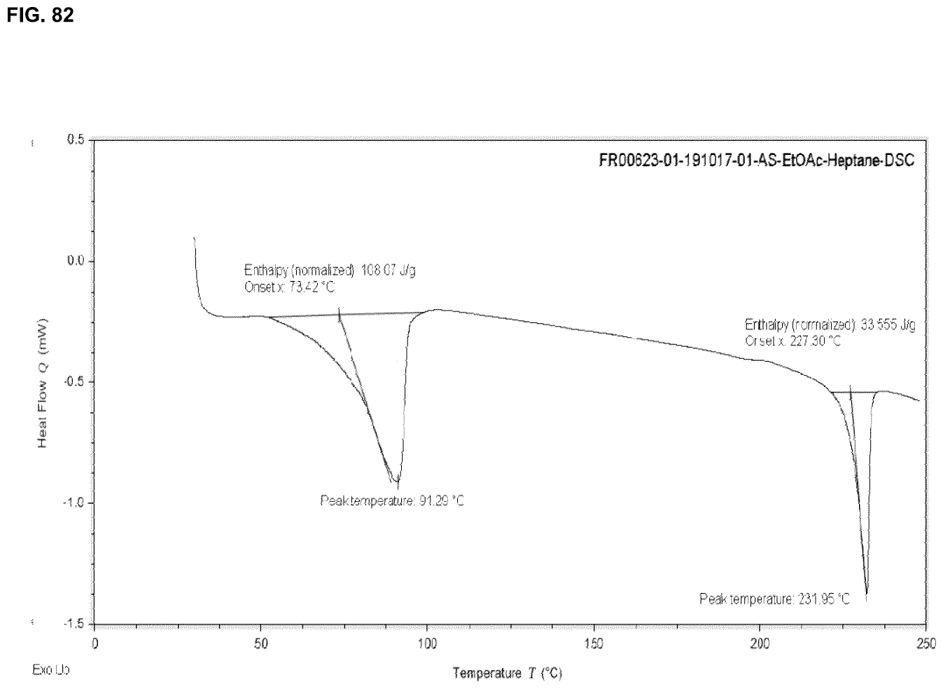

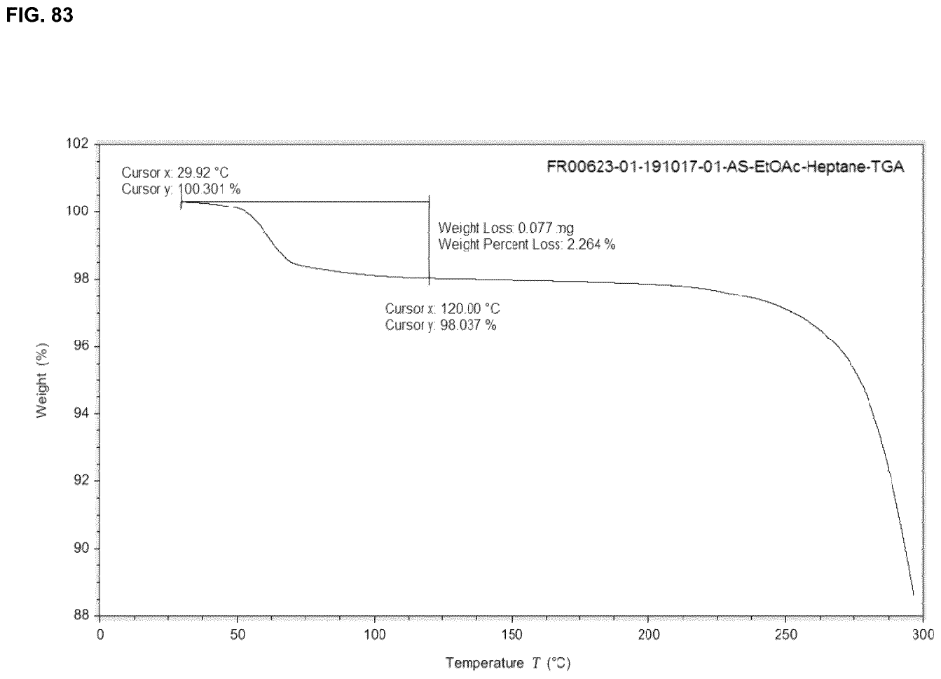

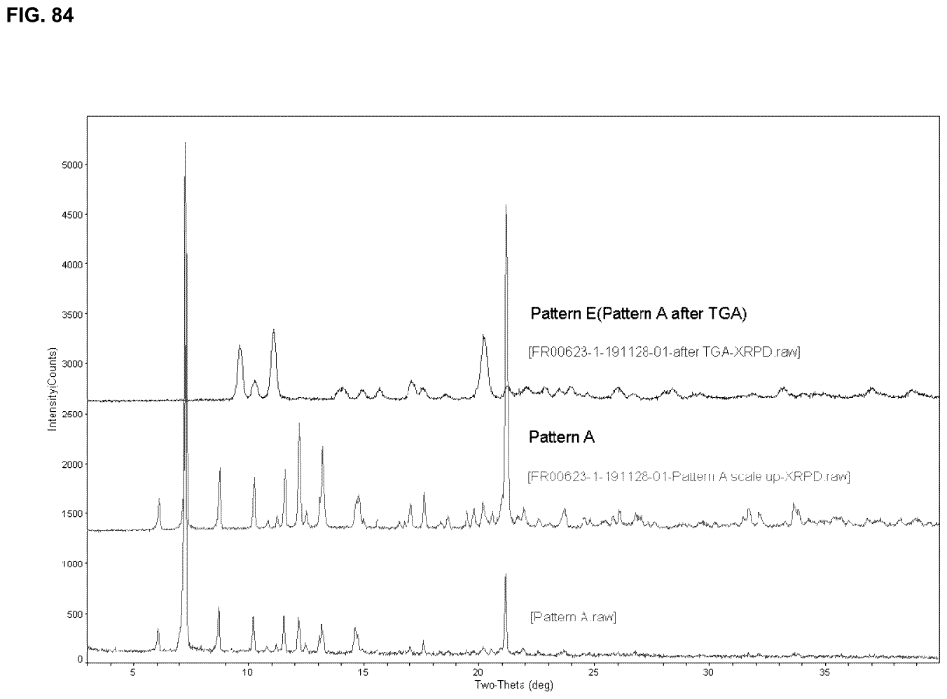

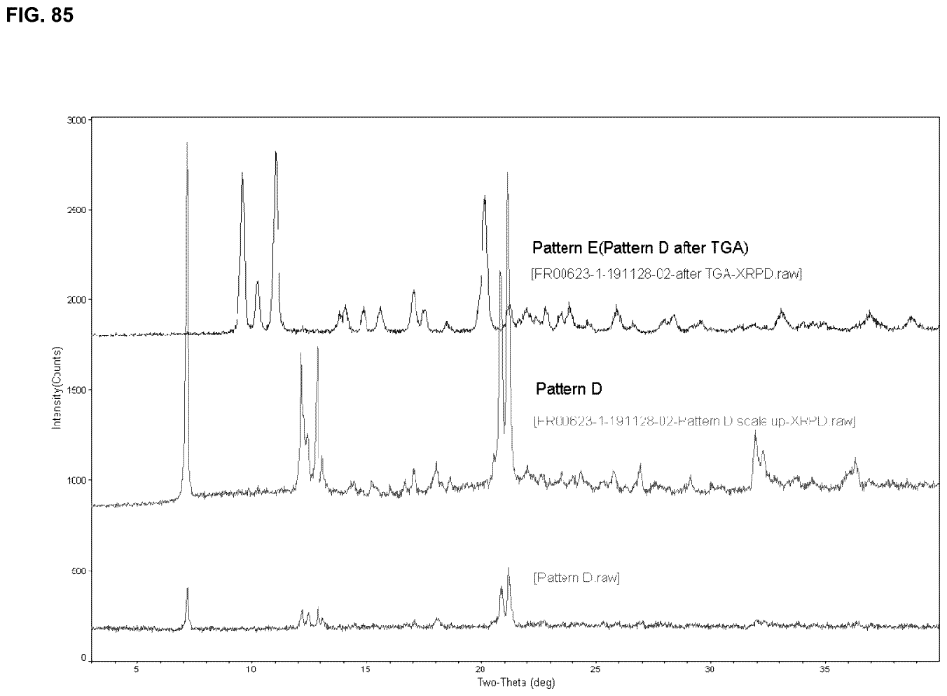

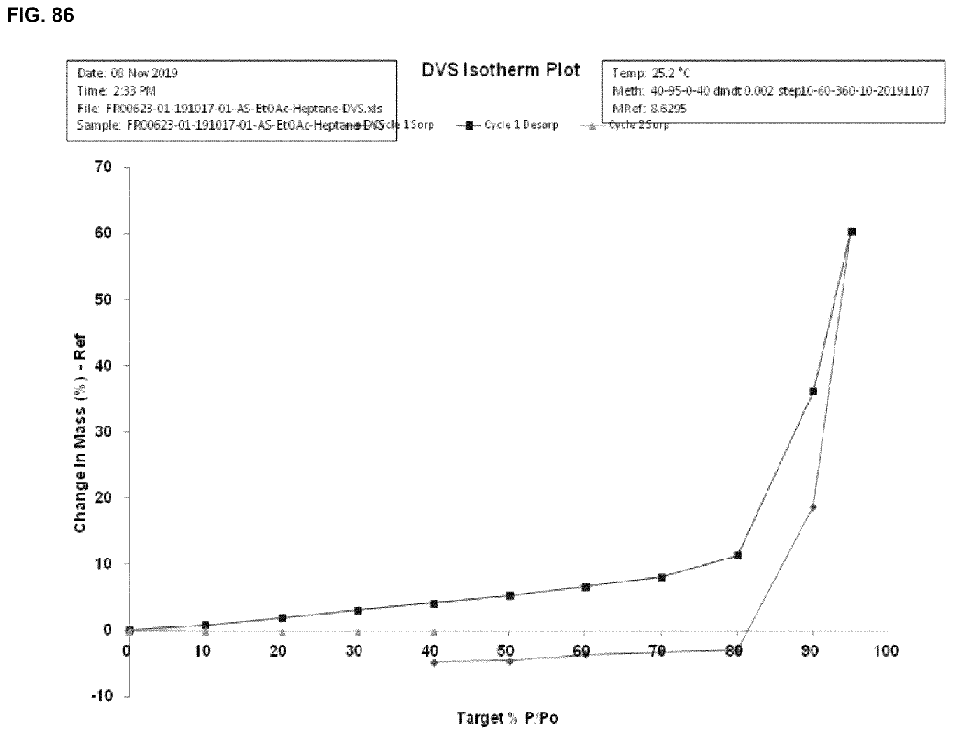

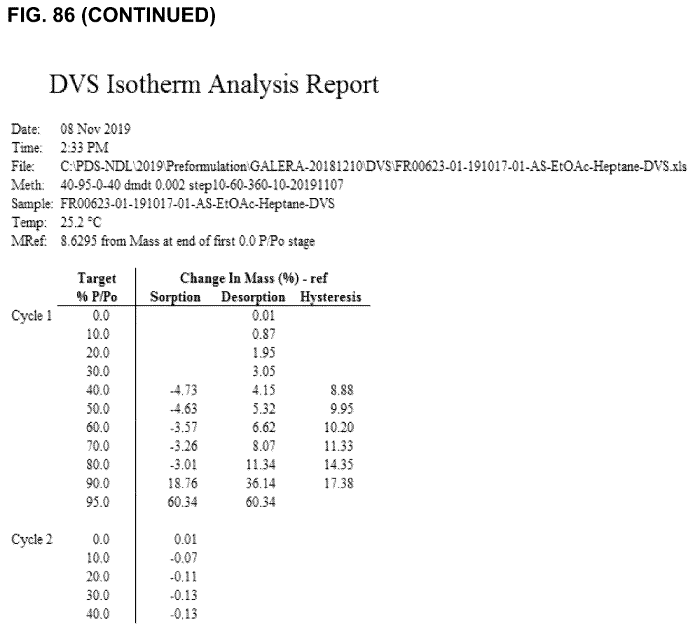

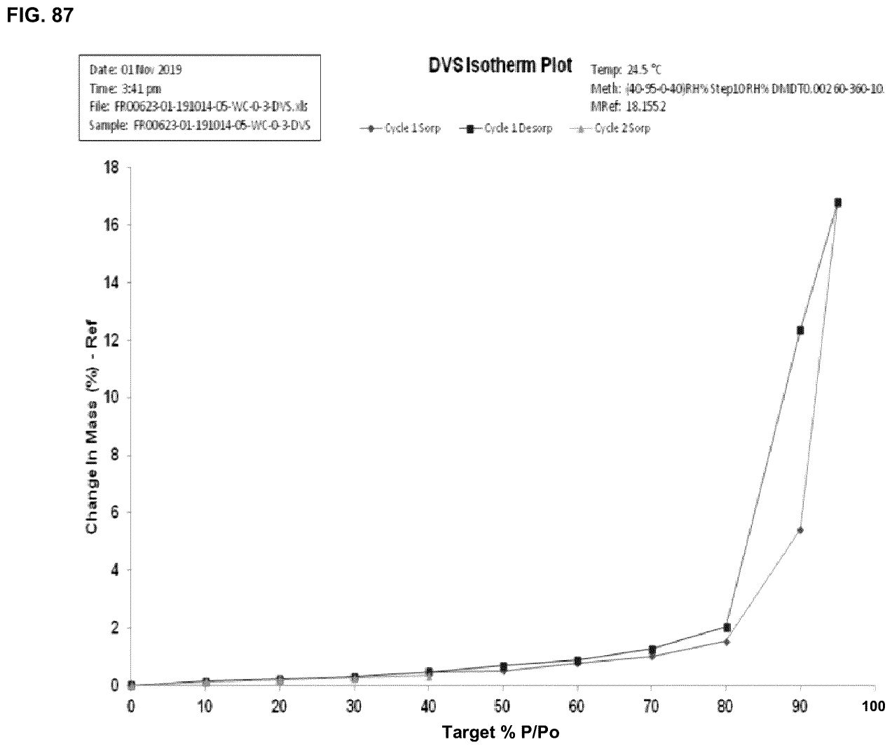

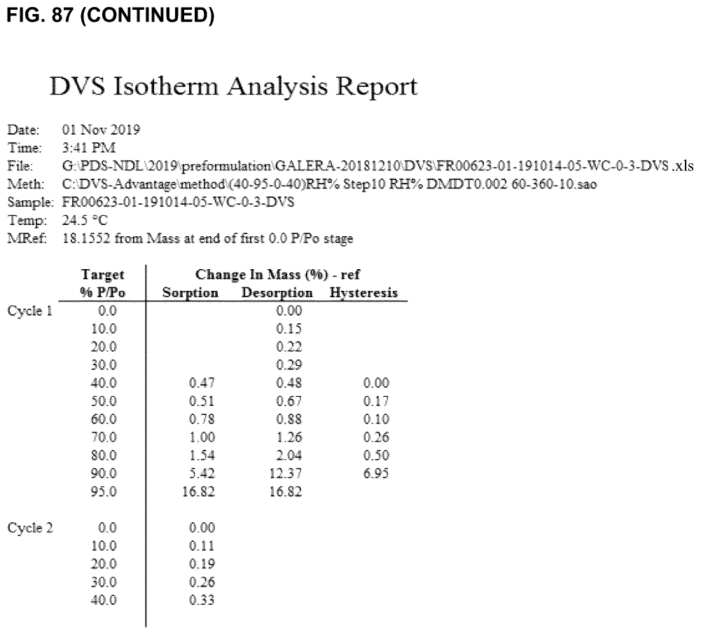

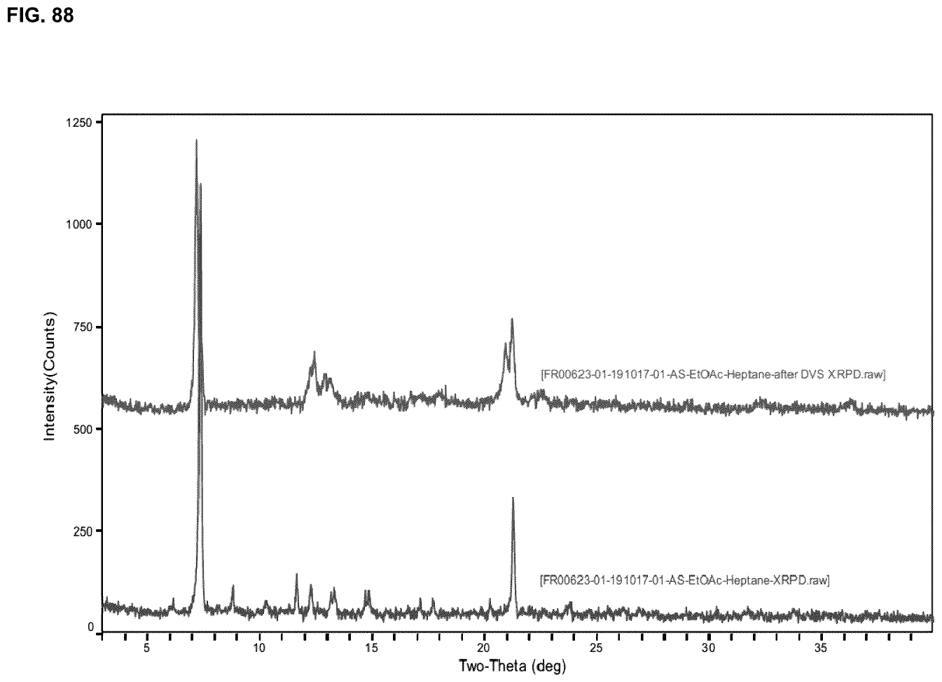

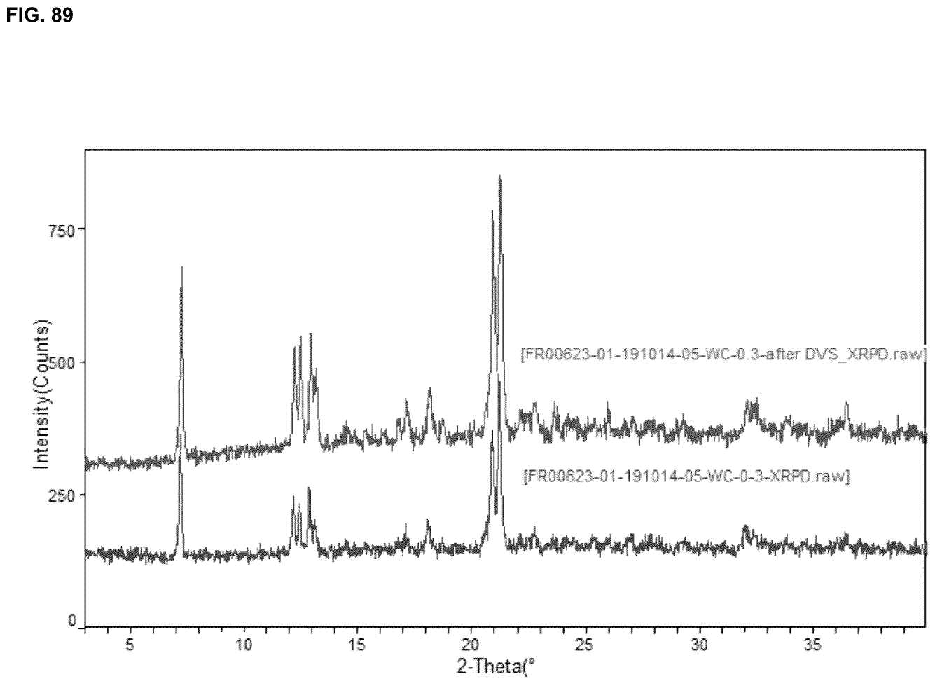

Other information including materials, methods, results, figures and raw data can be found in Table 46 and FIGS. 49-89.

TABLE 46
Instrumental methods.
X-ray Powder Diffractometer (XRPD)

| | |
|---|---|
| Instrument | Bruker D8 Advance |
| Radiation | Cu/K-Alpha1 (λ = 1.54179 Å) |
| X-ray generator power | 40 kV, 40 mA |
| Step size | 0.02° |
| Time per step | 0.45 second per step |
| Scan range | 3° to 40° |
| Sample rotation speed | 15 rpm |

Differential Scanning Calorimetric (DSC)

| | |
|---|---|
| Instrument | TA Discovery 2500 or Q2000 |
| Sample pan | Tzero pan and Tzero hermetic lid with a pin hole |
| Temperature range | RT to 250° C. or before decomposition |
| Heating rate | 10° C./min |
| Nitrogen flow | 50 mL/min |
| Sample mass | ~1-2 mg |

Thermal Gravimetric Analysis (TGA)

| | |
|---|---|
| Instrument | Discovery 5500 or Q5000 |
| Sample pan | Aluminum, open |
| Nitrogen flow | Balance 10 mL/min; sample 25 mL/min |
| Start temperature | 30° C. |
| Final temperature | 300° C. |
| Heating rate | 10° C./min |
| Sample mass | ~2-10 mg |

Dynamic Vapor Sorption (DVS)
Method

| | |
|---|---|
| Instrument | Intrinsic |
| Total gas flow | 200 sccm |
| Oven temperature | 25° C. |
| Solvent | Water |
| Method | Cycle: 40-95-0-95-40% RH |
| | Stage Step: 10% |
| | Equilibrium: 0.002 dm/dt (%/min) |
| | Minimum dm/dt stability duration: 60 min |
| | Maximum dm/dt stage time: 360 min |

Polarized Light Microscope (PLM)

| | |
|---|---|
| Instrument | Nikon LV100POL |
| Method | Crossed polarizer |
| temperature 25° C. | |
| dm/dt | 0.002%/min |

High Performance Liquid Chromatograph (HPLC)

| Instrument | Shimadzu | | |
|---|---|---|---|
| HPLC method | Wave length: 265 nm | | |
| | Column: ACE C18, 250 mm × 4.6 μm, 5 μm | | |
| | Detector: UV | | |
| | Column temperature: 45° C. | | |
| | Flow rate: 1.0 mL/min | | |
| | Mobile phase A: 600 mM Ammonium Chloride in water, pH6.5 ± 0.1 | | |
| | Mobile phase B: Methanol | | |
| | Diluent: 0.1 mM Zinc Acetate, 26 mM NaHCO3 in 68% Mobile phase | | |
| | A/17% water/15% Methanol (v/v/v) (pH = 7.35 ± 0.05) | | |
| | Injection volume: 10 μL | | |
| | Gradient: | | |
| | Time (min) | Mobile Phase A (%) | Mobile Phase B (%) |
| | 0 | 87 | 13 |
| | 34.5 | 60 | 40 |
| | 40 | 60 | 40 |
| | 40.1 | 87 | 13 |
| | 50 | 87 | 13 |

TABLE 47
Acronyms.

| Acronyms | Full name |
|---|---|
| ACN | Acetonitrile |
| DCM | Dichloromethane |
| EtOAc | Ethyl acetate |
| EtOH | Ethanol |
| IPA | 2-Propanol |
| IPAC | Isopropyl acetate |
| MEK | Methyl ethyl ketone |
| MeOH | Methanol |
| MTBE | t-Butyl methyl ether |
| THF | Tetrahydrofuran |
| DSC | Differential Scanning Calorimetry |
| DVS | Dynamic Vapour Sorption |
| HPLC | High Performance Liquid Chromatograph |
| PLM | Polarized Light Microscope |
| TGA | Thermal Gravimetric Analysis |
| XRPD | X-ray Powder Diffractometer |

### Example 5

### Prepare and Characterize GC4711 Form E

Previous studies have provided different possible methods to crystallize Form E. These methods include trituration of amorphous GC4711 in various organic solvents, heat induced crystallization of amorphous GC4711, and desolvation of solvated and/or hydrated forms.

The generation of Form E through either the desolvation of solvated/hydrated forms or heat induced crystallization of amorphous GC4711 is considered non-ideal. Desolvation induces crystal defects and disorder. Exposure to elevated temperatures was shown to cause discoloration, suggestive of chemical degradation at that condition.

The trituration of amorphous GC4711 in an organic solvent was considered the most suitable method. Diethyl ether was used for this purpose because of its volatility and high supersaturation and precipitation potential for GC4711.

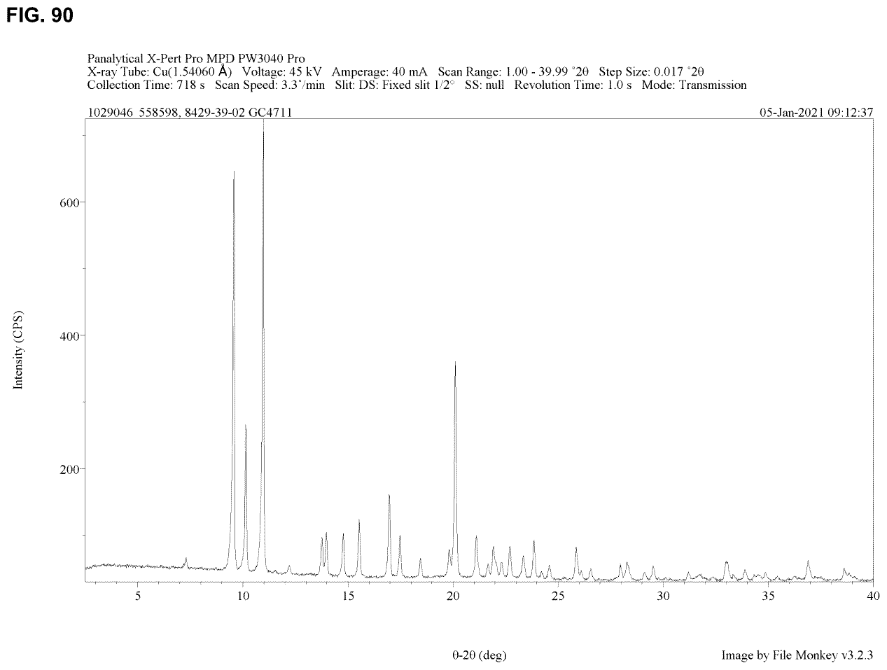

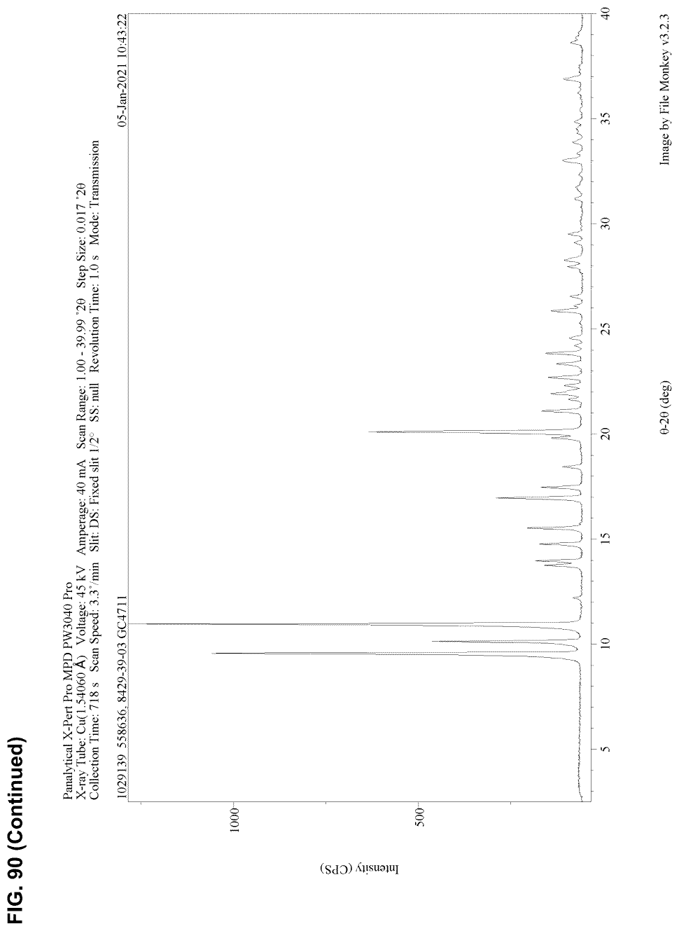

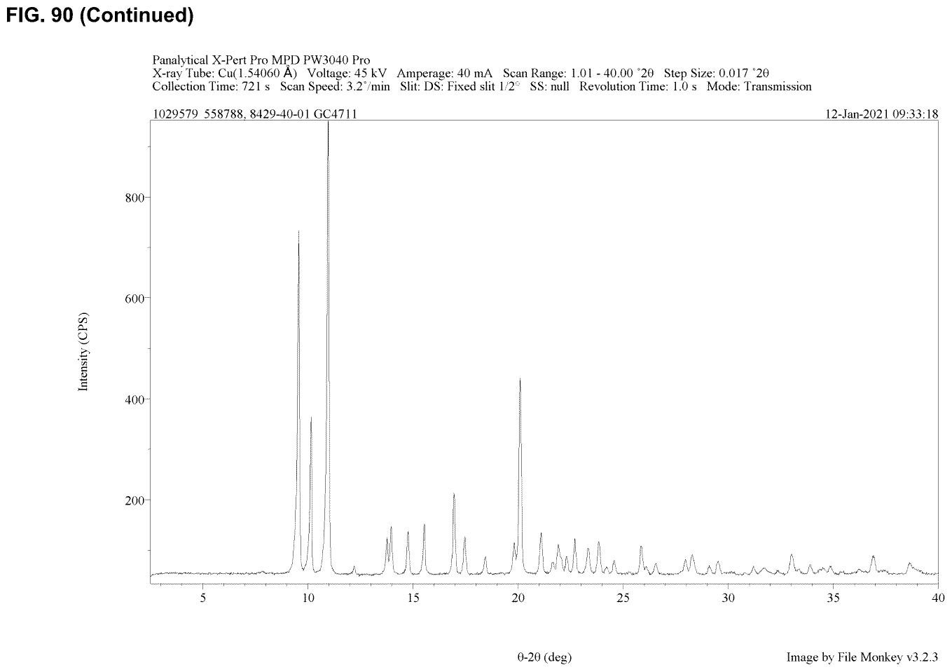

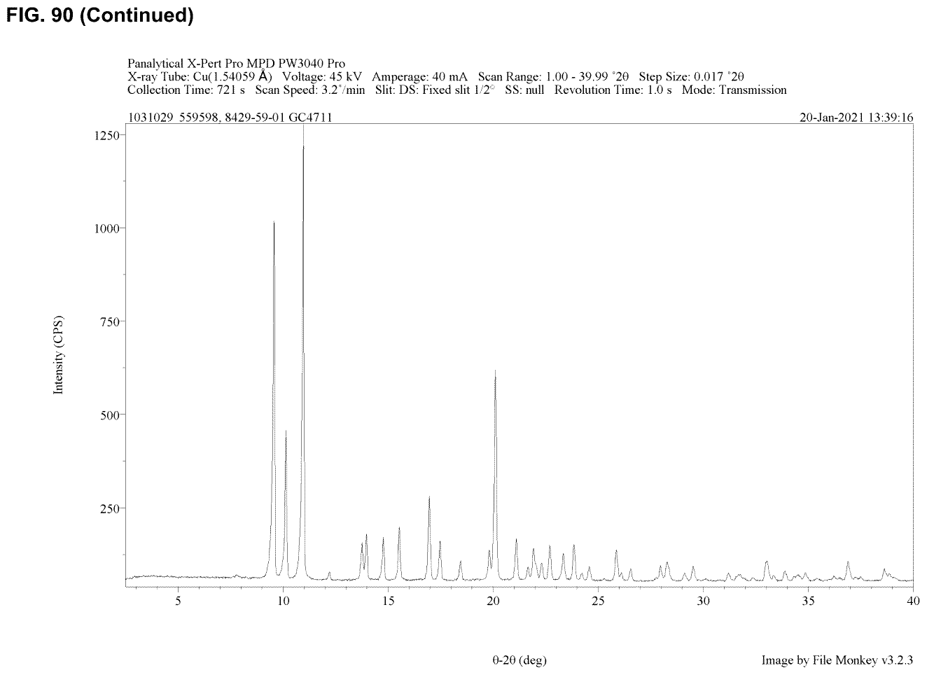

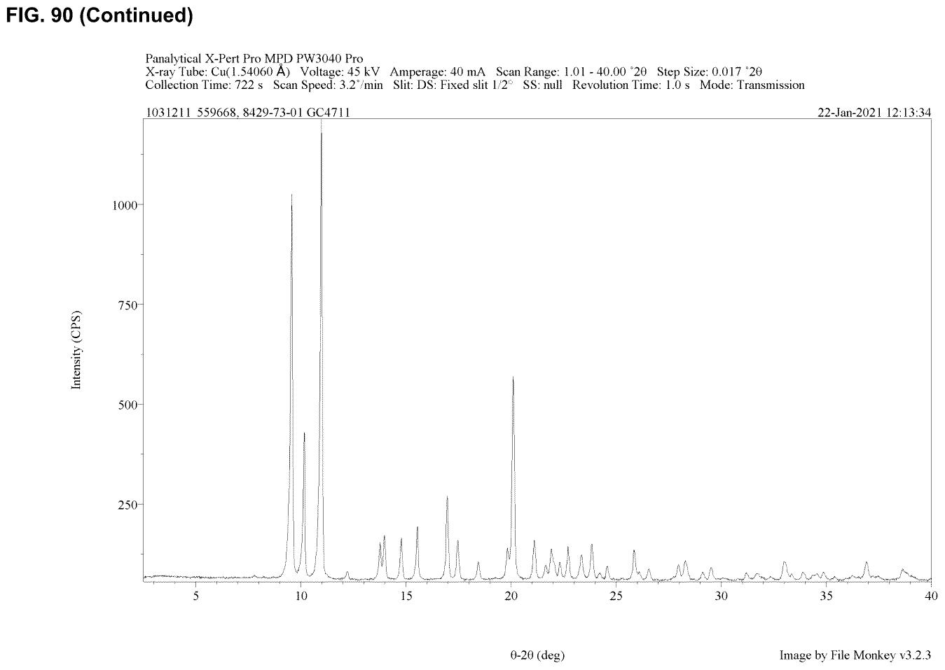

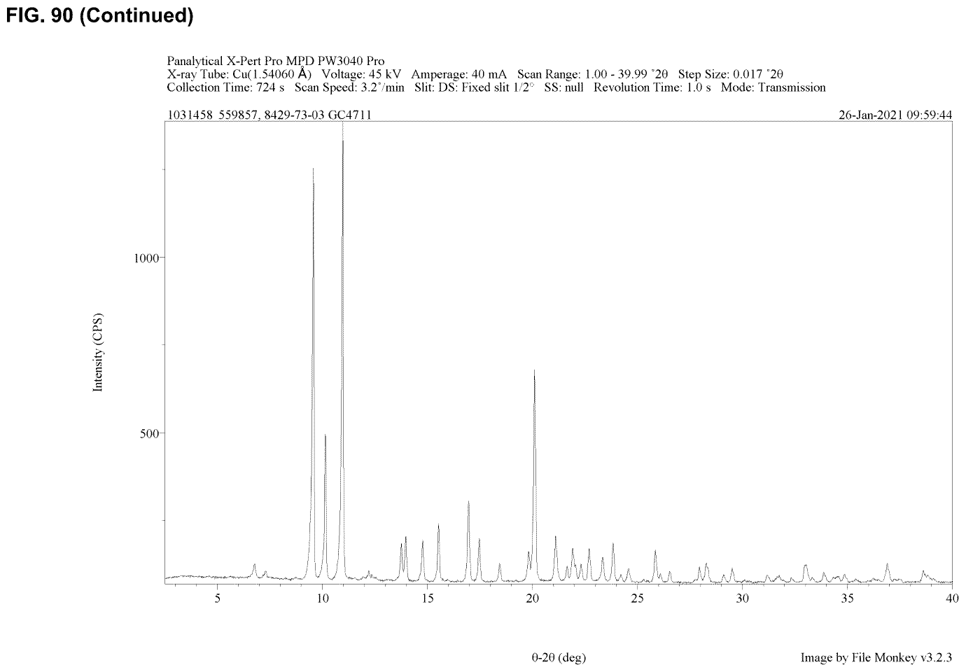

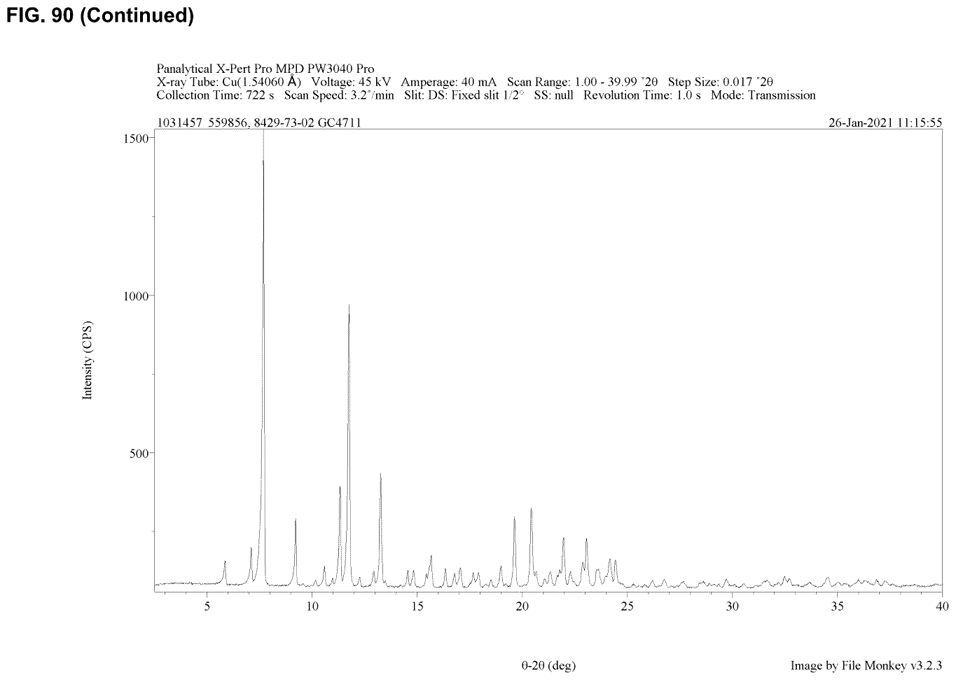

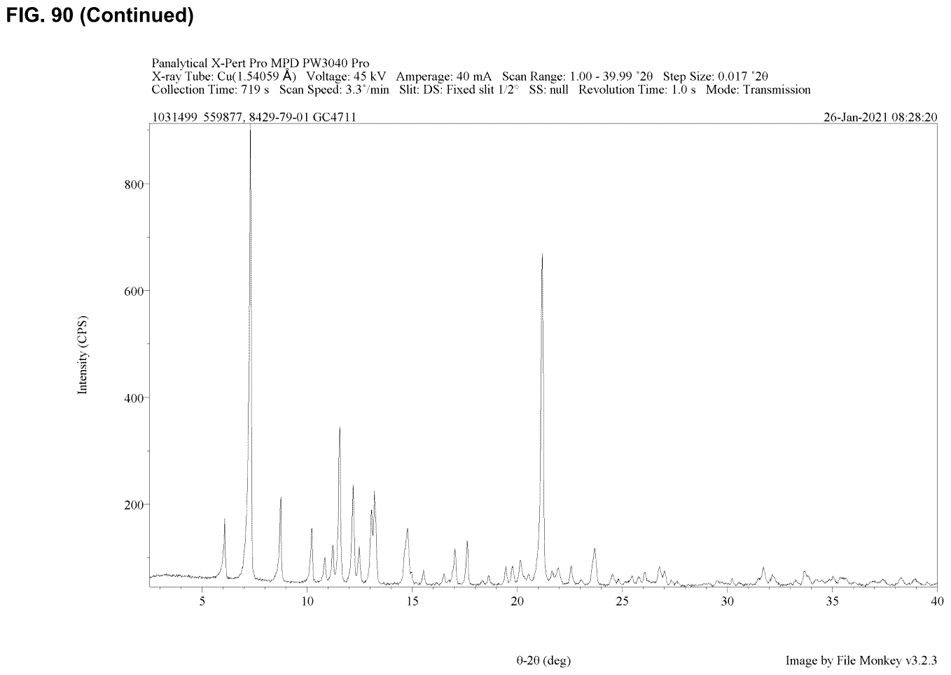

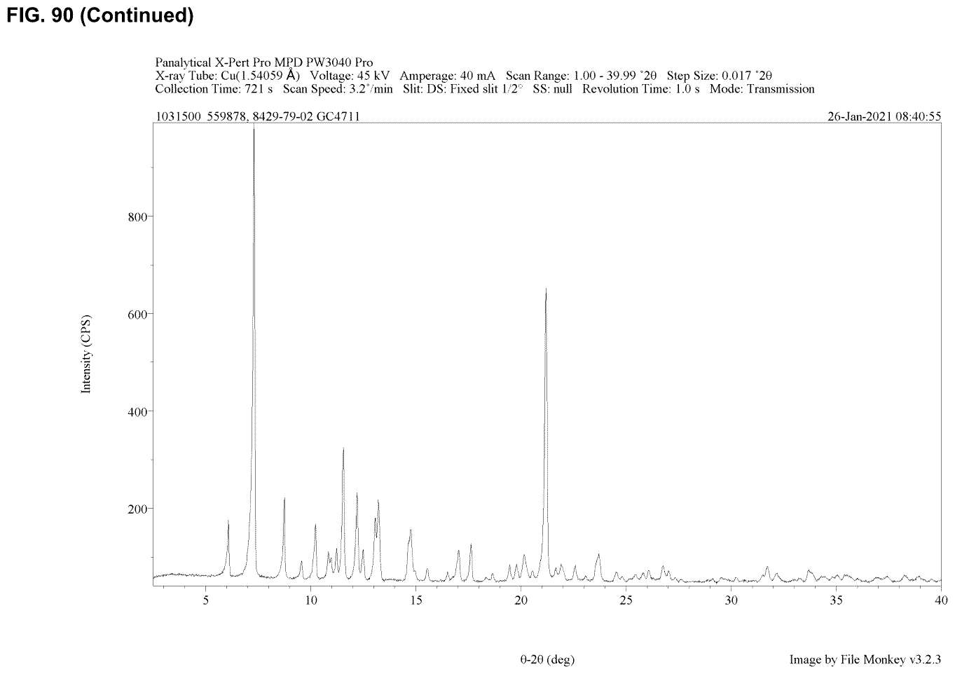

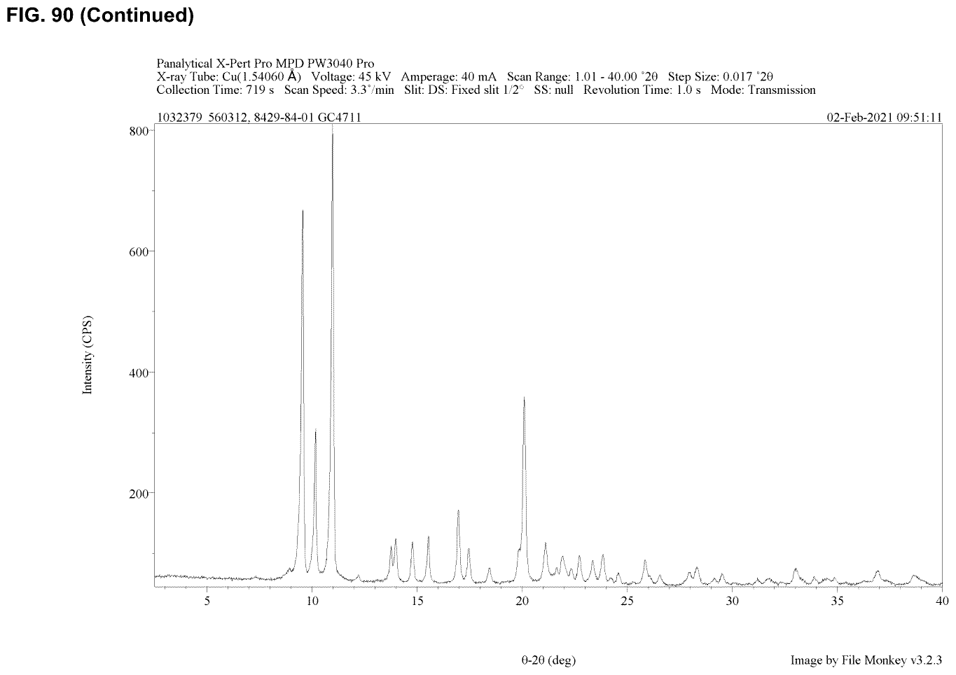

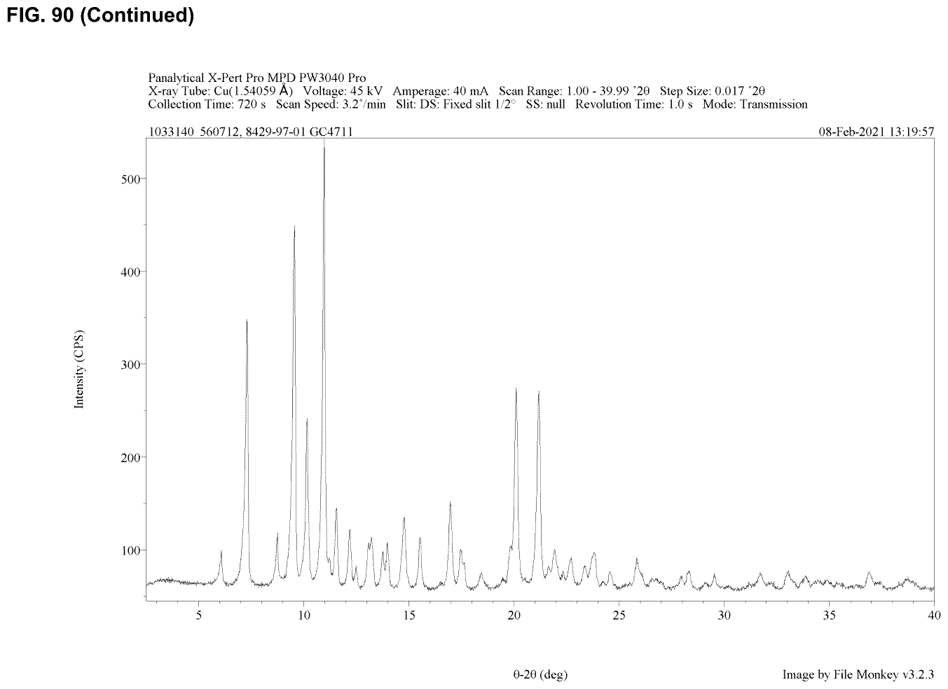

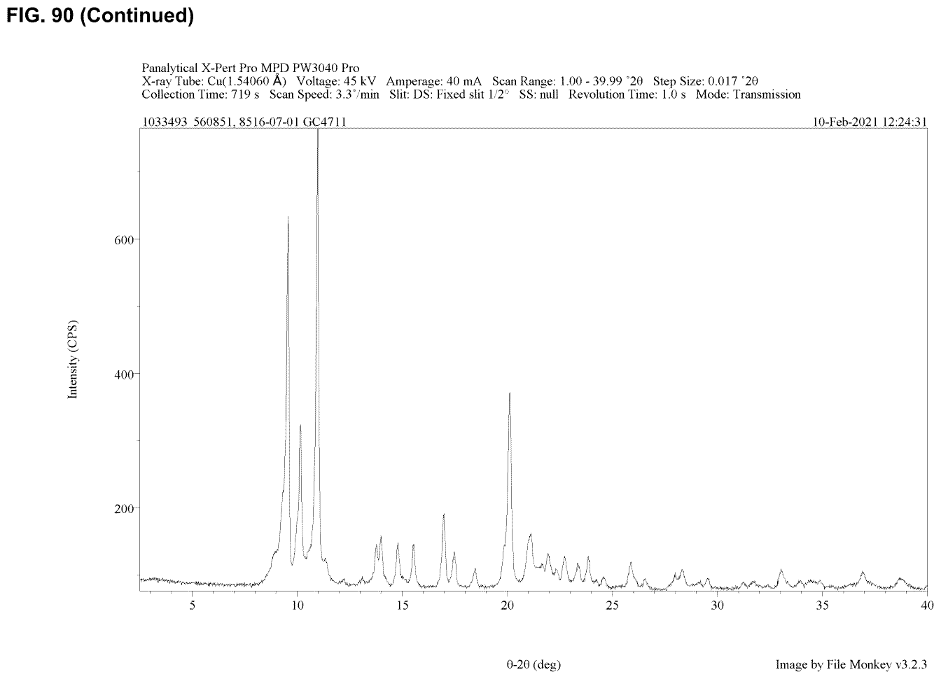

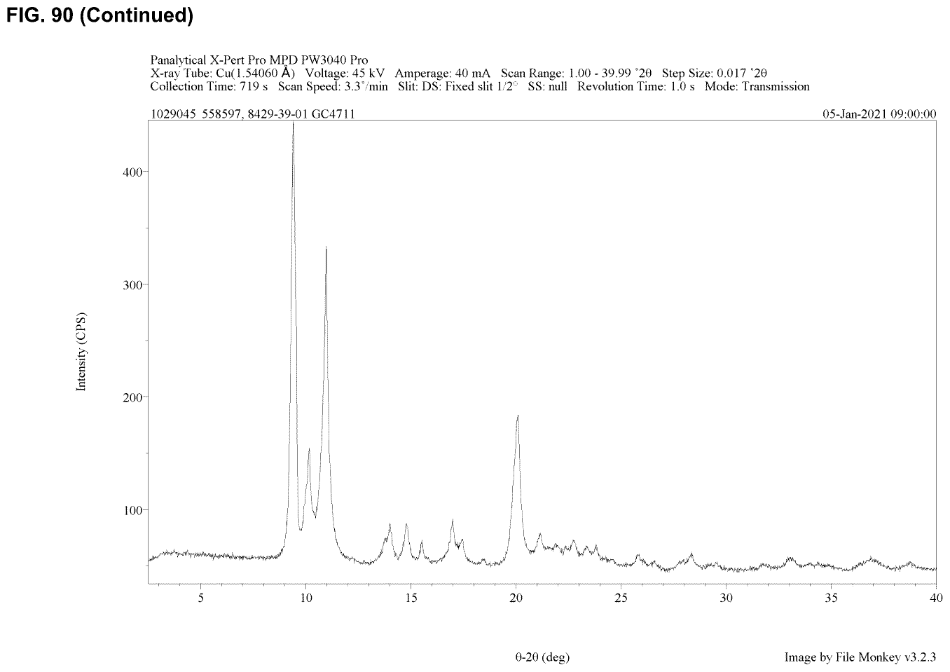

Raw data of this example can be found in FIG. 90.

### Experimental Settings

### Materials

GC4711 received from Galera Therapeutics for use in screening activities. Solvents and other reagents were purchased from commercial suppliers.

Samples were protected from light for all experiments (e.g. lights were turned off in the fume hood during handling and samples were covered with foil).

### Fast Evaporation

Solutions were prepared in various solvents and, typically, filtered through a 0.2-μm nylon or PTFE filter. Each solution was allowed to evaporate from an open vial at ambient conditions, unless otherwise stated. Solutions were allowed to evaporate to dryness unless designated as partial evaporations (solid present with a small amount of solvent remaining), in which case solids were isolated as described herein.

### Slurry Experiments

Suspensions were prepared by adding enough solids to a given solvent at the stated conditions so that undissolved solids were present. The mixture was then agitated (typically by stirring or oscillation) in a sealed vial at a given temperature for an extended period of time. The solids were isolated as described herein.

Differential Scanning calorimetry (DSC)

DSC was performed using a Mettler-Toledo DSC3+ differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, the weight was accurately recorded, and the sample was inserted into the DSC cell. A weighed aluminum pan configured as the sample pan was placed on the reference side of the cell. The pan lid was pierced prior to sample analysis. The samples were analyzed from −25° C. to 250° C. at 10° C./min.

A cycling DSC experiment was conducted for as-received amorphous, in which the sample was analyzed from −25° C. to 200° C., then cooled to −25° C. and reheated to 250° C. at 10° C./min.

### Optical Microscopy

Samples were observed under a Motic or Wolfe optical microscope with crossed polarizers or under a Leica stereomicroscope with a first order red compensator with crossed polarizers.

### Thermogravimetry (TGA)

Thermogravimetric analyses were performed using a Mettler-Toledo TGA/DSC3+ analyzer. Temperature and enthalpy adjustments were performed using indium, tin, and zinc, and then verified with indium. The balance was verified with calcium oxalate. The sample was placed in an aluminum pan. The pan was hermetically sealed, the lid pierced, and the pan was then inserted into the TG furnace. A weighed aluminum pan configured as the sample pan was placed on the reference platform. The furnace was heated under nitrogen. Samples were analyzed from 25° C. to 350° C. at 10° C./min.

Thermogravimetric analyses typically experience a period of equilibration at the start of each analysis, indicated by red parentheses on the thermograms. The starting temperature for relevant weight loss calculations is selected at a point beyond this region (typically above 35° C.) for accuracy.

DSC analysis on this instrument is less sensitive than on the DSC3+ differential scanning calorimeter. Therefore, samples with sufficient solids were analyzed by both instruments and only the TGA thermogram from this instrument is reported.

### X-Ray Powder Diffraction (XRPD)

### Transmission Geometry (Most Samples)

XRPD patterns were collected with a PANalytical X'Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e or 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 5.5. The data acquisition parameters for each pattern are displayed above the image in the Data section of this report. All images have the instrument labeled as X'Pert PRO MPD regardless of the instrument.

### Results and Discussion

### A. Received Materials

Two grams of amorphous GC4711 lot JR-C17092208-G19001 was received for use and stored under USP freezer conditions (Table 48).

TABLE 48
GC4711 as Received.

| Lot No. | Description | Storage | Quantity | LIMS |
|---|---|---|---|---|
| JR-C17092208-G19001 | amorphous | freezer | 2 g | 558111 |

### B. Attempts to Generate GC4711 Form E

a. Desolvation and Heat Induced Crystallization

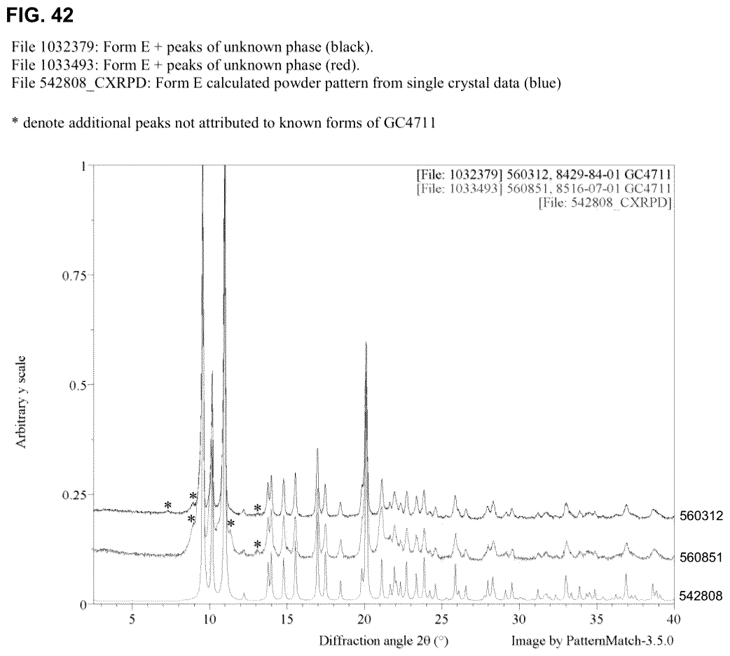

Experiments to isolate Form E through the dehydration of GC4711 Form A Sesquihydrate are described in Tables 49 and 50. These attempts were not successful in providing Form E as a pure crystalline phase. Mixtures primarily composed of Form E and other unidentified crystalline phase(s) were obtained (FIG. 42).

TABLE 49
Generation of GC4711 Forms.

| Solvent | Method^{a} | Observation^{b} | Result | Sample | LIMS | File | Page |
|---|---|---|---|---|---|---|---|
| acetone | 1. 51 mg/0.2 mL | 1. slurry | E + A | 8429-39-02 | 558598 | 1029046 | 17 |
| | 2. ambient, 5 min. | 2. tablets, birefringent | | | | | |
| | 3. evaporated under N₂ | 3. — | | | | | |
| | while vial warmed | | | | | | |
| Et₂O | 1. 192.7 mg/14 mL | 1. — | E | 8429-39-03 | 558636 | 1029139 | 18 |
| | 2. slurry, ambient, 1 day | 2. — | | | | | |
| | 3. filtered, stored over | 3. — | | | | | |
| | P₂O₅ | | | | | | |
| | 8429-39-03 filtrate fast | retained for reuse if | — | 8429-40-02 | — | — | — |
| | evaporation | needed | | | | | |
| | 1. 1 g/20 ml, seeded with | 1. — | E + | 8429-40-01 | 558788 | 1029579 | 19 |
| | 8429-39-03 | | peaks | | | | |
| | 2. slurry, ambient, 1 day | 2. — | | | | | |
| | 3. filtered, vacuum dried | 3. 925.6 mg recovered | | | | | |
| | ambient, 1 d | | | | | | |
| | 4. stored over desiccant | 4. — | | | | | |
| | 8429-40-01 | 1. off-white | E + | 8429-59-01 | 559598 | 1031029 | 20 |
| | 1. slurry, ambient, 5days | 2. — | peaks | | | | |
| | 2. filtered, N₂ dried 1 day | | | | | | |
| | 1. 770 mg/7 ml | 1. — | E + | 8429-73-01 | 559668 | 1031211 | 21 |
| | 2. purged with N₂ | 2. — | peaks | | | | |
| | 3. slurry [symbol] ambient, 3.5 hrs | 3. off-white | | | | | |
| | 8429-73-01 filtrate + | 1. — | E, A + | 8429-73-03 | 559857 | 1031458 | 22 |
| | 8429-73-02 filtrate | 2. pale tan | peak @ | | | | |
| | 1. refrigerated 4 days | | 6.7° | | | | |
| | 2. filtered | | | | | | |
| EtOH | reused multiple samples | 1. yellow solution | C | 8429-73-02 | 559856 | 1031457 | 23 |
| | 1. added solvent | 2. no changes | | | | | |
| | 2. filtered in to ether | 3. oily | | | | | |
| | 3. partial evap under Na | 4. micleation observed | | | | | |
| | 4. ether added | S. increased nucleation | | | | | |
| | 5. heptane added | 6. off white solids | | | | | |
| | 4. filtered | | | | | | |
| EtOAc/ | 8429-59-01 sub sample | partial dissolution, | A | 8429-79-01 | 559877 | 1031499 | 24 |
| wet | sonicated briefly, filtered | clumped, then broke | | | | | |
| | 8429-79-01 filtrate | fines, B | A + E | 8429-79-02 | 559878 | 1031500 | 25 |
| | fast evaporation | | | | | | |
| | 8429-59-01sub sample | clumped and slowly | — | 8429-97-01 | — | — | — |
| | slurry/sonicated 5 min | broke apart | | | | | |

[symbol]

indicates data missing or illegible when filed

TABLE 50
GC4711 Form E Generation Attempts via Desolvation or Heating.

| Method^{a} | Observation^{b} | Result | Sample | LIMS | File | Page |
|---|---|---|---|---|---|---|
| 8429-79-01 and 8429-79-02, Form A + E | — | E + A | 8429-84-01 | 560312 | 1032379 | 26 |
| combined and stored over P₂O₅, 5 days | | | | | | |
| P₂O₅, 4 days | free flowing, off-white | E + A | 8429-97-03 | 560712 | 1033140 | 27 |
| 8429-97-01 Forn E + Form A | off-white | peaks | 8516-07-01 | 560851 | 1033493 | 28 |
| vacuum dried, ambient, 2 days | | | | | | |
| 1. amorphous material vial purged with N₂ | 1. pale light yellow | E | 8429-39-01 | 558597 | 1029045 | 29 |
| 2. capped | 2. — | disorder | | | | |
| 3. held @ 100° C., 5 min. | 3. peach in color | | | | | |

^{a}Times and temperatures are approximate unless noted.
^{b}B =birefringent and
NB = non birefringent when material viewed using polarized light microscopy.

Heat induced crystallization of amorphous GC4711 is described in Table 50. Exposure to elevated temperatures caused discoloration, suggesting that chemical degradation occurred.

b. Trituration in Diethyl Ether

The trituration experiments of amorphous GC4711 in diethyl ether are summarized in Table 49. Characterization data of the resulting materials are provided in Table 51.

TABLE 51
Characterization Data for Form E Anhydrate Samples.

| Technique | Details | Result^{c} | Sample | LIMS | File | Page |
|---|---|---|---|---|---|---|
| XRPD | — | Form E + peaks | 8429-40-01 | 558788 | 1029597 | 19 |
| TGA | ambient—350° C. | 0.18% wt. loss up to 250° C. | 8429-40-01 | 558788 | 1029581 | 11 |
| DSC | −30 to 250° C. | minor endotherm onset 193° C. | 8429-40-01 | 558788 | 1029580 | |
| | | endotherm onset of 233° C. | | | | |
| XRPD | | Form E + peaks | 8429-73-01 | 559668 | 1031211 | 21 |
| DSC | −30 to 250° C. | minor endotherm near 197° C. | 8429-73-01 | 559668 | 1031459 | 12 |
| | | endotherm onset of 232° C. | | | | |

^{c}Reported weight loss is rounded to nearest hundredth and temperatures to the nearest whole number.

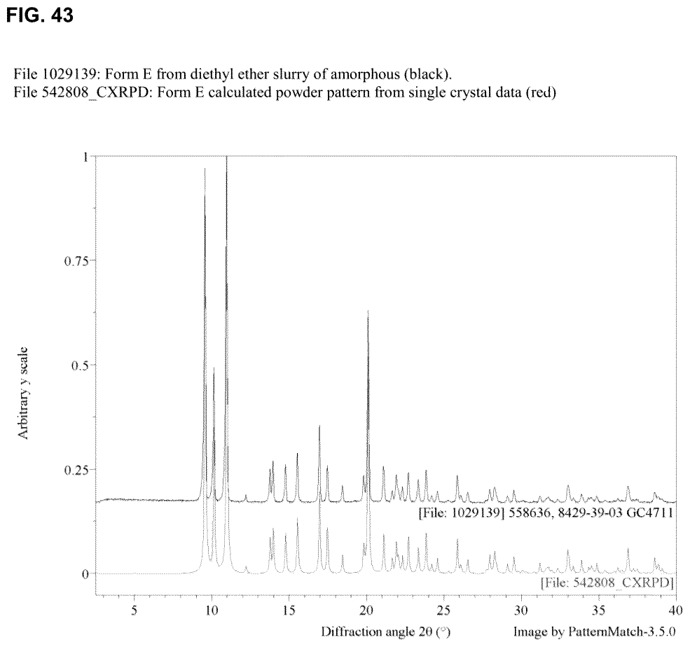

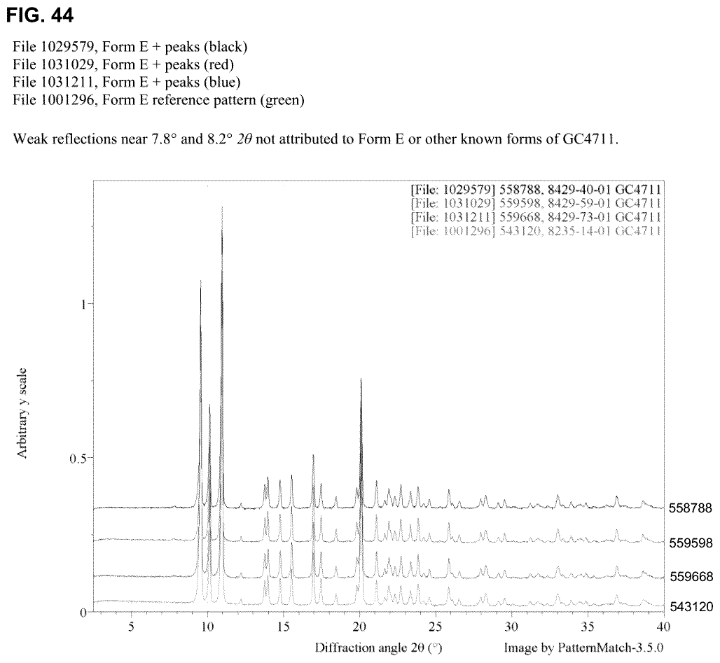

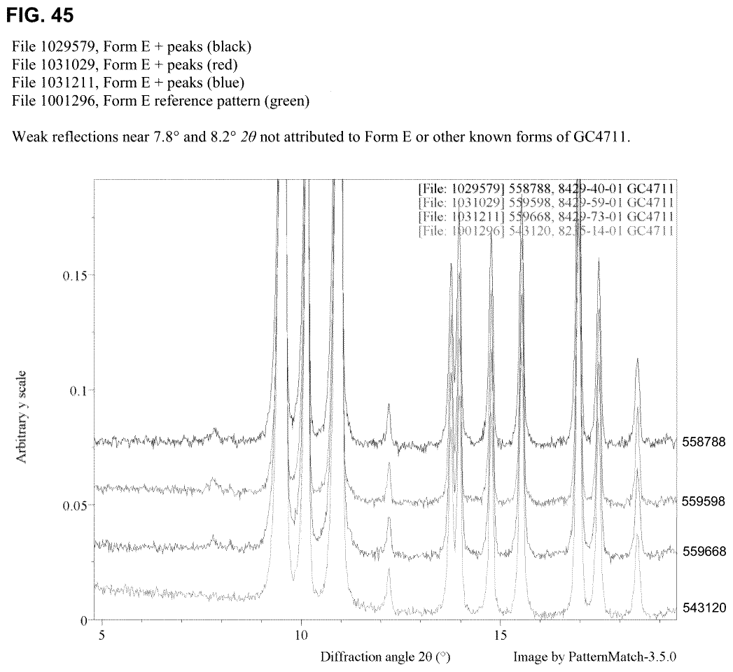

A scoping experiment to determine the feasibility of crystallizing Form E through the trituration of amorphous GC4711 in diethyl ether was performed at ˜200-mg scale. Based on the X-ray powder diffraction (XRPD) pattern, the scoping experiment was successful (FIG. 43). However, attempts at ˜1-g scale using the same conditions did not provide Form E as a pure crystalline phase. Additional, weak reflections were observed at 7.8° and 8.2° 2θ (FIG. 44 and FIG. 45). These additional, weak reflections do not match any known forms of GC4711.

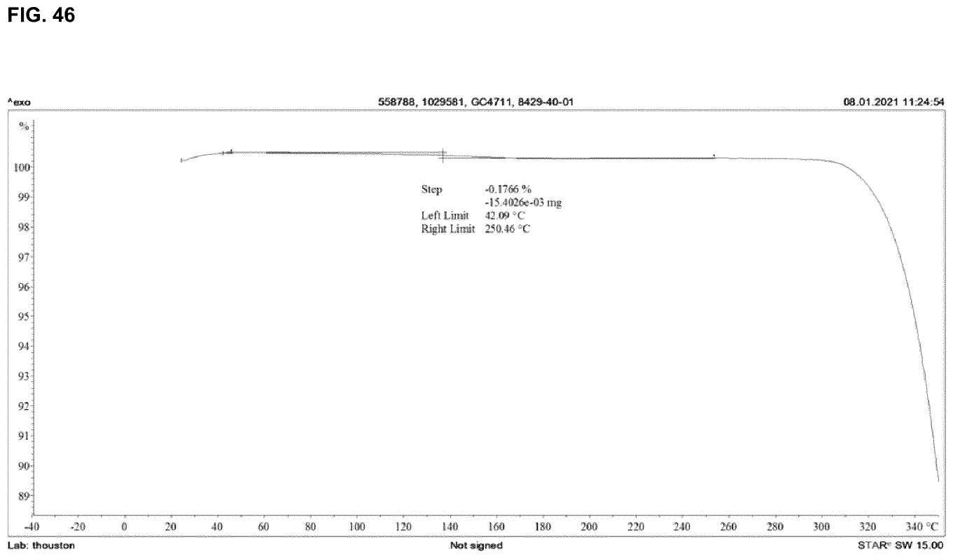

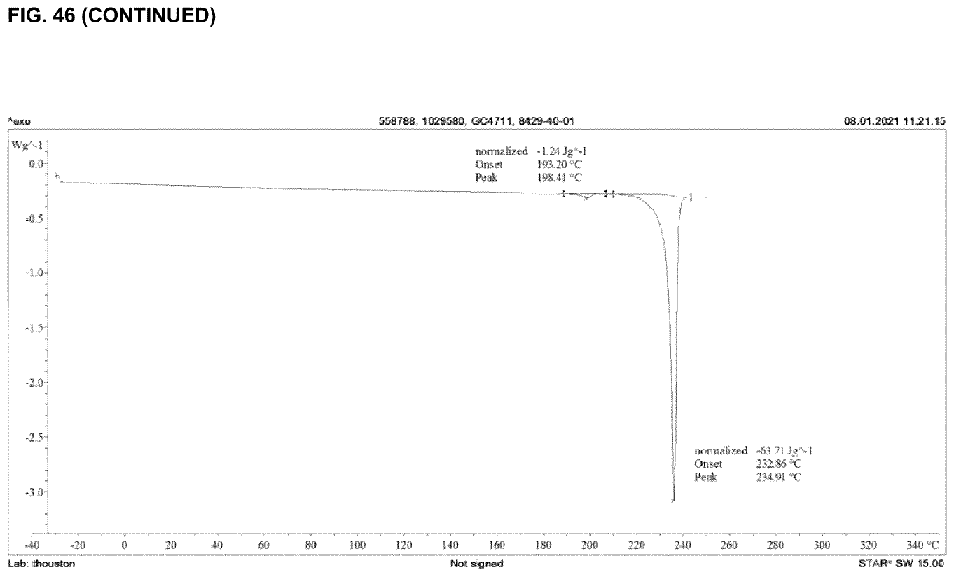

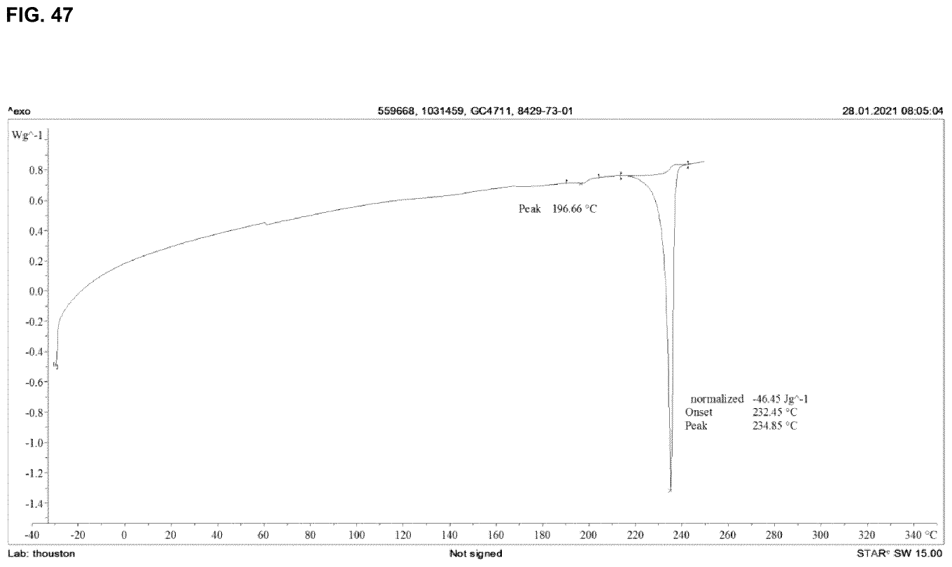

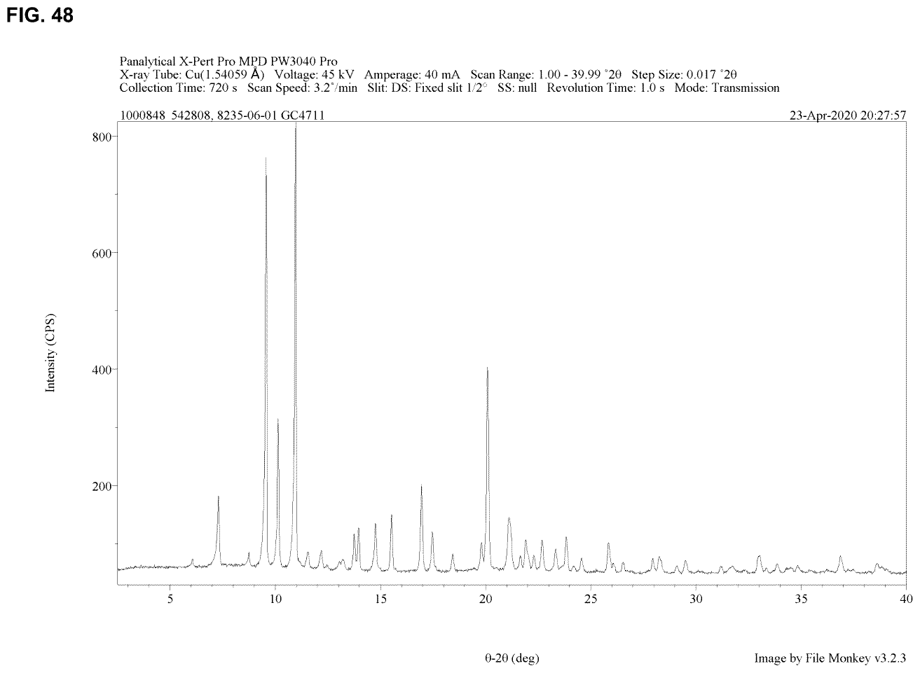

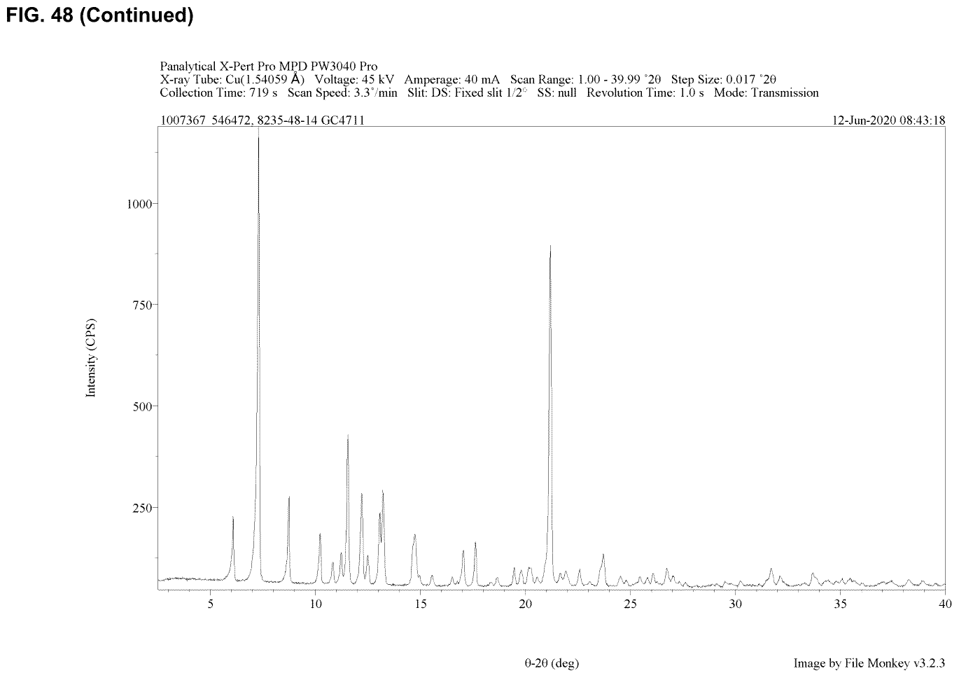

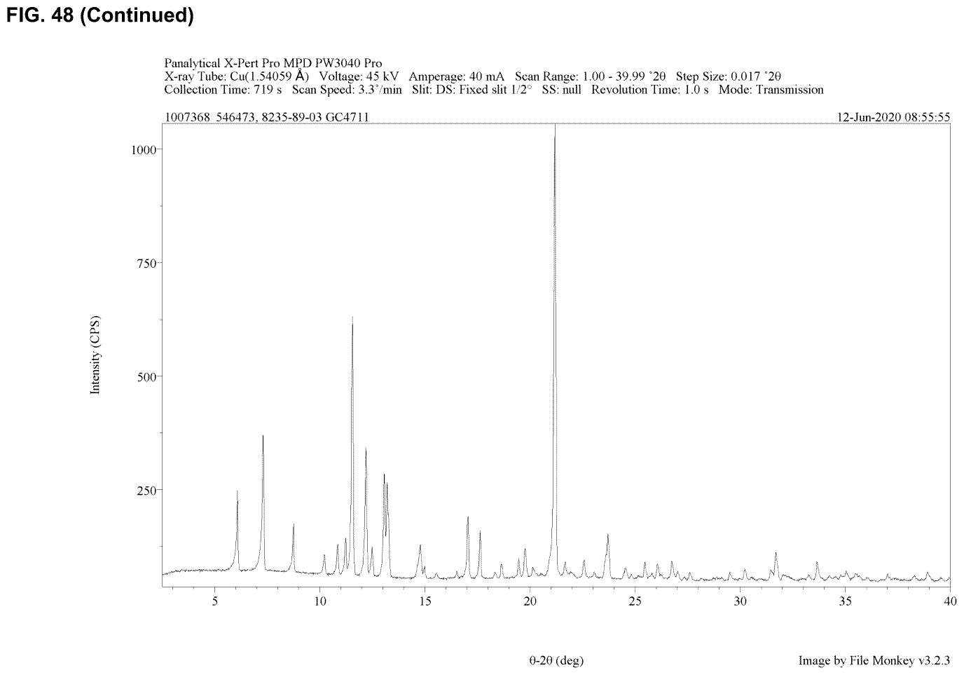

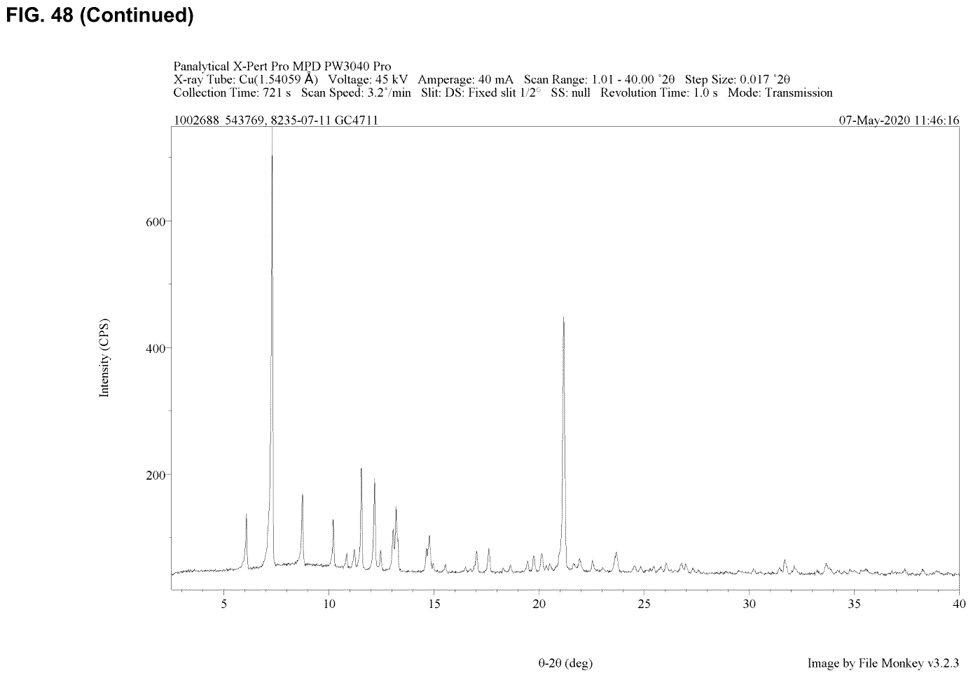

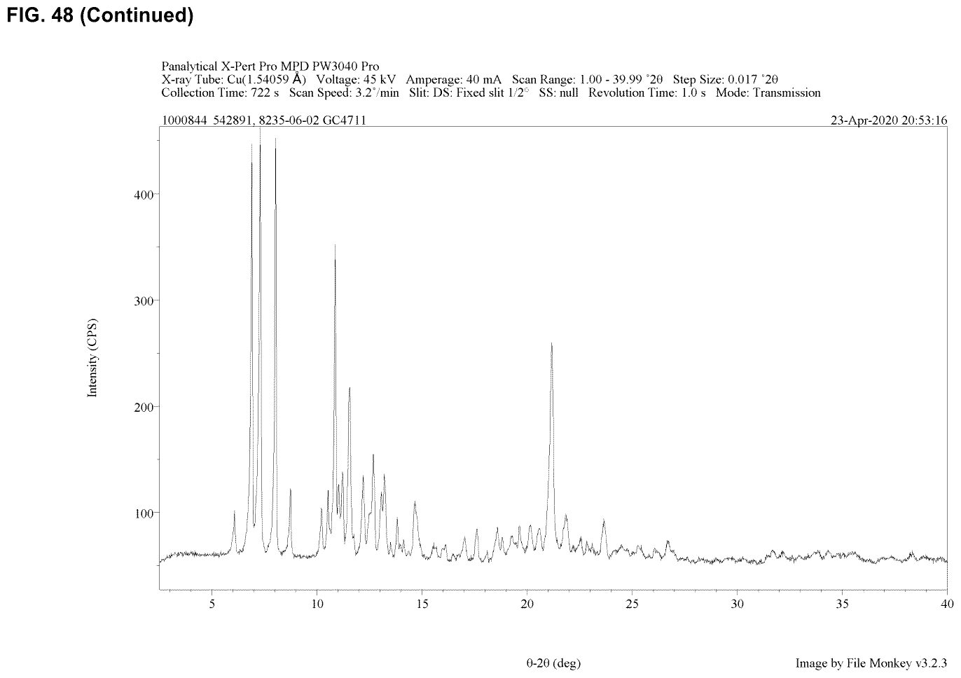

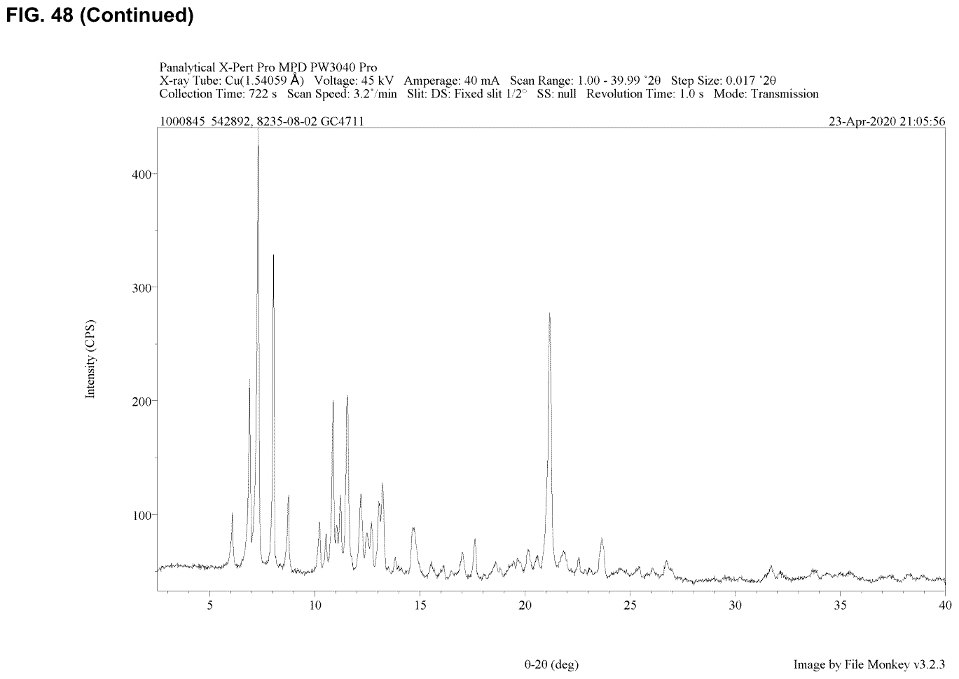

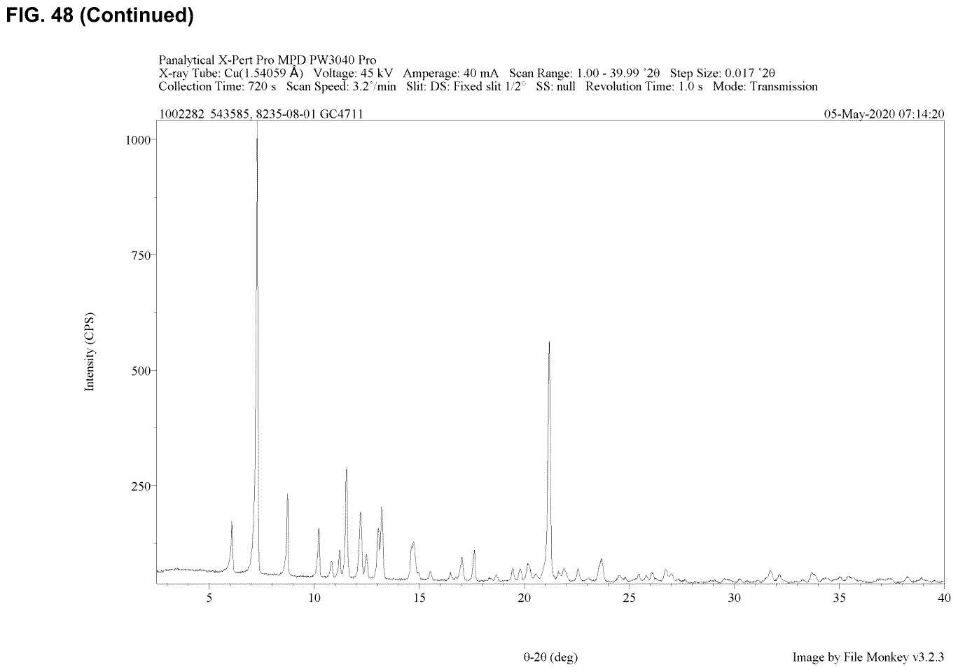

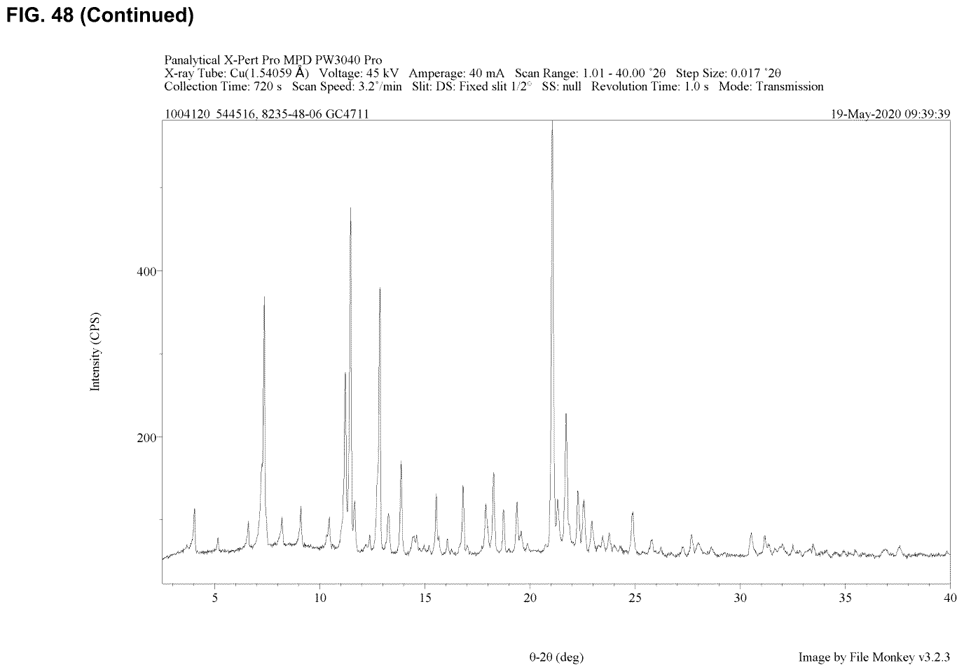

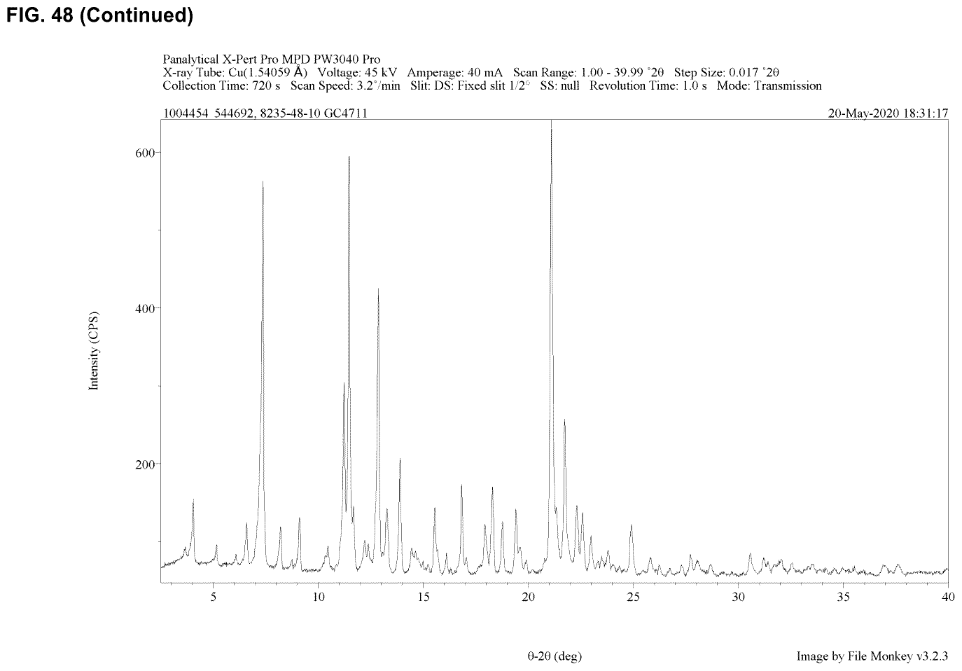

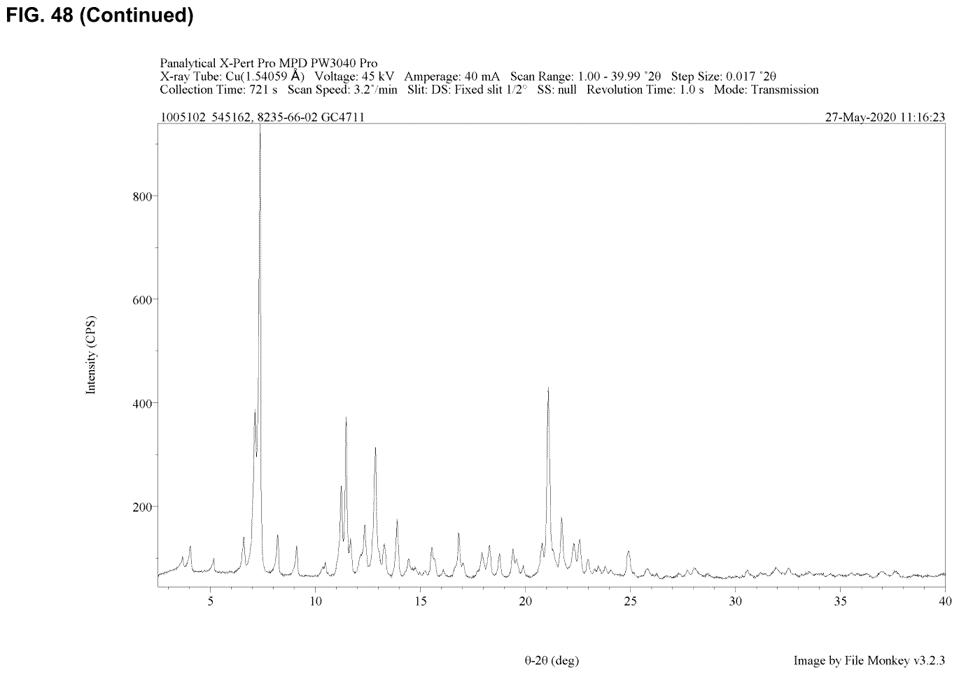

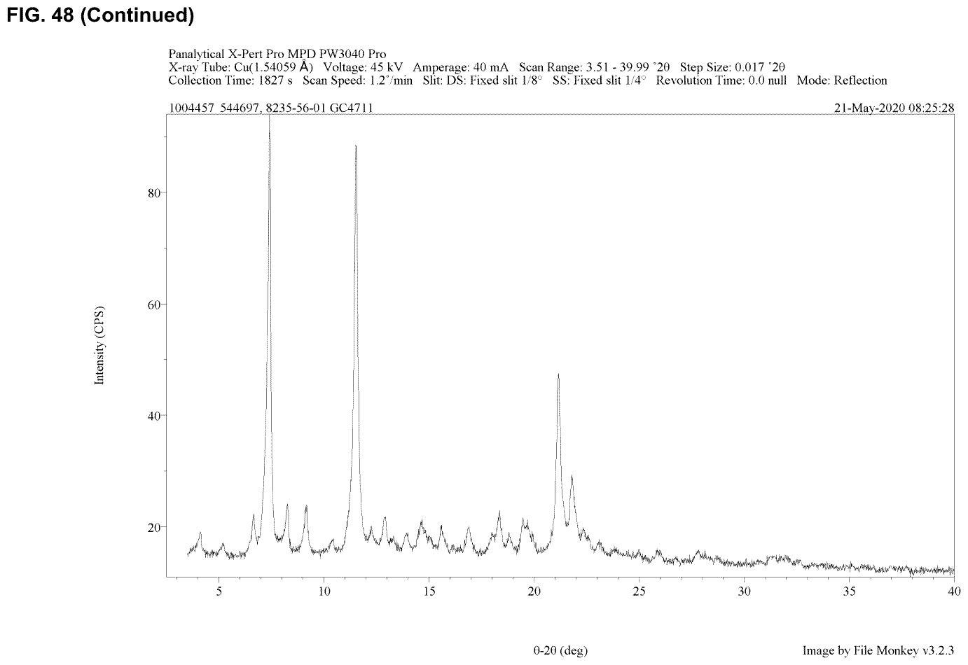

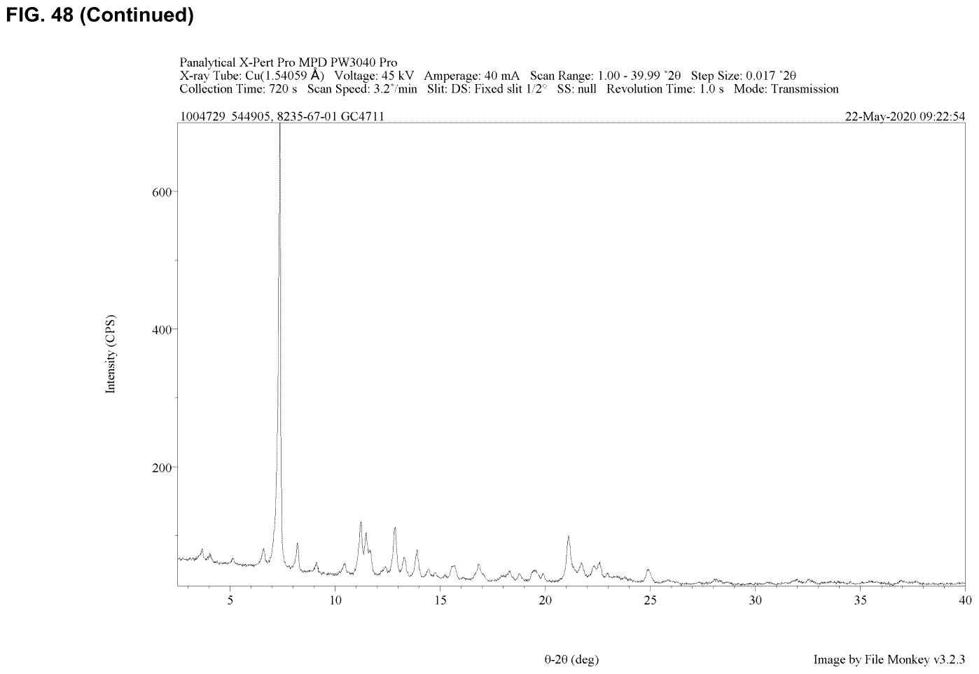

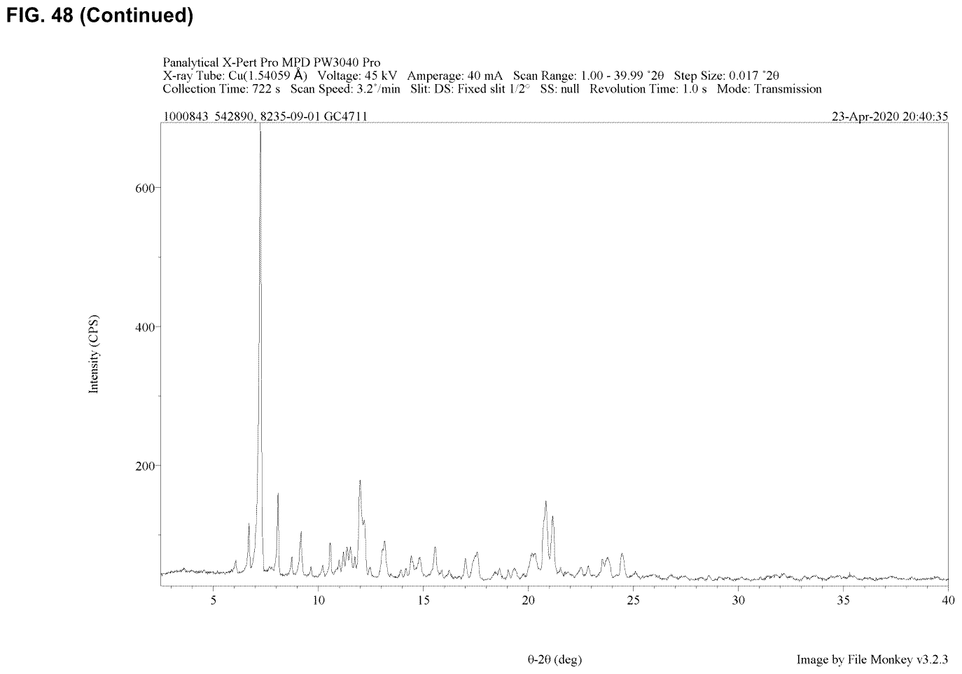

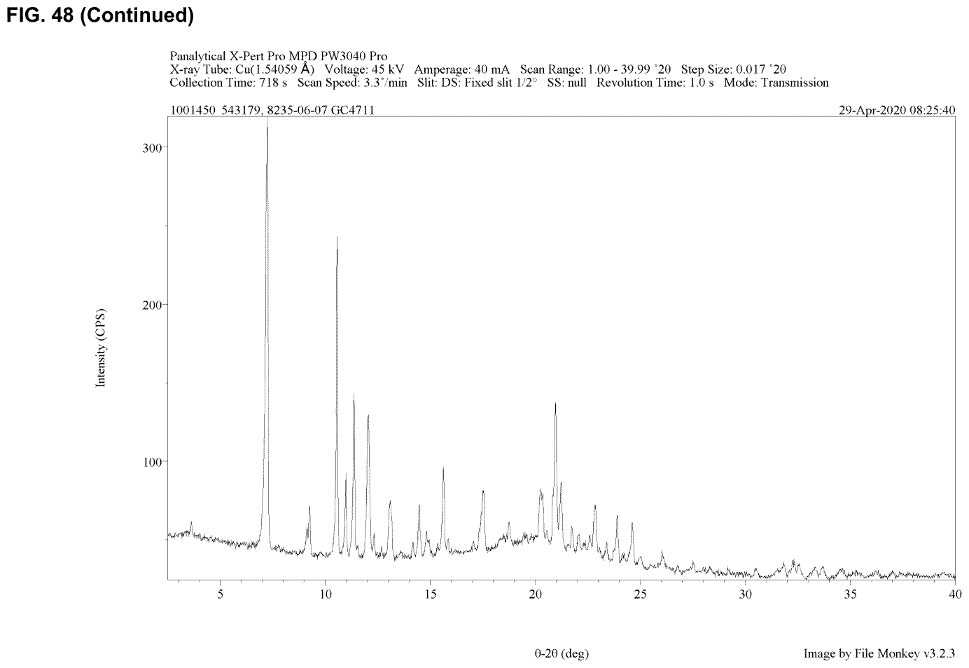

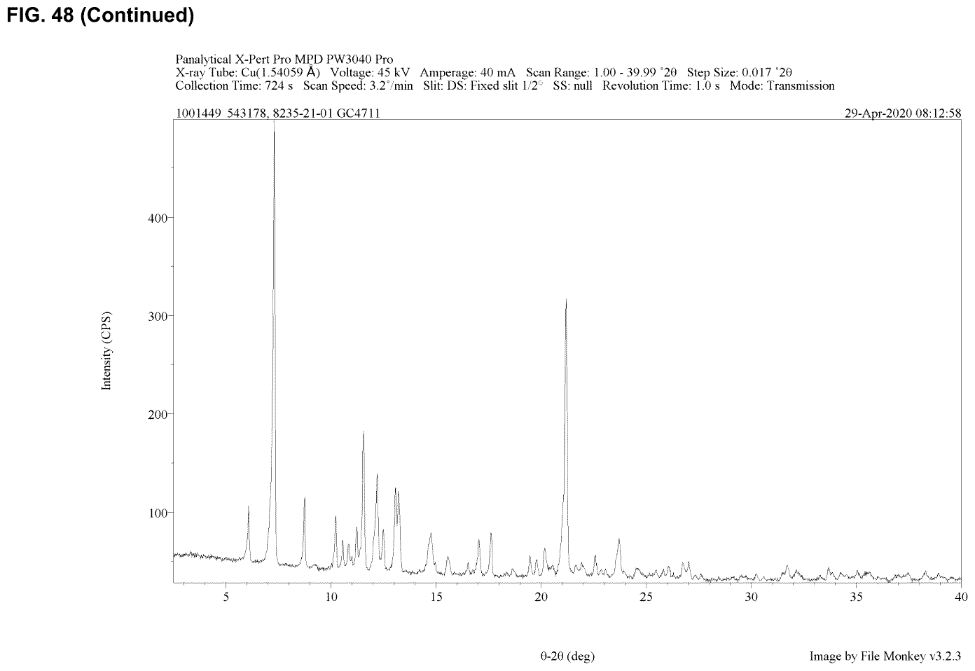

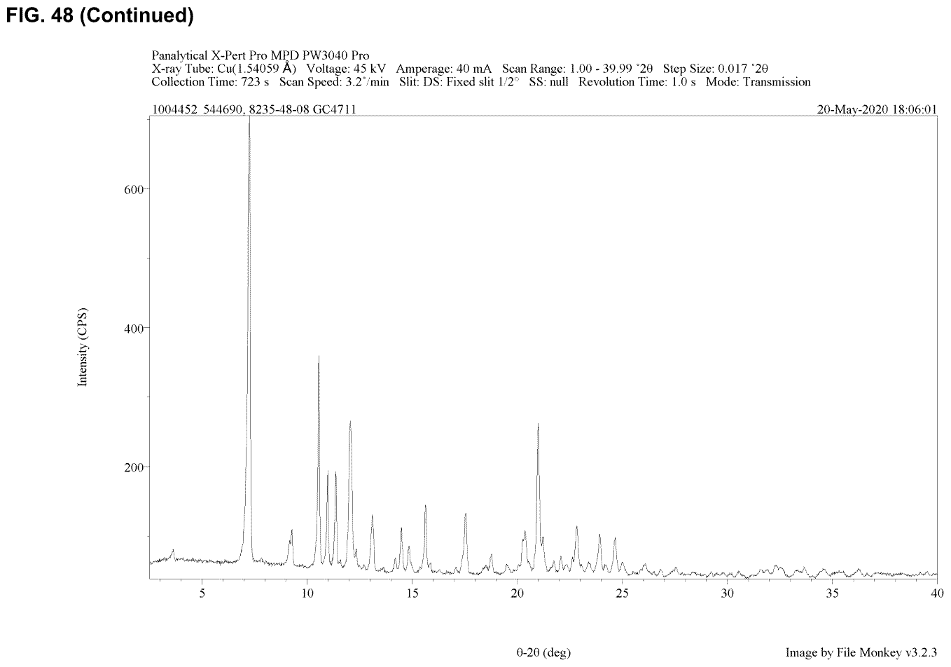

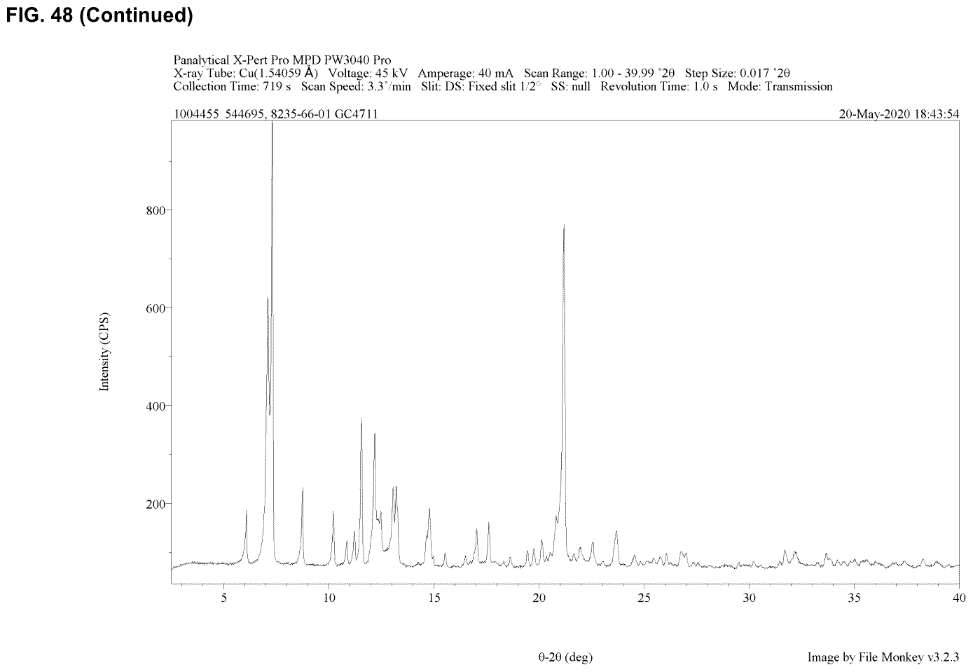

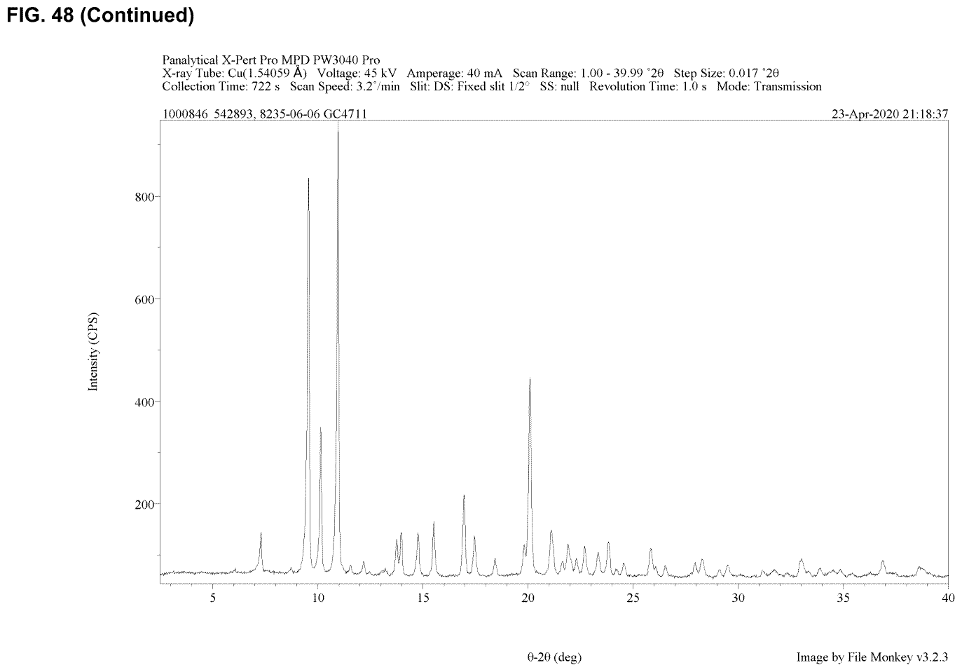

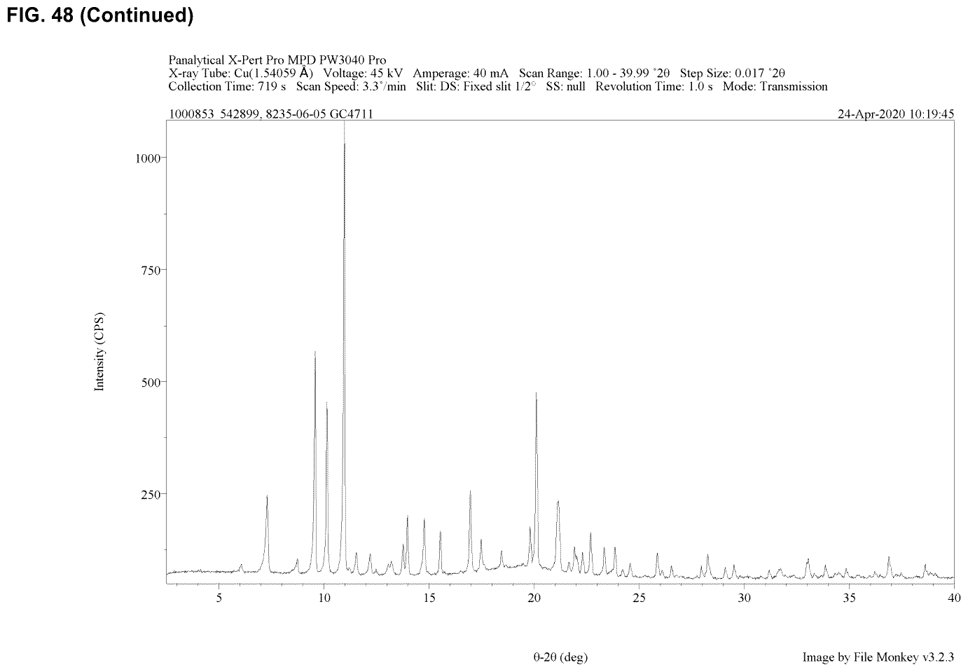

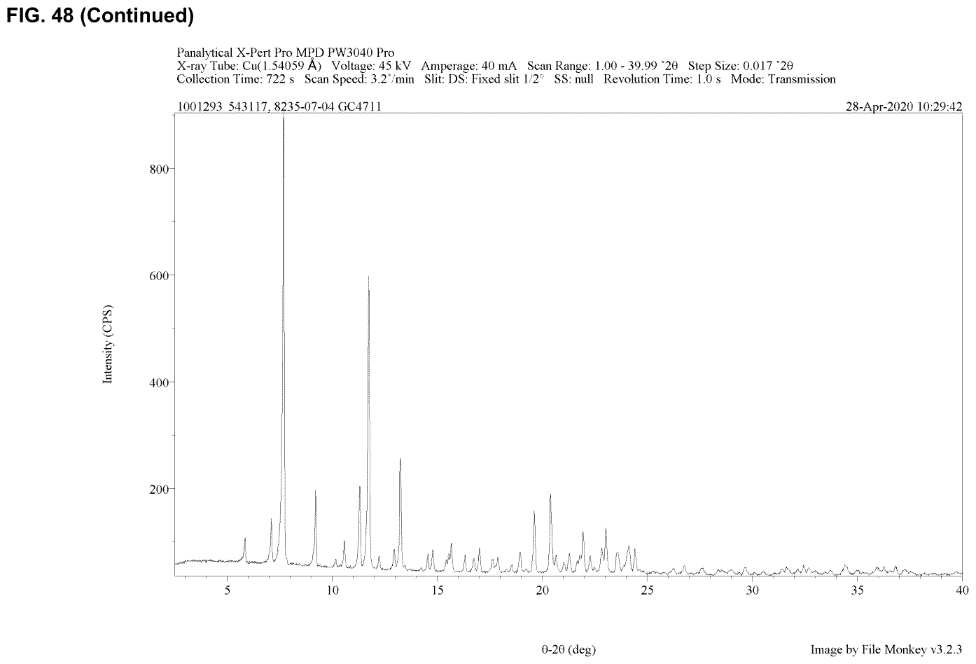

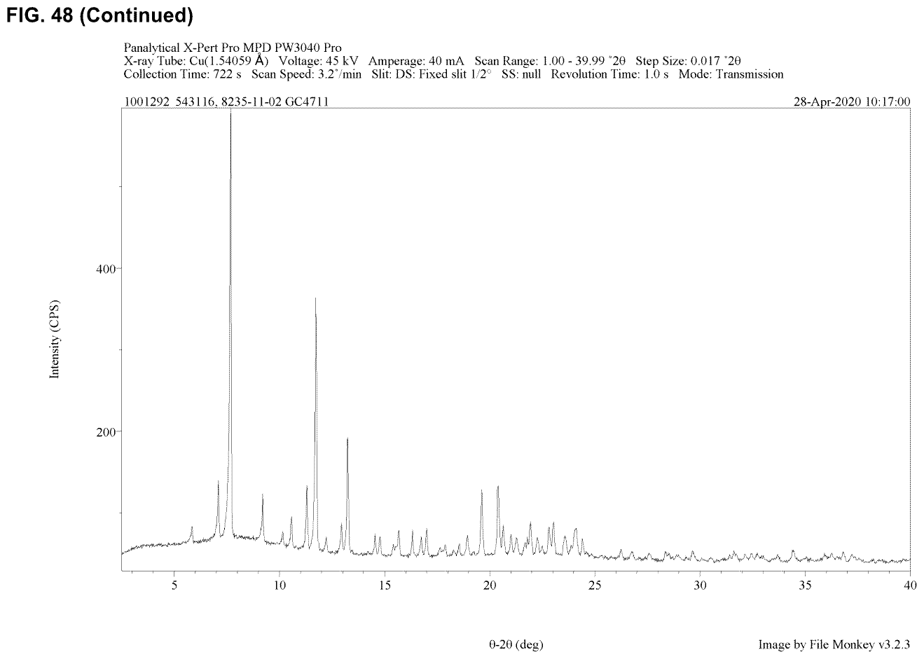

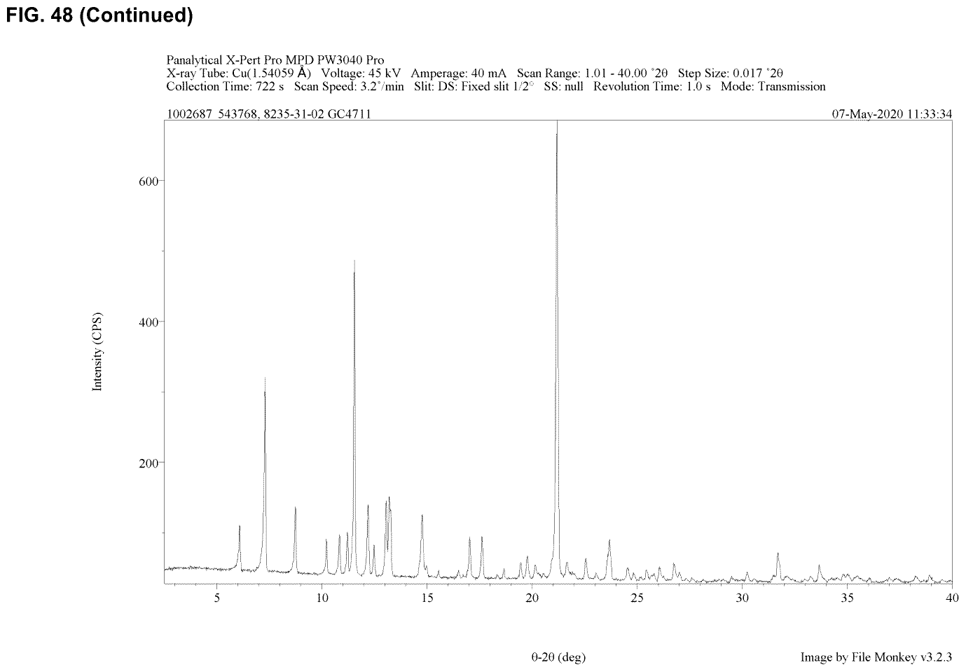

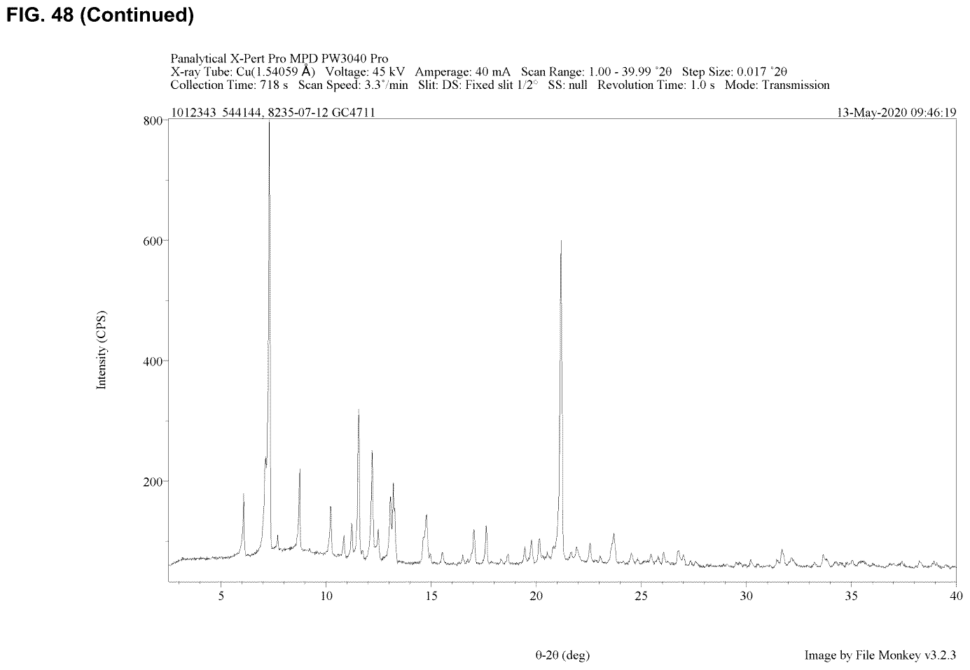

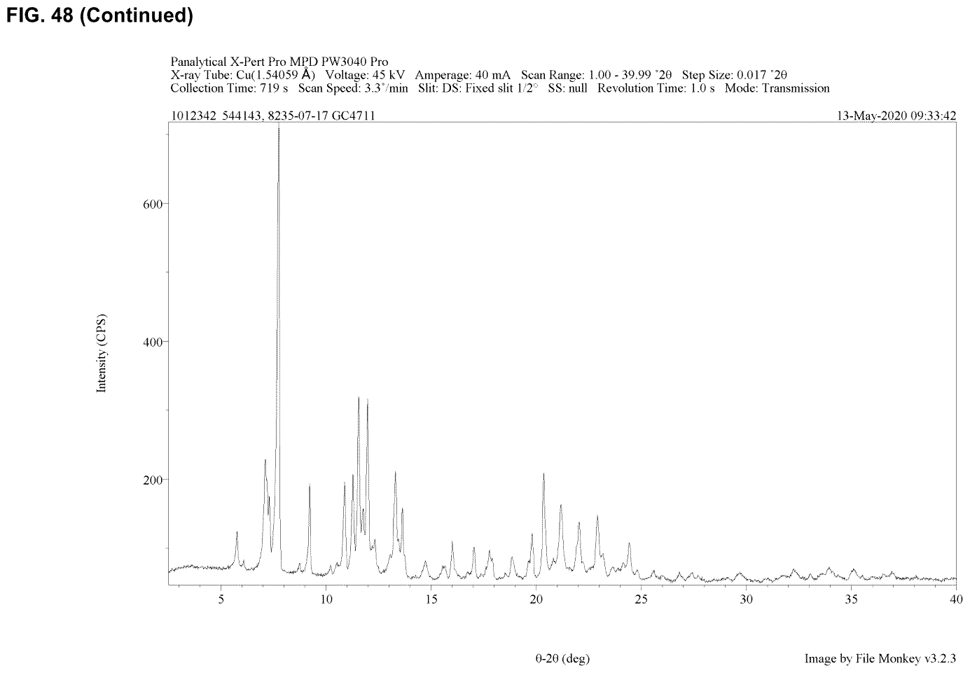

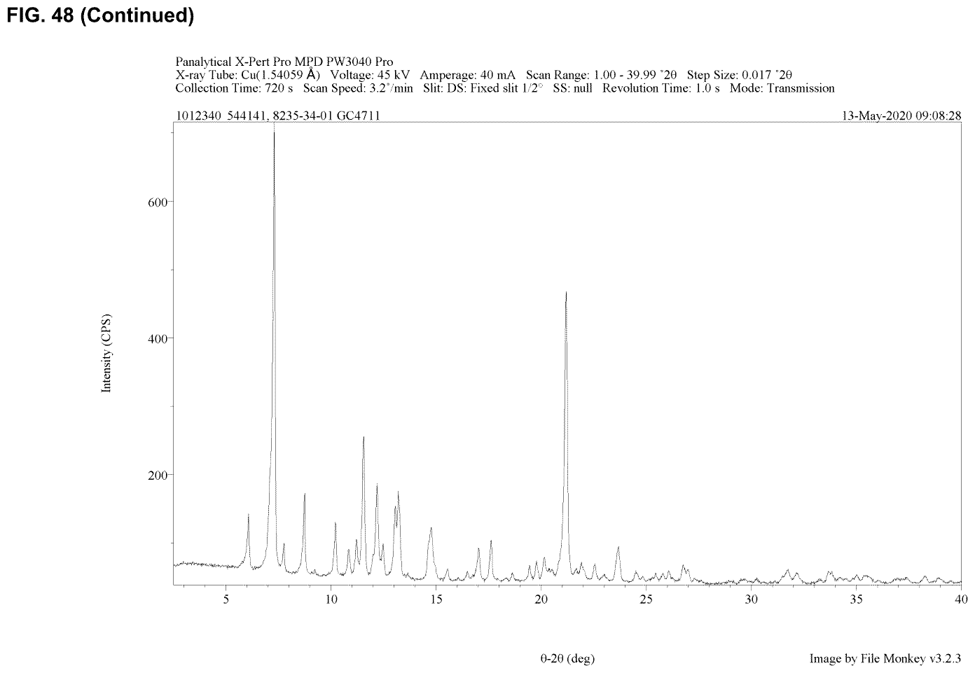

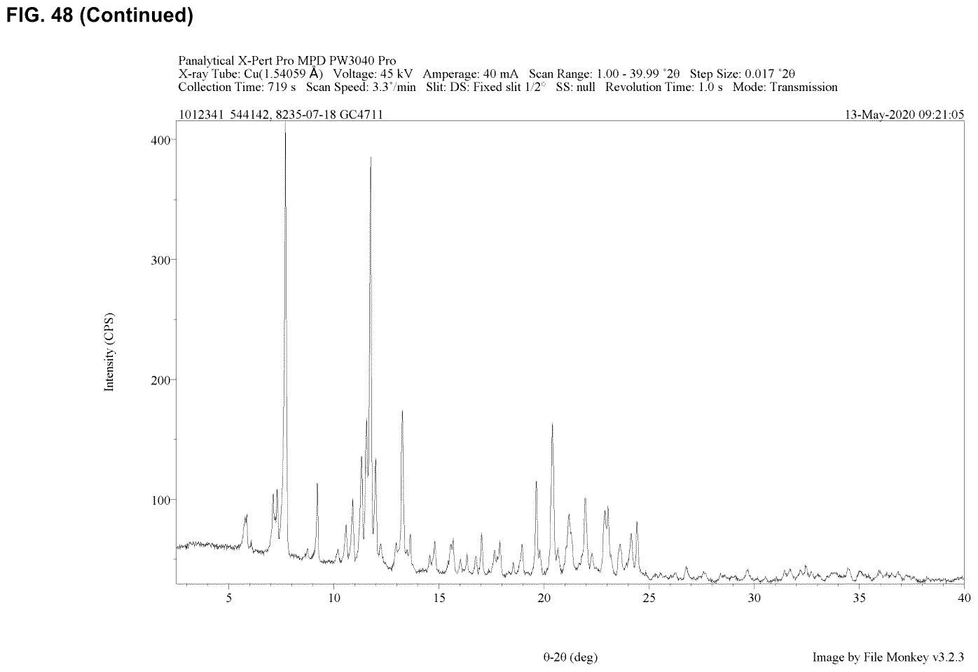

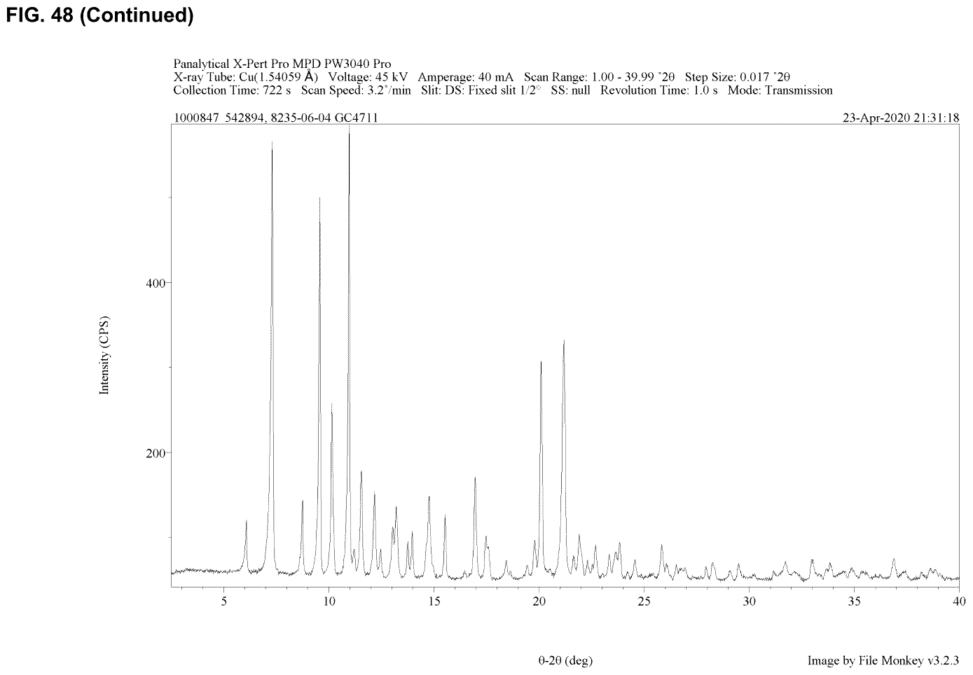

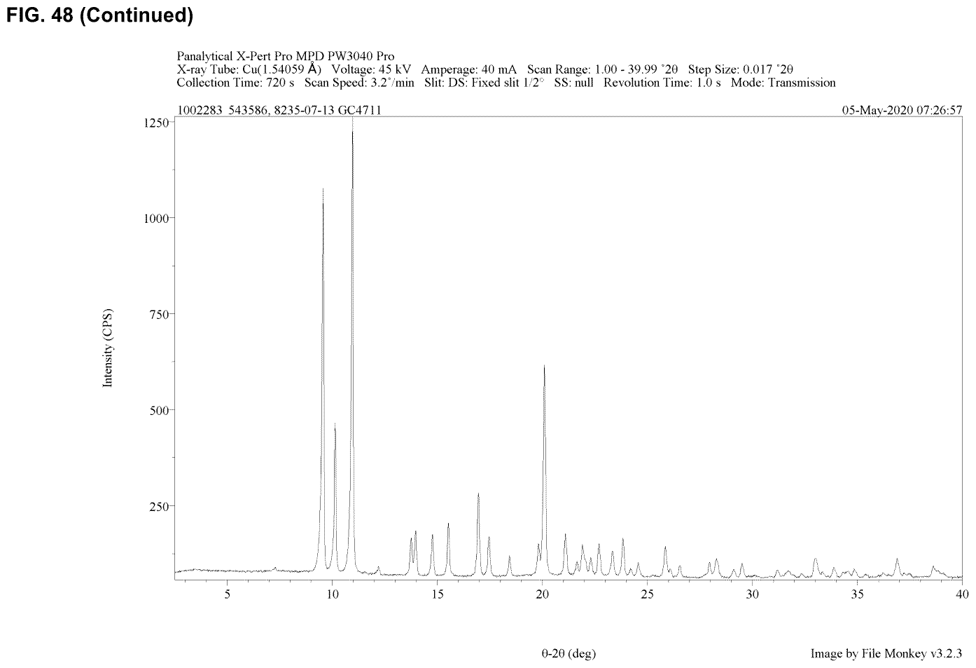

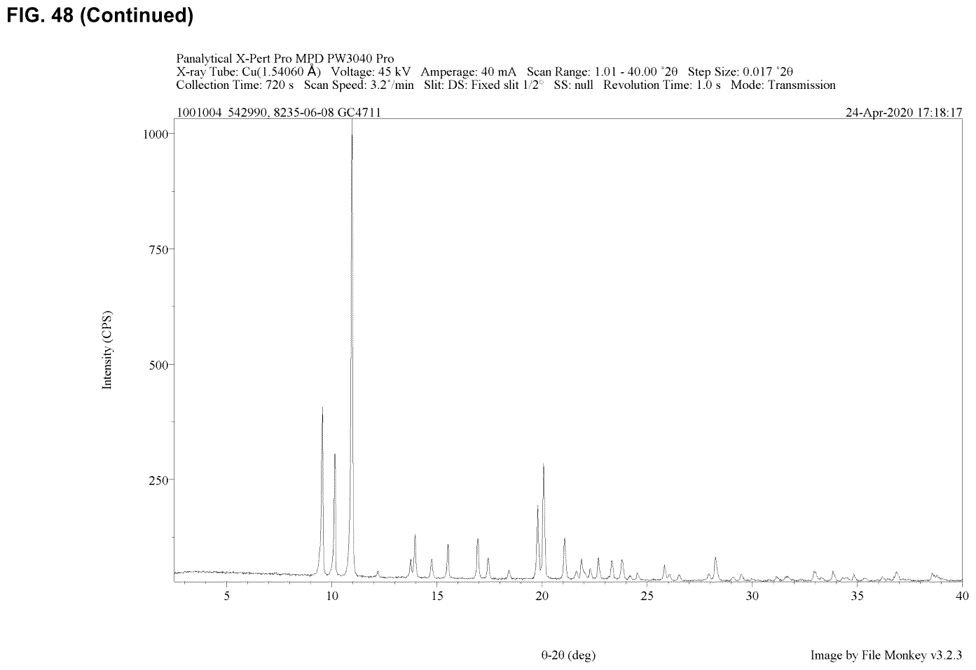

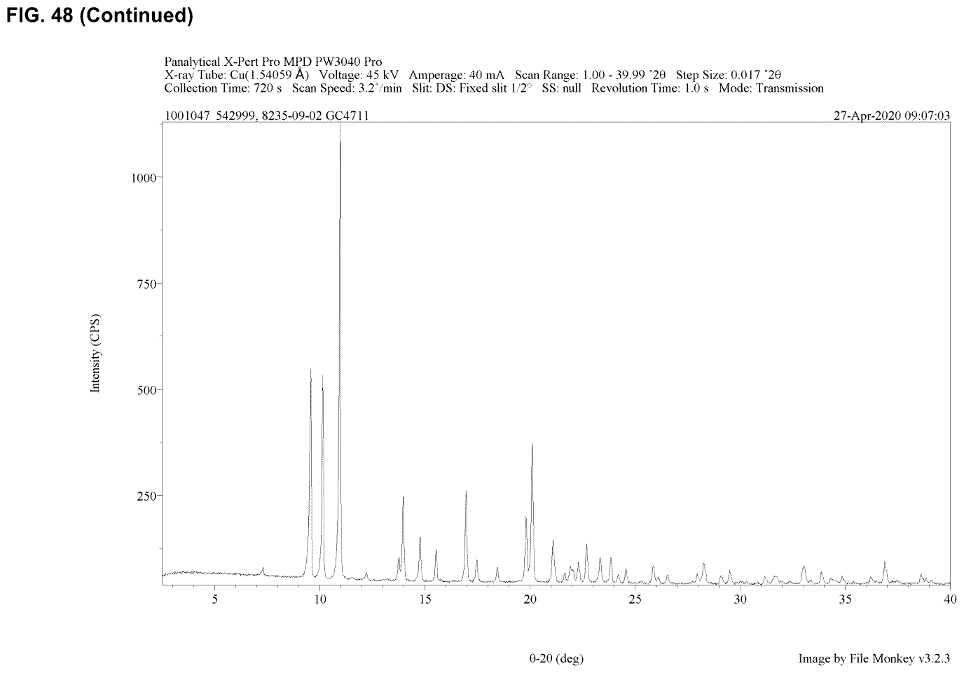

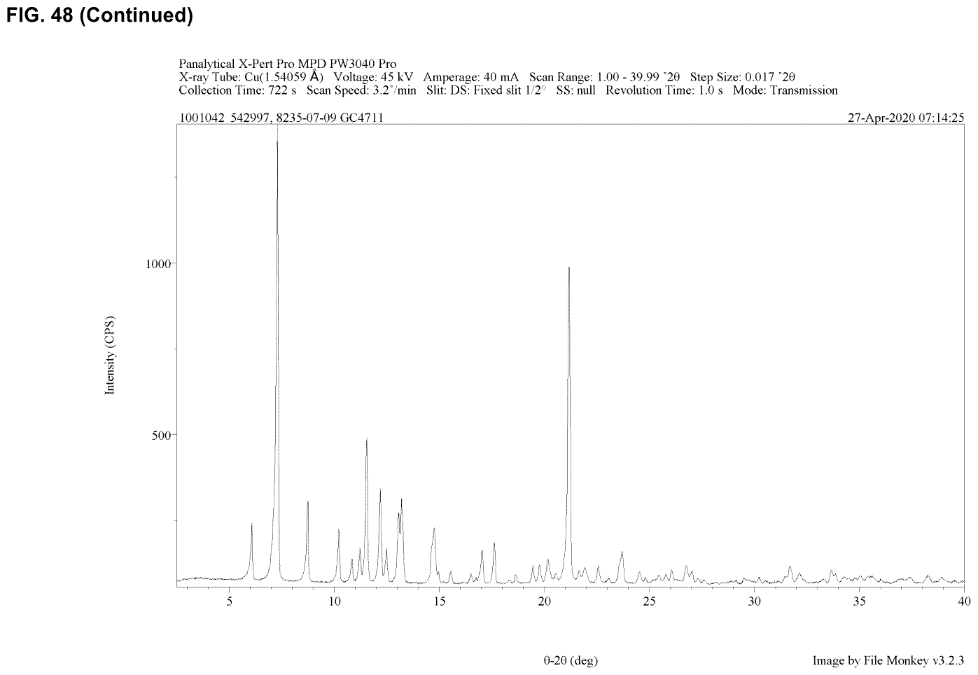

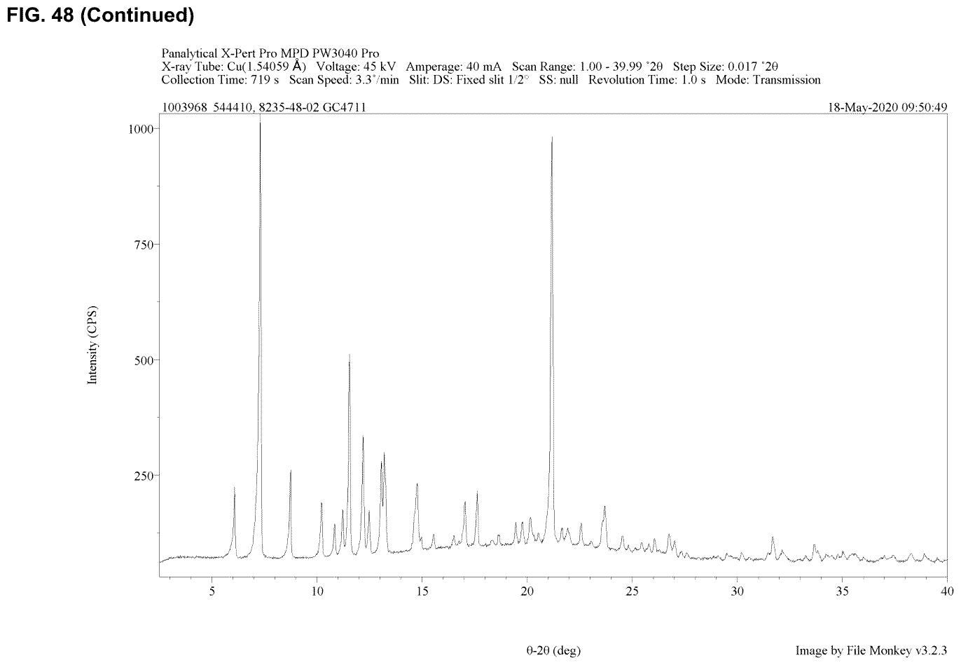

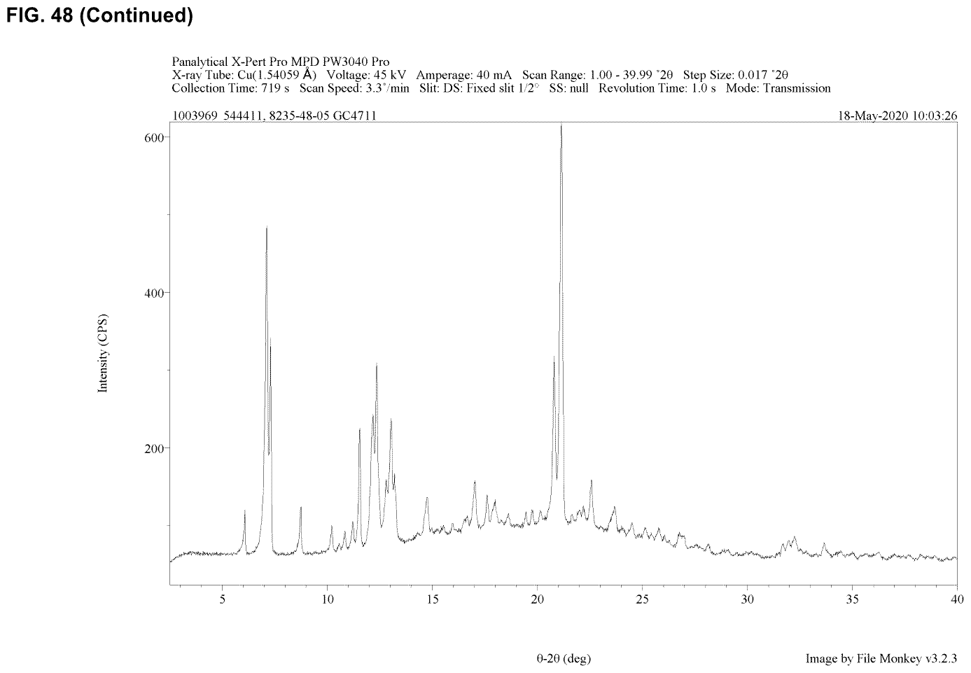

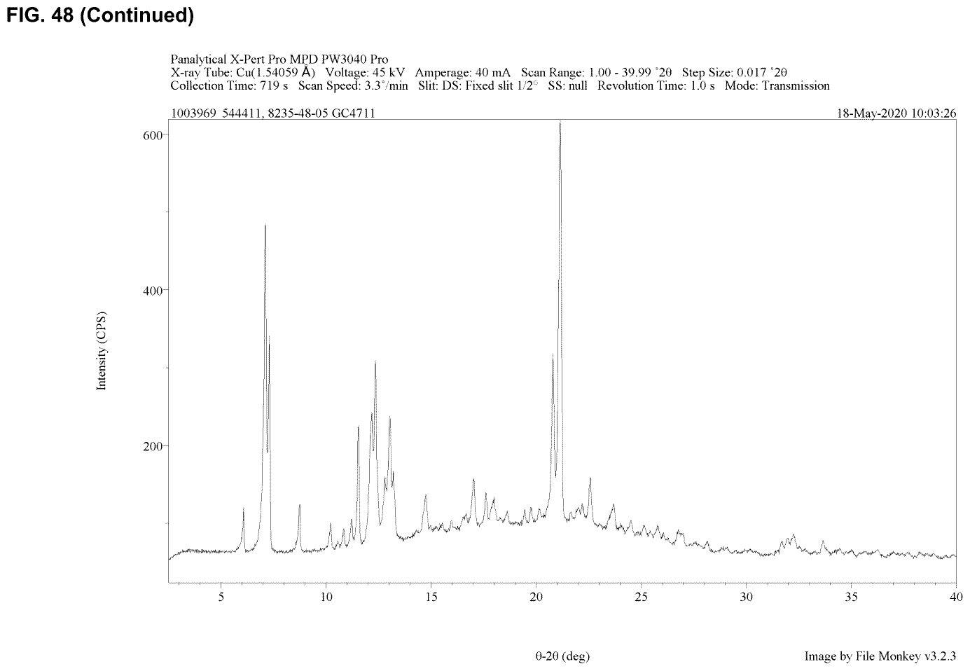

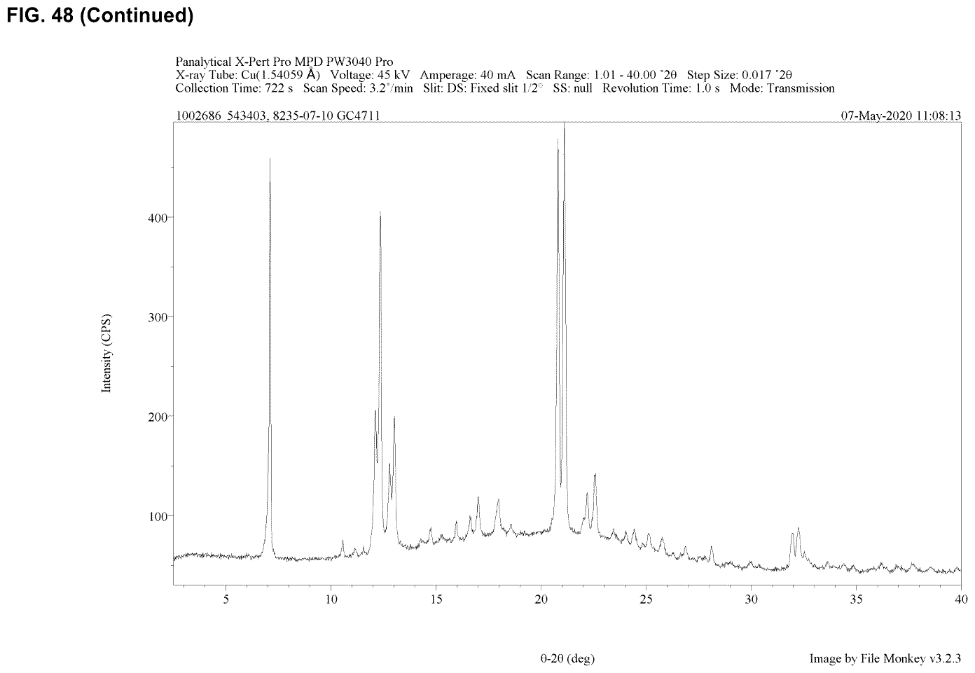

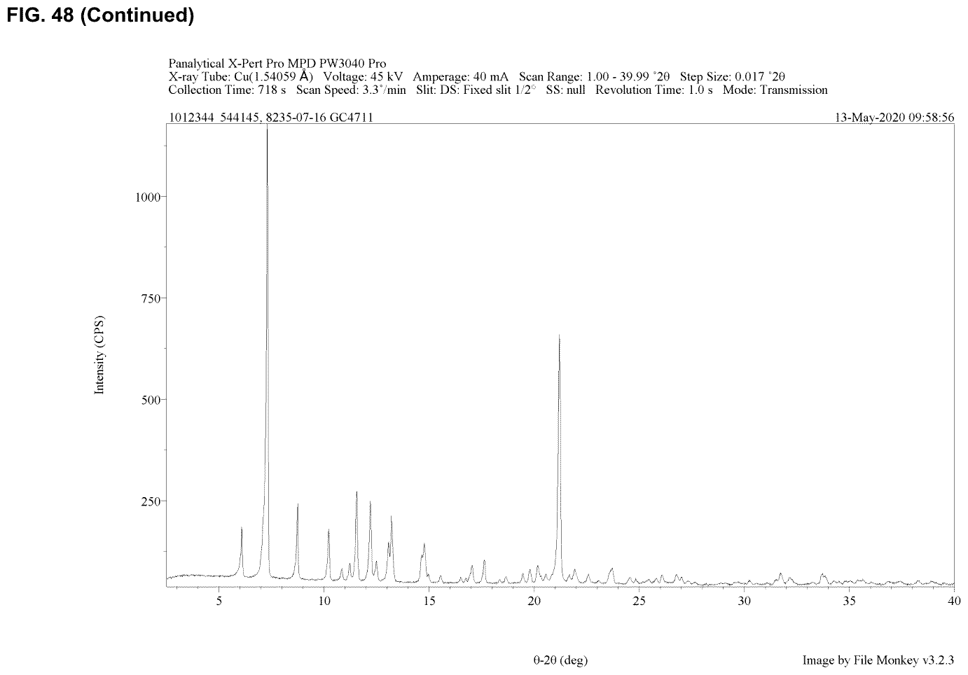

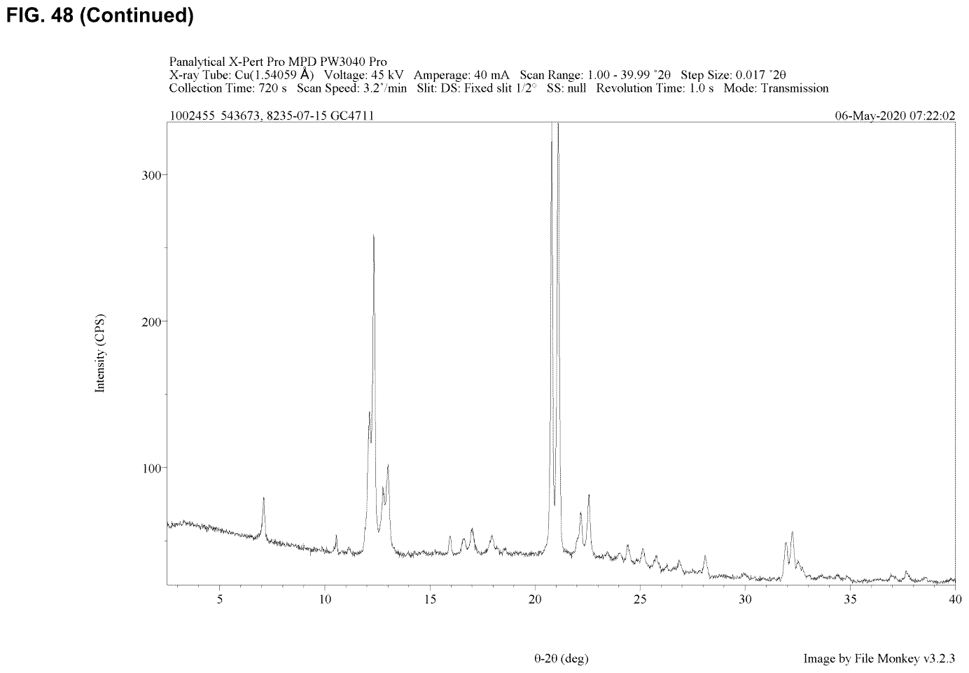

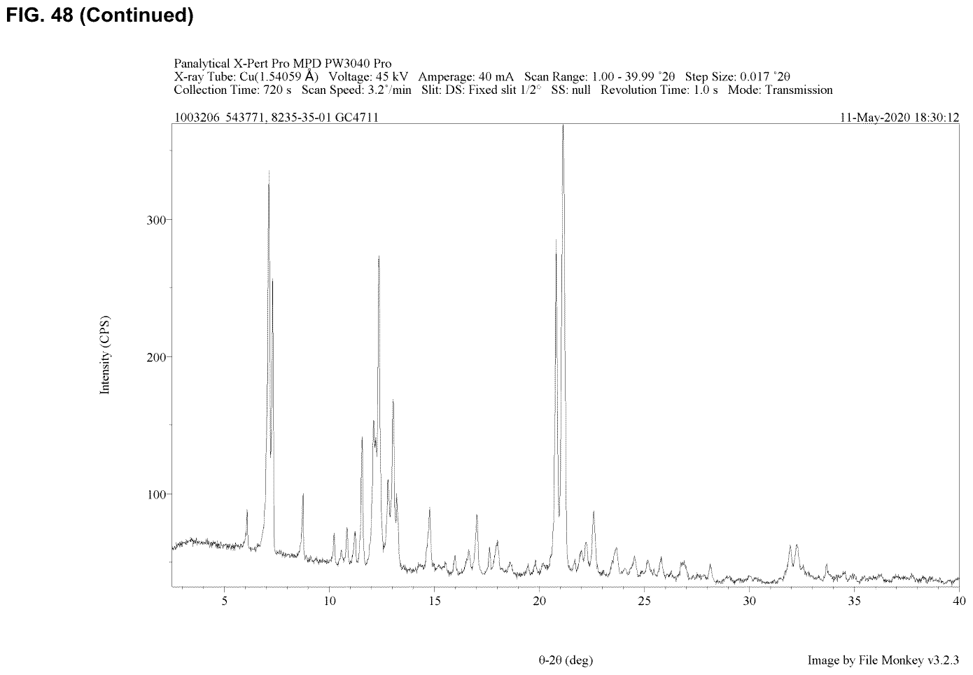

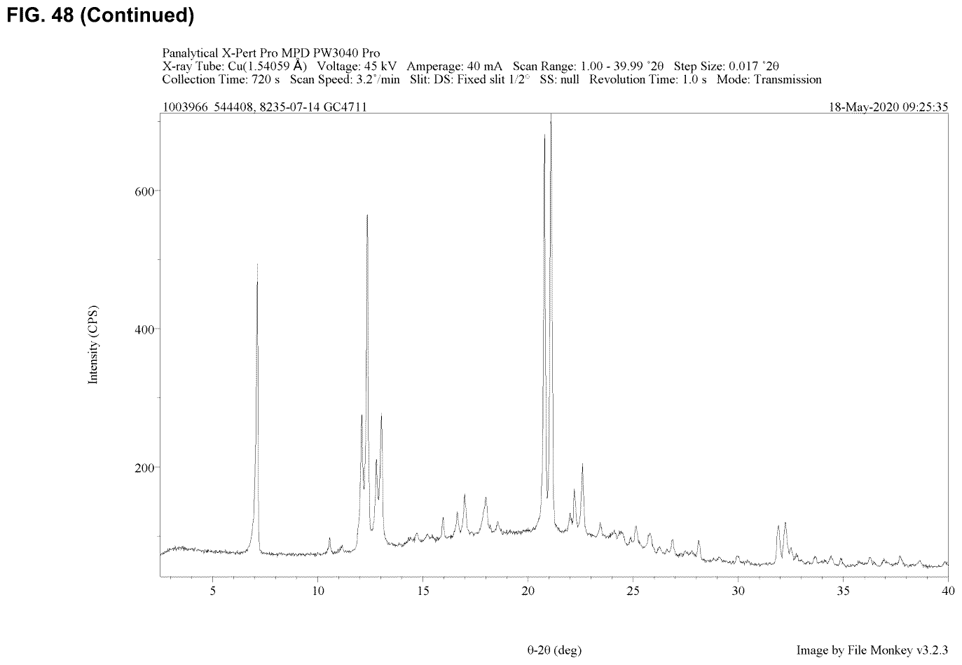

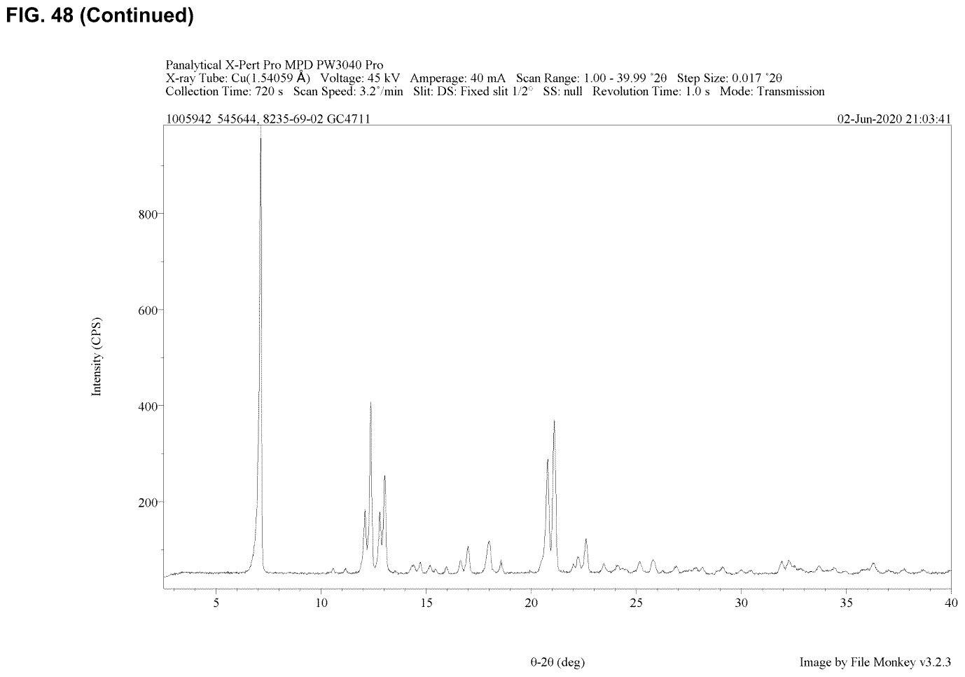

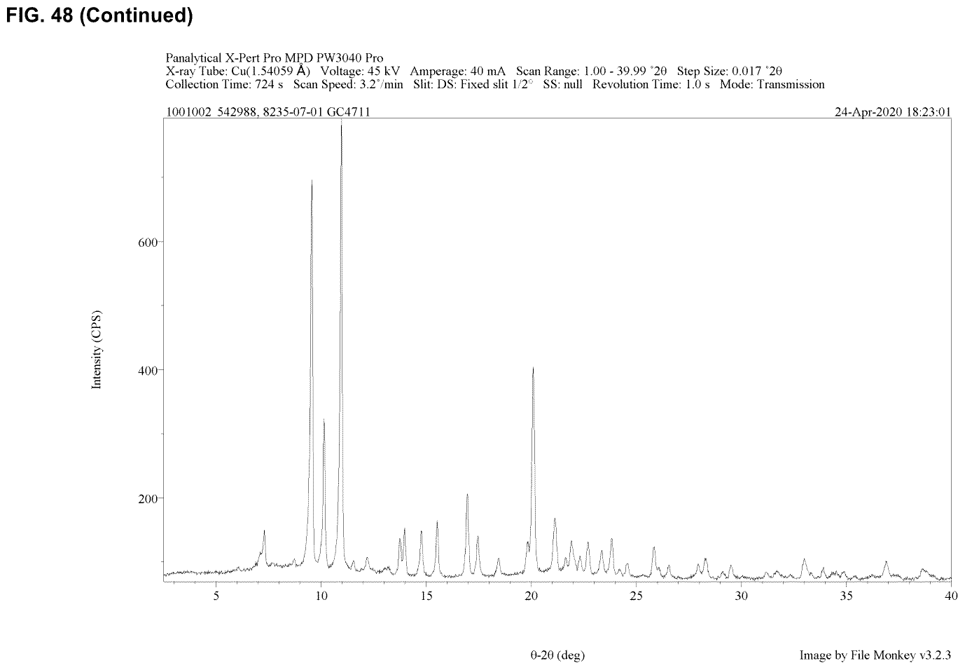

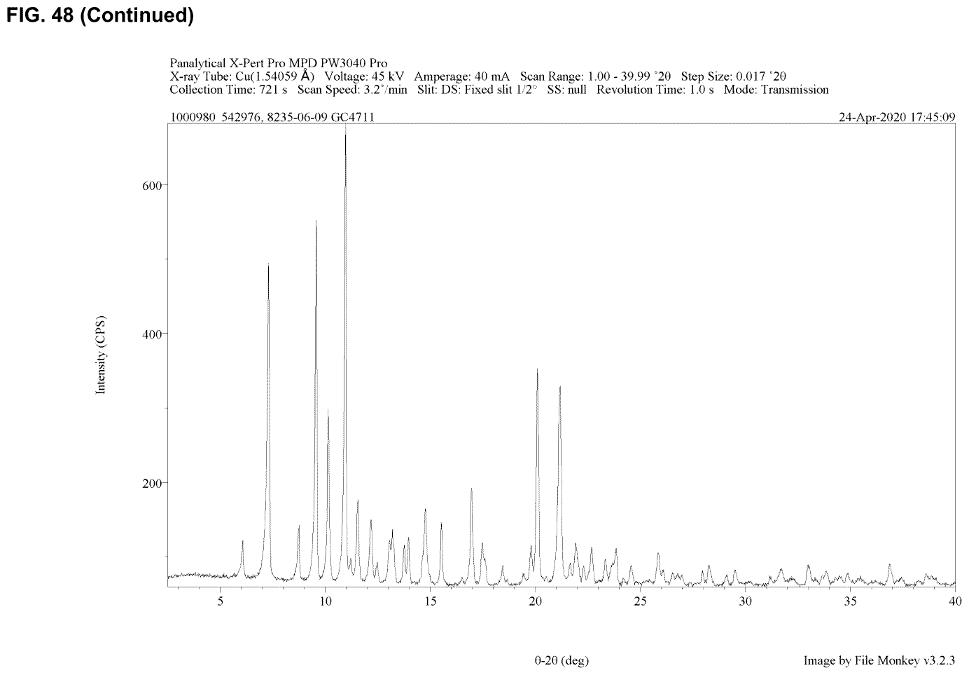

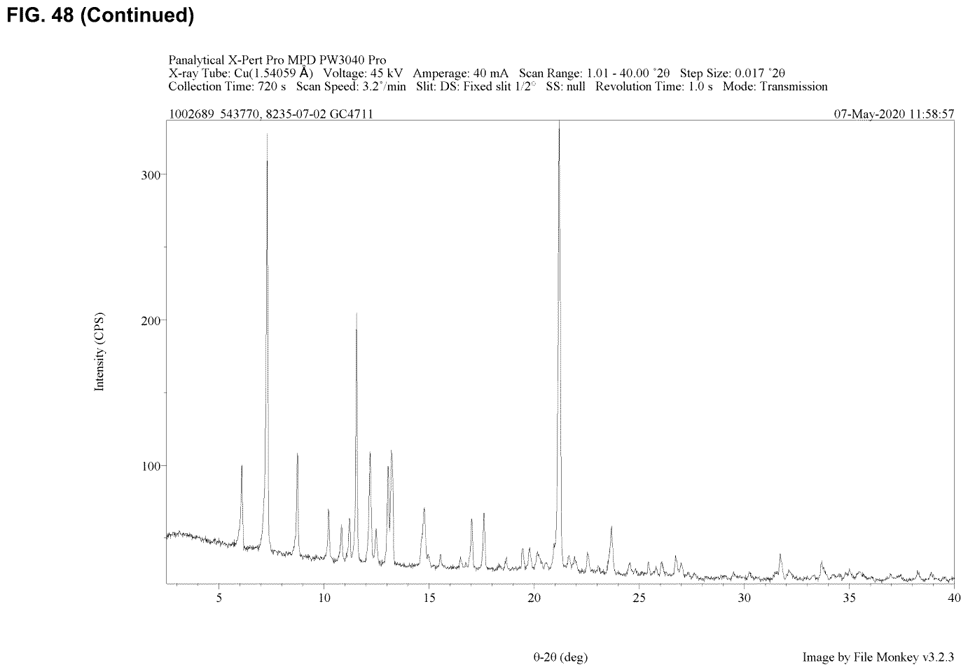

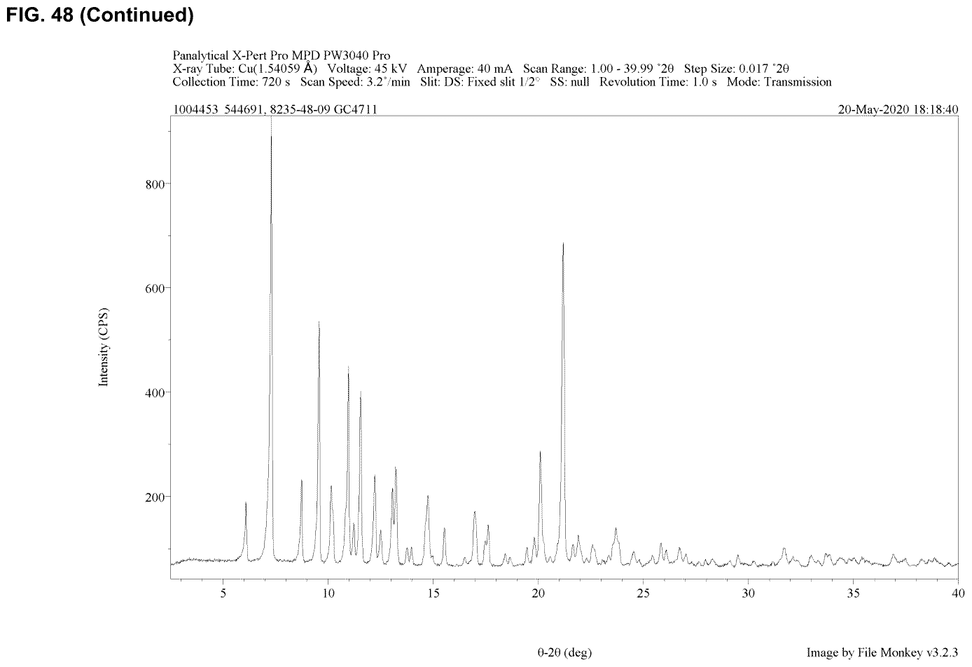

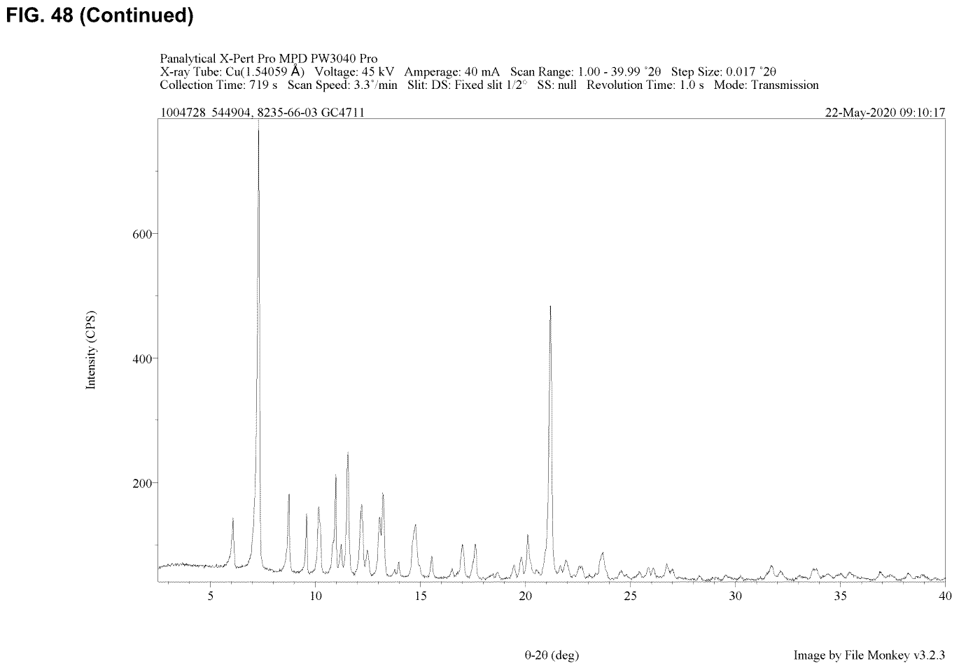

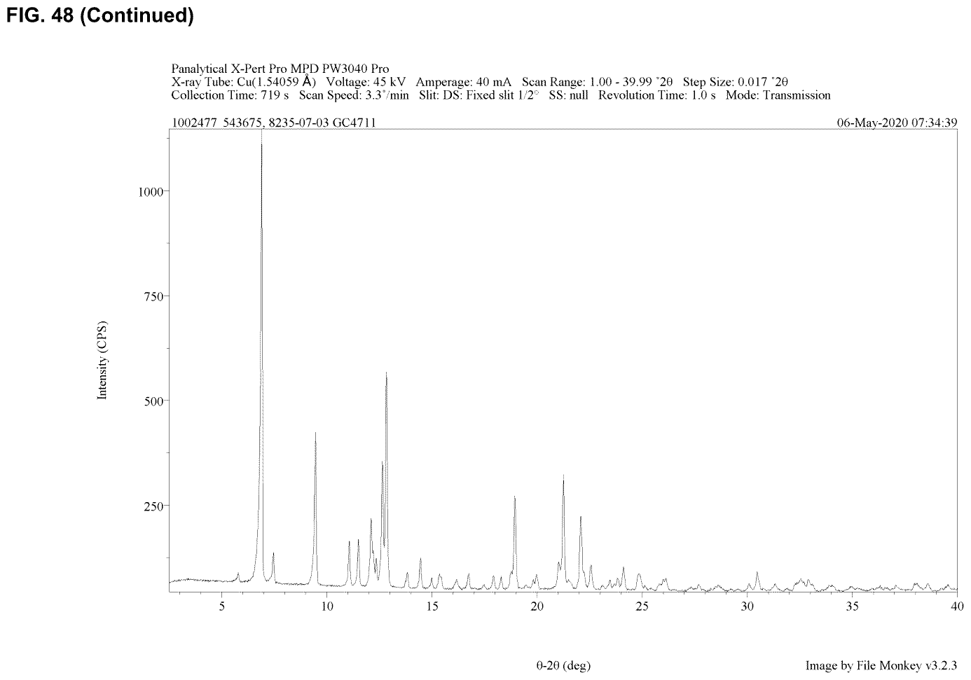

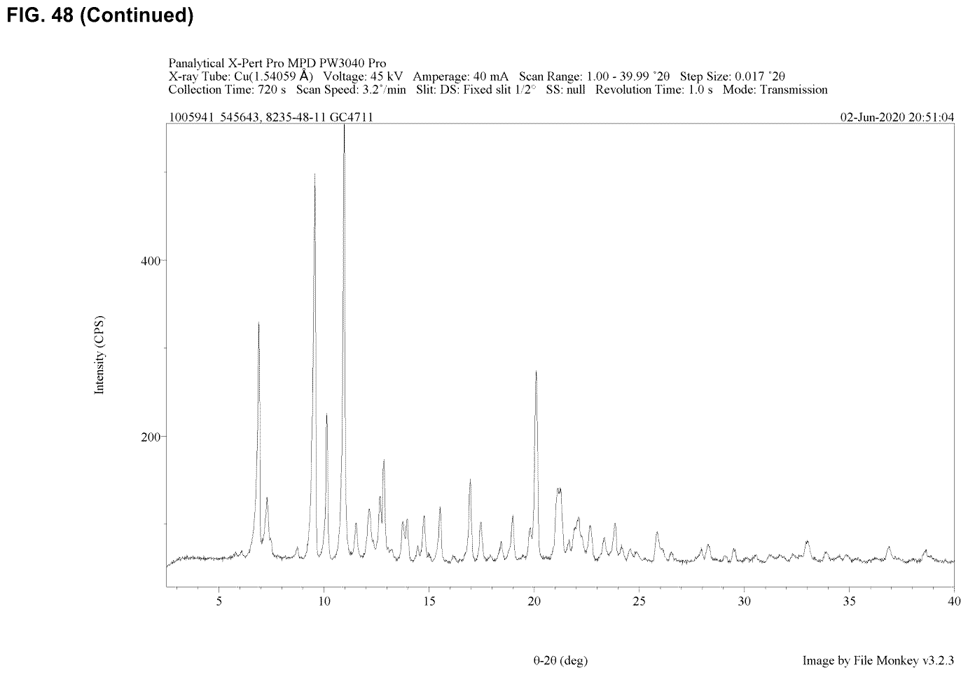

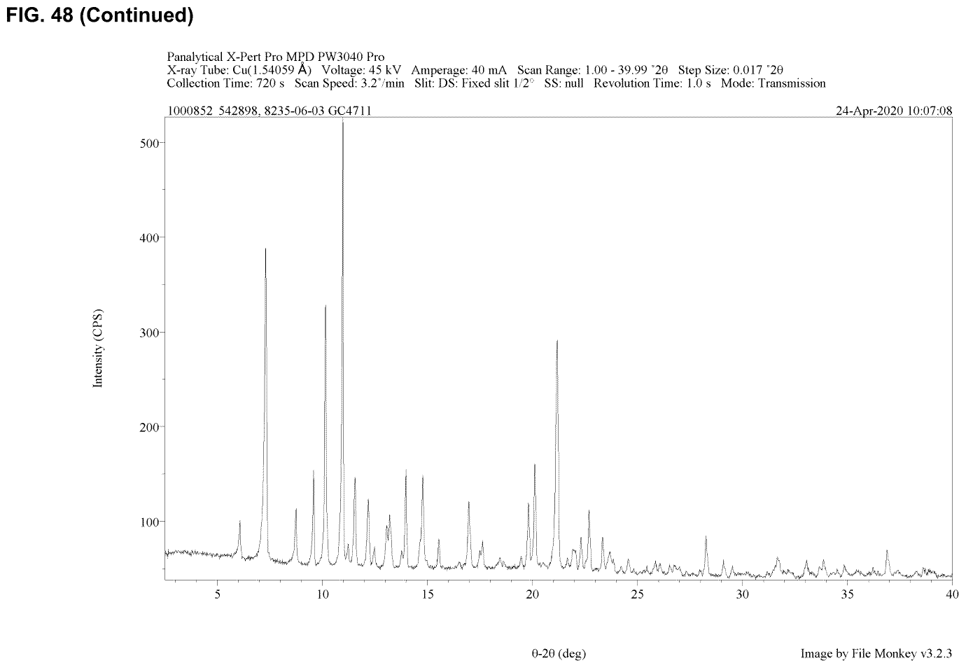

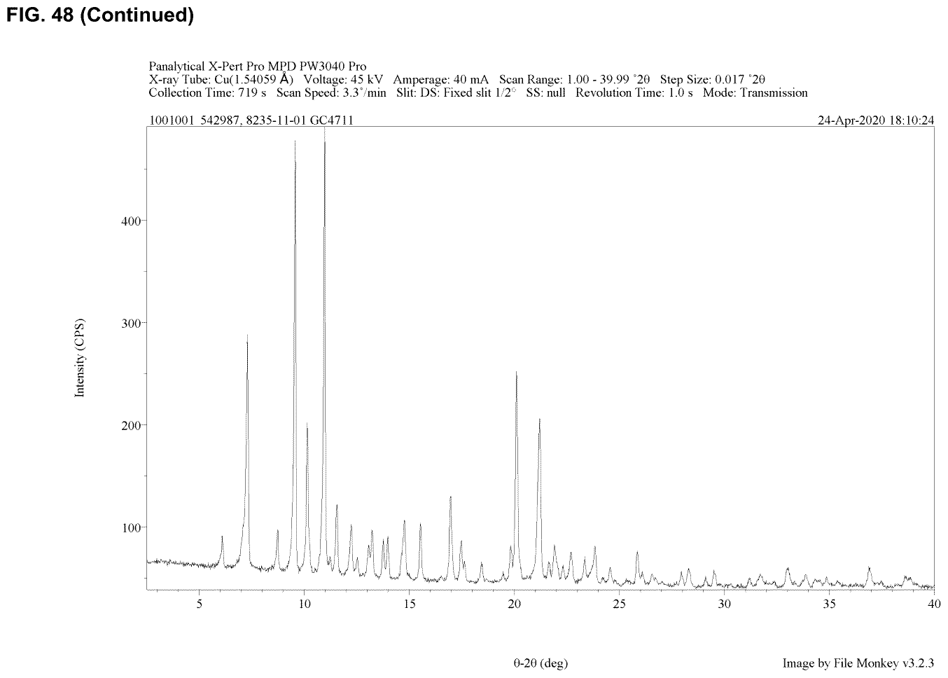

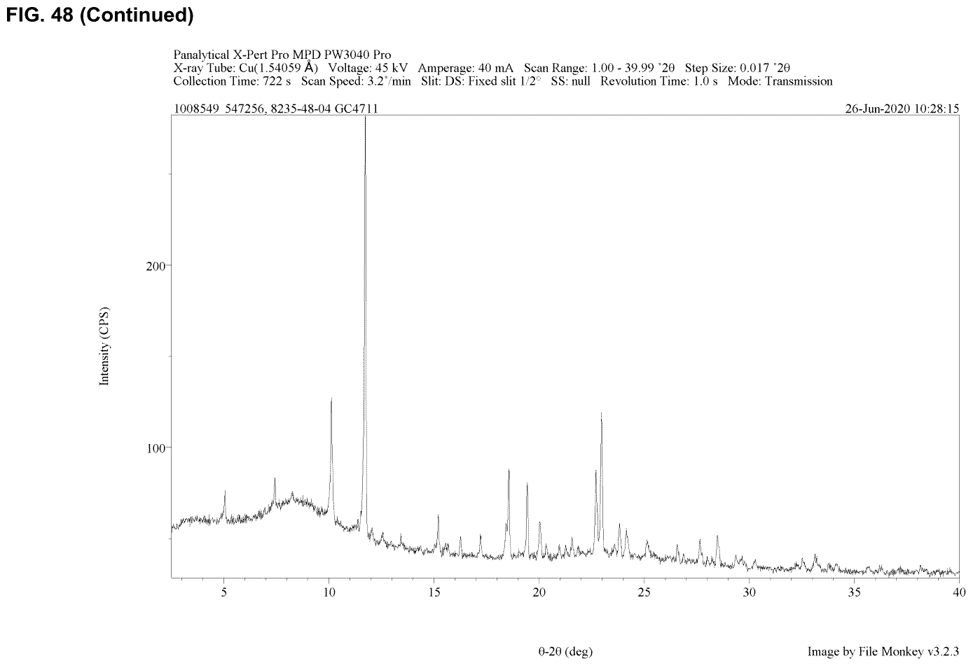

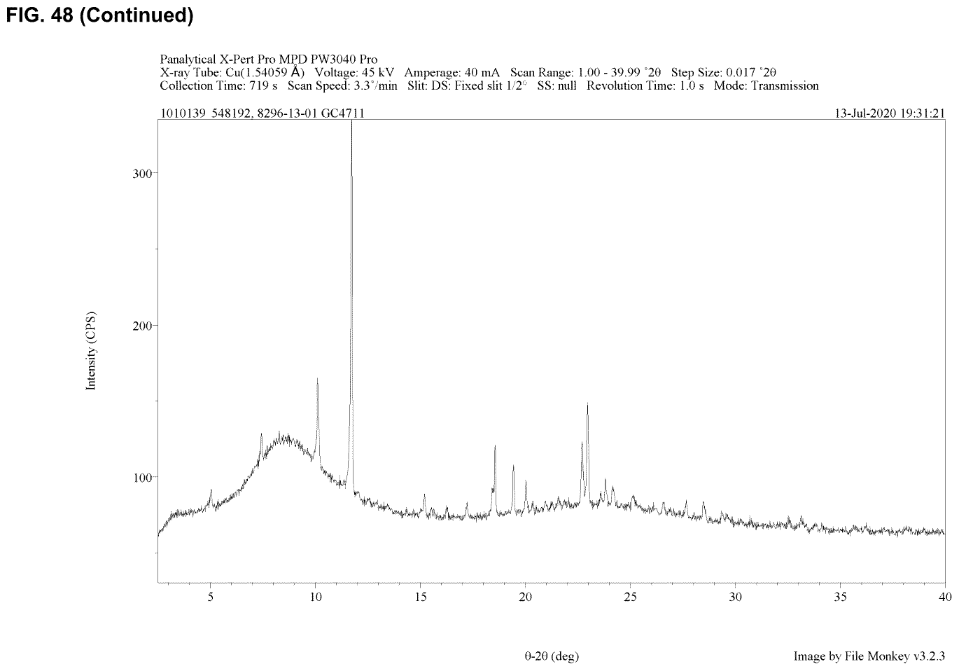

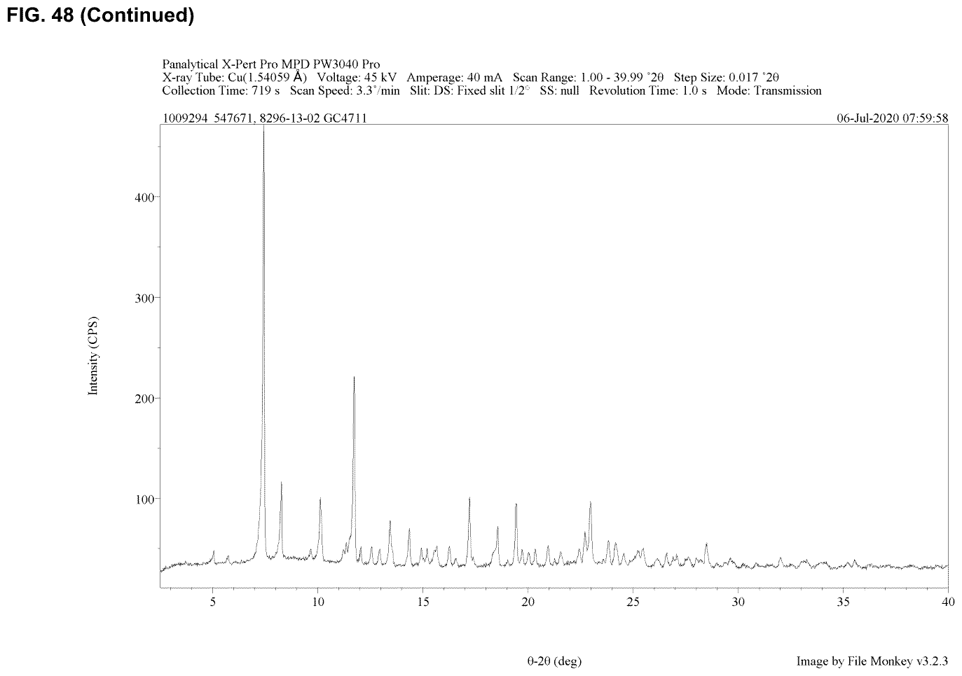

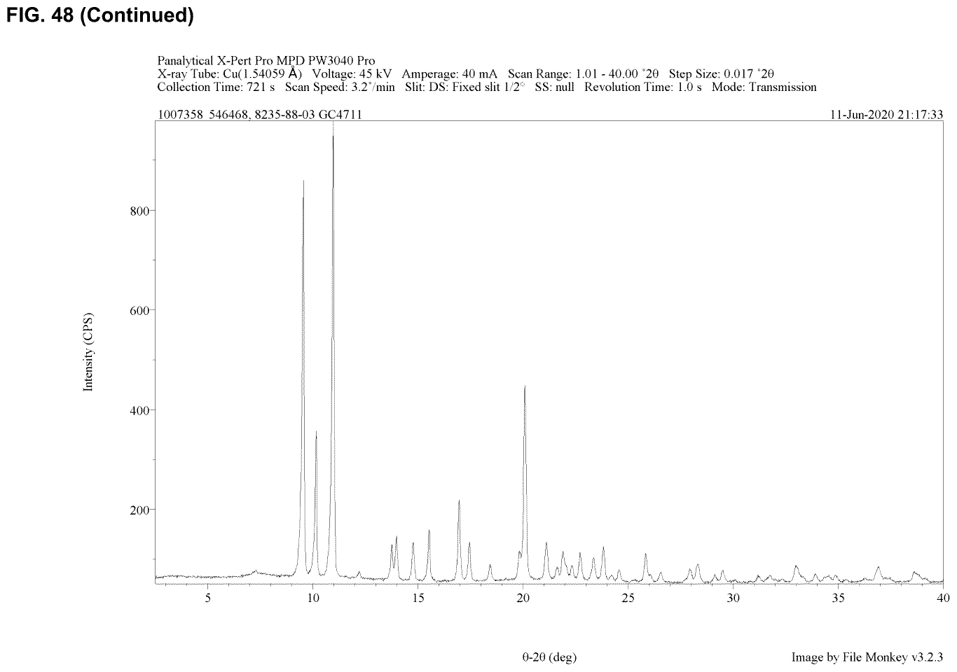

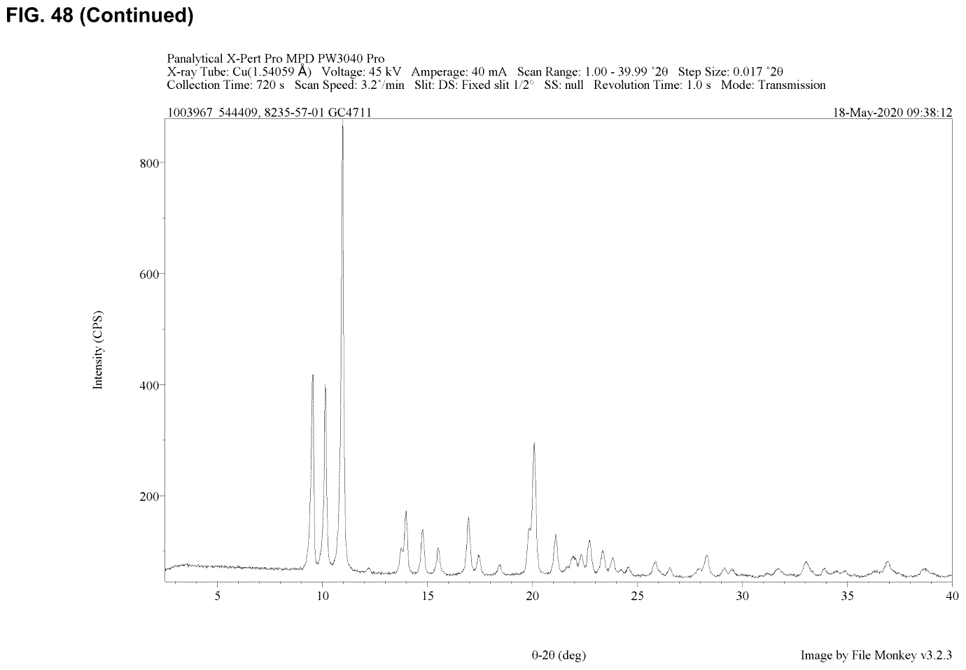

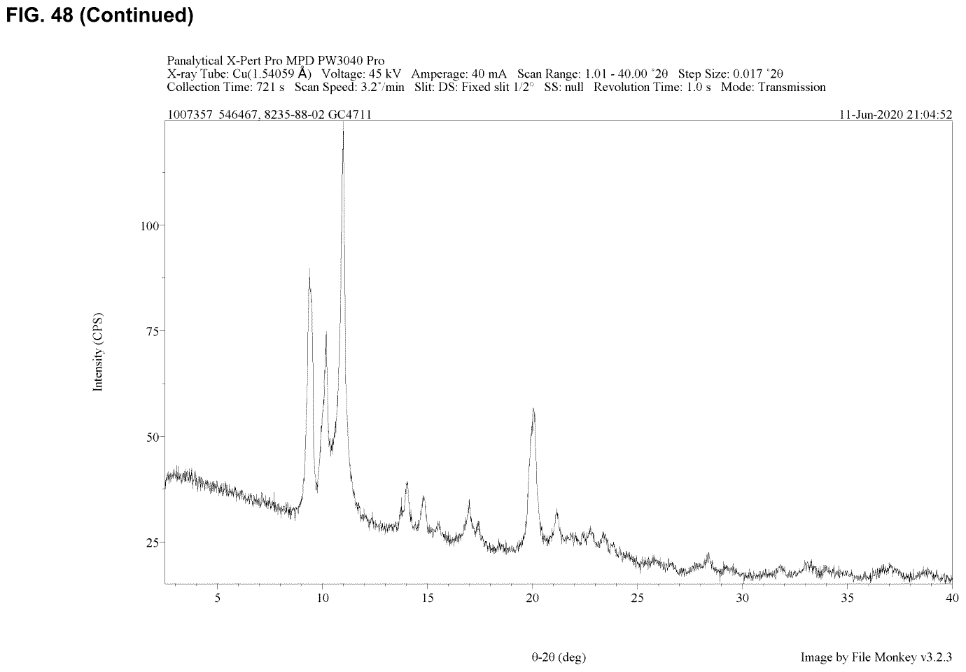

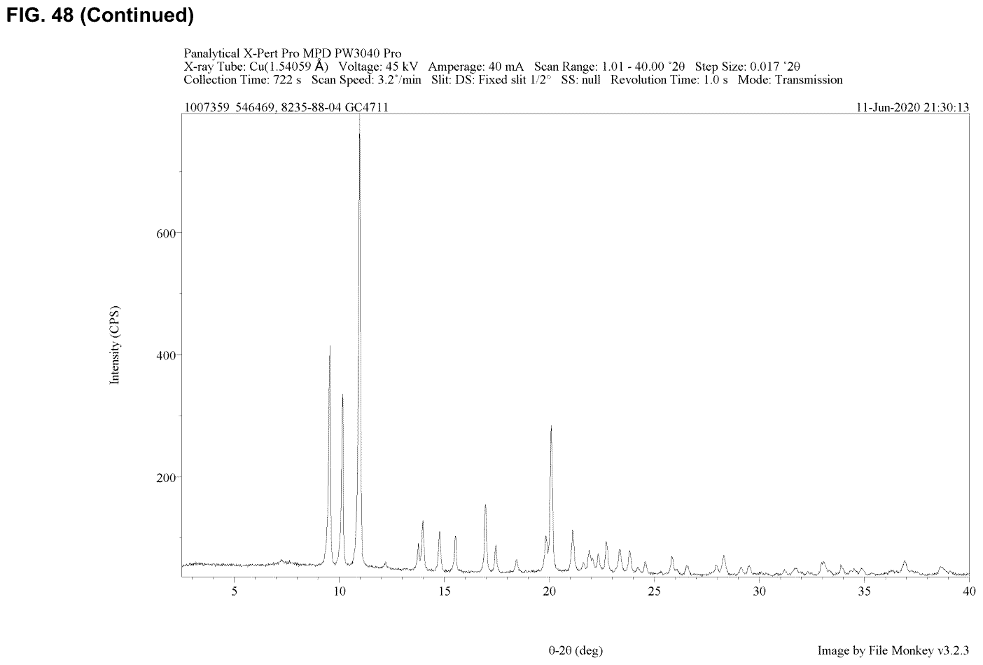

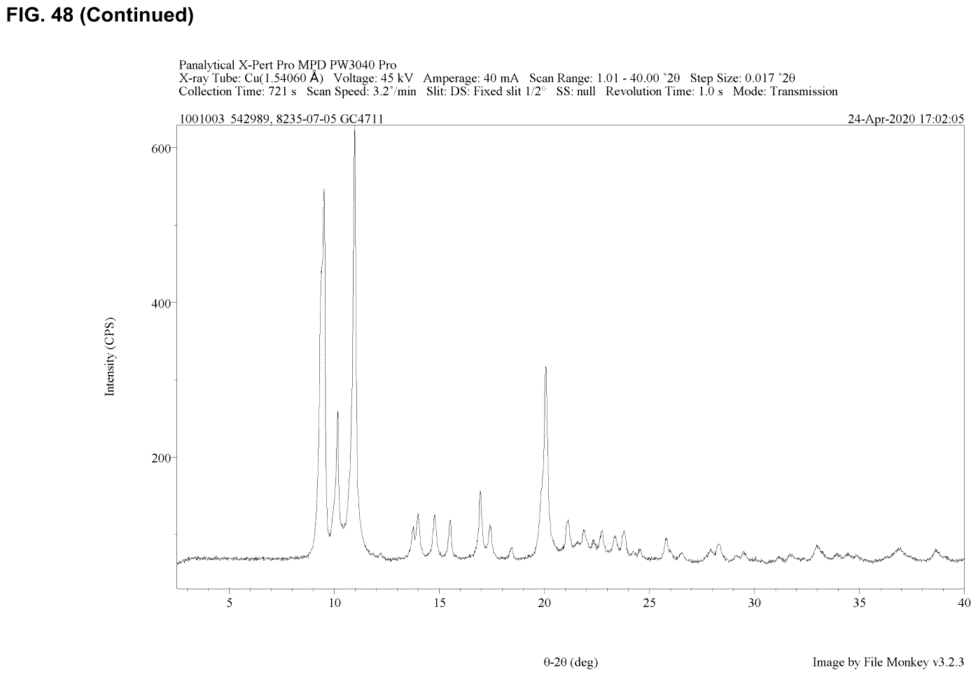

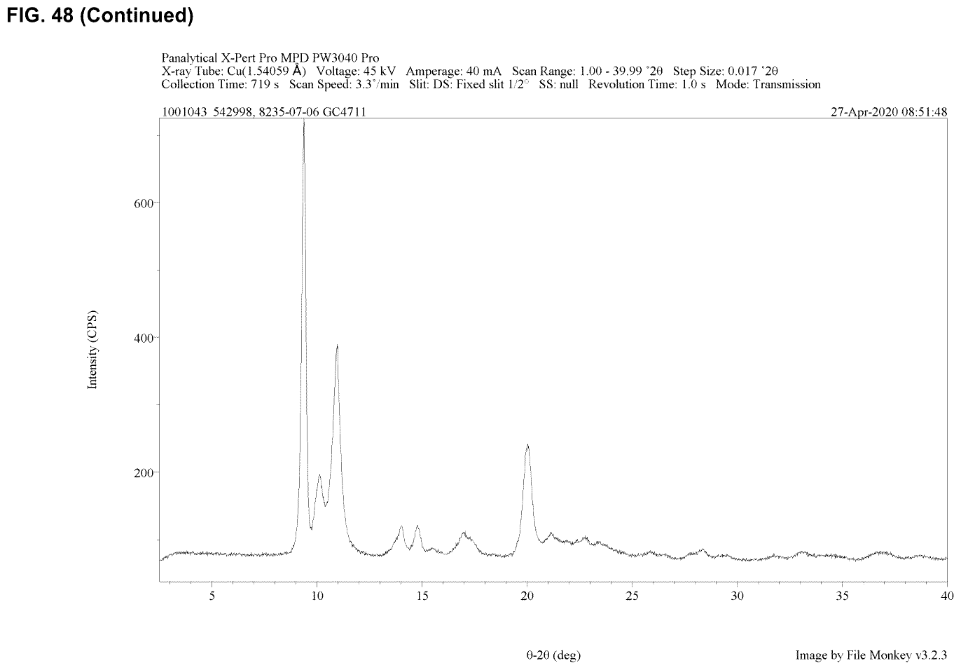

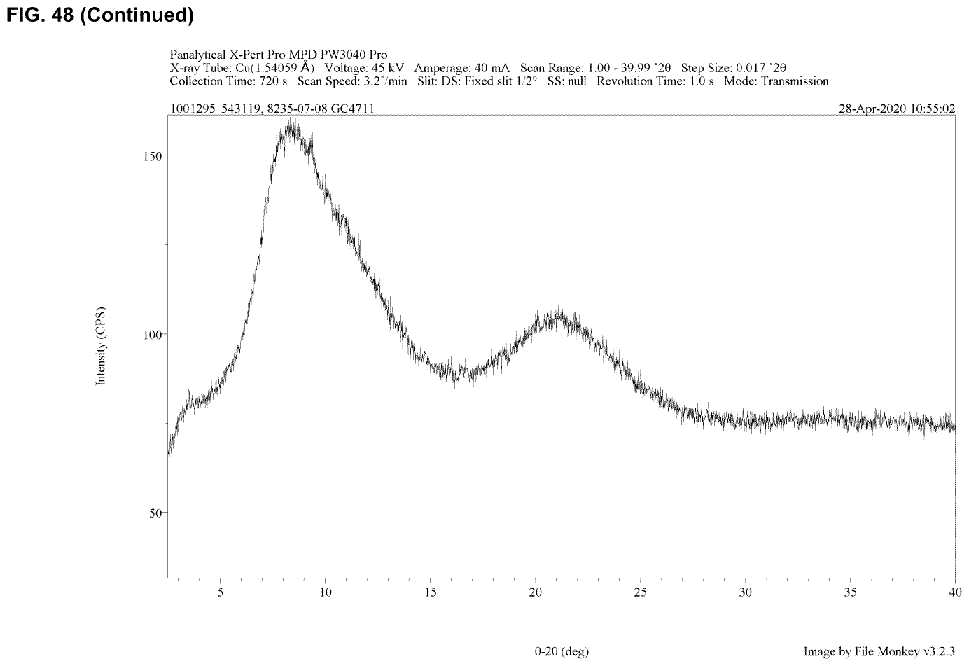

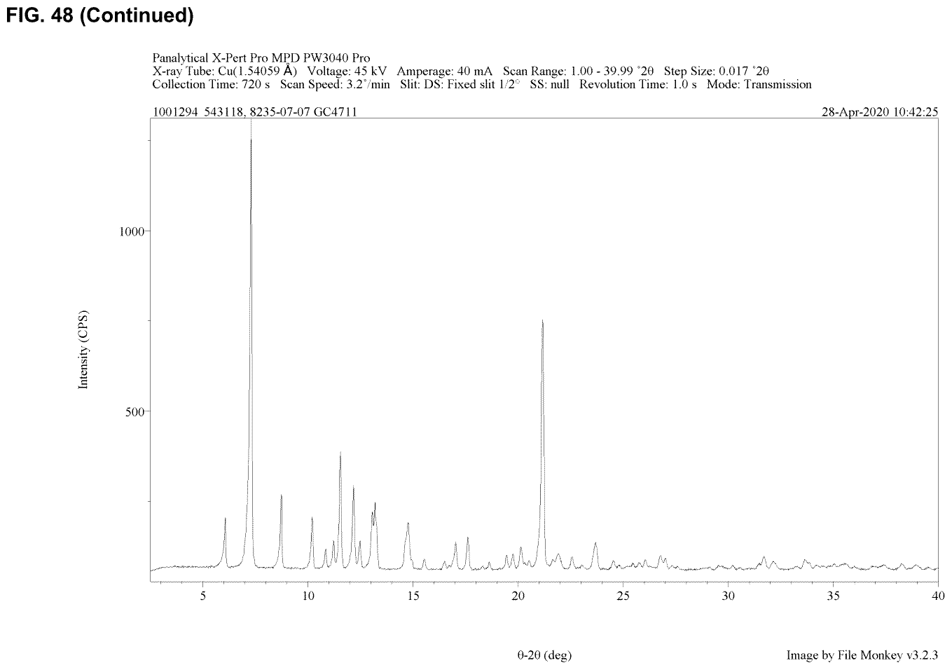

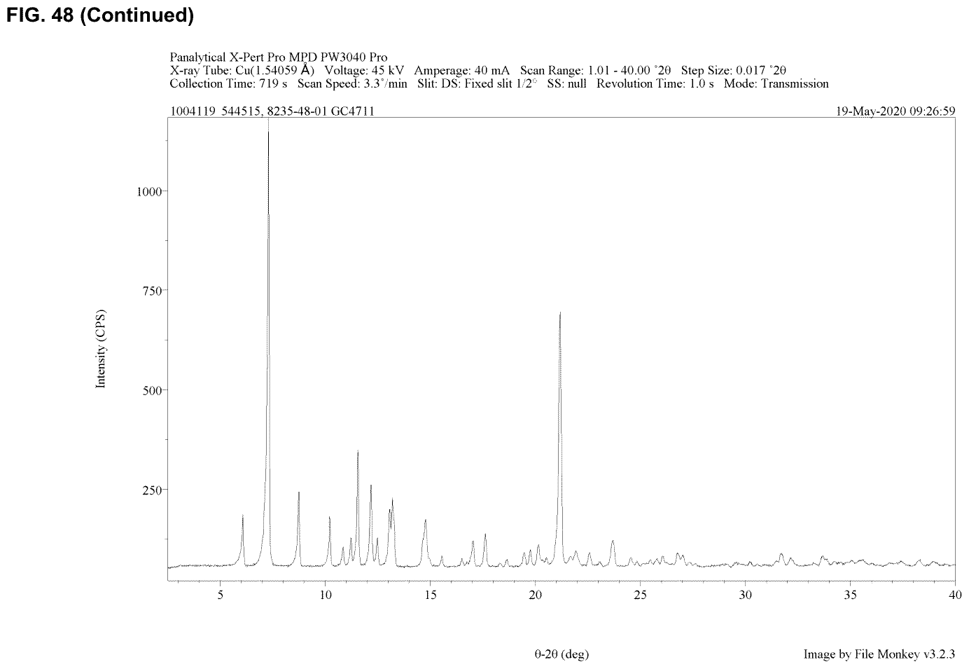

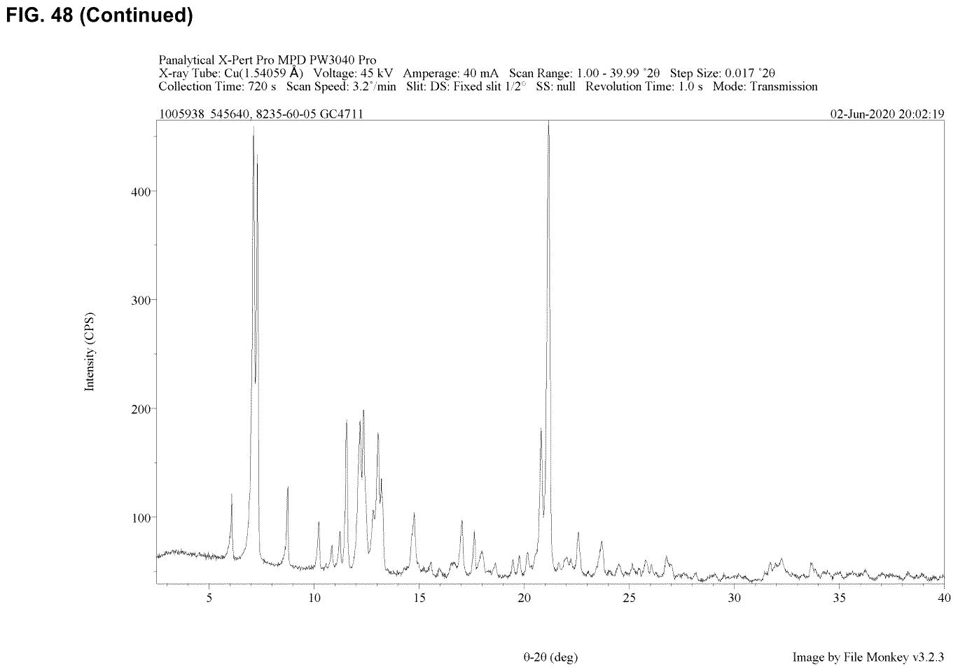

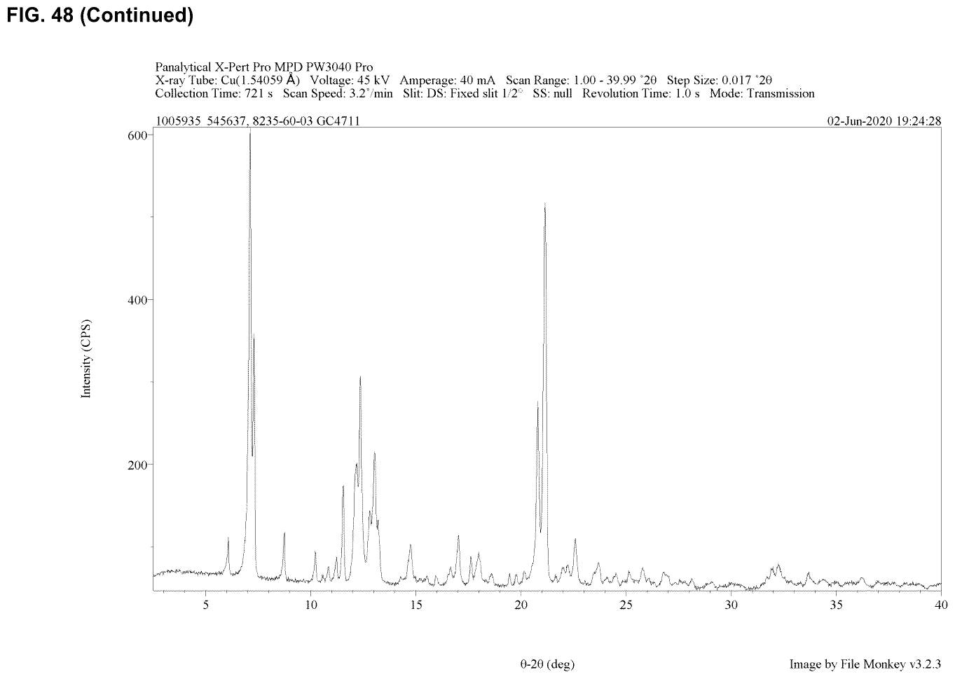

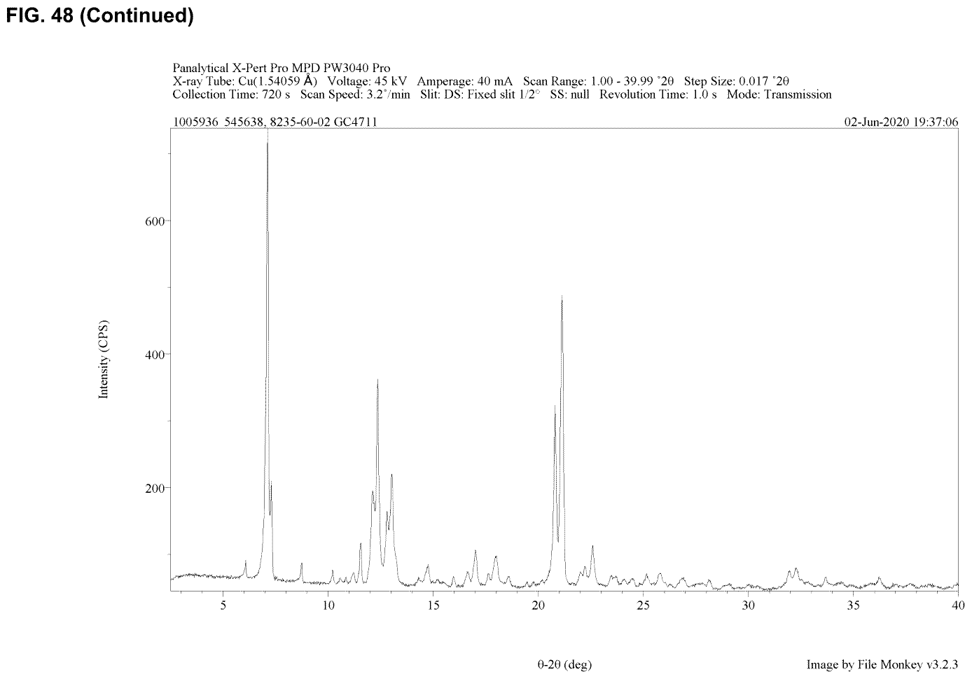

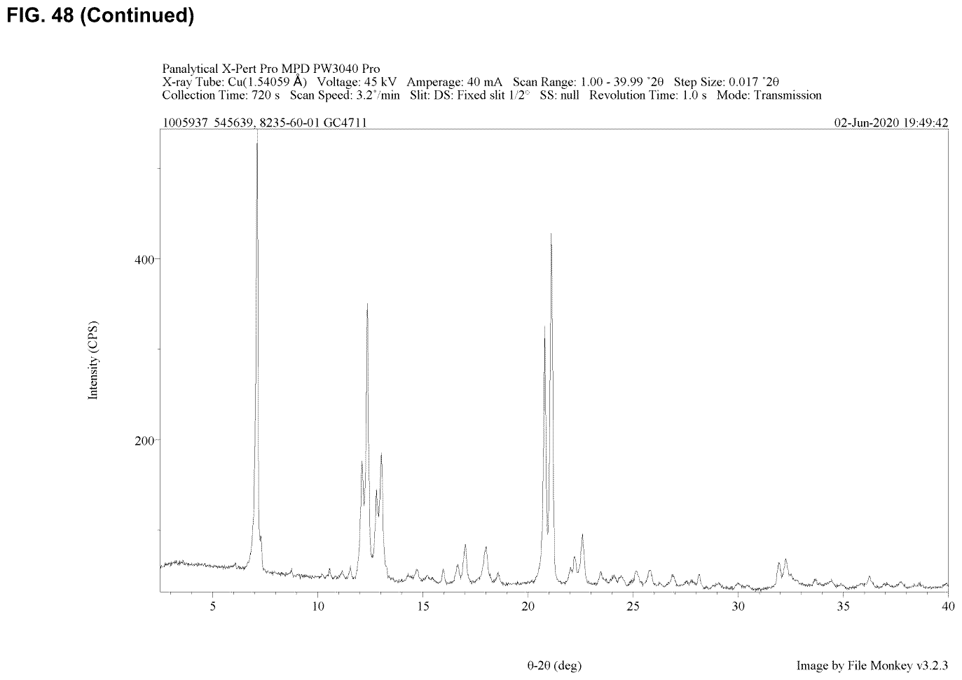

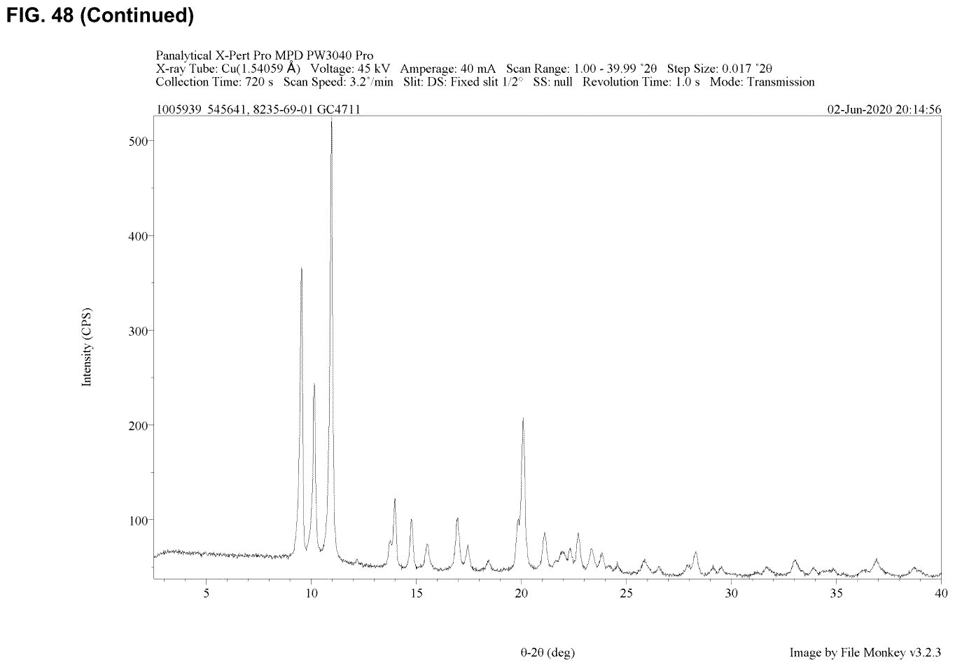

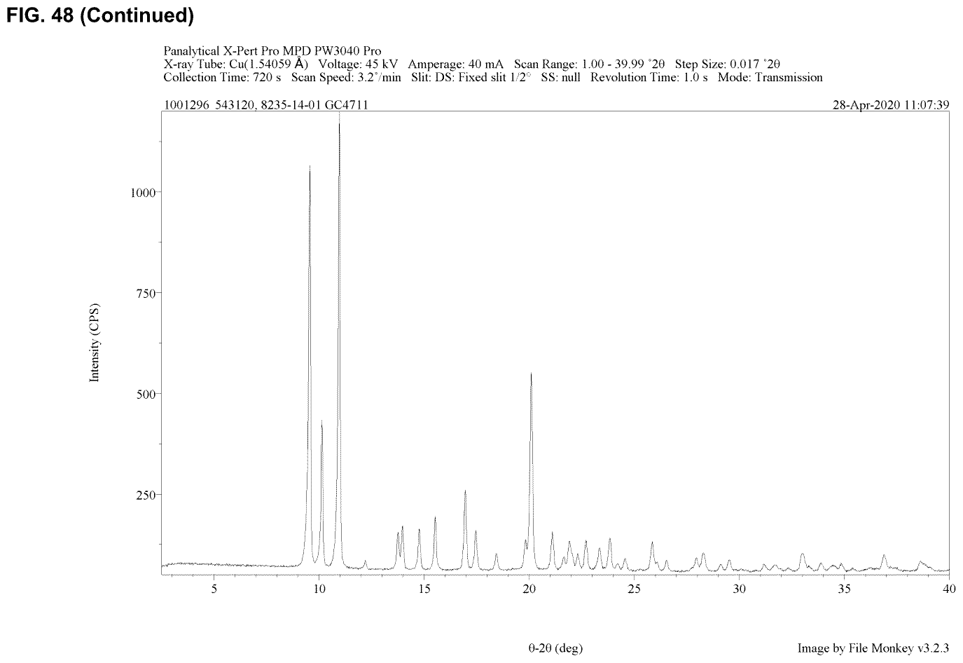

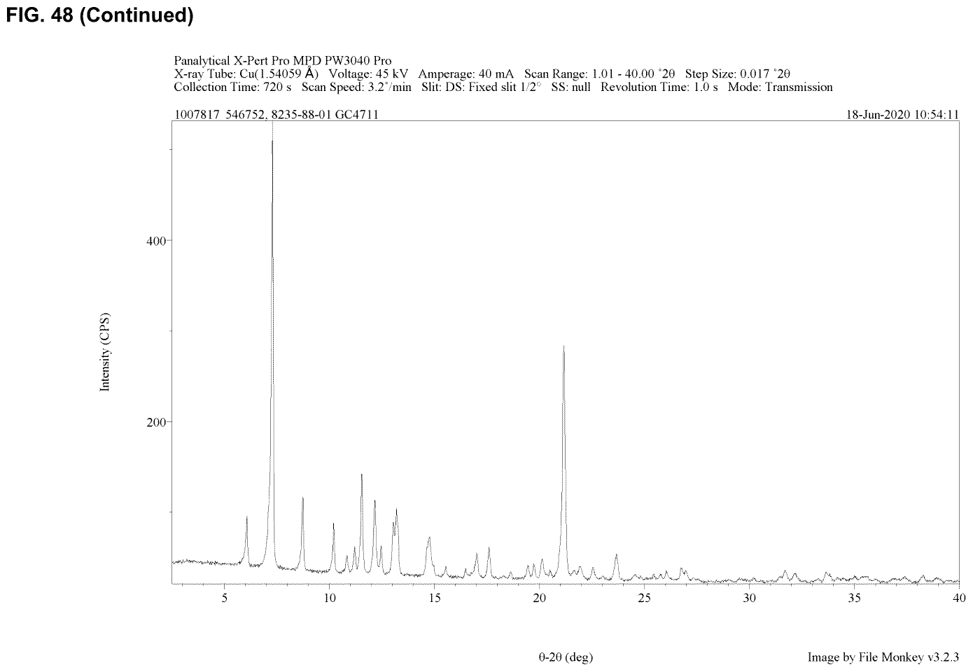

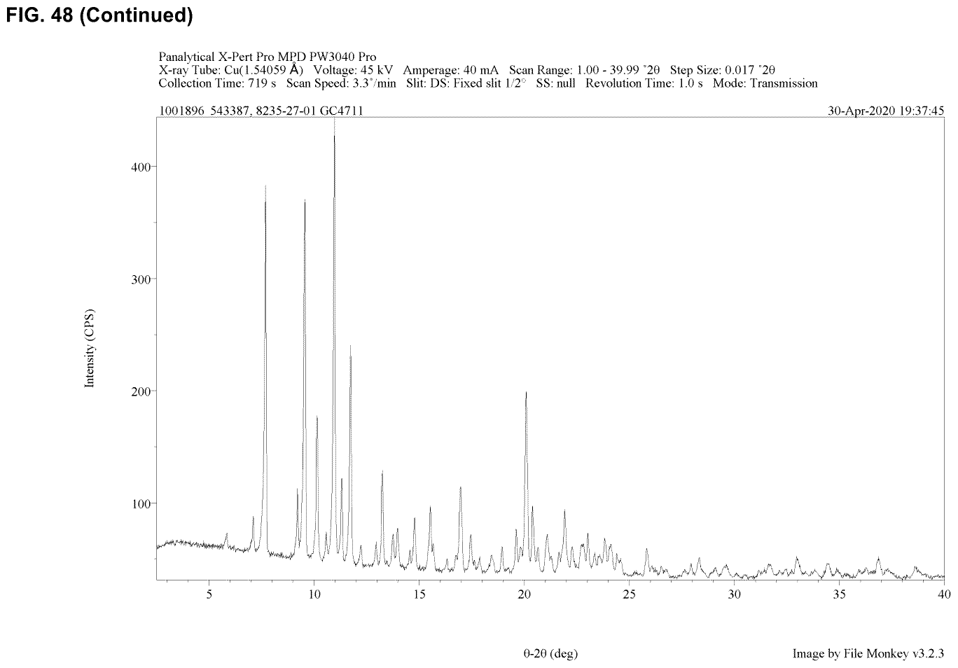

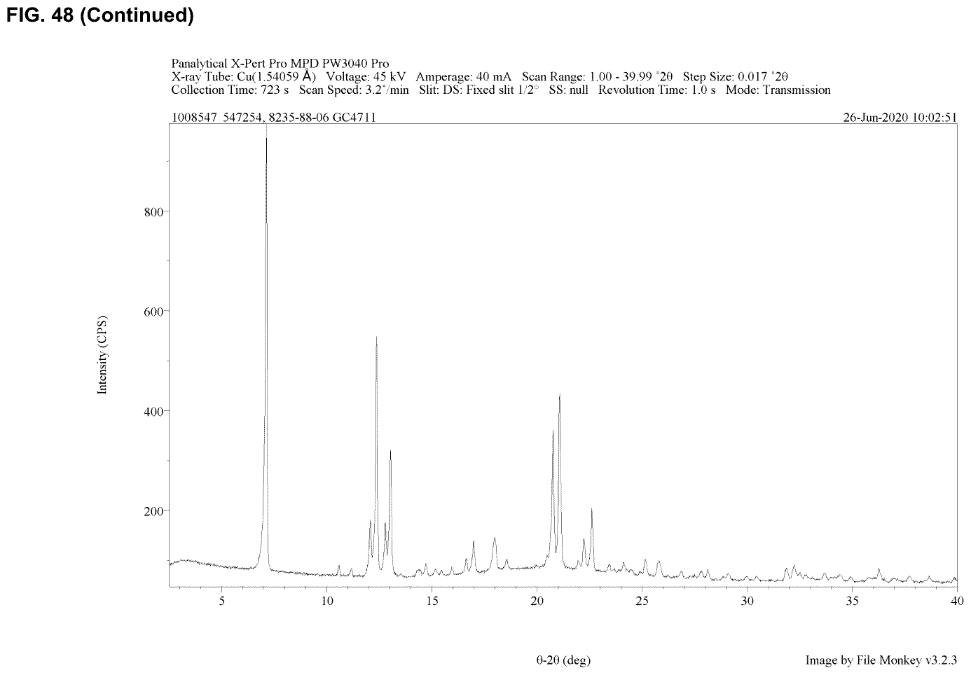

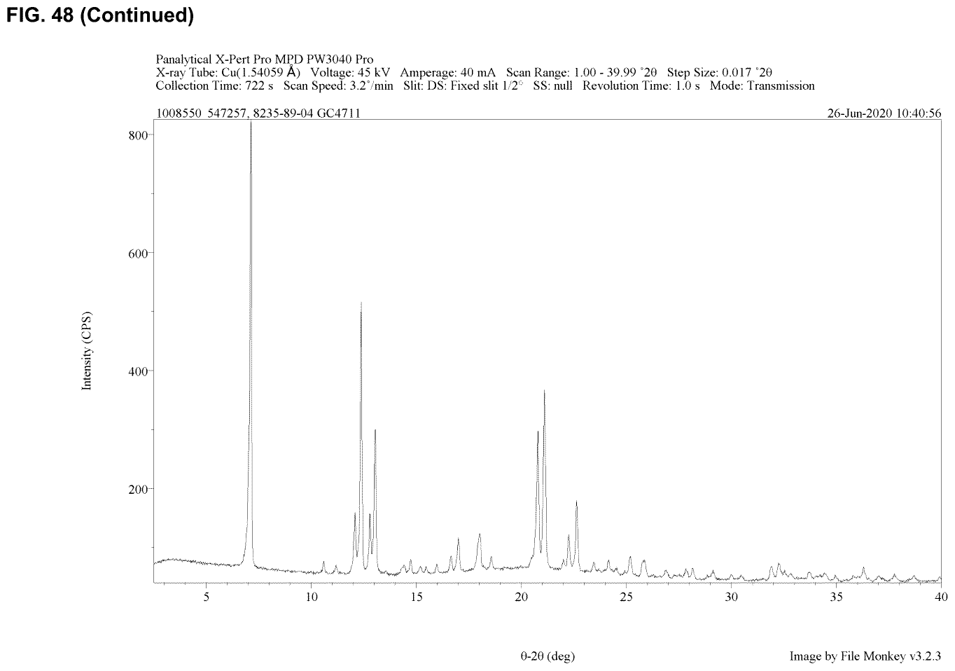

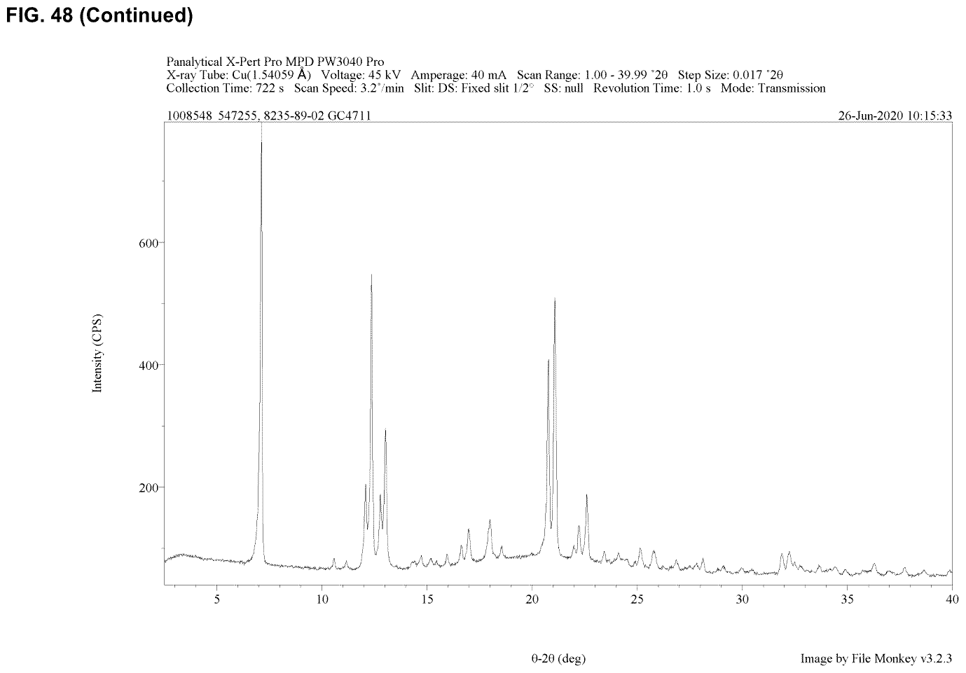

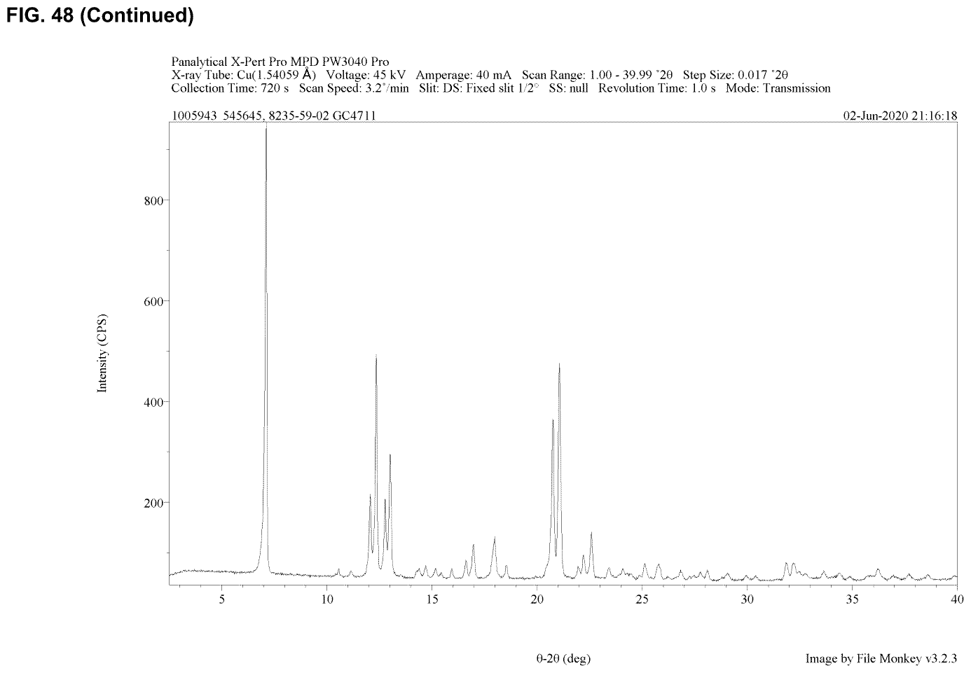

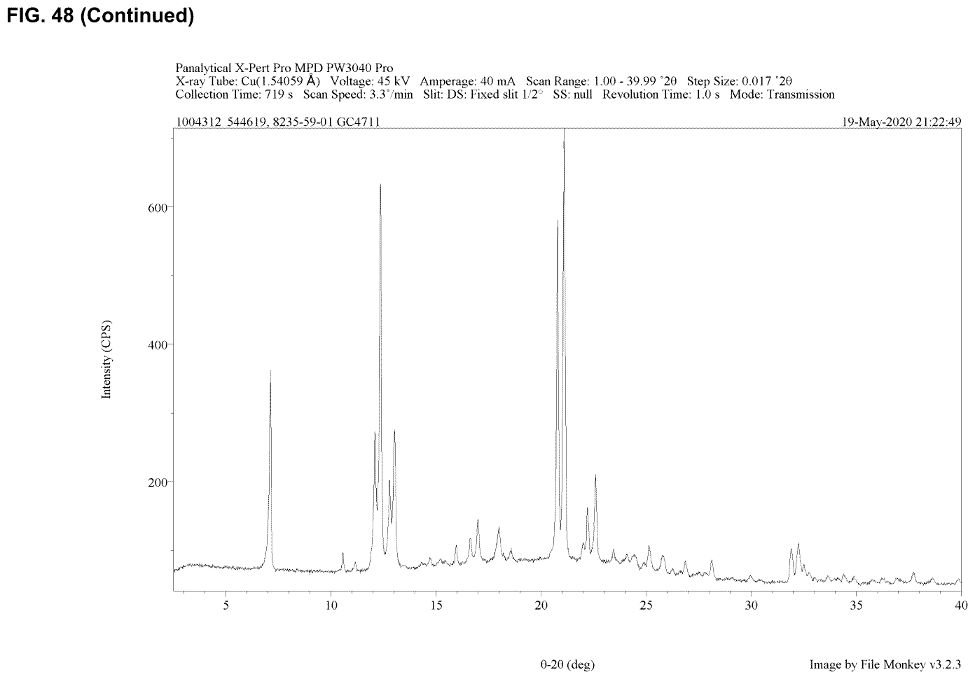

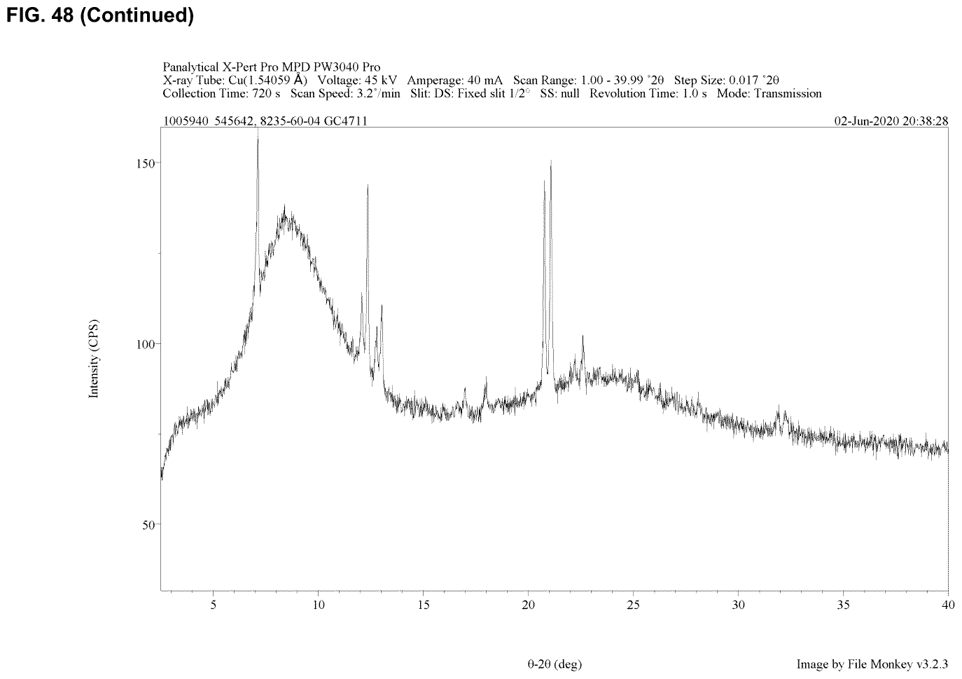

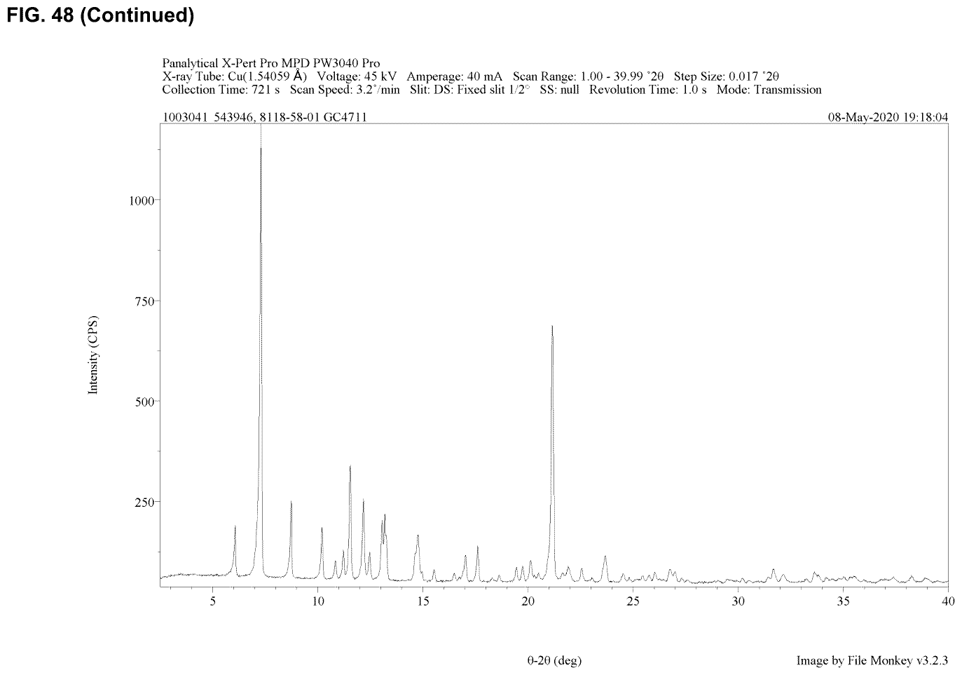

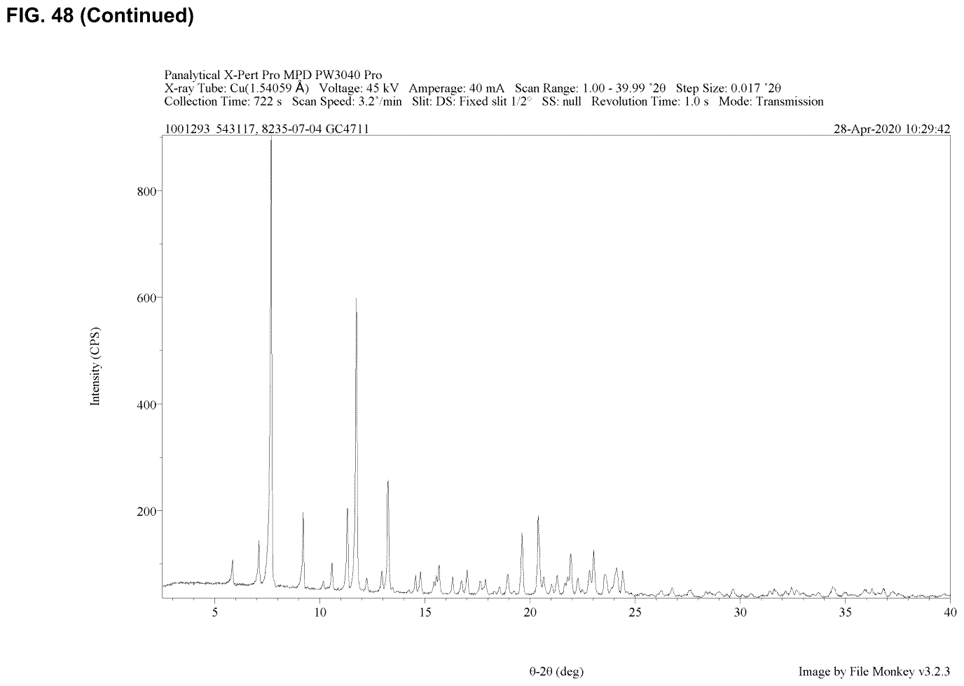

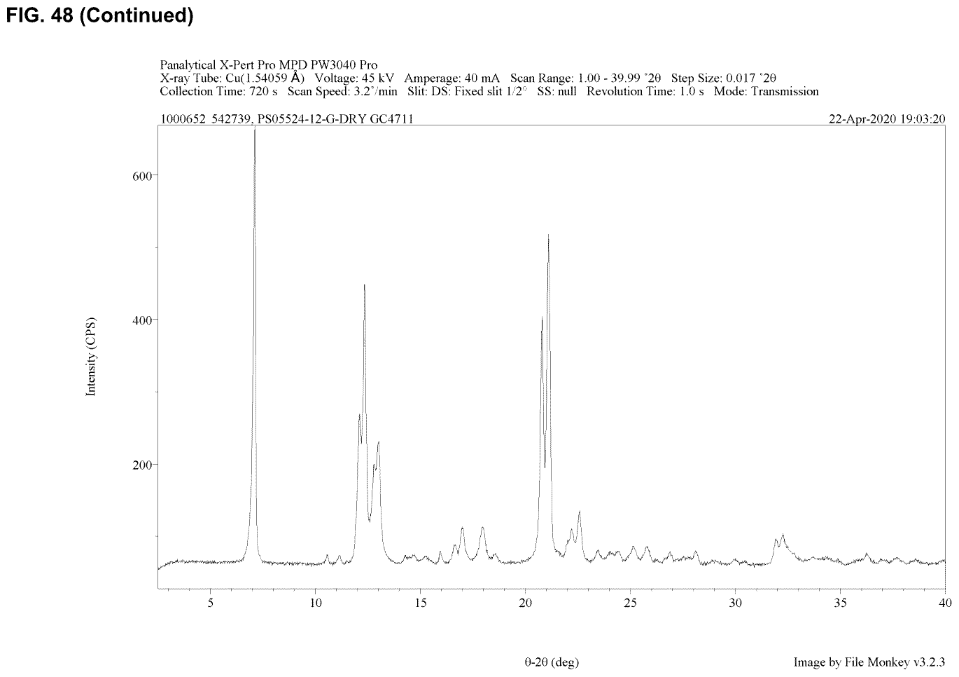

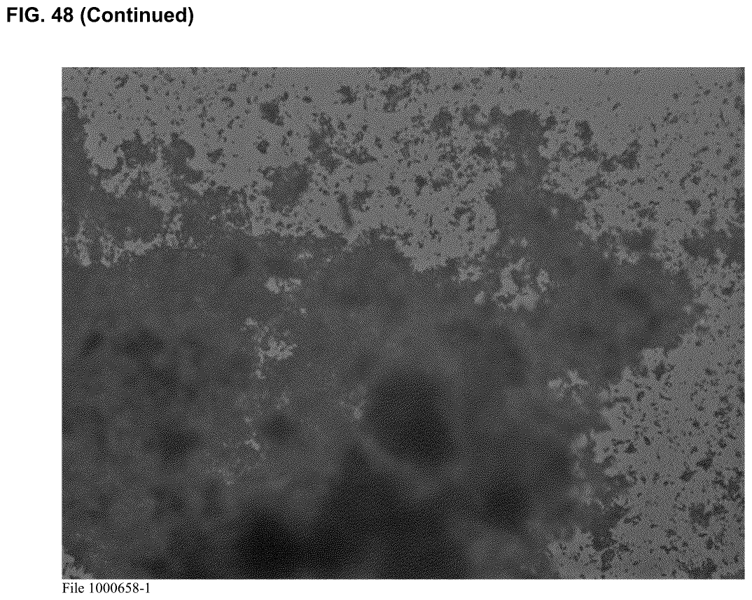

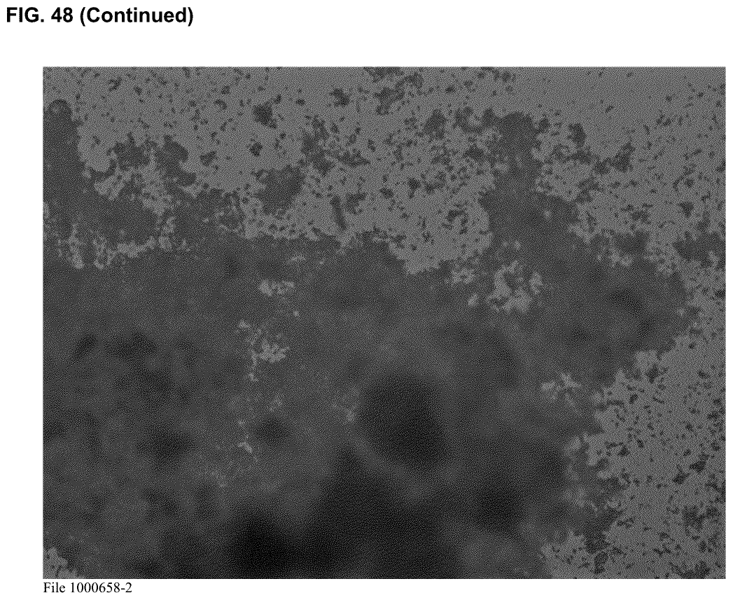

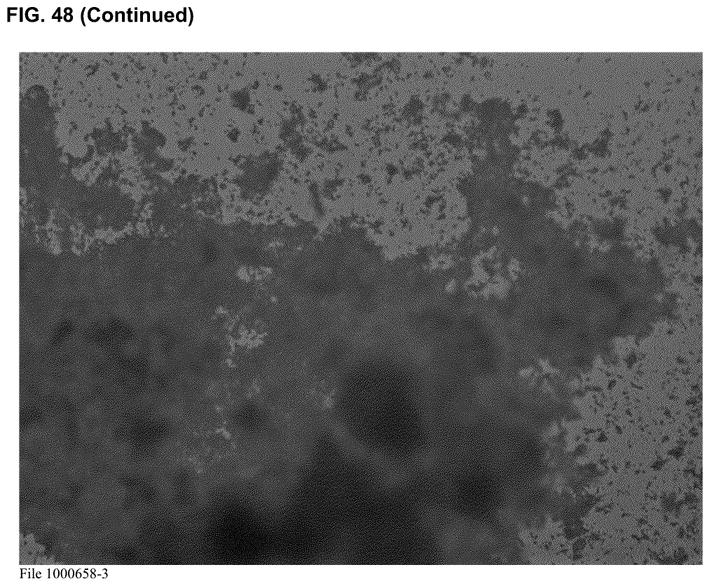

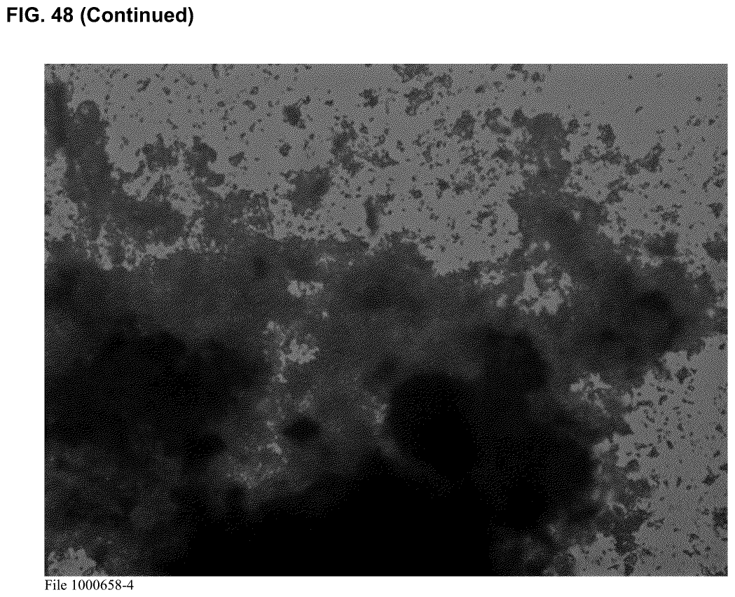

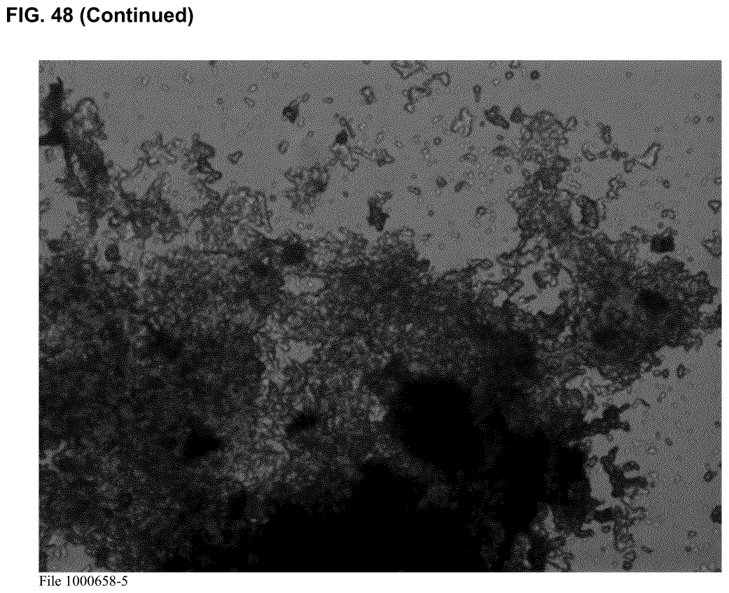

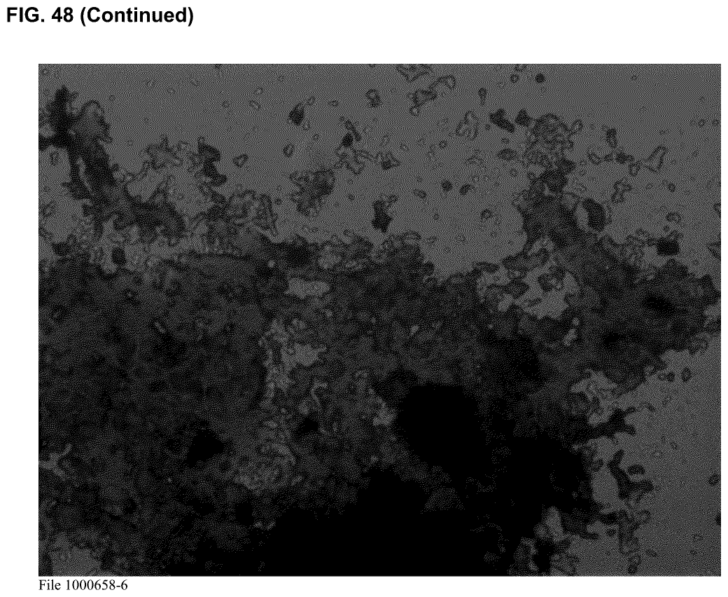

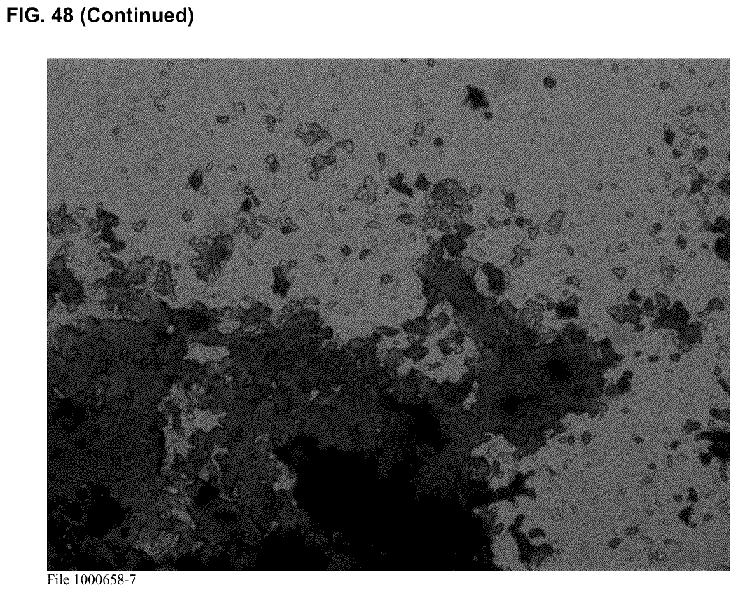

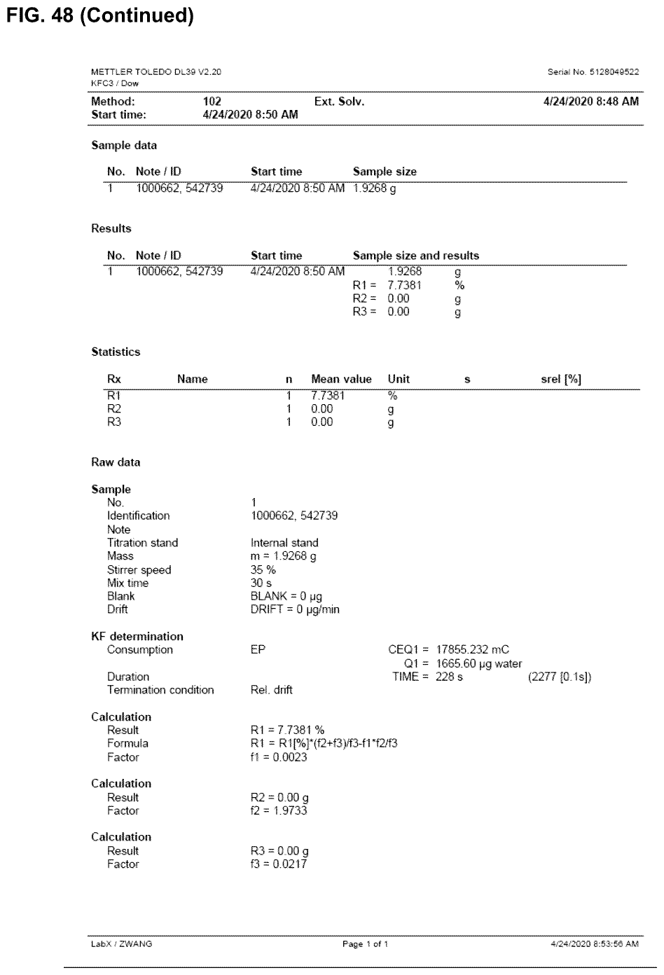

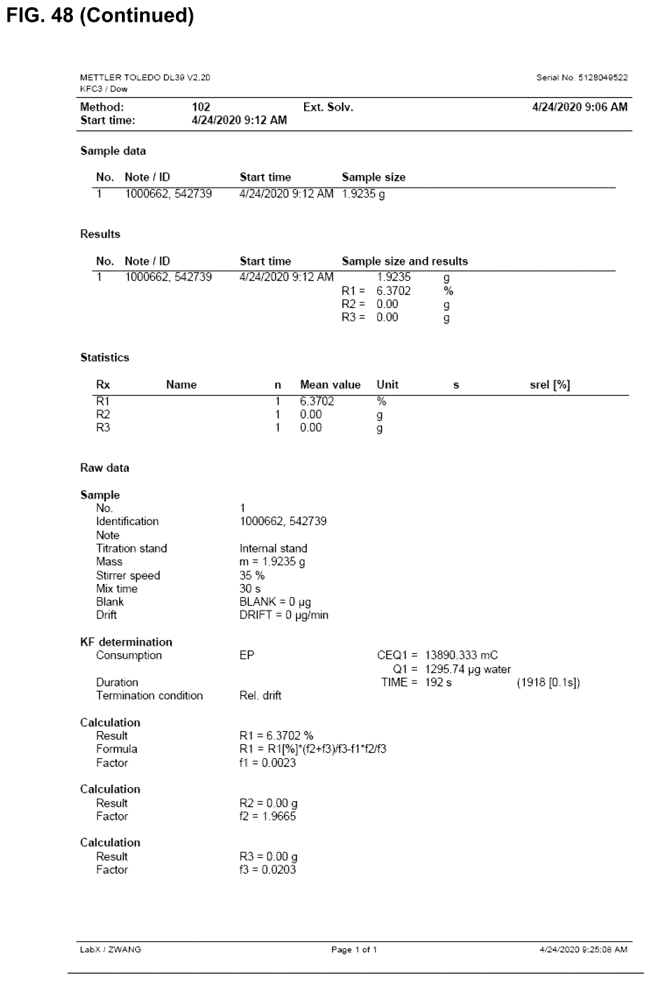

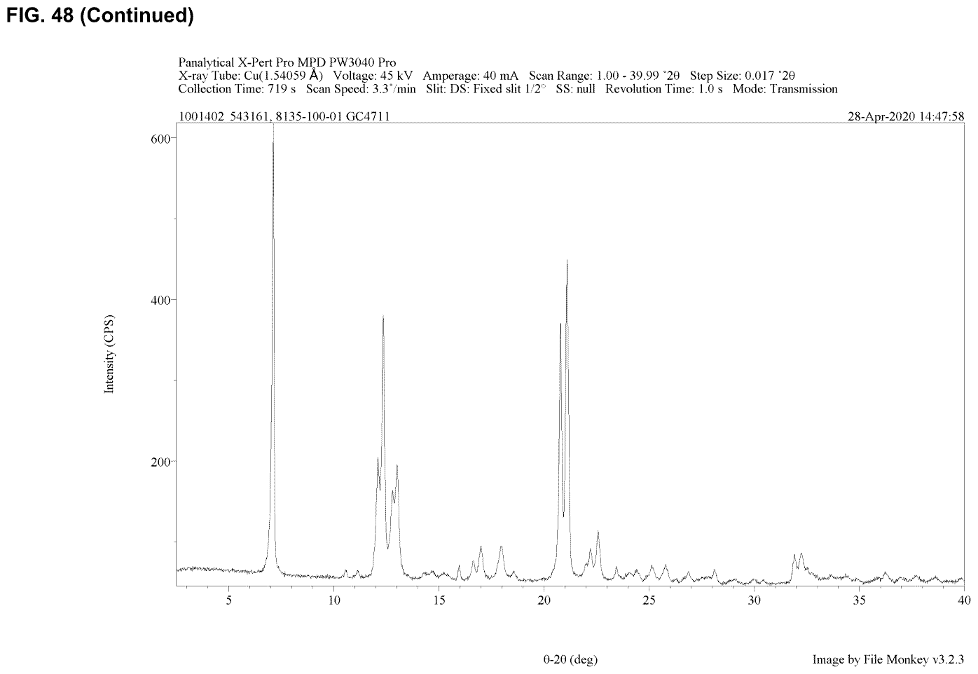

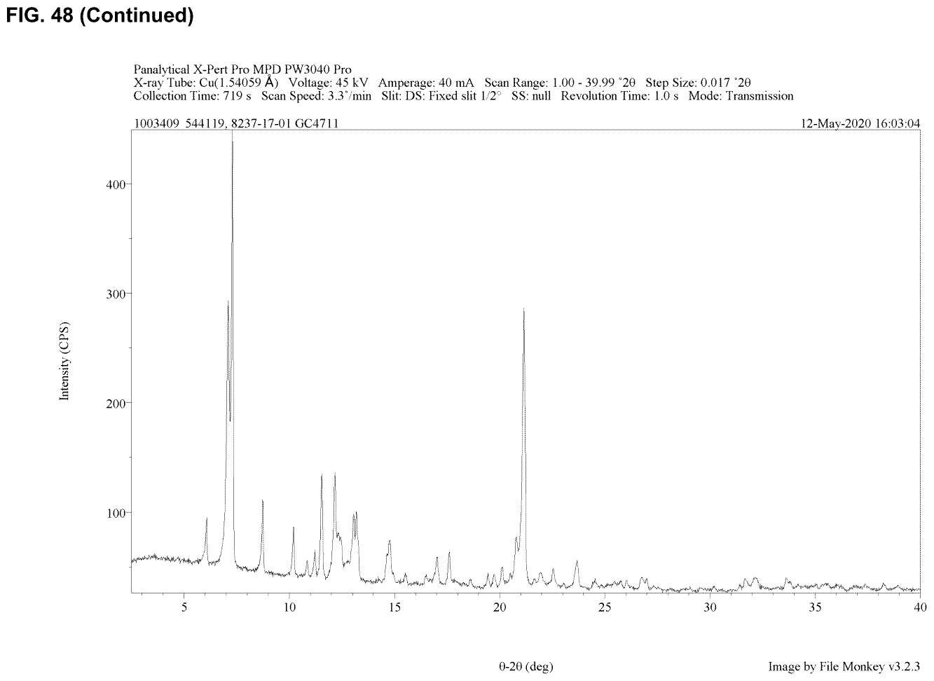

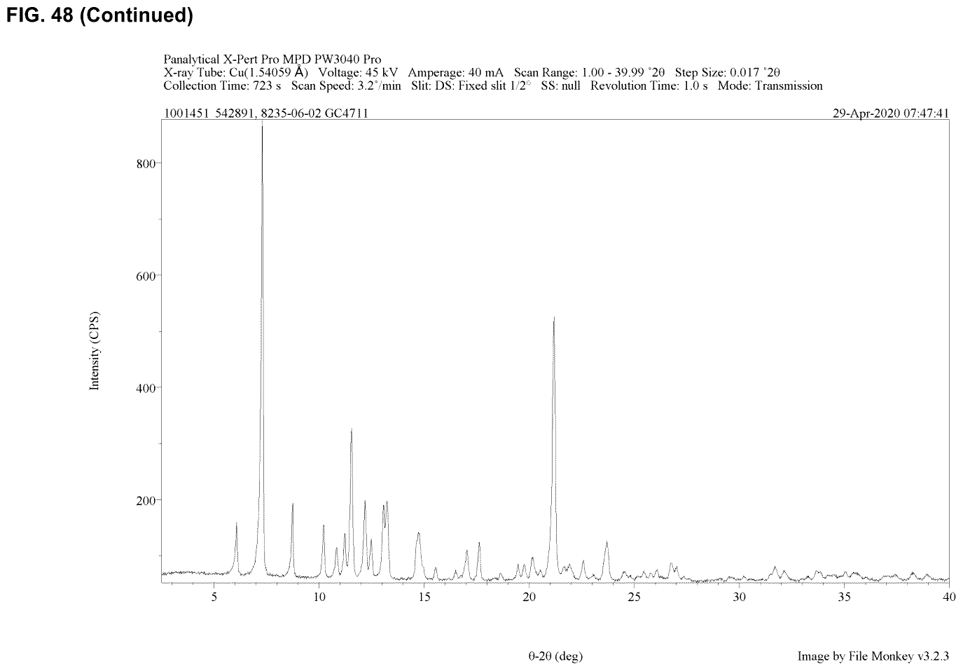

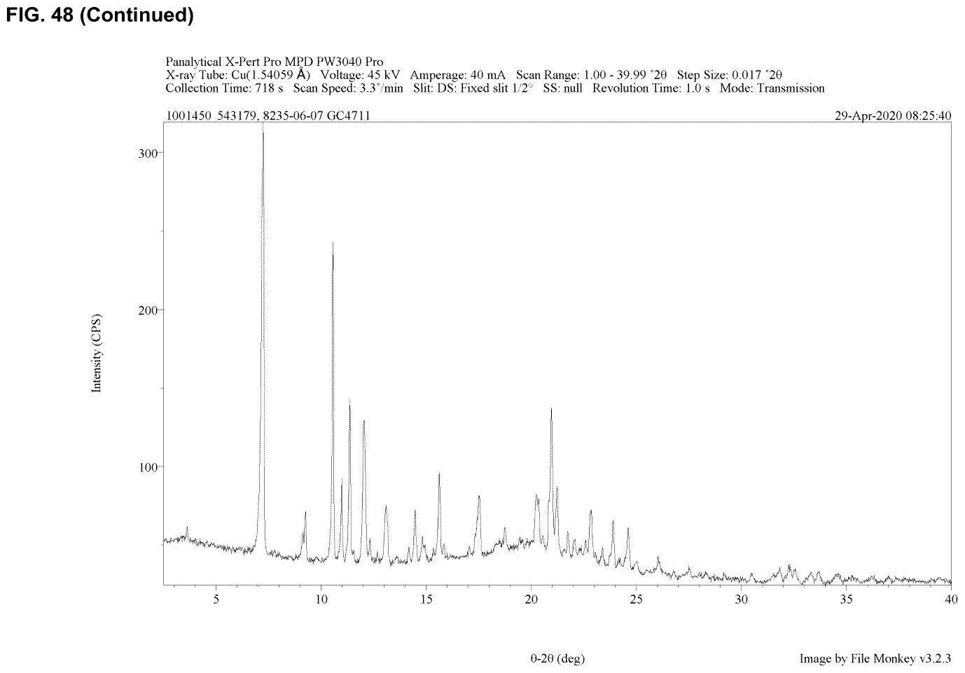

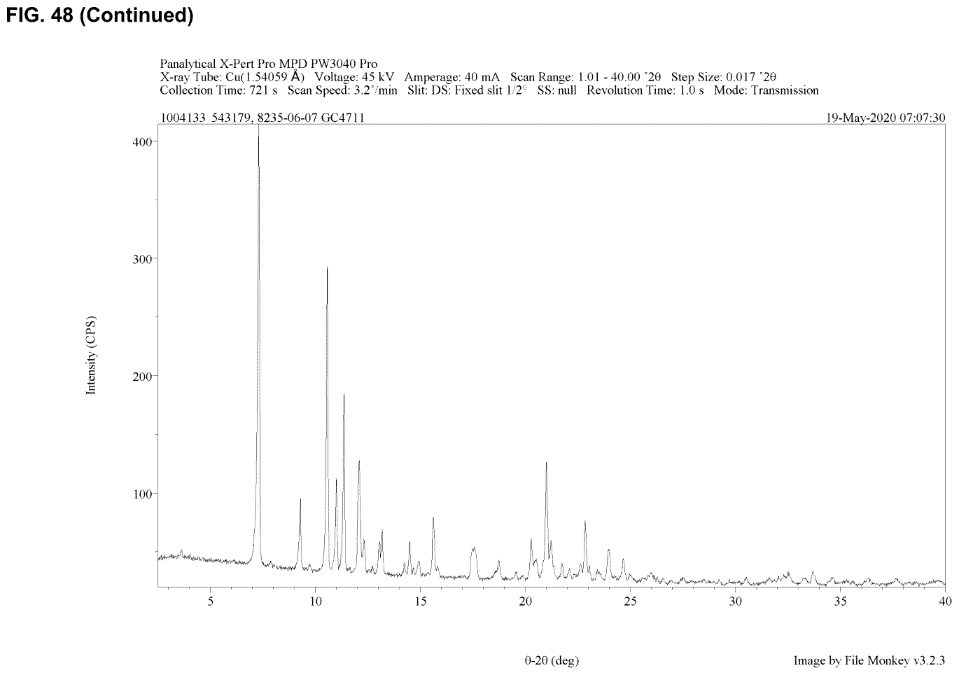

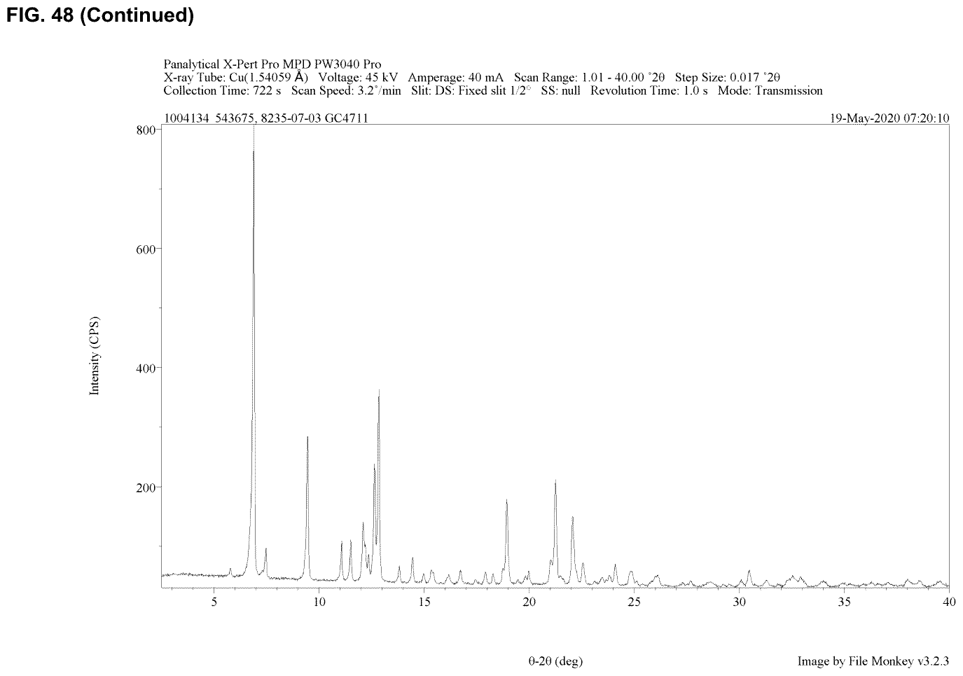

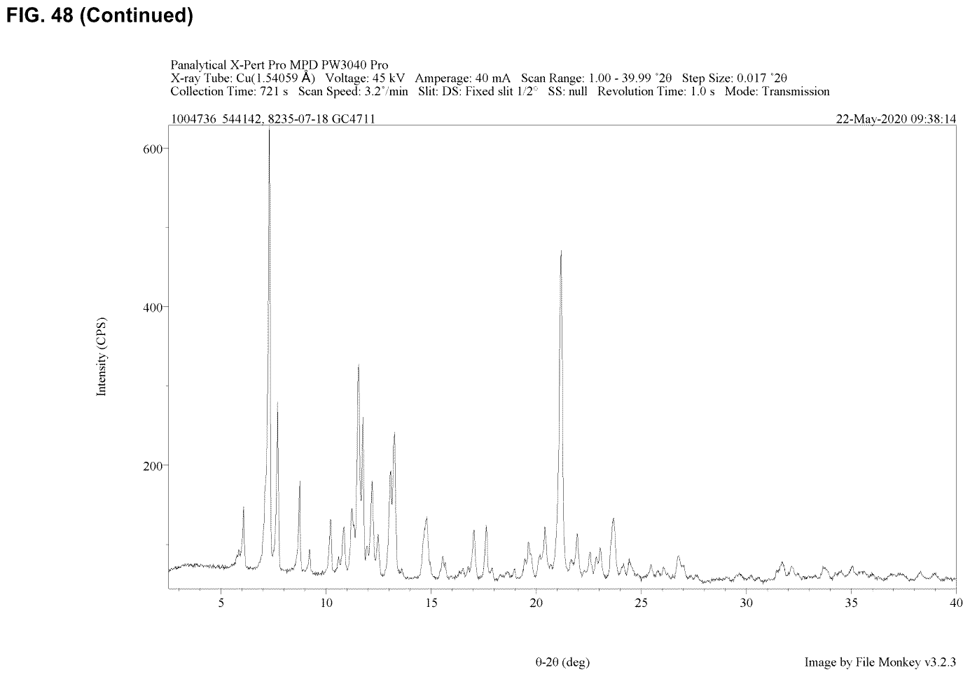

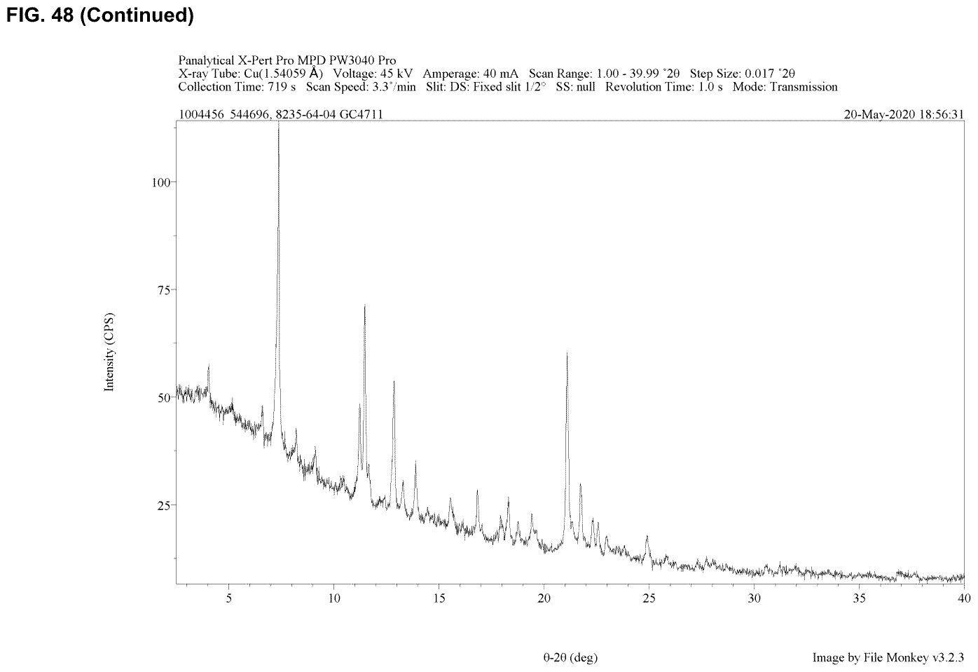

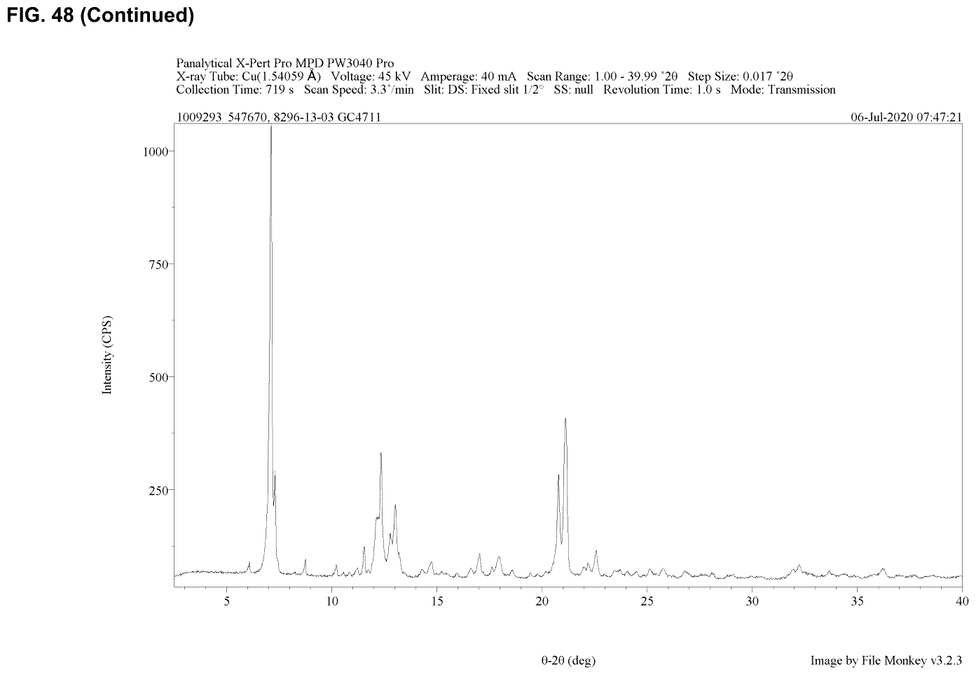

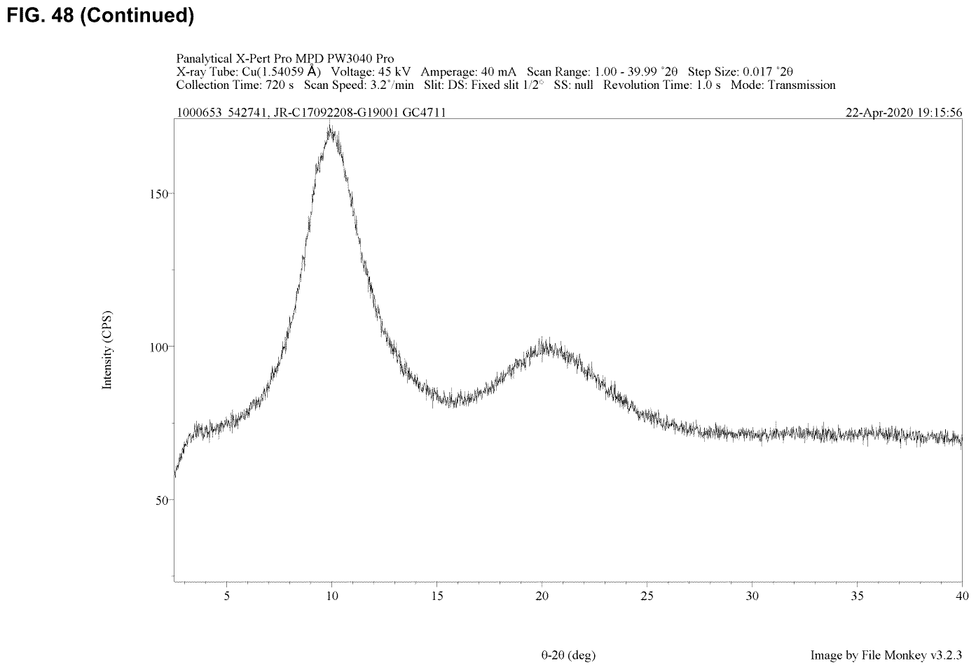

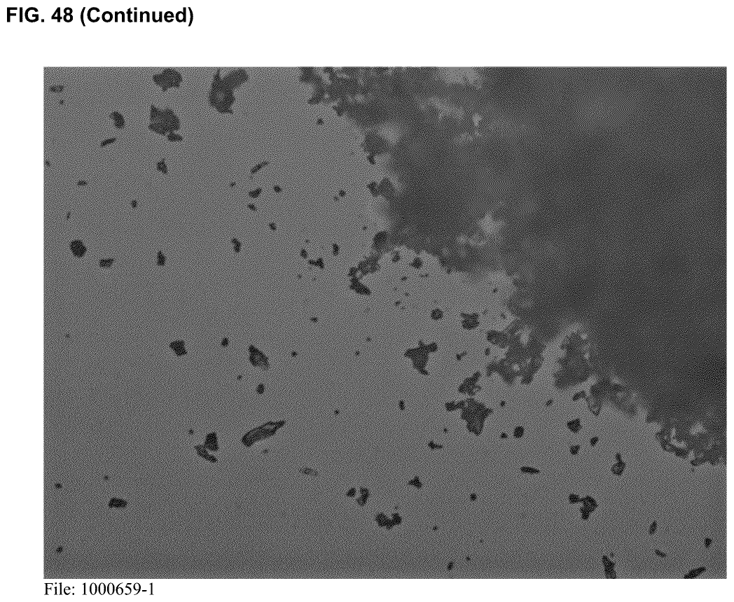

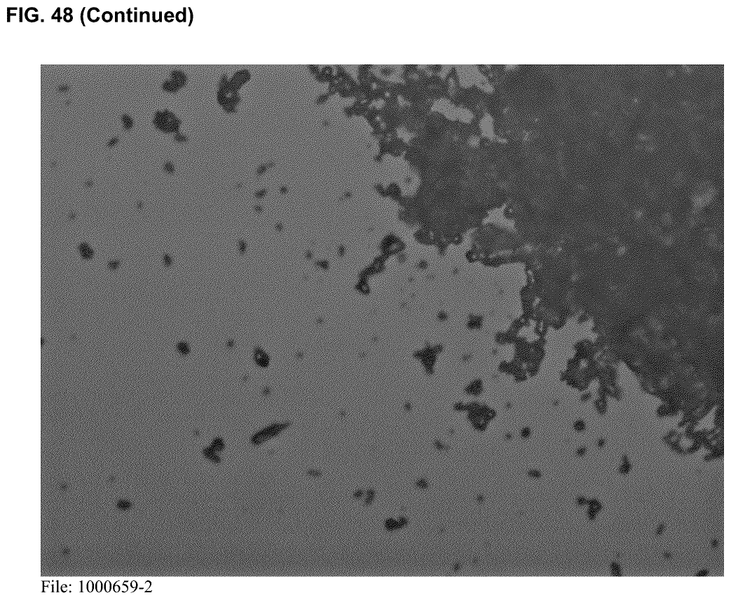

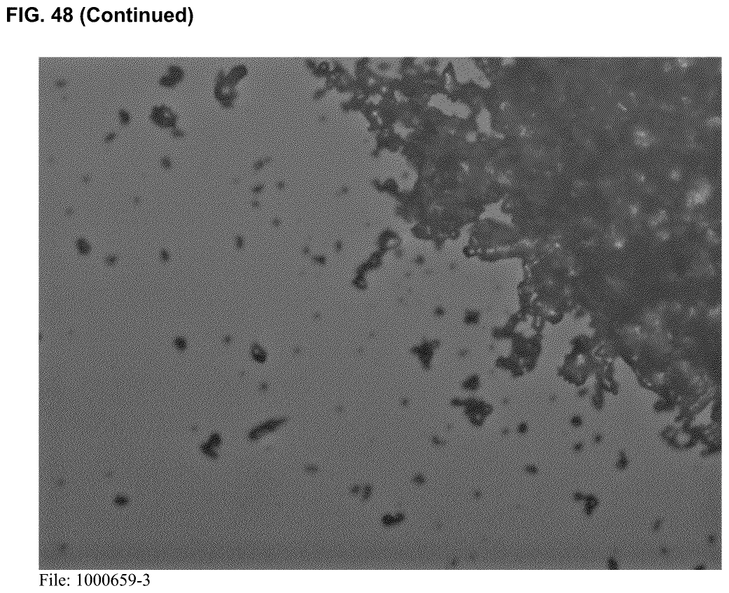

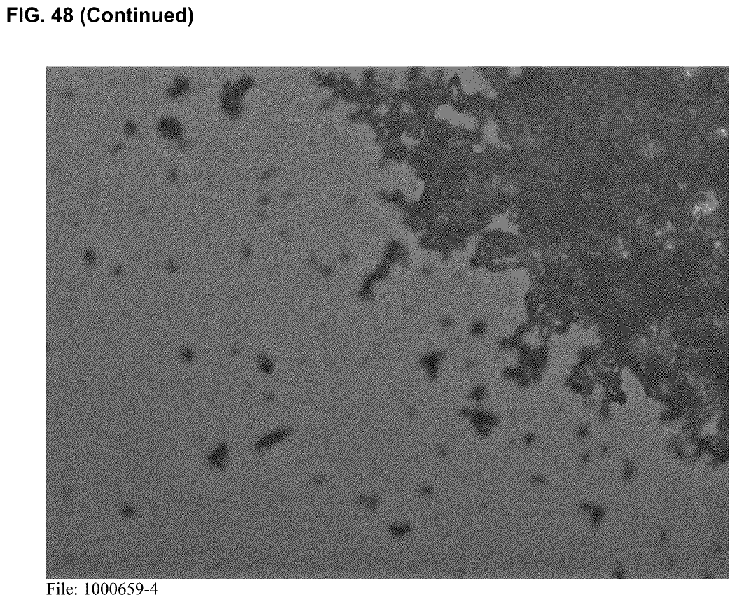

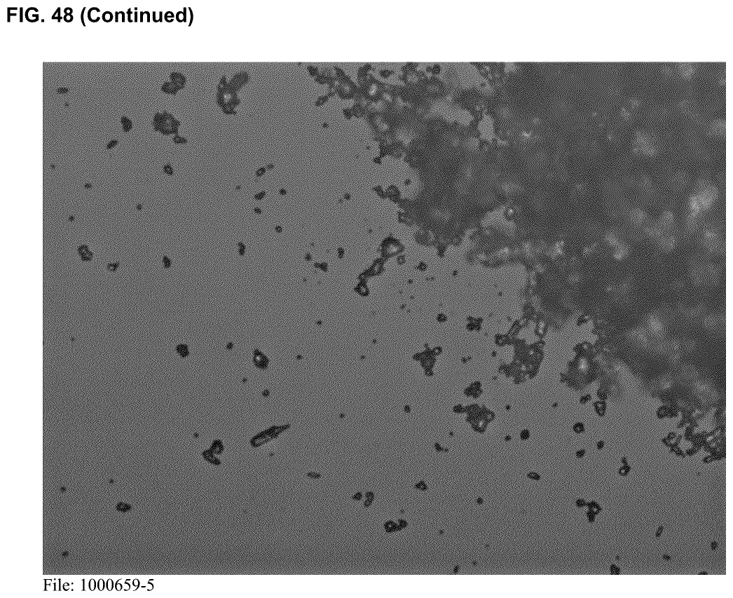

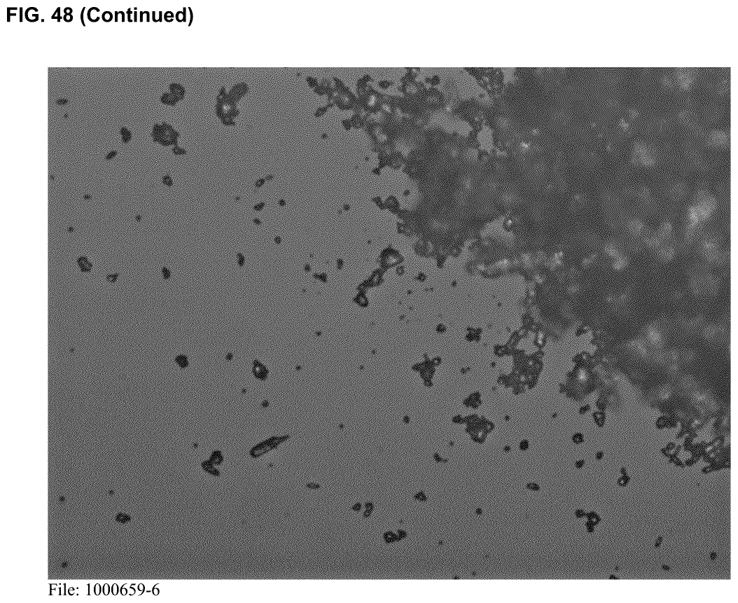

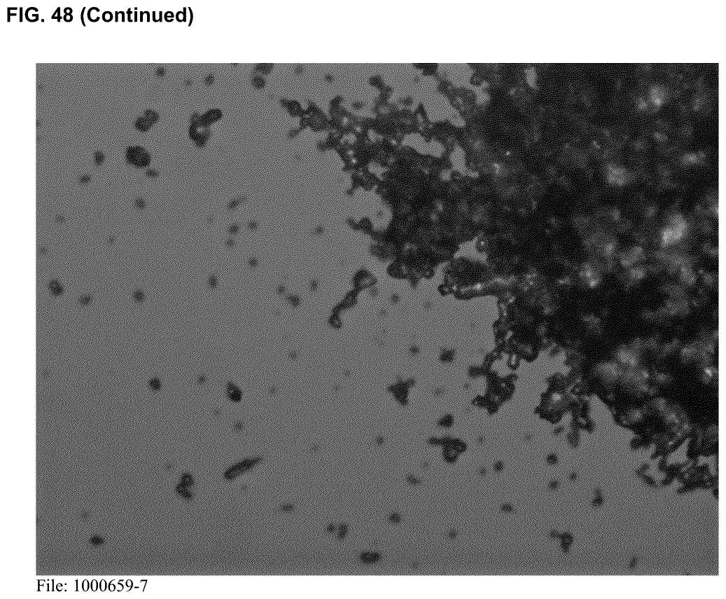

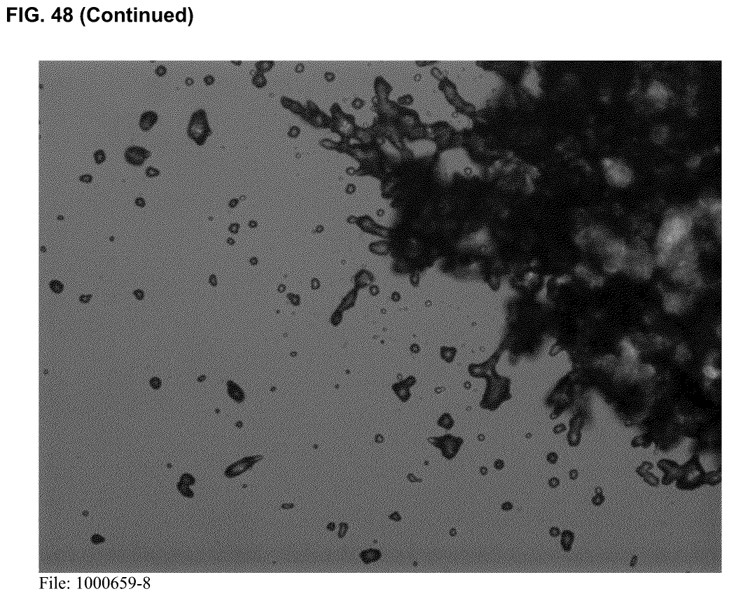

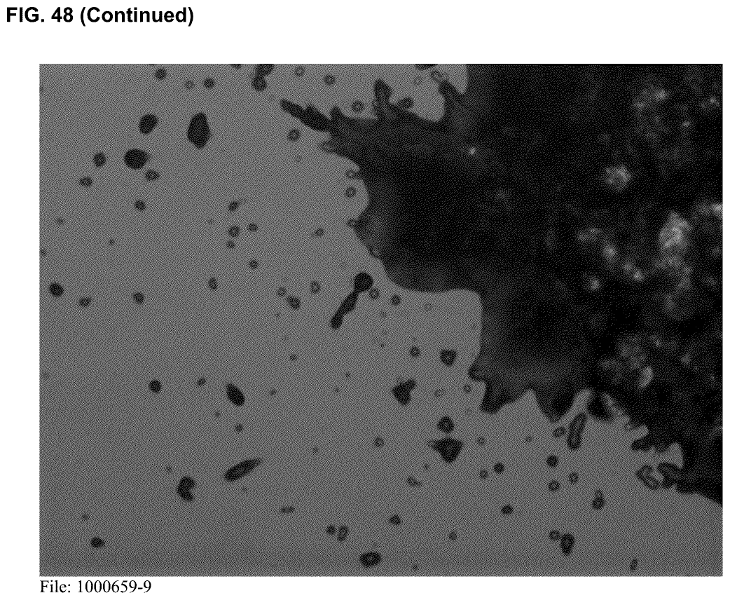

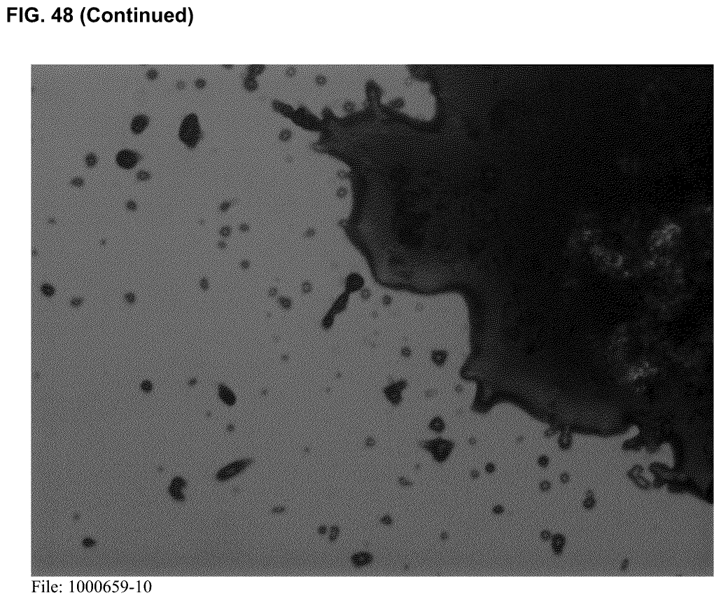

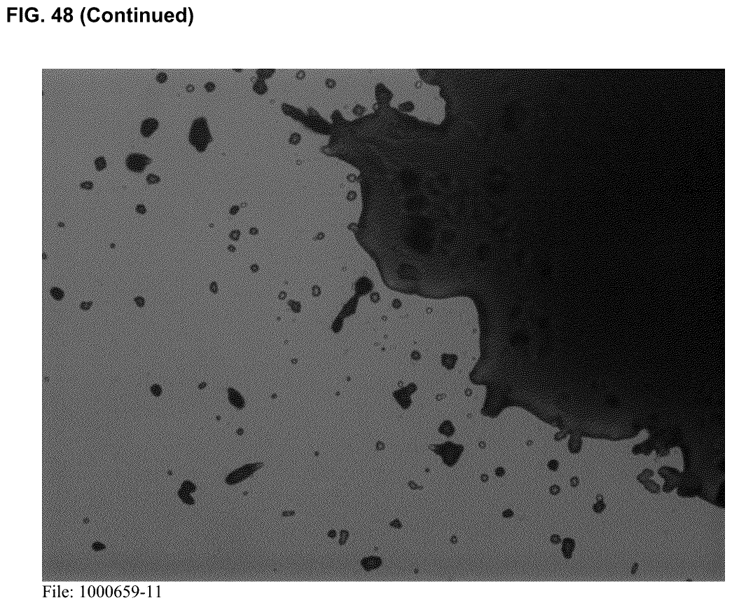

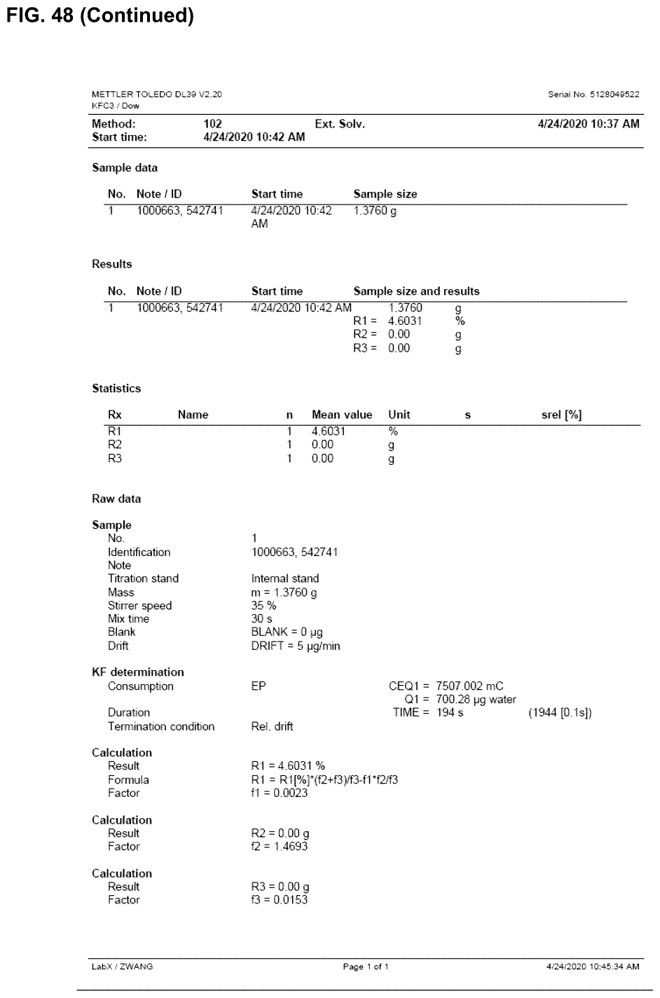

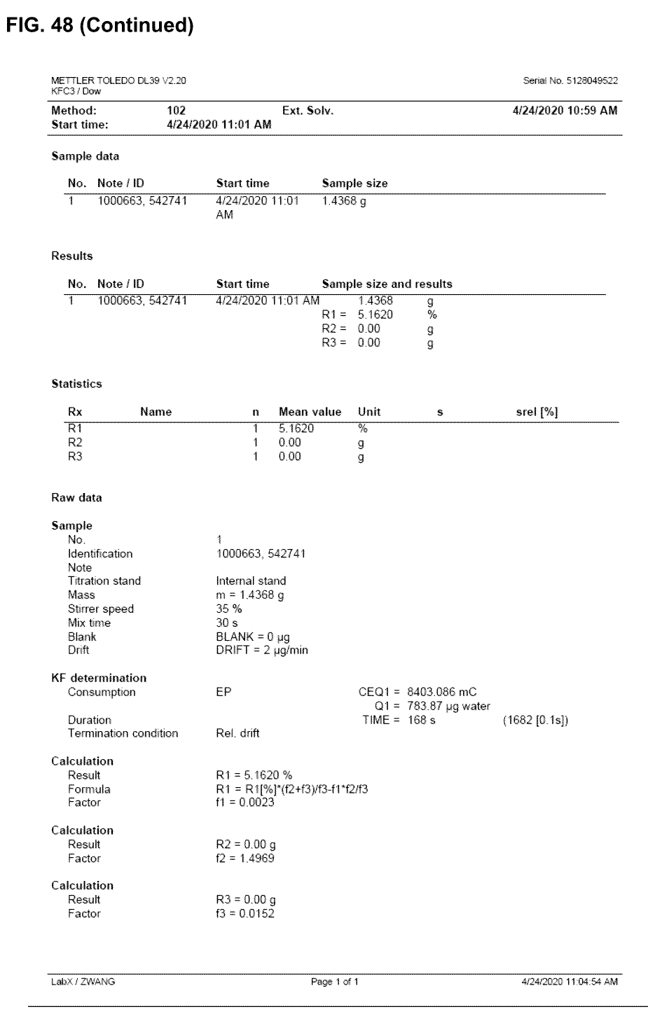

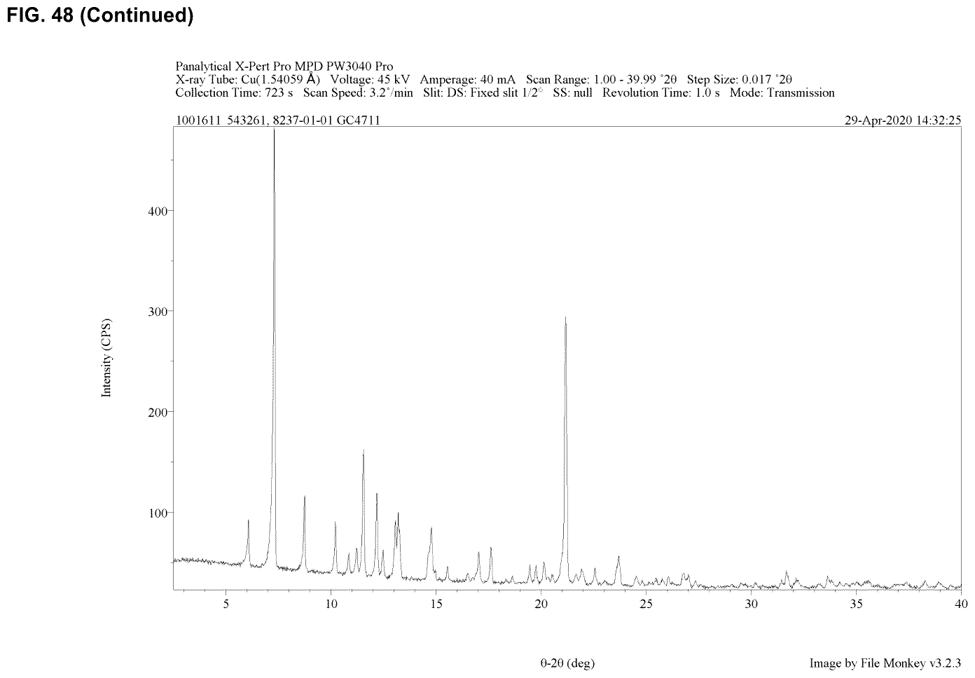

Thermal analyses of the materials from the larger scale attempts are provided in FIGS. 46 and 47. No significant weight loss is observed by thermal gravimetric analysis (TGA), as expected. However, the DSC thermograms exhibit two endotherms. The smaller endotherm near 197° C. is not characteristic for Form E. The second, larger endotherm with an onset of approximately 233° C. is expected.

The materials from the larger scale experiments were triturated a second time under nitrogen in attempts to remove the phase impurity. However, these attempts were unsuccessful.

### Conclusions

GC4711 Form E could not be generated as a pure crystalline phase in sufficient quantity within this study. Attempts conducted at ˜1-g scale provided material composed primarily of Form E and a minor phase impurity, not attributed to known forms of GC4711, detected by both XRPD and DSC. Approximately 550 mg of this mixture was provided as sample 8429-73-01.

### Example 6

An example of a method of preparing the crystalline Form E (anhydrate) of GC4711, as described herein, is provided. According to some embodiments, the Form E crystalline of GC4711, and/or other forms of GC4711 described herein, are prepared with a solvent system having a water activity (a_{w}) of less than 0.11, such as about 0. According to some embodiments, the Form E crystalline of GC4711, and/or other forms of GC4711 described herein, are prepared under conditions of less than 11% relative humidity (RH) at room temperature, such as about 0% RH at room temperature. According to certain embodiments, the Form E crystalline of GC4711, and/or other forms of GC4711 described herein, are prepared with solvent system comprising a single solvent, such as for example acetone. According to yet another embodiment, the Form E crystalline of GC4711 and/or other forms of GC4711 described herein, are prepared with an anhydrous solvent system. According to yet another embodiment, the Form E crystalline of GC4711 and/or other forms of GC4711 described herein, are prepared with a solvent system comprising a tri-solvent system of tetrahydrofuran, 2-methyl tetrahydrofuran, and heptane.

According to one embodiment of a method of preparing the Form E crystalline of GC4711 described herein, a crude solution of GC4711 was recrystallized using a combination of tetrahydrofuran, 2-methyl tetrahydrofuran, and heptane. 2-methyl tetrahydrofuran was charged to the crude solution under nitrogen atmosphere, after which THF was added, and the mixture stirred for 30 minutes to 2 hours at a temperature in the range of 20-28° C. while maintained under nitrogen. Heptane was charged to the mixture, and stirring was continued for 9-14 hours at a temperature in the range of 20-28° C. under nitrogen atmosphere. The resulting crystals were filtered.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

### Example 7

### Sample Preparation

An exemplary method for the synthesis of GC4711 is as follows. As a first part of the synthetic route, the precursor to GC4711, namely GC4419, is prepared. GC4419 has the same stereochemistry and contains all of the same structural elements as GC4711, with the exception that the chloro axial ligands of GC4419 are replaced with the propionato ligands of GC4711. The method of preparing GC4711 from GC4419 is described in U.S. Pat. No. 9,738,670, issued on Aug. 22, 2017 (Example 9), which is hereby incorporated by reference herein in its entirety. Structures for GC4711 and GC4419 are shown below:

### GC4711

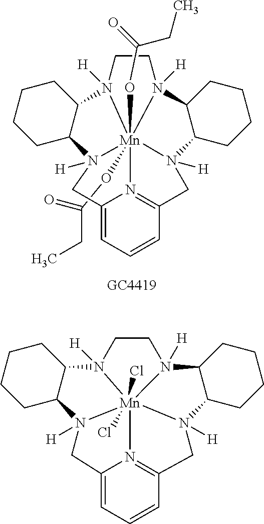

GC4419 is the mirror stereoisomer of GC4403 (depicted in the synthetic scheme below), and thus GC4419 can be prepared using substantially the same synthesis shown below for GC4403, but substituting the mirror image tetraamine starting material (e.g., mirror image of M40400 depicted below), to arrive at the mirror image stereoisomer of GC4403.

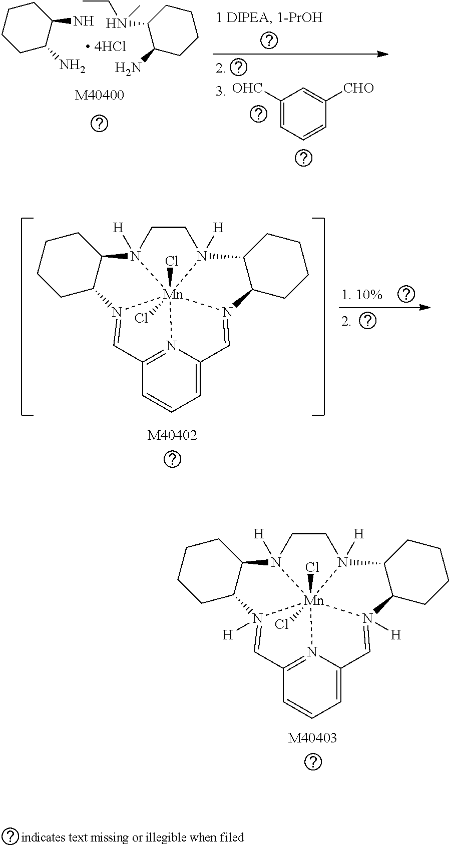

An exemplary synthesis of GC4403 (and thus an exemplary synthesis of GC4419 using the mirror image of M40400 as a starting material) is provided as follows.

Chemicals, materials and methods: Ultra-dry manganese (II) chloride (99.99%, 42844) and palladium on carbon (10% Pd content, standard, reduced, nominally 50% H₂O content, 38304) were purchased from Alfa-Æsar. N,N″-Diisopropylethylamine (DIPEA, re-distilled, 99.5%, 38,764-9), 1-propanol (99.5+%, HPLC grade, 29,328-8) and filter agent, Celpure® P65 (USP-NF, pharmaceutical grade, 52,523-5) were purchased from Aldrich Chemical Co. 2,6-Pyridinedicarboxaldehyde (2,6-PDCA) was manufactured by ABC Laboratories, Columbia, Missouri. Tetraamine tetrahydrochioride M40400 was manufactured by either of CarboGen Laboratories AG, Aarau, Switzerland, Gateway Chemical Technology, St. Louis, Missouri or ABC Laboratories, Columbia, Missouri. 2-Propanol (99.9%, HPLC grade, A451-4), as well as all other solvents (HPLC-grade unless otherwise indicated) and reagents were purchased from VWR Scientific Products or Fisher Scientific and were of the finest grade available. “Reduced pressure” refers to operations carried out using a rotary evaporator and vacuum provided by a circulating water pump. “In vacuo” refers to high vacuum operations (≤0.5 torr), achieved with the use of an efficient, high capacity vacuum pump and an on-line dry-ice/2-propanol trap (capable of maintaining a temperature of ca. −40° C.). Elemental analysis was performed by Desert Analytics in Tucson, AZ (Mn and Na) or by Atlantic Microlab in Norcross, GA (all others). Reaction progress and product homogeneity was determined by HPLC analysis using a Varian ProStar system coupled to a Waters Symmetry-Shield™ RPS-18, 5 μm (4.6×250 mm) column. Solvent systems A and B consisted of 0.5 M LiCl/0.125 M TBAC in water and 1:4 (v/v) water: acetonitrile (CH₃CN), respectively. Elution was accomplished over a 20-min period using a 95:5 (v/v) A: B solvent mixture, run isocratically at a flow rate of 1.0 mL/min and UV detection at 265 nm. Samples for HPLC analysis were diluted/dissolved using mobile phase to afford typical analyte concentrations ca. 1 mg/mL and ca. 20 μL of this solution was injected.

### Cyclization Stage

An exemplary three-component template cyclization stage is described. Tetraamine hydrochloride M40400 (15.06 g, 37.6 mmol) was suspended in 1-propanol (110 mL, rendered a ca. 0.35 M mixture in M40400), thoroughly blanketed with Ar for 15 minutes, and stirred using a 19×35 mm Teflon blade (overhead stirrer, 700 rpm). DIPEA (26.2 mL, 4 equiv, 150.4 mmol) was added in three portions as a stream to the white suspension, which in less than a minute turned into a nearly colorless light syrup. After 10 min, MnCl₂ (4.78 g, 1 equiv, 38.0 mmol) was added in one portion. Thirty minutes after MnCl₂ addition, a nearly clear faint yellow solution had resulted (depending on the scale and efficiency of Ar purging the color may vary) and 2,6-pyridinedicarboxaldehyde (5.08 g, 1 equiv, 37.6 mmol) was added in one portion. The solution turned yellow-orange and heating commenced immediately, reaching 95° C. within 20 min. One hour later, 95% product conversion was detected by HPLC. Following a total of 4 h at 95+2° C., the brownish solution was cooled to near rt over 30 min. HPLC showed ca. 97% M40402 and the crude mixture was used as such immediately.

### Reduction Stage

An exemplary catalytic hydrogenation of bisimine M40402 is described. Once cool (ca. 30° C.), the mixture resulting from the cyclization stage described above was transferred to a Parr stainless steel reactor vessel for catalytic hydrogenation. The dark mixture was blanketed with Ar for a 2-3 minutes, then 10% Pd/C (3.0 g total, 50% water wet, 10 wt % dry catalyst w/respect to M40400) was carefully added. The reactor was assembled and purged of air by pressurizing/depressurizing with N₂ (0 to 150 to 0 psig) five times. Next, the procedure was repeated three times using H₂ (0 to 150 to 0 psig), and finally the reactor was charged with H₂ (150 psig). The suspension was stirred (700 rpm) and heating commenced immediately. The set temperature, 85° C., was reached within 15 min. The reaction was stirred under pressure overnight for a total of 18 h. At this time, HPLC showed the reaction stream contained ca. 99% M40403. After bringing to rt over 1 h and purging with N₂ (5× as before), the reactor was disassembled and the suspension filtered through a bed of 1-propanol-washed celite (10 g) using a 25-50μ fritted funnel. The catalyst/celite bed was washed with 1-propanol (2×20 mL). The yellow solution was evaporated under reduced pressure until an opaque light yellow semi-solid remained (water bath temperature ≤35° C.). This material was stirred in water (700 mL, total solution volume ca. 800 mL) until dissolved (ca. 10 min). The pH of the faint yellow solution was measured as 5.82 and adjusted to 7.78 using 10% aq. NaOH (ca. 0.5 mL). NaCl (210 g, yields a ca. 25% solution in NaCl) was added to the slightly hazy light yellow solution (total volume after NaCl addition ca. 900 mL). After stirring for 20 minutes, the off-white suspension was filtered using a 25-50u fritted funnel. The cake was sucked dry under reduced pressure for approximately 5 minutes (at this time, no more foam dripped), transferred to a beaker, stirred (700 rpm using a 0.25×1 inch magnetic bar) in 20% aqueous NaCl (75 mL) for 15 min, and filtered as above. This washing/filtering procedure was repeated twice more in exactly the same manner. After the second wash, the left over off-white wet material was dried in vacuo (40° C., 17 h, 0.1 torr, cooled to rt over 1 h). Crude M40403 was isolated (19.06 g, 105% mass yield from M40400) as an off-white solid with a purity >99% by HPLC (This crude product contains ca. 5% NaCl and a water content of 5.8% corresponding to a 1.5 hydrated species). Once cool, the solid was broken down to a free-flowing powder prior to 2-propanol extraction. The solid was magnetically stirred (0.25×2 in bar, 800 rpm) in 200 mL of 2-propanol for 30 min and filtered through a 0.5-in bed of celite (pre-washed with 2-propanol immediately before filtration to avoid any possible water absorption). Water (100 mL) was added to the clear light-yellow filtrate and the mixture was briefly swirled to homogenize. Solvents were then removed under reduced pressure (water bath temperature ≤35° C.)⁸ to afford an off-white solid that was further dried in vacuo (40° C., 18 h, 0.1 torr). M40403 was isolated as an off-white solid (16.63 g, 91% yield from M40400) with a purity >99% by HPLC. The material was broken down to a powder and stored in a freezer at 2-8° C.

### Embodiments

Embodiments of the invention herein include, but are not limited to, the following:

Embodiment 1: Crystalline forms of GC4711 as characterized by any of the figures and/or tables herein.

Embodiment 2: Crystalline forms of GC4711 as prepared by any process described herein.

Embodiment 3: Crystalline forms of GC4711 according to any of Embodiments 1 and 2, wherein the crystalline forms are prepared with a solvent system having a water activity (a_{w}) of less than 0.11.

Embodiment 4: Crystalline forms of GC4711 according to any of Embodiments 1 through 3, wherein the crystalline forms are prepared with a solvent system having a water activity (a_{w}) of about 0.

Embodiment 5: Crystalline forms of GC4711 according to any of Embodiments 1 and 2, wherein the crystalline forms are prepared under conditions of less than 11% relative humidity (RH) at room temperature.

Embodiment 6: Crystalline forms of GC4711 according to any of Embodiments 1 and 2, wherein the crystalline forms are prepared under conditions of about 0% relative humidity (RH) at room temperature.

Embodiment 7: Crystalline forms of GC4711 according to any preceding Embodiment, wherein the crystalline forms are prepared with a solvent system comprising a single solvent.

Embodiment 8: Crystalline forms of GC4711 according to any of Embodiments 1-6, wherein the crystalline forms are prepared with a solvent system comprising a tri-solvent system of tetrahydrofuran, 2-methyl tetrahydrofuran, and heptane.

Embodiment 9: Crystalline forms of GC4711 as characterized by any of the data shown or described herein.

Embodiment 10: A Form E anhydrate crystalline form of GC4711 according to any of Embodiments 1-9.

### REFERENCES

1. Greenspan, Lewis. Humidity Fixed Points of Binary Saturated Aqueous Solutions, Journal of Research, 1977, 81A, 89-96.
2. Choy, B.; Reible, D. (1996). UNIFAC Activity Coefficient Calculator (Version 3.0) [Software]. University of Sydney, Australia and Louisiana State University, USA.
3. Macrae, C. F. Edgington, P. R. McCabe, P. Pidcock, E. Shields, G. P. Taylor, R. Towler M. and van de Streek, J. J. Appl. Cryst., 2006, 39, 453-457.
4. Glusker, Jenny Pickworth; Trueblood, Kenneth N. Crystal Structure Analysis: A Primer, 2nd ed.; Oxford University press: New York, 1985; p. 87.
5. CrysAlisPro 1.171.38.41r (Rigaku Oxford Diffraction, 2015).
6. Sheldrick, G. M. Acta Cryst., 2015, A71, 3-8.
7. Sheldrick, G. M. Acta Cryst., 2008, A64, 112-122.
8. International Tables for Crystallography, Vol. C, Kluwer Academic Publishers: Dordrecht, The Netherlands, 1992, Tables 4.2.6.8 and 6.1.1.4.
9. McClurg, Richard B.; Smit, Jared P. X-ray Powder Diffraction Pattern Indexing for Pharmaceutical Applications. Pharm. Tech. Europe, January 2013.
10. X'Pert High Score Plus 2.2a (2.2.1).
11. Hygroscopicity terms and definitions are based in part on concepts presented in the following: Newman, A. W.; Reutzel-Edens, S. M.; Zografi, G. Characterization of the “Hygroscopic” Properties of Active Pharmaceutical Ingredients, J. Pharm. Sci. 2008, 97, 1047-1059.
12. Hofmann, D W M. Fast Estimation of Crystal Densities. Acta. Cryst. 2002; B57(3-2): 489-93.
13. Ticehurst M D, Storey R A, Claire W. Application of slurry bridging experiments at controlled water activities to predict the solid-state conversion between anhydrous and hydrated forms using theophylline as a model drug. Int J Pharm. 2002; 247:1-10.
14. Sacchetti M. Determining the relative physical stability of anhydrous and hydrous crystal forms of GW2016. Int J Pharm. 2004; 273:195-202.
15. Zhu H, Yuen C, Grant D J W. Influence of water activity in organic solvent+water mixtures on the nature of the crystallizing drug phase. 1. Theophylline. Int J Pharm. 1996; 135:151-160.
16. Zhu H, Grant D J W. Influence of water activity in organic solvent+water mixtures on the nature of the crystallizing drug phase. 2. Ampicillin. Int J Pharm. 1996; 139:33-43.
17. International Tables for Crystallography, Vol. C, Kluwer Academic Publishers: Dordrecht, The Netherlands, 1992, Tables 4.2.6.8 and 6.1.1.4.

What is claimed is:

1. A Form E anhydrate crystalline form of GC4711 according to the following formula: 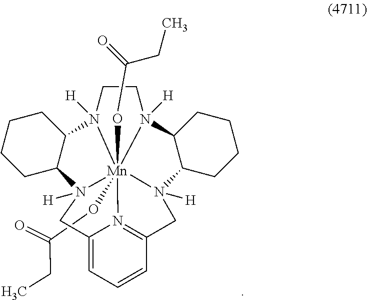
having one or more characteristic XRPD (X-Ray Powder Diffraction) peaks as shown in FIG. 10.

2. A pharmaceutical composition comprising the Form E anhydrate crystalline form of GC4711 according to claim 1.

3. A method of treating cancer comprising administering a composition of claim 2 and one or more pharmaceutically acceptable excipients to a patient in need thereof.

4. A method of treating an inflammatory condition comprising administering a composition of claim 2 and one or more pharmaceutically acceptable excipients to a patient in need thereof.

5. The method of claim 4, wherein the inflammatory condition is oral mucositis.
